(19) United States
(12) Patent Application Publication
(10) Pub. No.: US 2025/0051363 A1
(43) Pub. Date: Feb. 13, 2025

(54) Substituted Pyrazolopyridines

(71) Applicant: Neuro3 Therapeutics, Inc., Cambridge, MA (US)

(72) Inventors: Mark Seefeld, Collegeville, PA (US); Zheng Li, Lyme, CT (US); Xinyan Huang, Paramus, NJ (US)

(21) Appl. No.: 18/793,487

(22) Filed: Aug. 2, 2024

Related U.S. Application Data
(63) Continuation of application No. PCT/US2024/040352, filed on Jul. 31, 2024.
(60) Provisional application No. 63/517,413, filed on Aug. 3, 2023.
(60) Provisional application No. 63/517,413, filed on Aug. 3, 2023.

(51) Int. Cl. C07D 519/00; A61K 31/444; A61K 31/4545; A61K 31/496; A61K 31/4995; A61K 31/5377; C07D 471/04

(52) U.S. Cl. CPC C07D 519/00; A61K 31/444; A61K 31/4545; A61K 31/496; A61K 31/4995; A61K 31/5377; C07D 471/04

(57) ABSTRACT

Disclosed herein are substituted pyrazolopyridines, methods for their preparation, and use thereof.

### CROSS-REFERENCE TO RELATED APPLICATIONS

This application claims priority from U.S. Provisional Application No. 63/517,413, filed Aug. 3, 2023, and this application is a continuation of International Application No. PCT/US2024/040352, filed Jul. 31, 2024, which claims priority from U.S. Provisional Application No. 63/517,413, filed Aug. 3, 2023, the disclosure of each of which is hereby incorporated by reference in its entirety.

### FIELD

This disclosure is directed to substituted pyrozalopyridines, and more particularly to such compounds that inhibit LRRK2 kinase activity and therefore useful in the treatment of diseases or disorders associated with LRRK2 kinase activity. The disclosure is directed to compounds and compositions that inhibit LRRK2 kinase activity, methods of treating a disease or disorder associated with LRRK2 kinase activity (e.g., Parkinson's disease, Alzheimer's disease, multiple sclerosis, HIV-induced dementia, amyotrophic lateral sclerosis, ischemic stroke, traumatic brain injury, and spinal cord injury), and methods of using LRRK2 kinase inhibitors in combination with one or more additional disorder therapy.

### BACKGROUND

Parkinson's disease (PD) is a neurodegenerative disorder charactered by selective degeneration and cell death of dopaminergic neurons in the substantial nigra region of the brain. Globally, the prevalence of PD has doubled in the past 25 years, with a global estimate in 2019 of over 8.5 million individuals living with PD. Disability and death due to PD are increasing faster than for any other neurological disorders.

PD was generally considered to be sporadic and of unknown etiology, but in the past 15 years, there has been an important development of the understanding of the genetic basis of this disease and the pathogenic mechanisms associated therewith. In general, PD is a degenerative condition of the brain associated with motor symptoms (e.g., slow movement, tremor, rigidity, and walking imbalance) and a wide variety of non-motor complications (e.g., cognitive impairment, mental health disorders, pain, and other sensory disturbances). Motor impairments, including involuntary movements (i.e., dyskinesias) and painful involuntary muscle contractions (i.e., dystonias), both contribute to limitations in speech and mobility, and thus restrictions in many areas of life. Progression of these symptoms and complications markedly decrease functioning and quality of life, which result in high rates of disability and care requirements. Pathologically, the disease is identified by the loss of dopaminergic neurons, with a consequent decrease in dopamine levels in the brain and by aggregation of the protein α-synuclein in the dopaminergic neurons. Current PD therapies aim at increasing the dopamine levels in areas innervated by dopaminergic neurons in the brain; however, none address the underlying disease-causing problem. Many alternative approaches to treating PD are therefore under investigation, one of which is inhibition of leucine-rich repeat kinase 2 (LRRK2).

LRRK2 kinase is a 286 kDa cytoplasmic protein containing kinase and guanine nucleotide-binding proteins (GTPase) domains as well as multiple protein-protein interaction domains. In vitro biochemical studies have demonstrated that LRRK2 proteins harboring the PD-associated proteins generally confer increased kinase activity and decreased GTP hydrolysis compared to the wild type protein, suggesting that small molecule inhibitors may be used to inhibit aberrant LRRK2-dependent signaling in PD. In support of this notion, it has been reported that inhibitors of LRRK2 are protective in models of PD.

Approximately 10% of PD cases are attributed to inherited genetic mutations, while the rest are considered idiopathic or sporadic. The G2019S mutation in the LRRK2 protein is the most common pathogenic mutation, accounting for about 1 to 6% of sporadic and about 3 to 19% of familial PD cases. Additionally, common variants in the LRRK2 locus are known risk factors for idiopathic PD (IPD). Indeed, it has been argued that LRRK2 provides a link between genetic and idiopathic forms of PD. Oxidative stress and endolysosomal impairment, both of which have critical roles in PD, activate LRRK2 kinase activity. Similarly, environmental toxicants, implicated epidemiologically in PD risks, also activate LRRK2. Moreover, in vivo blockade of LRRK2 activity is protective in genetic and toxicant models of PD. As noted, early-stage clinical trials of LRRK2-based therapeutics are already underway.

A growing body of evidence suggests a role for LRRK2 in immune cell function in the brain with LRRK2 inhibitors demonstrated to attenuate microglial inflammatory responses. As neuroinflammation is a hallmark of a number of neurodegenerative diseases (e.g., PD, Alzhemimer's disease (AD), multiple sclerosis (MS), HIV-induced dementia, amyotrophic lateral sclerosis (ALS), ischemic stroke, traumatic brain injury, and spinal cord injury), LRRK2 kinase inhibitors may have utility in the treatment of neuroinflammation in these disorders. LRRK2 mutations have been associated with Alzheimer's-like pathology, and the LRRK2 RI628P variant has been associated with an increased risk of developing AD. Mutations in LRRK2 have also been identified that are clinically associated with the transition from mild cognitive impairment to Alzheimer's disease. Together these data suggest that LRRK2 inhibitors may be useful in the treatment of Alzheimer's disease and other dementias and related neurodegenerative disorders.

As such, LRRK2 kinase is a prime target for drug development.

### SUMMARY

In one aspect, the present disclosure provides compounds of Formula (I):

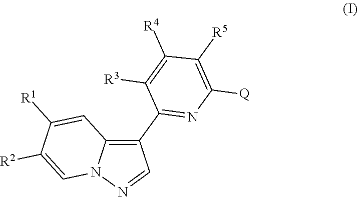

or a pharmaceutically acceptable salt, prodrug, solvate, hydrate, isomer, deuterated form, or tautomer thereof, wherein:
R¹ is hydrogen, C₁-C₆ alkyl, C₃-C₆ cycloalkyl, halogen, halo C₁-C₆ alkyl, —OR⁶, cyano, —NR⁷R⁸, or —NHC(O)R⁹, wherein
   R⁶ is hydrogen, C₁-C₆ alkyl, C₃-C₆ cycloalkyl, C₃-C₆ cycloalkyl C₁-C₆ alkyl, 3-6 membered heterocyclyl, or 3-6 membered heterocyclyl C₁-C₆ alkyl;
   each of R⁷ and R⁸ is independently hydrogen, C₁-C₆ alkyl, or together with the nitrogen to which they are attached form a 3-8 membered monocyclic heterocyclyl group; and
   R⁹ is C₆-C₁₀ aryl, 5- or 6-membered heteroaryl, or C₁-C₆ alkyl, wherein
      the C₆-C₁₀ aryl is optionally substituted with C₁-C₆ alkyl, halogen, halo C₁-C₆ alkyl, —OR^{9A}, cyano, or —NR^{9B}R^{9C}, wherein
         R^{9A} is hydrogen or C₁-C₆ alkyl, and
         each of R^{9B} and R^{9C} is independently hydrogen or C₁-C₆ alkyl;
      the 5- or 6-membered heteraryl comprises one nitrogen atom and is optionally substituted with C₁-C₆ alkyl, halogen, halo C₁-C₆ alkyl, —OR^{9A}, cyano, or —NR^{9B}R^{9C}, and
      the C₁-C₆ alkyl is optionally substituted with halogen, —SO₂CH₃, cyano, 5- or 6-membered heteroaryl, —OR^{9D}, or —NR^{9E}R^{9F}, wherein
         R^{9D} is hydrogen, C₁-C₆ alkyl, or 5- or 6-membered heteroaryl comprising one nitrogen atom and optionally substituted with C₁-C₆ alkyl, halogen, halo C₁-C₆ alkyl, —OR^{9A}, cyano, or —NR^{9B}R^{9C}, and
         each of R^{9E} and R^{9F} is independently hydrogen, C₁-C₆ alkyl, —SO₂CH₃, or 5- or 6-membered heteroaryl comprising one nitrogen atom;
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen; and
Q is a 3-8 membered heterocyclyl group, wherein
   the heterocyclyl group includes at least one nitrogen atom;
   the heterocyclyl group is optionally fused to a non-aromatic ring containing 3-6 ring members of which 1 or 2 are optionally nitrogen, oxygen, or sulfur atoms and the remainder are carbons;
   the heterocyclyl group is optionally substituted with 1-4 R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.

In another aspect, the present disclosure provides for a pharmaceutical composition comprising a compound or salt as otherwise described herein together with a pharmaceutically acceptable carrier, excipient, or diluent.

In another aspect, the present disclosure provides for a method of treating a disease or disorder associated with LRRK2 kinase activity, which comprises administering to a patient in need thereof a therapeutically effective amount of a compound as otherwise described herein or a pharmaceutical composition as otherwise described herein.

In another aspect, the present disclosure provides for a method of inhibiting LRRK2 kinase activity, which comprises administering to a patient in need thereof a therapeutically effective amount of a compound as otherwise described herein or a pharmaceutical composition as otherwise described herein.

In another aspect, the present disclosure provides for a method of treating Parkinson's disease, which comprises administering to a patient in need thereof a therapeutically effective amount of a compound as otherwise described herein or a pharmaceutical composition as otherwise described herein.

In another aspect, the present disclosure provides intermediates as described herein, such intermediates being suitable for use in a process for preparing a compound as described herein.

Other aspects and embodiments of the disclosure are evident in view of the detailed description provided herein.

### DETAILED DESCRIPTION OF THE INVENTION

The present invention relates to LRRK2 kinase inhibitors. In particular, the present invention relates to compounds that inhibit LRRK2 kinase activity, pharmaceutical compositions comprising a therapeutically effective amount of the compounds, and methods of use therefor.

### Definitions

Unless defined otherwise, all technical and scientific terms used herein have the same meaning as is commonly understood by one of skill in the art to which this invention belongs.

All patents, patent applications, and publications referred to herein are incorporated by reference to the extent they are consistent with the present disclosure. Terms and ranges have their generally defined definition unless expressly defined otherwise.

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which a divalent moiety is required and is stated as being “aryl,” those skilled in the art will understand that the term “aryl” refers to the corresponding divalent moiety, arylene. All atoms are understood to have their normal number of valences for bond formation (i.e., 4 for carbon, 3 for N, 2 for O, and 2, 4, or 6 for S, depending on the oxidation state of the S).

The term “amino” refers to —NH₂.

The term “acetyl” refers to —C(O)CH₃.

As herein employed, the term “acyl” refers to an alkylcarbonyl or arylcarbonyl substituent wherein the alkyl and aryl portions are as defined herein.

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁, and C₁₂ groups. Alkyl groups may be branched or unbranched. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁, and C₁₂ groups. Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁, and C₁₂ groups. Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Examples of alkenylene groups include, without limitation, ethenylene, propenylene, and butenylene. Examples of alkynylene groups include, without limitation, ethynylene, propynylene, and butynylene.

The term “alkoxy” refers to —O(C₁-C₆ alkyl).

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁, and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “C₃-C₆ cycloalkyloxy” refers to groups of the formula —O(C₃-C₆ cycloalkyl).

The term “heteroalkyl” refers to an alkyl group, as defined hereinabove, wherein one or more carbon atoms in the chain are independently replaced O, S, or NR^{x}, wherein R^{x} is hydrogen or C₁-C₃ alkyl. Examples of heteroalkyl groups include methoxymethyl, methoxyethyl, and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. A representative aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl group covalently linked to an alkyl group wherein the moiety is linked to another group via the alkyl moiety. An representative aralkyl group is —(C₁-C₆)alkyl(C₆-C₁)aryl, including, without limitation, benzyl, phenethyl, and naphthylmethyl. For example, an arC₁-C₃alkyl is an aryl group covalently linked to a C₁-C₃ alkyl.

As used herein, the term “fused” when used to define rings, e.g., bicyclic fused ring systems, refers to bicyclic, tricyclic, etc. ring systems sharing 2 or more atoms, including bridged systems. Examples of such fused ring systems are (1S,4R)-2-azabicyclo[2.2.1]heptane; 2-azabicyclo[2.2.2]octane; 2,5-diazabicyclo[2.2.2]octane; 2-oxa-5-azabicyclo[2.2.2]octane; isoindoline; 1,2,3,4-tetrahydro-2,6-naphthyridine; 1,2,3,4-tetrahydroisoquinoline; 1,2,3,4-tetrahydro-1,4-(epiminomethano)naphthalene; and 3-azabicyclo[3.1.0]hexane.

A “heterocyclyl” or “heterocyclic” or “heterocycloalkyl” group is a mono- or bicyclic (e.g., fused) ring structure having from 3 to 12 atoms, (3, 4, 5, 6, 7, 8, 9, 10, 11 or 12 atoms), for example 4 to 8 atoms, wherein one or more ring atoms are independently N, O, or S, and the remainder of the ring atoms are quaternary or carbonyl carbons. Examples of heterocyclic groups include, without limitation, epoxy, oxiranyl, oxetanyl, azetidinyl, aziridinyl, tetrahydrofuranyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl. Specifically excluded from the scope of this term are compounds having adjacent ring O and/or S atoms. The heterocyclic groups can be attached to a parent group (i.e., the point of attachment) via any ring atom, including one of the heteroatoms or one of the carbon atoms, in the heterocyclic ring group. As chemically required, the heterocyclic ring may be attached to one or more other groups, for instance if operating as a bridging group. The term “heterocyclyl” also includes fused multicyclic (e.g., bicyclic) ring systems in which one or more of the fused rings is aromatic or non-aromatic, provided that at least one ring is non-aromatic contains an N, O, or S ring atom. Examples of such fused multicyclic ring systems are indolinyl, indolin-2-yl, 2,3-dihydrobenzofuran-2-yl, and 2,3,4,5-tetrahydrobenzo[d]oxazol-2-yl. Each of these examples is a 9-membered heterocyclyl.

As used herein, the term “heteroaryl” refers to a group having 5 to 14 ring atoms, preferably 5, 6, 10, 13, or 14 ring atoms; having 6, 10, or 14 π electrons shared in a cyclic array; and having, in addition to carbon atoms, from one to three heteroatoms that are each independently N, O, or S. “Heteroaryl” also includes fused multicyclic (e.g., bicyclic) ring systems in which one or more of the fused rings is non-aromatic, provided that at least one ring is aromatic and at least one ring contains an N, O, or S ring atom. The heteroaryl groups can be attached to a parent group (i.e., the point of attachment) via any ring atom, including one of the heteroatoms or one of the carbon atoms, in the heteroaryl ring group. As chemically required, the heteroaryl may be attached to one or more other groups, for instance if operating as a bridging group.

Examples of heteroaryl groups include acridinyl, azocinyl, benzimidazolyl, benzofuranyl, benzo[d]oxazol-2(3H)-one, 2H-benzo[b][1,4]oxazin-3(4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

An “arylene,” “heteroarylene,” or “heterocyclylene” group is a bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents, it is meant that the group optionally has multiple non-hydrogen substituents, for example, from one to five, or from one to four, or from one to three, or one, or two non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Representative haloalkyls are trifluoromethyl, difluoromethyl, fluorochloromethyl, chloromethyl, and fluoromethyl.

The term “hydroxyalkyl” refers to -alkylene-OH.

It is to be understood that each individual atom present in Formula (I) and the compounds within Formula (I), may be present in the form of any of its naturally occurring isotopes, with the most abundant isotope(s) being preferred. Thus, by way of example, each individual hydrogen atom present in Formula (I), or in the formulae depicted hereinafter, may be present as a ¹H, ²H (deuterium; D), or ³H (tritium; T) atom, preferably ¹H. Similarly, by way of example, each individual carbon atom present in formula (I), or in the formulae depicted hereinafter, may be present as a ¹²C, ¹³C, or ¹⁴C atom, preferably ¹²C.

As used herein, “an effective amount” of a compound is an amount that is sufficient to negatively modulate or inhibit the activity of LRRK2 kinase.

As used herein, a “therapeutically effective amount” of a compound is an amount that is sufficient to ameliorate or in some manner reduce a symptom or stop or reverse progression of a condition, or negatively modulate or inhibit the activity of LRRK2 kinase. Such amount may be administered as a single dosage or may be administered according to a regimen, whereby it is effective.

As used herein, “treatment” means any manner in which the symptoms or pathology of a condition, disorder or disease in a patient are ameliorated or otherwise beneficially altered.

As used herein, “amelioration of the symptoms of a particular disorder by administration of a particular compound or pharmaceutical composition” refers to any lessening, whether permanent or temporary, lasting or transient, that can be attributed to or associated with administration of the composition.

### Compounds

In one aspect, the present disclosure provides compounds of Formula (I):

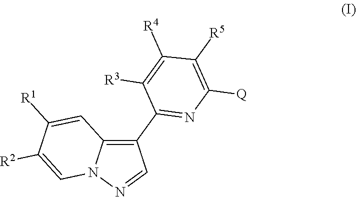

or pharmaceutically acceptable salts, prodrugs, solvates, hydrates, isomers, deuterated forms, and tautomers thereof, wherein:
R¹ is hydrogen, C₁-C₆ alkyl, C₃-C₆ cycloalkyl, halogen, halo C₁-C₆ alkyl, —OR⁶, cyano, —NR⁷R⁸, or —NHC(O)R⁹, wherein
   R⁶ is hydrogen, C₁-C₆ alkyl, C₃-C₆ cycloalkyl, C₃-C₆ cycloalkyl C₁-C₆ alkyl, 3-6 membered heterocyclyl, or 3-6 membered heterocyclyl C₁-C₆ alkyl;
   each of R⁷ and R⁸ is independently hydrogen, C₁-C₆ alkyl, or together with the nitrogen to which they are attached form a 3-8 membered monocyclic heterocyclyl group; and
   R⁹ is C₆-C₁₀ aryl, 5- or 6-membered heteroaryl, or C₁-C₆ alkyl, wherein
      the C₆-C₁₀ aryl is optionally substituted with C₁-C₆ alkyl, halogen, halo C₁-C₆ alkyl, —OR^{9A}, cyano, or —NR^{9B}R^{9C}, wherein
         R^{9A} is hydrogen or C₁-C₆ alkyl, and
         each of R^{9B} and R^{9C} is independently hydrogen or C₁-C₆ alkyl;
      the 5- or 6-membered heteraryl comprises one nitrogen atom and is optionally substituted with C₁-C₆ alkyl, halogen, halo C₁-C₆ alkyl, —OR^{9A}, cyano, or —NR^{9B}R^{9C}, and
      the C₁-C₆ alkyl is optionally substituted with halogen, —SO₂CH₃, cyano, 5- or 6-membered heteroaryl, —OR^{9D}, or —NR^{9E}R^{9F}, wherein
         R^{9D} is hydrogen, C₁-C₆ alkyl, or 5- or 6-membered heteroaryl comprising one nitrogen atom and optionally substituted with C₁-C₆ alkyl, halogen, halo C₁-C₆ alkyl, —OR^{9A}, cyano, or —NR^{9B}R^{9C}, and
         each of R^{9E} and R^{9F} is independently hydrogen, C₁-C₆ alkyl, —SO₂CH₃, or 5- or 6-membered heteroaryl comprising one nitrogen atom;
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen; and
Q is a 3-8 membered heterocyclyl group, wherein
   the heterocyclyl group includes at least one nitrogen atom;
   the heterocyclyl group is optionally fused to a non-aromatic ring containing 3-6 ring members of which 1 or 2 are optionally nitrogen, oxygen, or sulfur atoms and the remainder are carbons;
   the heterocyclyl group is optionally substituted with 1-4 R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.

In certain embodiments of Formula (I) as otherwise described herein, R¹ is hydrogen.

In certain embodiments of Formula (I) as otherwise described herein, R¹ is methyl.

In certain embodiments of Formula (I) as otherwise described herein, R¹ is cyclopropyl.

In certain embodiments of Formula (I) as otherwise described herein, R¹ is fluoro, chloro or bromo.

In certain embodiments of Formula (I) as otherwise described herein, R¹ is trifluoromethyl.

In certain embodiments of Formula (I) as otherwise described herein, R¹ is —OR⁶, wherein R⁶ is hydrogen.

In certain embodiments of Formula (I) as otherwise described herein, R¹ is —OR⁶, wherein R⁶ is isopropyl.

In certain embodiments of Formula (I) as otherwise described herein, R¹ is —OR⁶, wherein R⁶ is cyclopropylmethyl.

In certain embodiments of Formula (I) as otherwise described herein, R¹ is —OR⁶, wherein R⁶ is methyloxetane.

In certain embodiments of Formula (I) as otherwise described herein, R¹ is cyano.

In certain embodiments of Formula (I) as otherwise described herein, R¹ is —NR⁷R⁸, wherein each of R⁷ and R⁸ is independently hydrogen.

In certain embodiments of Formula (I) as otherwise described herein, R¹ is —NR⁷R⁸, wherein each of R⁷ and R⁸ is independently C₁-C₃ alkyl.

In certain embodiments of Formula (I) as otherwise described herein, R¹ is —NR⁷R⁸, wherein R⁷ and R⁸ together with the nitrogen to which they are attached form a 3-7 membered monocyclic heterocyclyl group.

In certain embodiments of Formula (I) as otherwise described herein, R¹ is morpholine.

In certain embodiments of Formula (I) as otherwise described herein, R¹ is —NHC(O)R⁹, wherein R⁹ is C₆-C₁₀ aryl.

In certain embodiments of Formula (I) as otherwise described herein, R¹ is —NHC(O)R⁹, wherein R⁹ is phenyl.

In certain embodiments of Formula (I) as otherwise described herein, R¹ is —NHC(O)R⁹, wherein R⁹ is 5- or 6-membered heteroaryl.

In certain embodiments of Formula (I) as otherwise described herein, R¹ is —NHC(O)R⁹, wherein R⁹ is 2-pyridyl.

In certain embodiments of Formula (I) as otherwise described herein, R¹ is —NHC(O)R⁹, wherein R⁹ is C₁-C₃ alkyl substituted with —NHSO₂CH₃.

In certain embodiments of Formula (I) as otherwise described herein, R¹ is —NHC(O)R⁹, wherein R⁹ is C₁-C₃ alkyl substituted with —SO₂CH₃.

In certain embodiments of Formula (I) as otherwise described herein, R¹ is —NHC(O)R⁹, wherein R⁹ is C₁-C₃ alkyl substituted with cyano.

In certain embodiments of Formula (I) as otherwise described herein, R¹ is —NHC(O)R⁹, wherein R⁹ is C₁-C₃ alkyl substituted with 5- or 6-membered heteroaryl.

In certain embodiments of Formula (I) as otherwise described herein, R¹ is —NHC(O)R⁹, wherein R⁹ is 2-pyridyl methyl or 3-pyridyl methyl.

In certain embodiments of Formula (I) as otherwise described herein, R¹ is —NHC(O)R⁹ and R⁹ is C₁-C₃ alkyl substituted with —OR^{9D}, wherein R^{9D} is hydrogen, C₁-C₆ alkyl, or 5- or 6-membered heteroaryl.

In certain embodiments of Formula (I) as otherwise described herein, R¹ is —NHC(O)R⁹ and R⁹ is methyl substituted with —OR^{9D}, wherein R^{9D} is 5- or 6-membered heteroaryl.

In certain embodiments of Formula (I) as otherwise described herein, R¹ is —NHC(O)R⁹ and R⁹ is methyl substituted with —OR^{9D}, wherein R^{9D} is pyridyl.

In certain embodiments of Formula (I) as otherwise described herein, R¹ is —NHC(O)R⁹ and R⁹ is C₁-C₃ alkyl substituted with —NR^{9E}R^{9F}, wherein each of R^{9E} and R^{9F} is independently hydrogen, C₁-C₆ alkyl, or 5- or 6-membered heteroaryl.

In certain embodiments of Formula (I) as otherwise described herein, R¹ is —NHC(O)R⁹ and R⁹ is methyl substituted with —NR^{9E}R^{9F}, wherein R^{9E} is hydrogen and R^{9F} is pyridyl.

In certain embodiments of Formula (I) as otherwise described herein, R¹ is —NHC(O)R⁹ and R⁹ is methyl substituted with —NR^{9E}R^{9F}, wherein R^{9E} is pyridyl and R^{9F} is hydrogen.

In certain embodiments of Formula (I) as otherwise described herein, R² is hydrogen.

In certain embodiments of Formula (I) as otherwise described herein, R² is —NHC(O)R¹⁰, wherein R¹⁰ is hydrogen or C₁-C₃ alkyl substituted with —NHSO₂CH₃.

In certain embodiments of Formula (I) as otherwise described herein, R² is —C(O)NHR¹¹, wherein R¹¹ is hydrogen or C₁-C₃ alkyl substituted with —NHSO₂CH₃.

In certain embodiments of Formula (I) as otherwise described herein, R³ is hydrogen.

In certain embodiments of Formula (I) as otherwise described herein, R³ is fluoro, chloro or bromo.

In certain embodiments of Formula (I) as otherwise described herein, R⁴ is hydrogen.

In certain embodiments of Formula (I) as otherwise described herein, R⁴ is fluoro, chloro, or bromo.

In certain embodiments of Formula (I) as otherwise described herein, R⁴ is C₁-C₃ alkoxy.

In certain embodiments of Formula (I) as otherwise described herein, R⁵ is hydrogen.

In certain embodiments of Formula (I) as otherwise described herein, R⁵ is fluoro, chloro or bromo.

In certain embodiments of Formula (I) as otherwise described herein, Q is a 3-7 membered heterocyclyl group including at least one nitrogen atom.

In certain embodiments of Formula (I) as otherwise described herein, Q is a 3-7 membered heterocyclyl group, wherein the heterocyclyl group includes at least one nitrogen atom and is fused to a non-aromatic ring containing 3-6 ring members of which 1 or 2 are optionally nitrogen atoms and the remainder are carbons.

In certain embodiments of Formula (I) as otherwise described herein, Q is a 3-7 membered heterocyclyl group, wherein the heterocyclyl group includes at least one nitrogen atom and is substituted with one to four R¹² groups, wherein each R¹² is independently C₁-C₃ alkyl.

In certain embodiments of Formula (I) as otherwise described herein, Q is a 3-7 membered heterocyclyl group, wherein the heterocyclyl group includes at least one nitrogen atom and is substituted with one R¹³ group, wherein R¹³ is hydrogen, C₁-C₃ alkyl, —SO₂R¹⁴, or —C(O)R¹⁵, and wherein R¹⁴ is hydrogen or C₁-C₃ alkyl, and R¹⁵ is hydrogen or C₁-C₃ alkyl substituted with —NHSO₂CH₃.

In certain embodiments of Formula (I) as otherwise described herein, Q is a 3-7 membered heterocyclyl group, wherein the heterocyclyl group includes at least one nitrogen atom and is substituted with one or two R¹² groups and with one R¹³ group. Each R¹² is independently C₁-C₃ alkyl. R¹³ is hydrogen, C₁-C₃ alkyl, —SO₂R¹⁴, or —C(O)R¹⁵, wherein R¹⁴ is hydrogen or C₁-C₃ alkyl, and R¹⁵ is hydrogen or C₁-C₃ alkyl substituted with —NHSO₂CH₃.

In another aspect, the present disclosure provides compounds of Formula (II):

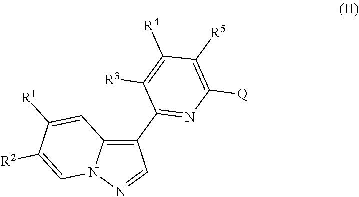

or pharmaceutically acceptable salts, prodrugs, solvates, hydrates, isomers, deuterated forms, and tautomers thereof, wherein:
R¹ is fluoro, chloro, or bromo;
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen; and
Q is a 3-8 membered heterocyclyl group, wherein the heterocyclyl group includes at least one nitrogen atom;
   the heterocyclyl group is optionally fused to a non-aromatic ring containing 3-6 ring members of which 1 or 2 are optionally nitrogen, oxygen or sulfur atoms and the remainder are carbons;
   the heterocyclyl group is optionally substituted with one to four R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.

In certain embodiments of Formula (II) as otherwise described herein, R² is hydrogen.

In certain embodiments of Formula (II) as otherwise described herein, R² is —NHC(O)R¹⁰, wherein R¹⁰ is hydrogen or C₁-C₃ alkyl substituted with —NHSO₂CH₃.

In certain embodiments of Formula (II) as otherwise described herein, R² is —C(O)NHR¹¹, wherein R¹¹ is hydrogen or C₁-C₃ alkyl substituted with —NHSO₂CH₃.

In certain embodiments of Formula (II) as otherwise described herein, R³ is hydrogen.

In certain embodiments of Formula (II) as otherwise described herein, R³ is fluoro, chloro or bromo.

In certain embodiments of Formula (II) as otherwise described herein, R⁴ is hydrogen.

In certain embodiments of Formula (II) as otherwise described herein, R⁴ is fluoro, chloro, or bromo.

In certain embodiments of Formula (II) as otherwise described herein, R⁴ is C₁-C₃ alkoxy.

In certain embodiments of Formula (II) as otherwise described herein, R⁵ is hydrogen.

In certain embodiments of Formula (II) as otherwise described herein, R⁵ is fluoro, chloro or bromo.

In certain embodiments of Formula (II) as otherwise described herein, Q is a 3-7 membered heterocyclyl group including at least one nitrogen atom.

In certain embodiments of Formula (II) as otherwise described herein, Q is a 3-7 membered heterocyclyl group, wherein the heterocyclyl group includes at least one nitrogen atom and is fused to a non-aromatic ring containing 3-6 ring members of which 1 or 2 are optionally nitrogen atoms and the remainder are carbons.

In certain embodiments of Formula (II) as otherwise described herein, Q is a 3-7 membered heterocyclyl group, wherein the heterocyclyl group includes at least one nitrogen atom and is substituted with one to four R¹² groups, wherein each R¹² is independently C₁-C₃ alkyl.

In certain embodiments of Formula (II) as otherwise described herein, Q is a 3-7 membered heterocyclyl group, wherein the heterocyclyl group includes at least one nitrogen atom and is substituted with one R¹³ group, wherein R¹³ is hydrogen, C₁-C₃ alkyl, —SO₂R¹⁴, or —C(O)R¹⁵, and wherein R¹⁴ is hydrogen or C₁-C₃ alkyl, and R¹⁵ is hydrogen or C₁-C₃ alkyl substituted with —NHSO₂CH₃.

In certain embodiments of Formula (II) as otherwise described herein, Q is a 3-7 membered heterocyclyl group, wherein the heterocyclyl group includes at least one nitrogen atom and is substituted with one or two R¹² groups and with one R¹³ group. Each R¹² is independently C₁-C₃ alkyl. R¹³ is hydrogen, C₁-C₃ alkyl, —SO₂R¹⁴, or —C(O)R¹⁵, wherein R¹⁴ is hydrogen or C₁-C₃ alkyl, and R¹⁵ is hydrogen or C₁-C₃ alkyl substituted with —NHSO₂CH₃.

In another aspect, the present disclosure provides compounds of Formula (III):

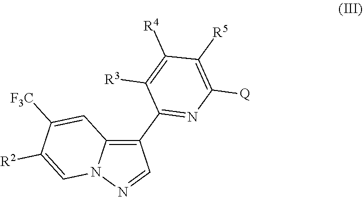

or pharmaceutically acceptable salts, prodrugs, solvates, hydrates, isomers, deuterated forms, and tautomers thereof, wherein:
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen; and
Q is a 3-8 membered heterocyclyl group, wherein
   the heterocyclyl group includes at least one nitrogen atom;
   the heterocyclyl group is optionally fused to a non-aromatic ring containing 3-6 ring members of which 1 or 2 are optionally nitrogen, oxygen or sulfur atoms and the remainder are carbons;
   the heterocyclyl group is optionally substituted with one to four R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.

In certain embodiments of Formula (III) as otherwise described herein, R² is hydrogen.

In certain embodiments of Formula (III) as otherwise described herein, R² is —NHC(O)R¹⁰, wherein R¹⁰ is hydrogen or C₁-C₃ alkyl substituted with —NHSO₂CH₃.

In certain embodiments of Formula (III) as otherwise described herein, R² is —C(O)NHR¹¹, wherein R¹¹ is hydrogen or C₁-C₃ alkyl substituted with —NHSO₂CH₃.

In certain embodiments of Formula (III) as otherwise described herein, R³ is hydrogen.

In certain embodiments of Formula (III) as otherwise described herein, R³ is fluoro, chloro or bromo.

In certain embodiments of Formula (III) as otherwise described herein, R⁴ is hydrogen.

In certain embodiments of Formula (III) as otherwise described herein, R⁴ is fluoro, chloro, or bromo.

In certain embodiments of Formula (III) as otherwise described herein, R⁴ is C₁-C₃ alkoxy.

In certain embodiments of Formula (III) as otherwise described herein, R⁵ is hydrogen.

In certain embodiments of Formula (III) as otherwise described herein, R⁵ is fluoro, chloro or bromo.

In certain embodiments of Formula (III) as otherwise described herein, Q is a 3-7 membered heterocyclyl group including at least one nitrogen atom.

In certain embodiments of Formula (III) as otherwise described herein, Q is a 3-7 membered heterocyclyl group, wherein the heterocyclyl group includes at least one nitrogen atom and is fused to a non-aromatic ring containing 3-6 ring members of which 1 or 2 are optionally nitrogen atoms and the remainder are carbons.

In certain embodiments of Formula (III) as otherwise described herein, Q is a 3-7 membered heterocyclyl group, wherein the heterocyclyl group includes at least one nitrogen atom and is substituted with one to four R¹² groups, wherein each R¹² is independently C₁-C₃ alkyl.

In certain embodiments of Formula (III) as otherwise described herein, Q is a 3-7 membered heterocyclyl group, wherein the heterocyclyl group includes at least one nitrogen atom and is substituted with one R¹³ group, wherein R¹³ is hydrogen, C₁-C₃ alkyl, —SO₂R¹⁴, or —C(O)R¹⁵, and wherein R¹⁴ is hydrogen or C₁-C₃ alkyl, and R¹⁵ is hydrogen or C₁-C₃ alkyl substituted with —NHSO₂CH₃.

In certain embodiments of Formula (III) as otherwise described herein, Q is a 3-7 membered heterocyclyl group, wherein the heterocyclyl group includes at least one nitrogen atom and is substituted with one or two R¹² groups and with one R¹³ group. Each R¹² is independently C₁-C₃ alkyl. R¹³ is hydrogen, C₁-C₃ alkyl, —SO₂R¹⁴, or —C(O)R¹⁵, wherein R¹⁴ is hydrogen or C₁-C₃ alkyl, and R¹⁵ is hydrogen or C₁-C₃ alkyl substituted with —NHSO₂CH₃.

In another aspect, the present disclosure provides compounds of Formula (IV):

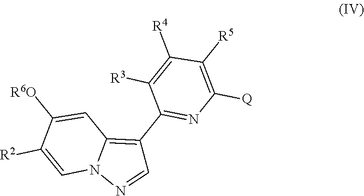

or pharmaceutically acceptable salts, prodrugs, solvates, hydrates, isomers, deuterated forms, and tautomers thereof, wherein:
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen;
R⁶ is hydrogen, C₁-C₆ alkyl, C₃-C₆ cycloalkyl, C₃-C₆ cycloalkyl C₁-C₆ alkyl, 3-6 membered heterocyclyl, or 3-6 membered heterocyclyl C₁-C₆ alkyl; and
Q is a 3-8 membered heterocyclyl group, wherein
   the heterocyclyl group includes at least one nitrogen atom;
   the heterocyclyl group is optionally fused to a non-aromatic ring containing 3-6 ring members of which 1 or 2 are optionally nitrogen, oxygen or sulfur atoms and the remainder are carbons;
   the heterocyclyl group is optionally substituted with one to four R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.

In certain embodiments of Formula (IV) as otherwise described herein, R² is hydrogen.

In certain embodiments of Formula (IV) as otherwise described herein, R² is —NHC(O)R¹⁰, wherein R¹⁰ is hydrogen or C₁-C₃ alkyl substituted with —NHSO₂CH₃.

In certain embodiments of Formula (IV) as otherwise described herein, R² is —C(O)NHR¹¹, wherein R¹¹ is hydrogen or C₁-C₃ alkyl substituted with —NHSO₂CH₃.

In certain embodiments of Formula (IV) as otherwise described herein, R³ is hydrogen.

In certain embodiments of Formula (IV) as otherwise described herein, R³ is fluoro, chloro or bromo.

In certain embodiments of Formula (IV) as otherwise described herein, R⁴ is hydrogen.

In certain embodiments of Formula (IV) as otherwise described herein, R⁴ is fluoro, chloro, or bromo.

In certain embodiments of Formula (IV) as otherwise described herein, R⁴ is C₁-C₃ alkoxy.

In certain embodiments of Formula (IV) as otherwise described herein, R⁵ is hydrogen.

In certain embodiments of Formula (IV) as otherwise described herein, R⁵ is fluoro, chloro or bromo.

In certain embodiments of Formula (IV) as otherwise described herein, R⁶ is hydrogen.

In certain embodiments of Formula (IV) as otherwise described herein, R⁶ is isopropyl.

In certain embodiments of Formula (IV) as otherwise described herein, R⁶ is cyclopropylmethyl.

In certain embodiments of Formula (IV) as otherwise described herein, R⁶ is methyloxetane.

In certain embodiments of Formula (IV) as otherwise described herein, Q is a 3-7 membered heterocyclyl group including at least one nitrogen atom.

In certain embodiments of Formula (IV) as otherwise described herein, Q is a 3-7 membered heterocyclyl group, wherein the heterocyclyl group includes at least one nitrogen atom and is fused to a non-aromatic ring containing 3-6 ring members of which 1 or 2 are optionally nitrogen atoms and the remainder are carbons.

In certain embodiments of Formula (IV) as otherwise described herein, Q is a 3-7 membered heterocyclyl group, wherein the heterocyclyl group includes at least one nitrogen atom and is substituted with one to four R¹² groups, wherein each R¹² is independently C₁-C₃ alkyl.

In certain embodiments of Formula (IV) as otherwise described herein, Q is a 3-7 membered heterocyclyl group, wherein the heterocyclyl group includes at least one nitrogen atom and is substituted with one R¹³ group, wherein R¹³ is hydrogen, C₁-C₃ alkyl, —SO₂R¹⁴, or —C(O)R¹⁵, and wherein R¹⁴ is hydrogen or C₁-C₃ alkyl, and R¹⁵ is hydrogen or C₁-C₃ alkyl substituted with —NHSO₂CH₃.

In certain embodiments of Formula (IV) as otherwise described herein, Q is a 3-7 membered heterocyclyl group, wherein the heterocyclyl group includes at least one nitrogen atom and is substituted with one or two R¹² groups and with one R¹³ group. Each R¹² is independently C₁-C₃ alkyl. R¹³ is hydrogen, C₁-C₃ alkyl, —SO₂R¹⁴, or —C(O)R¹⁵, wherein R¹⁴ is hydrogen or C₁-C₃ alkyl, and R¹⁵ is hydrogen or C₁-C₃ alkyl substituted with —NHSO₂CH₃.

In another aspect, the present disclosure provides compounds of Formula (V):

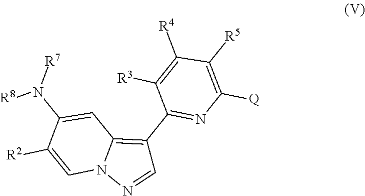

or pharmaceutically acceptable salts, prodrugs, solvates, hydrates, isomers, deuterated forms, and tautomers thereof, wherein:
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen;
R⁷ and R⁸ are independently hydrogen or C₁-C₆ alkyl, or together with the nitrogen to which they are attached form a 3-8 membered monocyclic heterocyclyl group; and
Q is a 3-8 membered heterocyclyl group, wherein
   the heterocyclyl group includes at least one nitrogen atom;
   the heterocyclyl group is optionally fused to a non-aromatic ring containing 3-6 ring members of which 1 or 2 are optionally nitrogen, oxygen or sulfur atoms and the remainder are carbons;
   the heterocyclyl group is optionally substituted with one to four R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.

In certain embodiments of Formula (V) as otherwise described herein, R² is hydrogen.

In certain embodiments of Formula (V) as otherwise described herein, R² is —NHC(O)R¹⁰, wherein R¹⁰ is hydrogen or C₁-C₃ alkyl substituted with —NHSO₂CH₃.

In certain embodiments of Formula (V) as otherwise described herein, R² is —C(O)NHR¹¹, wherein R¹¹ is hydrogen or C₁-C₃ alkyl substituted with —NHSO₂CH₃.

In certain embodiments of Formula (V) as otherwise described herein, R³ is hydrogen.

In certain embodiments of Formula (V) as otherwise described herein, R³ is fluoro, chloro or bromo.

In certain embodiments of Formula (V) as otherwise described herein, R⁴ is hydrogen.

In certain embodiments of Formula (V) as otherwise described herein, R⁴ is fluoro, chloro, or bromo.

In certain embodiments of Formula (V) as otherwise described herein, R⁴ is C₁-C₃ alkoxy.

In certain embodiments of Formula (V) as otherwise described herein, R⁵ is hydrogen.

In certain embodiments of Formula (V) as otherwise described herein, R⁵ is fluoro, chloro or bromo.

In certain embodiments of Formula (V) as otherwise described herein, each of R⁷ and R⁸ is independently hydrogen.

In certain embodiments of Formula (V) as otherwise described herein, each of R⁷ and R⁸ is independently C₁-C₃ alkyl.

In certain embodiments of Formula (V) as otherwise described herein, R⁷ and R⁸ together with the nitrogen to which they are attached form a 3-7 membered monocyclic heterocyclyl group.

In certain embodiments of Formula (V) as otherwise described herein, R⁷ and R⁸ together with the nitrogen to which they are attached form a morpholine ring.

In certain embodiments of Formula (V) as otherwise described herein, Q is a 3-7 membered heterocyclyl group including at least one nitrogen atom.

In certain embodiments of Formula (V) as otherwise described herein, Q is a 3-7 membered heterocyclyl group, wherein the heterocyclyl group includes at least one nitrogen atom and is fused to a non-aromatic ring containing 3-6 ring members of which 1 or 2 are optionally nitrogen atoms and the remainder are carbons.

In certain embodiments of Formula (V) as otherwise described herein, Q is a 3-7 membered heterocyclyl group, wherein the heterocyclyl group includes at least one nitrogen atom and is substituted with one to four R¹² groups, wherein each R¹² is independently C₁-C₃ alkyl.

In certain embodiments of Formula (V) as otherwise described herein, Q is a 3-7 membered heterocyclyl group, wherein the heterocyclyl group includes at least one nitrogen atom and is substituted with one R¹³ group, wherein R¹³ is hydrogen, C₁-C₃ alkyl, —SO₂R¹⁴, or —C(O)R¹⁵, and wherein R¹⁴ is hydrogen or C₁-C₃ alkyl, and R¹⁵ is hydrogen or C₁-C₃ alkyl substituted with —NHSO₂CH₃.

In certain embodiments of Formula (V) as otherwise described herein, Q is a 3-7 membered heterocyclyl group, wherein the heterocyclyl group includes at least one nitrogen atom and is substituted with one or two R¹² groups and with one R¹³ group. Each R¹² is independently C₁-C₃ alkyl. R¹³ is hydrogen, C₁-C₃ alkyl, —SO₂R¹⁴, or —C(O)R¹⁵, wherein R¹⁴ is hydrogen or C₁-C₃ alkyl, and R¹⁵ is hydrogen or C₁-C₃ alkyl substituted with —NHSO₂CH₃.

In another aspect, the present disclosure provides compounds of Formula (VI):

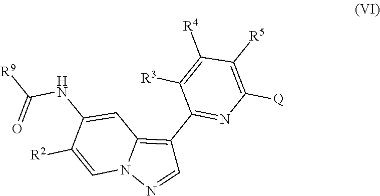

or a pharmaceutically acceptable salt, prodrug, solvate, hydrate, isomer, deuterated form, or tautomer thereof, wherein:
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen;
R⁹ is C₆-C₁₀ aryl, 5- or 6-membered heteroaryl, or C₁-C₆ alkyl, wherein
   the C₆-C₁₀ aryl is optionally substituted with C₁-C₆ alkyl, halogen, halo C₁-C₆ alkyl, —OR^{9A}, cyano, or —NR^{9B}R^{9C}, wherein
      R^{9A} is hydrogen or C₁-C₆ alkyl, and
      each of R^{9B} and R^{9C} is independently hydrogen or C₁-C₆ alkyl;
   the 5- or 6-membered heteraryl comprises one nitrogen atom and is optionally substituted with C₁-C₆ alkyl, halogen, halo C₁-C₆ alkyl, —OR^{9A}, cyano, or —NR^{9B}R^{9C}, and
   the C₁-C₆ alkyl is optionally substituted with halogen, —SO₂CH₃, cyano, 5- or 6-membered heteroaryl, —OR^{9D}, or —NR^{9E}R^{9F}, wherein
      R^{9D} is hydrogen, C₁-C₆ alkyl, or 5- or 6-membered heteroaryl comprising one nitrogen atom and optionally substituted with C₁-C₆ alkyl, halogen, halo C₁-C₆ alkyl, —OR^{9A}, cyano, or —NR^{9B}R^{9C}, and
      each of R^{9E} and R^{9F} is independently hydrogen, C₁-C₆ alkyl, —SO₂CH₃, or 5- or 6-membered heteroaryl comprising one nitrogen atom;
Q is a 3-8 membered heterocyclyl group, wherein
   the heterocyclyl group includes at least one nitrogen atom;
   the heterocyclyl group is optionally fused to a non-aromatic ring containing 3-6 ring members of which 1 or 2 are optionally nitrogen, oxygen or sulfur atoms and the remainder are carbons;
   the heterocyclyl group is optionally substituted with one to four R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.

In certain embodiments of Formula (IV) as otherwise described herein, R² is hydrogen.

In certain embodiments of Formula (VI) as otherwise described herein, R² is —NHC(O)R¹⁰, wherein R¹⁰ is hydrogen or C₁-C₃ alkyl substituted with —NHSO₂CH₃.

In certain embodiments of Formula (VI) as otherwise described herein, R² is —C(O)NHR¹¹, wherein R¹¹ is hydrogen or C₁-C₃ alkyl substituted with —NHSO₂CH₃.

In certain embodiments of Formula (VI) as otherwise described herein, R³ is hydrogen.

In certain embodiments of Formula (VI) as otherwise described herein, R³ is fluoro, chloro or bromo.

In certain embodiments of Formula (VI) as otherwise described herein, R⁴ is hydrogen.

In certain embodiments of Formula (VI) as otherwise described herein, R⁴ is fluoro, chloro, or bromo.

In certain embodiments of Formula (VI) as otherwise described herein, R⁴ is C₁-C₃ alkoxy.

In certain embodiments of Formula (VI) as otherwise described herein, R⁵ is hydrogen.

In certain embodiments of Formula (VI) as otherwise described herein, R⁵ is fluoro, chloro or bromo.

In certain embodiments of Formula (VI) as otherwise described herein, R⁹ is phenyl.

In certain embodiments of Formula (VI) as otherwise described herein, R⁹ is 2-pyridyl.

In certain embodiments of Formula (VI) as otherwise described herein, R⁹ is C₁-C₃ alkyl substituted with —NHSO₂CH₃.

In certain embodiments of Formula (VI) as otherwise described herein, R⁹ is C₁-C₃ alkyl substituted with —SO₂CH₃.

In certain embodiments of Formula (VI) as otherwise described herein, R⁹ is C₁-C₃ alkyl substituted with cyano.

In certain embodiments of Formula (VI) as otherwise described herein, R⁹ is 2-pyridyl methyl or 3-pyridyl methyl.

In certain embodiments of Formula (VI) as otherwise described herein, R⁹ is methyl substituted with —OR^{9D}, wherein R^{9D} is pyridyl.

In certain embodiments of Formula (VI) as otherwise described herein, R⁹ is methyl substituted with —NR^{9E}R^{9F}, wherein R^{9E} is hydrogen and R^{9F} is pyridyl.

In certain embodiments of Formula (VI) as otherwise described herein, R⁹ is methyl substituted with —NR^{9E}R^{9F}, wherein R^{9E} is pyridyl and R^{9F} is hydrogen.

In certain embodiments of Formula (VI) as otherwise described herein, Q is a 3-7 membered heterocyclyl group including at least one nitrogen atom.

In certain embodiments of Formula (VI) as otherwise described herein, Q is a 3-7 membered heterocyclyl group, wherein the heterocyclyl group includes at least one nitrogen atom and is fused to a non-aromatic ring containing 3-6 ring members of which 1 or 2 are optionally nitrogen atoms and the remainder are carbons.

In certain embodiments of Formula (VI) as otherwise described herein, Q is a 3-7 membered heterocyclyl group, wherein the heterocyclyl group includes at least one nitrogen atom and is substituted with one to four R¹² groups, wherein each R¹² is independently C₁-C₃ alkyl.

In certain embodiments of Formula (VI) as otherwise described herein, Q is a 3-7 membered heterocyclyl group, wherein the heterocyclyl group includes at least one nitrogen atom and is substituted with one R¹³ group, wherein R¹³ is hydrogen, C₁-C₃ alkyl, —SO₂R¹⁴, or —C(O)R¹⁵, and wherein R¹⁴ is hydrogen or C₁-C₃ alkyl, and R¹⁵ is hydrogen or C₁-C₃ alkyl substituted with —NHSO₂CH₃.

In certain embodiments of Formula (VI) as otherwise described herein, Q is a 3-7 membered heterocyclyl group, wherein the heterocyclyl group includes at least one nitrogen atom and is substituted with one or two R¹² groups and with one R¹³ group. Each R¹² is independently C₁-C₃ alkyl. R¹³ is hydrogen, C₁-C₃ alkyl, —SO₂R¹⁴, or —C(O)R¹⁵, wherein R¹⁴ is hydrogen or C₁-C₃ alkyl, and R¹⁵ is hydrogen or C₁-C₃ alkyl substituted with —NHSO₂CH₃.

In certain embodiments of Formula (I), Formula (II), Formula (III), Formula (IV), Formula (V) or Formula (VI) as otherwise described herein, Q is a group of the formula:

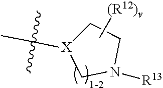

wherein
   each v represents 0, 1, 2, 3 or 4;
   each R¹² is independently hydrogen, C₁-C₆ alkyl, halogen, halo C₁-C₆ alkyl, or C₁-C₆ alkoxy;
   R¹³ is hydrogen, C₁-C₆ alkyl, —SO₂R¹⁴, or —C(O)R¹⁵, wherein R¹⁴ is hydrogen or C₁-C₆ alkyl, and R¹⁵ is hydrogen or C₁-C₆ alkyl substituted with —NHSO₂CH₃; and
   X is CR¹⁶ or nitrogen, wherein R¹⁶ is hydrogen or C₁-C₆ alkyl.

In certain embodiments of Formula (I), Formula (II), Formula (III), Formula (IV), Formula (V) or Formula (VI) as otherwise described herein, Q represents a nitrogen-containing ring selected from:

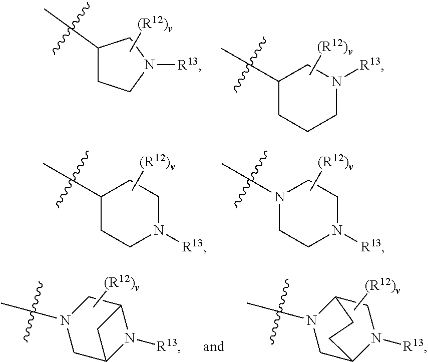

wherein
   each v represents 0, 1, 2, 3, or 4;
   R¹² is hydrogen, C₁-C₆ alkyl, halogen, halo C₁-C₆ alkyl, or C₁-C₆ alkoxy; and
   R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.

In certain embodiments of Formula (I), Formula (II), Formula (III), Formula (IV), Formula (V) or Formula (VI) as otherwise described herein, Q represents a nitrogen-containing ring selected from:

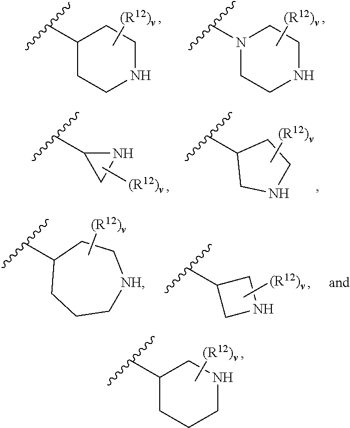

wherein
   each v represents 0, 1, 2, 3 or 4; and
   each R¹² is independently hydrogen, C₁-C₆ alkyl, halogen, halo C₁-C₆ alkyl, or C₁-C₆ alkoxy.

In certain embodiments of Formula (I), Formula (II), Formula (III), Formula (IV), Formula (V) or Formula (VI) as otherwise described herein, Q represents a fused bicyclic group selected from:

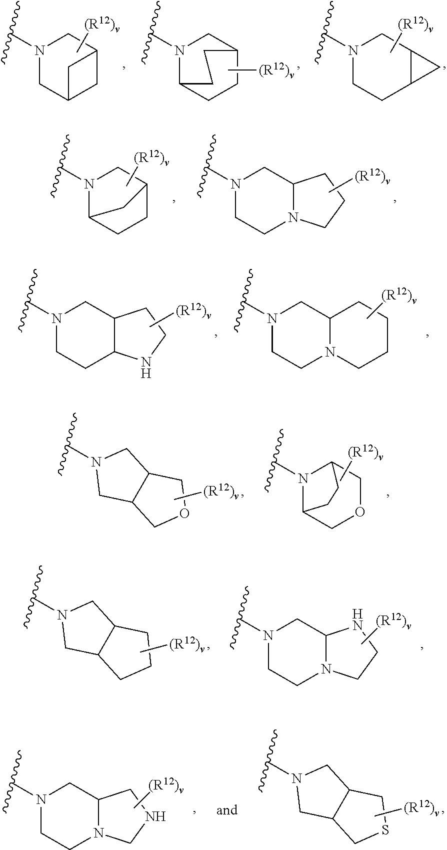

wherein
   each v represents 0, 1, 2, 3 or 4;
   each R¹² is independently hydrogen, C₁-C₆ alkyl, halogen, halo C₁-C₆ alkyl, or C₁-C₆ alkoxy, and can be on either ring.

In one aspect, the present disclosure provides compounds of Formula (VII):

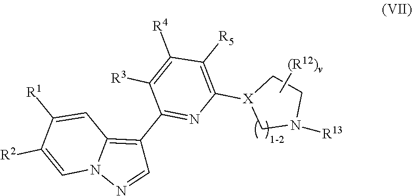

or pharmaceutically acceptable salts, prodrugs, solvates, hydrates, isomers, deuterated forms, and tautomers thereof, wherein:
R¹ is hydrogen, C₁-C₆ alkyl, C₃-C₆ cycloalkyl, halogen, halo C₁-C₆ alkyl, —OR⁶, cyano, —NR⁷R⁸, or —NHC(O)R⁹, wherein
   R⁶ is hydrogen, C₁-C₆ alkyl, C₃-C₆ cycloalkyl, C₃-C₆ cycloalkyl C₁-C₆ alkyl, 3-6 membered heterocyclyl, or 3-6 membered heterocyclyl C₁-C₆ alkyl;
   each of R⁷ and R⁸ is independently hydrogen, C₁-C₆ alkyl, or together with the nitrogen to which they are attached form a 3-8 membered monocyclic heterocyclyl group; and
   R⁹ is C₆-C₁₀ aryl, 5- or 6-membered heteroaryl, or C₁-C₆ alkyl, wherein
      the C₆-C₁₀ aryl is optionally substituted with C₁-C₆ alkyl, halogen, halo C₁-C₆ alkyl, —OR^{9A}, cyano, or —NR^{9B}R^{9C}, wherein
         R^{9A} is hydrogen or C₁-C₆ alkyl, and
         each of R^{9B} and R^{9C} is independently hydrogen or C₁-C₆ alkyl;
      the 5- or 6-membered heteraryl comprises one nitrogen atom and is optionally substituted with C₁-C₆ alkyl, halogen, halo C₁-C₆ alkyl, —OR^{9A}, cyano, or —NR^{9B}R^{9C}, and
      the C₁-C₆ alkyl is optionally substituted with halogen, —SO₂CH₃, cyano, 5- or 6-membered heteroaryl, —OR^{9D}, or —NR^{9E}R^{9F}, wherein
         R^{9D} is hydrogen, C₁-C₆ alkyl, or 5- or 6-membered heteroaryl comprising one nitrogen atom and optionally substituted with C₁-C₆ alkyl, halogen, halo C₁-C₆ alkyl, —OR^{9A}, cyano, or —NR^{9B}R^{9C}, and
         each of R^{9E} and R^{9F} is independently hydrogen, C₁-C₆ alkyl, —SO₂CH₃, or 5- or 6-membered heteroaryl comprising one nitrogen atom;
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen;
each R¹² is independently hydrogen, C₁-C₆ alkyl, halogen, halo C₁-C₆ alkyl, or C₁-C₆ alkoxy;
R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃;
X is CR¹⁶ or nitrogen, wherein R¹⁶ is hydrogen or C₁-C₆ alkyl; and
each v represents 0, 1, 2, 3 or 4.

In certain embodiments of Formula (VII) as otherwise described herein, R¹ is hydrogen.

In certain embodiments of Formula (VII) as otherwise described herein, R¹ is methyl.

In certain embodiments of Formula (VII) as otherwise described herein, R¹ is cyclopropyl.

In certain embodiments of Formula (VII) as otherwise described herein, R¹ is fluoro, chloro or bromo.

In certain embodiments of Formula (VII) as otherwise described herein, R¹ is trifluoromethyl.

In certain embodiments of Formula (VII) as otherwise described herein, R¹ is —OR⁶, wherein R⁶ is hydrogen.

In certain embodiments of Formula (VII) as otherwise described herein, R¹ is —OR⁶, wherein R⁶ is isopropyl.

In certain embodiments of Formula (VII) as otherwise described herein, R¹ is —OR⁶, wherein R⁶ is cyclopropylmethyl.

In certain embodiments of Formula (VII) as otherwise described herein, R¹ is —OR⁶, wherein R⁶ is methyloxetane.

In certain embodiments of Formula (VII) as otherwise described herein, R¹ is cyano.

In certain embodiments of Formula (VII) as otherwise described herein, R¹ is —NR⁷R⁸, wherein each of R⁷ and R⁸ is independently hydrogen.

In certain embodiments of Formula (VII) as otherwise described herein, R¹ is —NR⁷R⁸, wherein each of R⁷ and R⁸ is independently C₁-C₃ alkyl.

In certain embodiments of Formula (VII) as otherwise described herein, R¹ is —NR⁷R⁸, wherein R⁷ and R⁸ together with the nitrogen to which they are attached form a 3-7 membered monocyclic heterocyclyl group.

In certain embodiments of Formula (VII) as otherwise described herein, R¹ is morpholine.

In certain embodiments of Formula (VII) as otherwise described herein, R¹ is —NHC(O)R⁹, wherein R⁹ is C₆-C₁₀ aryl.

In certain embodiments of Formula (VII) as otherwise described herein, R¹ is —NHC(O)R⁹, wherein R⁹ is phenyl.

In certain embodiments of Formula (VII) as otherwise described herein, R¹ is —NHC(O)R⁹, wherein R⁹ is 5- or 6-membered heteroaryl.

In certain embodiments of Formula (VII) as otherwise described herein, R¹ is —NHC(O)R⁹, wherein R⁹ is 2-pyridyl.

In certain embodiments of Formula (VII) as otherwise described herein, R¹ is —NHC(O)R⁹, wherein R⁹ is C₁-C₃ alkyl substituted with —NHSO₂CH₃.

In certain embodiments of Formula (VII) as otherwise described herein, R¹ is —NHC(O)R⁹, wherein R⁹ is C₁-C₃ alkyl substituted with —SO₂CH₃.

In certain embodiments of Formula (VII) as otherwise described herein, R¹ is —NHC(O)R⁹, wherein R⁹ is C₁-C₃ alkyl substituted with cyano.

In certain embodiments of Formula (VII) as otherwise described herein, R¹ is —NHC(O)R⁹, wherein R⁹ is C₁-C₃ alkyl substituted with 5- or 6-membered heteroaryl.

In certain embodiments of Formula (VII) as otherwise described herein, R¹ is —NHC(O)R⁹, wherein R⁹ is 2-pyridyl methyl or 3-pyridyl methyl.

In certain embodiments of Formula (VII) as otherwise described herein, R¹ is —NHC(O)R⁹ and R⁹ is C₁-C₃ alkyl substituted with —OR^{9D}, wherein R^{9D} is hydrogen, C₁-C₆ alkyl, or 5- or 6-membered heteroaryl.

In certain embodiments of Formula (VII) as otherwise described herein, R¹ is —NHC(O)R⁹ and R⁹ is methyl substituted with —OR^{9D}, wherein R^{9A} is 5- or 6-membered heteroaryl.

In certain embodiments of Formula (VII) as otherwise described herein, R¹ is —NHC(O)R⁹ and R⁹ is methyl substituted with —OR^{9D}, wherein R^{9D} is pyridyl.

In certain embodiments of Formula (VII) as otherwise described herein, R¹ is —NHC(O)R⁹ and R⁹ is C₁-C₃ alkyl substituted with —NR^{9E}R^{9F}, wherein each of R^{9E} and R^{9F} is independently hydrogen, C₁-C₆ alkyl, or 5- or 6-membered heteroaryl.

In certain embodiments of Formula (VII) as otherwise described herein, R¹ is —NHC(O)R⁹ and R⁹ is methyl substituted with —NR^{9E}R^{9F}, wherein R^{9E} is hydrogen and R^{9F} is pyridyl.

certain embodiments of Formula (VII) as otherwise described herein, R¹ is —NHC(O)R⁹ and R⁹ is methyl substituted with —NR^{9E}R^{9F}, wherein R^{9E} is pyridyl and R^{9F} is hydrogen.

In certain embodiments of Formula (VII) as otherwise described herein, R² is hydrogen.

In certain embodiments of Formula (VII) as otherwise described herein, R² is —NHC(O)R¹⁰, wherein R¹⁰ is hydrogen or C₁-C₃ alkyl substituted with —NHSO₂CH₃.

In certain embodiments of Formula (VII) as otherwise described herein, R² is —C(O)NHR¹¹, wherein R¹¹ is hydrogen or C₁-C₃ alkyl substituted with —NHSO₂CH₃.

In certain embodiments of Formula (VII) as otherwise described herein, R³ is hydrogen.

In certain embodiments of Formula (VII) as otherwise described herein, R³ is fluoro, chloro or bromo.

In certain embodiments of Formula (VII) as otherwise described herein, R⁴ is hydrogen.

In certain embodiments of Formula (VII) as otherwise described herein, R⁴ is fluoro, chloro, or bromo.

In certain embodiments of Formula (VII) as otherwise described herein, R⁴ is C₁-C₃ alkoxy.

In certain embodiments of Formula (VII) as otherwise described herein, R⁵ is hydrogen.

In certain embodiments of Formula (VII) as otherwise described herein, R⁵ is fluoro, chloro or bromo.

In certain embodiments of Formula (VII) as otherwise described herein, R¹² is hydrogen.

In certain embodiments of Formula (VII) as otherwise described herein, R¹² is methyl.

In certain embodiments of Formula (VII) as otherwise described herein, R¹³ is hydrogen.

In certain embodiments of Formula (VII) as otherwise described herein, R¹³ is methyl.

In certain embodiments of Formula (VII) as otherwise described herein, R¹³ is —SO₂R¹⁴, wherein R¹⁴ is hydrogen or C₁-C₆ alkyl.

In certain embodiments of Formula (VII) as otherwise described herein, R¹³ is —SO₂R¹⁴, wherein R¹⁴ is methyl.

In certain embodiments of Formula (VII) as otherwise described herein, R¹³ is —C(O)R¹⁵, wherein R¹⁵ is C₁-C₆ alkyl substituted with —NHSO₂CH₃.

In certain embodiments of Formula (VII) as otherwise described herein, R¹³ is —C(O)R¹⁵, wherein R¹⁵ is —CH₂NHSO₂CH₃.

In certain embodiments of Formula (VII) as otherwise described herein, X is CR¹⁶, wherein R¹⁶ is hydrogen.

In certain embodiments of Formula (VII) as otherwise described herein, X is N.

In certain embodiments of Formula (VII) as otherwise described herein, each v is 0.

In certain embodiments of Formula (VII) as otherwise described herein, each v is 1.

In certain embodiments of Formula (VII) as otherwise described herein, each v is 2.

In certain embodiments of Formula (VII) as otherwise described herein, each v is 3.

In certain embodiments of Formula (VII) as otherwise described herein, each v is 4.

In one embodiment, the compound of Formula (I) is selected from:

TABLE 1

| Name | Structure |
|---|---|
| 3-(6-(piperidin-4-yl)pyridin-2- yl)pyrazolo[1,5-a]pyridine | 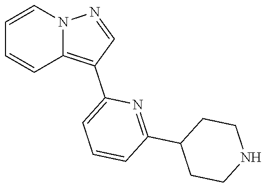 [SMILES: c1cc(-c2cnn3ccccc23)nc(C2CCNCC2)c1] |
| 3-(6-(1-methylpiperidin-4-yl)pyridin-2- yl)pyrazolo[1,5-a]pyridine | 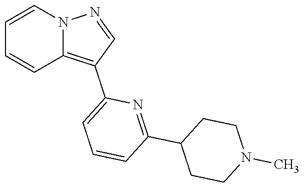 [SMILES: CN1CCC(c2cccc(-c3cnn4ccccc34)n2)CC1] |
| 3-(6-(pyrrolidin-3-yl)pyridin-2- yl)pyrazolo[1,5-a]pyridine | 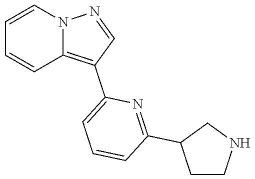 [SMILES: c1cc(-c2cnn3ccccc23)nc(C2CCNC2)c1] |
| 5-chloro-3-(6-(pyrrolidin-3-yl)pyridin- 2-yl)pyrazolo[1,5-a]pyridine | 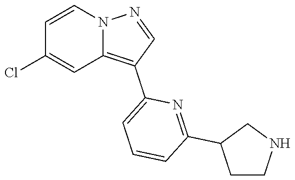 [SMILES: Clc1ccn2ncc(-c3cccc(C4CCNC4)n3)c2c1] |
| Rac-5-chloro-3-(6-(piperidin-3- yl)pyridin-2-yl)pyrazolo[1,5-a]pyridine | 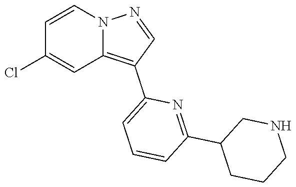 [SMILES: Clc1ccn2ncc(-c3cccc(C4CCCNC4)n3)c2c1] |
| (+)-5-chloro-3-(6-(piperidin-3- yl)pyridin-2-yl)pyrazolo[1,5-a]pyridine | 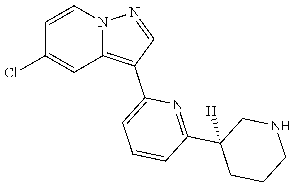 [SMILES: Clc1ccn2ncc(-c3cccc([C@@H]4CCCNC4)n3)c2c1] |
| (−)-5-chloro-3-(6-(piperidin-3- yl)pyridin-2-yl)pyrazolo[1,5-a]pyridine | 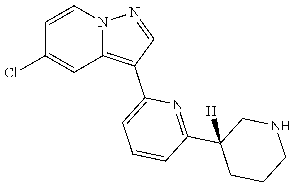 [SMILES: Clc1ccn2ncc(-c3cccc([C@H]4CCCNC4)n3)c2c1] |
| 5-bromo-3-(6-(piperidin-3-yl)pyridin- 2-yl)pyrazolo[1,5-a]pyridine | 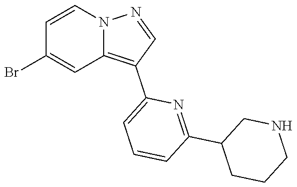 [SMILES: Brc1ccn2ncc(-c3cccc(C4CCCNC4)n3)c2c1] |
| 5-methyl-3-(6-(piperidin-3-yl)pyridin- 2-yl)pyrazolo[1,5-a]pyridine | 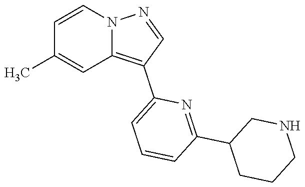 [SMILES: Cc1ccn2ncc(-c3cccc(C4CCCNC4)n3)c2c1] |
| 3-(6-(piperidin-3-yl)pyridin-2-yl)-5- (trifluoromethyl)pyrazolo[1,5- a]pyridine | 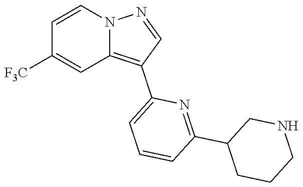 [SMILES: FC(F)(F)c1ccn2ncc(-c3cccc(C4CCCNC4)n3)c2c1] |
| 5-methoxy-3-(6-(piperidin-3- yl)pyridin-2-yl)pyrazolo[1,5-a]pyridine | 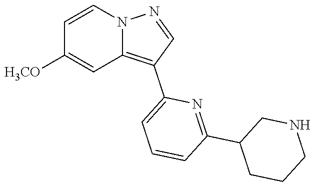 [SMILES: COc1ccn2ncc(-c3cccc(C4CCCNC4)n3)c2c1] |
| 3-(6-(piperidin-3-yl)pyridin-2- yl)pyrazolo[1,5-a]pyridin-5-ol | 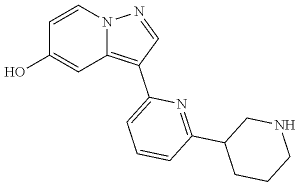 [SMILES: Oc1ccn2ncc(-c3cccc(C4CCCNC4)n3)c2c1] |
| 5-isopropoxy-3-(6-(piperidin-3- yl)pyridin-2-yl)pyrazolo[1,5-a]pyridine | 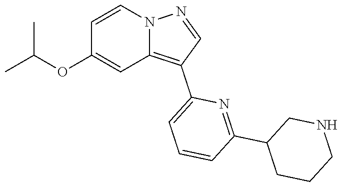 [SMILES: CC(C)Oc1ccn2ncc(-c3cccc(C4CCCNC4)n3)c2c1] |
| 5-(cyclopropylmethoxy)-3-(6- (piperidin-3-yl)pyridin-2- yl)pyrazolo[1,5-a]pyridine | 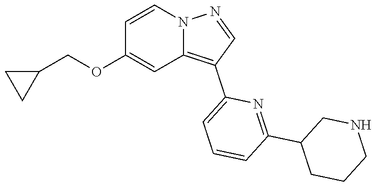 [SMILES: c1cc(-c2cnn3ccc(OCC4CC4)cc23)nc(C2CCCNC2)c1] |
| 5-(oxetan-3-ylmethoxy)-3-(6- (piperidin-3-yl)pyridin-2- yl)pyrazolo[1,5-a]pyridine | 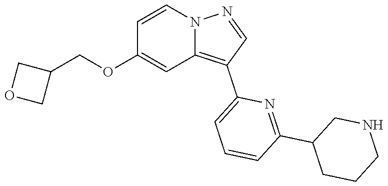 [SMILES: c1cc(-c2cnn3ccc(OCC4COC4)cc23)nc(C2CCCNC2)c1] |
| 4-(3-(6-(piperidin-3-yl)pyridin-2- yl)pyrazolo[1,5-a]pyridin-5- yl)morpholine | 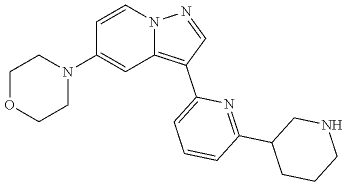 [SMILES: c1cc(-c2cnn3ccc(N4CCOCC4)cc23)nc(C2CCCNC2)c1] |
| 3-(6-(piperidin-3-yl)pyridin-2- yl)pyrazolo[1,5-a]pyridine-5- carbonitrile | 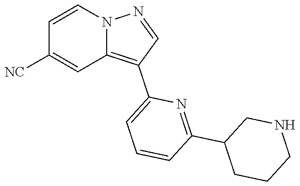 [SMILES: N#Cc1ccn2ncc(-c3cccc(C4CCCNC4)n3)c2c1] |
| 5-cyclopropyl-3-(6-(piperidin-3- yl)pyridin-2-yl)pyrazolo[1,5-a]pyridine | 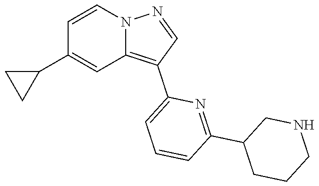 [SMILES: c1cc(-c2cnn3ccc(C4CC4)cc23)nc(C2CCCNC2)c1] |
| 3-(6-(piperidin-3-yl)pyridin-2- yl)pyrazolo[1,5-a]pyridin-5-amine | 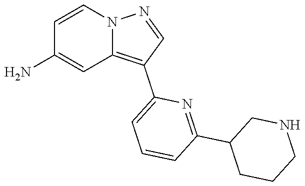 [SMILES: Nc1ccn2ncc(-c3cccc(C4CCCNC4)n3)c2c1] |
| N-methyl-3-(6-(piperidin-3-yl)pyridin- 2-yl)pyrazolo[1,5-a]pyridin-5-amine | 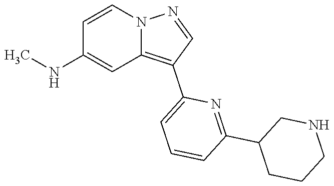 [SMILES: CNc1ccn2ncc(-c3cccc(C4CCCNC4)n3)c2c1] |
| N-ethyl-3-(6-(piperidin-3-yl)pyridin-2- yl)pyrazolo[1,5-a]pyridin-5-amine | 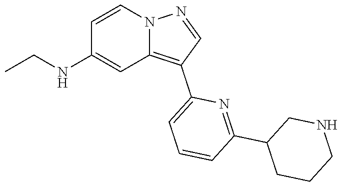 [SMILES: CCNc1ccn2ncc(-c3cccc(C4CCCNC4)n3)c2c1] |
| 3-(methylsulfonamido)-N-(3-(6- (piperidin-3-yl)pyridin-2- yl)pyrazolo[1,5-a]pyridin-5- yl)propanamide | 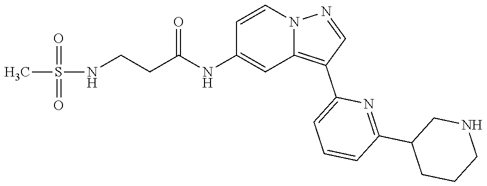 [SMILES: CS(=O)(=O)NCCC(=O)Nc1ccn2ncc(-c3cccc(C4CCCNC4)n3)c2c1] |
| 3-(methylsulfonamido)-N-(3-(6- (piperidin-3-yl)pyridin-2- yl)pyrazolo[1,5-a]pyridin-6- yl)propanamide | 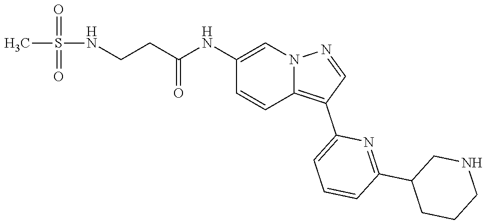 [SMILES: CS(=O)(=O)NCCC(=O)Nc1ccc2c(-c3cccc(C4CCCNC4)n3)cnn2c1] |
| N-(2-(methylsulfonamido)ethyl)-3-(6- (piperidin-3-yl)pyridin-2- yl)pyrazolo[1,5-a]pyridine-6- carboxamide | 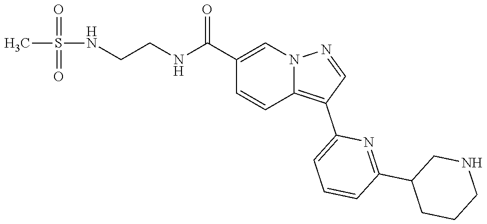 [SMILES: CS(=O)(=O)NCCNC(=O)c1ccc2c(-c3cccc(C4CCCNC4)n3)cnn2c1] |
| N-(2-oxo-2-(3-(6-(pyrazolo[1,5- a]pyridin-3-yl)pyridin-2-yl)piperidin-1- yl)ethyl)methanesulfonamide | 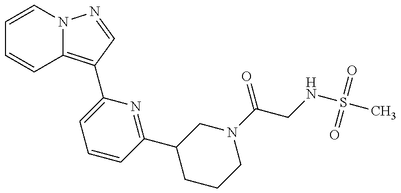 [SMILES: CS(=O)(=O)NCC(=O)N1CCCC(c2cccc(-c3cnn4ccccc34)n2)C1] |
| 3-(6-(1-(methylsulfonyl)piperidin-3- yl)pyridin-2-yl)pyrazolo[1,5-a]pyridine | 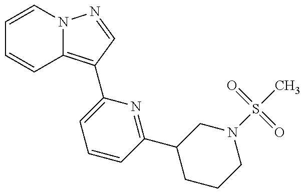 [SMILES: CS(=O)(=O)N1CCCC(c2cccc(-c3cnn4ccccc34)n2)C1] |
| 3-(4-methoxy-6-(piperidin-3- yl)pyridin-2-yl)pyrazolo[1,5-a]pyridine | 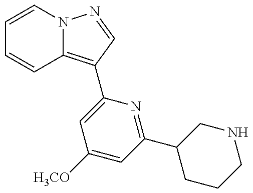 [SMILES: COc1cc(-c2cnn3ccccc23)nc(C2CCCNC2)c1] |
| 3-(4-chloro-6-(piperidin-3-yl)pyridin- 2-yl)pyrazolo[1,5-a]pyridine | 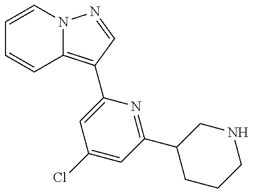 [SMILES: Clc1cc(-c2cnn3ccccc23)nc(C2CCCNC2)c1] |
| 3-(5-chloro-6-(piperidin-3-yl)pyridin- 2-yl)pyrazolo[1,5-a]pyridine | 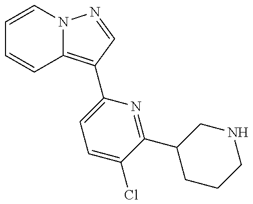 [SMILES: Clc1ccc(-c2cnn3ccccc23)nc1C1CCCNC1] |
| 3-(3-chloro-6-(piperidin-3-yl)pyridin- 2-yl)pyrazolo[1,5-a]pyridine | 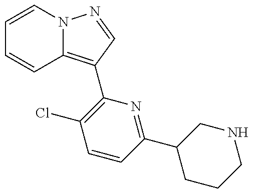 [SMILES: Clc1ccc(C2CCCNC2)nc1-c1cnn2ccccc12] |
| 3-(4-fluoro-6-(piperidin-3-yl)pyridin-2- yl)pyrazolo[1,5-a]pyridine | 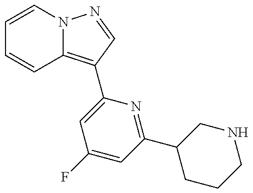 [SMILES: Fc1cc(-c2cnn3ccccc23)nc(C2CCCNC2)c1] |
| 3-(3-fluoro-6-(piperidin-3-yl)pyridin-2- yl)pyrazolo[1,5-a]pyridine | 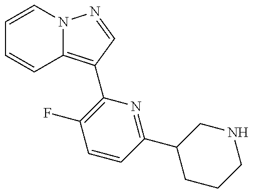 [SMILES: Fc1ccc(C2CCCNC2)nc1-c1cnn2ccccc12] |
| 3-(5-fluoro-6-(piperidin-3-yl)pyridin-2- yl)pyrazolo[1,5-a]pyridine | 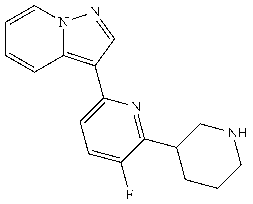 [SMILES: Fc1ccc(-c2cnn3ccccc23)nc1C1CCCNC1] |
| 5-chloro-3-(4-chloro-6-(piperidin-3- yl)pyridin-2-yl)pyrazolo[1,5-a]pyridine | 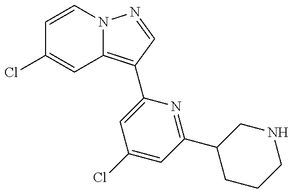 [SMILES: Clc1cc(-c2cnn3ccc(Cl)cc23)nc(C2CCCNC2)c1] |
| 5-chloro-3-(4-fluoro-6-(piperidin-3- yl)pyridin-2-yl)pyrazolo[1,5-a]pyridine | 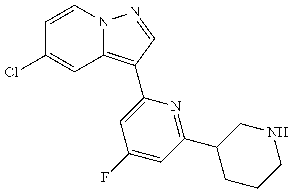 [SMILES: Fc1cc(-c2cnn3ccc(Cl)cc23)nc(C2CCCNC2)c1] |
| 3-(4-chloro-6-(piperidin-3-yl)pyridin- 2-yl)-5-(trifluoromethyl)pyrazolo[1,5- a]pyridine | 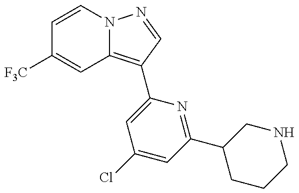 [SMILES: FC(F)(F)c1ccn2ncc(-c3cc(Cl)cc(C4CCCNC4)n3)c2c1] |
| 3-(6-(piperazin-1-yl)pyridin-2- yl)pyrazolo[1,5-a]pyridine | 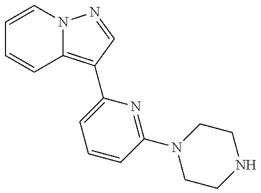 [SMILES: c1cc(-c2cnn3ccccc23)nc(N2CCNCC2)c1] |
| N-methyl-3-(6-(piperazin-1-yl)pyridin- 2-yl)pyrazolo[1,5-a]pyridin-5-amine | 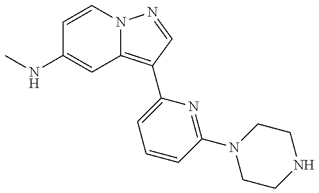 [SMILES: CNc1ccn2ncc(-c3cccc(N4CCNCC4)n3)c2c1] |
| 3-(4-chloro-6-(piperazin-1-yl)pyridin- 2-yl)-N-methylpyrazolo[1,5-a]pyridin- 5-amine | 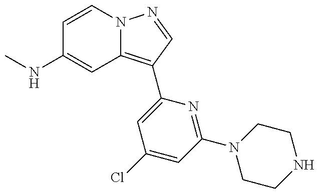 [SMILES: CNc1ccn2ncc(-c3cc(Cl)cc(N4CCNCC4)n3)c2c1] |
| 3-(6-(piperazin-1-yl)pyridin-2-yl)-5- (trifluoromethyl)pyrazolo[1,5- a]pyridine | 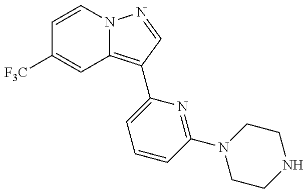 [SMILES: FC(F)(F)c1ccn2ncc(-c3cccc(N4CCNCC4)n3)c2c1] |
| 3-(4-chloro-6-(piperazin-1-yl)pyridin- 2-yl)-5-(trifluoromethyl)pyrazolo[1,5- a]pyridine | 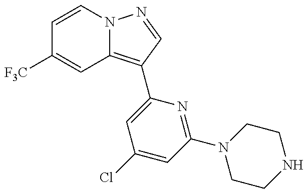 [SMILES: FC(F)(F)c1ccn2ncc(-c3cc(Cl)cc(N4CCNCC4)n3)c2c1] |
| 5-chloro-3-(6-(piperazin-1-yl)pyridin- 2-yl)pyrazolo[1,5-a]pyridine | 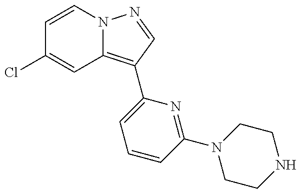 [SMILES: Clc1ccn2ncc(-c3cccc(N4CCNCC4)n3)c2c1] |
| 5-chloro-3-(4-chloro-6-(piperazin-1- yl)pyridin-2-yl)pyrazolo[1,5-a]pyridine | 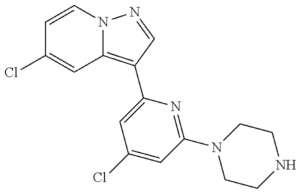 [SMILES: Clc1cc(-c2cnn3ccc(Cl)cc23)nc(N2CCNCC2)c1] |
| 5-chloro-3-(4-fluoro-6-(piperazin-1- yl)pyridin-2-yl)pyrazolo[1,5-a]pyridine | 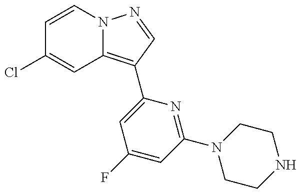 [SMILES: Fc1cc(-c2cnn3ccc(Cl)cc23)nc(N2CCNCC2)c1] |
| 3-(6-(pyrazolo[1,5-a]pyridin-3- yl)pyridin-2-yl)-3,6- diazabicyclo[3.1.1]heptane | 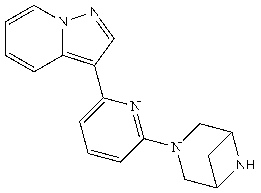 [SMILES: c1cc(-c2cnn3ccccc23)nc(N2CC3CC(C2)N3)c1] |
| 3-(6-(5-chloropyrazolo[1,5-a]pyridin- 3-yl)pyridin-2-yl)-3,6- diazabicyclo[3.1.1]heptane | 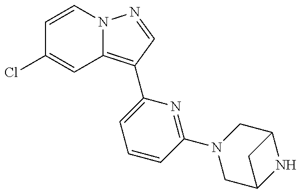 [SMILES: Clc1ccn2ncc(-c3cccc(N4CC5CC(C4)N5)n3)c2c1] |
| 3-(4-chloro-6-(5-chloropyrazolo[1,5- a]pyridin-3-yl)pyridin-2-yl)-3,6- diazabicyclo[3.1.1]heptane | 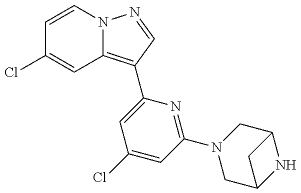 [SMILES: Clc1cc(-c2cnn3ccc(Cl)cc23)nc(N2CC3CC(C2)N3)c1] |
| 2-(6-(pyrazolo[1,5-a]pyridin-3- yl)pyridin-2-yl)-2,5- diazabicyclo[2.2.2]octane | 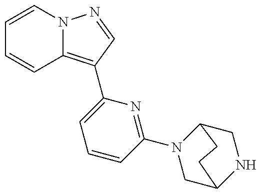 [SMILES: c1cc(-c2cnn3ccccc23)nc(N2CC3CCC2CN3)c1] |
| 3-(6-((3S,5R)-3,5-dimethylpiperazin- 1-yl)pyridin-2-yl)pyrazolo[1,5- a]pyridine | 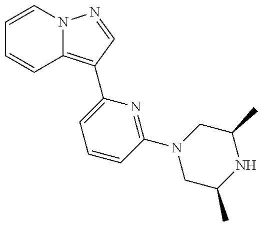 [SMILES: C[C@@H]1CN(c2cccc(-c3cnn4ccccc34)n2)C[C@H](C)N1] |
| 3-(6-((3R,5R)-3,5-dimethylpiperazin- 1-yl)pyridin-2-yl)pyrazolo[1,5- a]pyridine | 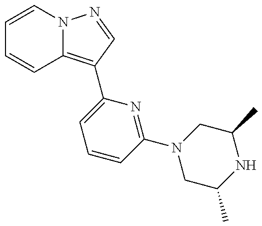 [SMILES: C[C@@H]1CN(c2cccc(-c3cnn4ccccc34)n2)C[C@@H](C)N1] |
| 3-(4-chloro-6-((3S,5R)-3,5- dimethylpiperazin-1-yl)pyridin-2- yl)pyrazolo[1,5-a]pyridine | 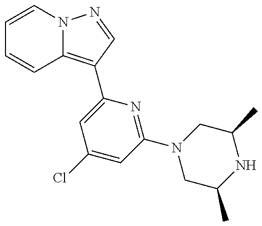 [SMILES: C[C@@H]1CN(c2cc(Cl)cc(-c3cnn4ccccc34)n2)C[C@H](C)N1] |
| 3-(6-((3S,5R)-3,5-dimethylpiperazin- 1-yl)-4-fluoropyridin-2- yl)pyrazolo[1,5-a]pyridine | 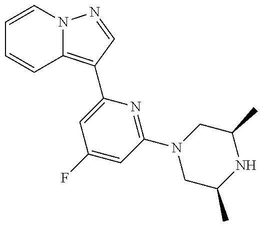 [SMILES: C[C@@H]1CN(c2cc(F)cc(-c3cnn4ccccc34)n2)C[C@H](C)N1] |
| 3-(6-((3S,5R)-3,5-dimethylpiperazin- 1-yl)pyridin-2-yl)-N- methylpyrazolo[1,5-a]pyridin-5-amine | 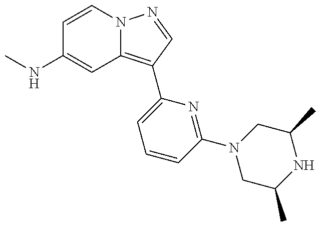 |
| 3-(4-chloro-6-((3S,5R)-3,5- dimethylpiperazin-1-yl)pyridin-2-yl)- N-methylpyrazolo[1,5-a]pyridin-5- amine | 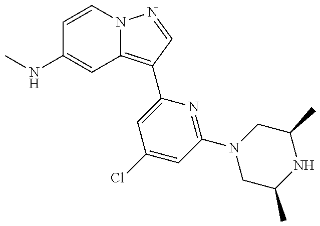 |
| 3-(6-((3S,5R)-3,5-dimethylpiperazin- 1-yl)pyridin-2-yl)-5- (trifluoromethyl)pyrazolo[1,5- a]pyridine | 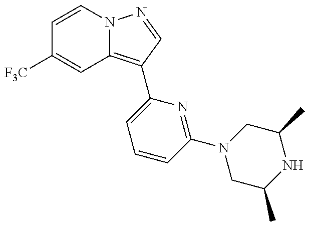 [SMILES: C[C@@H]1CN(c2cccc(-c3cnn4ccc(C(F)(F)F)cc34)n2)C[C@H](C)N1] |
| 3-(4-chloro-6-((3S,5R)-3,5- dimethylpiperazin-1-yl)pyridin-2-yl)-5- (trifluoromethyl)pyrazolo[1,5- a]pyridine | 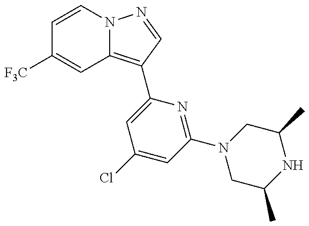 [SMILES: C[C@@H]1CN(c2cc(Cl)cc(-c3cnn4ccc(C(F)(F)F)cc34)n2)C[C@H](C)N1] |
| 5-chloro-3-(6-((3S,5R)-3,5- dimethylpiperazin-1-yl)pyridin-2- yl)pyrazolo[1,5-a]pyridine | 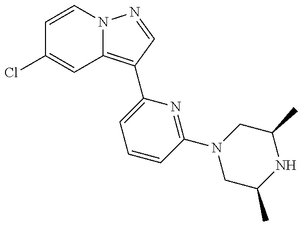 [SMILES: C[C@@H]1CN(c2cccc(-c3cnn4ccc(Cl)cc34)n2)C[C@H](C)N1] |
| 5-chloro-3-(6-((3S,5R)-3,5- dimethylpiperazin-1-yl)-4- fluoropyridin-2-yl)pyrazolo[1,5- a]pyridine | 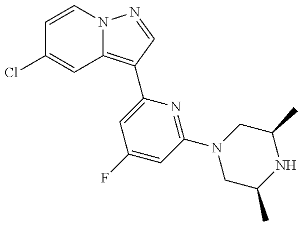 [SMILES: C[C@@H]1CN(c2cc(F)cc(-c3cnn4ccc(Cl)cc34)n2)C[C@H](C)N1] |
| 5-chloro-3-(4-chloro-6-((3S,5R)-3,5- dimethylpiperazin-1-yl)pyridin-2- yl)pyrazolo[1,5-a]pyridine | 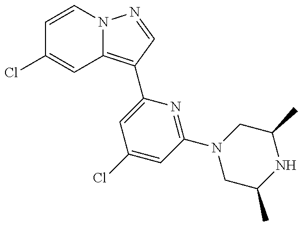 [SMILES: C[C@@H]1CN(c2cc(Cl)cc(-c3cnn4ccc(Cl)cc34)n2)C[C@H](C)N1] |
| 5-chloro-3-(5-chloro-6-((3S,5R)-3,5- dimethylpiperazin-1-yl)pyridin-2- yl)pyrazolo[1,5-a]pyridine | 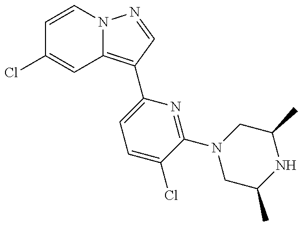 [SMILES: C[C@@H]1CN(c2nc(-c3cnn4ccc(Cl)cc34)ccc2Cl)C[C@H](C)N1] |
| 5-chloro-3-(6-((3S,5R)-3,5- dimethylpiperazin-1-yl)-5- fluoropyridin-2-yl)pyrazolo[1,5- a]pyridine | 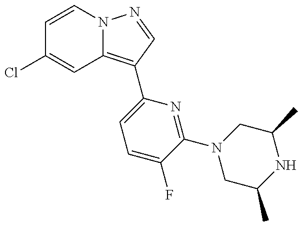 [SMILES: C[C@@H]1CN(c2nc(-c3cnn4ccc(Cl)cc34)ccc2F)C[C@H](C)N1] |
| 3-(4-chloro-6-(piperidin-3-yl)pyridin- 2-yl)-N-methylpyrazolo[1,5-a]pyridin- 5-amine | 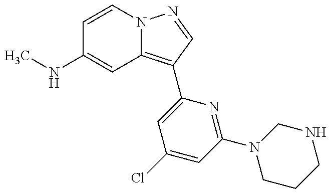 [SMILES: CNc1ccn2ncc(-c3cc(Cl)cc(N4CCCNC4)n3)c2c1] |
| 5-chloro-3-(3-chloro-6-((3S,5R)-3,5- dimethylpiperazin-1-yl)pyridin-2- yl)pyrazolo[1,5-a]pyridine | 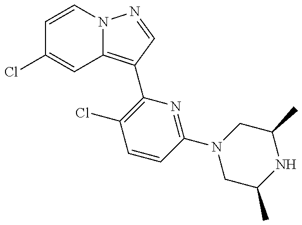 [SMILES: C[C@@H]1CN(c2ccc(Cl)c(-c3cnn4ccc(Cl)cc34)n2)C[C@H](C)N1] |
| 5-chloro-3-(6-((3S,5R)-3,5- dimethylpiperazin-1-yl)-3- fluoropyridin-2-yl)pyrazolo[1,5- a]pyridine | 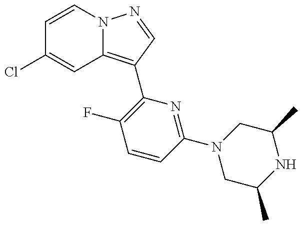 [SMILES: C[C@@H]1CN(c2ccc(F)c(-c3cnn4ccc(Cl)cc34)n2)C[C@H](C)N1] |
| 3-(4-chloro-6-(piperidin-3-yl)pyridin- 2-yl)-5-isopropoxypyrazolo[1,5- a]pyridine | 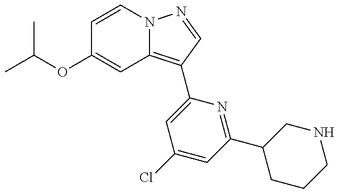 [SMILES: CC(C)Oc1ccn2ncc(-c3cc(Cl)cc(C4CCCNC4)n3)c2c1] |
| 5-chloro-3-(6-(3,3,5,5- tetramethylpiperazin-1-yl)pyridin-2- yl)pyrazolo[1,5-a]pyridine | 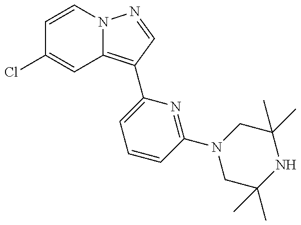 [SMILES: CC1(C)CN(c2cccc(-c3cnn4ccc(Cl)cc34)n2)CC(C)(C)N1] |
| 2-(methylsulfonyl)-N-(3-(6-(piperidin- 3-yl)pyridin-2-yl)pyrazolo[1,5- a]pyridin-5-yl)acetamide | 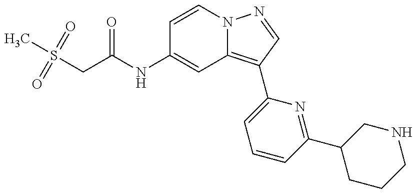 [SMILES: CS(=O)(=O)CC(=O)Nc1ccn2ncc(-c3cccc(C4CCCNC4)n3)c2c1] |
| 2-(methylsulfonamido)-N-(3-(6- (piperidin-3-yl)pyridin-2- yl)pyrazolo[1,5-a]pyridin-5- yl)acetamide | 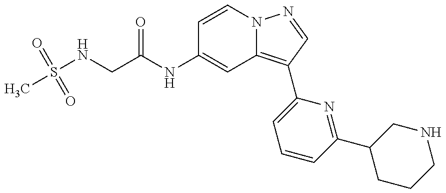 [SMILES: CS(=O)(=O)NCC(=O)Nc1ccn2ncc(-c3cccc(C4CCCNC4)n3)c2c1] |
| (R)-3-(6-(3-methylpiperazin-1- yl)pyridin-2-yl)pyrazolo[1,5-a]pyridine | 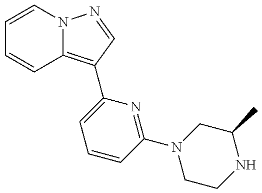 [SMILES: C[C@@H]1CN(c2cccc(-c3cnn4ccccc34)n2)CCN1] |
| (S)-3-(6-(3-methylpiperazin-1- yl)pyridin-2-yl)pyrazolo[1,5-a]pyridine | 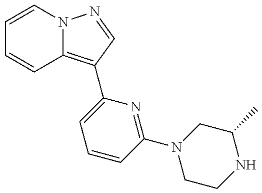 [SMILES: C[C@H]1CN(c2cccc(-c3cnn4ccccc34)n2)CCN1] |
| 5-chloro-3-(6-((3S,5R)-3,4,5- trimethylpiperazin-1-yl)pyridin-2- yl)pyrazolo[1,5-a]pyridine | 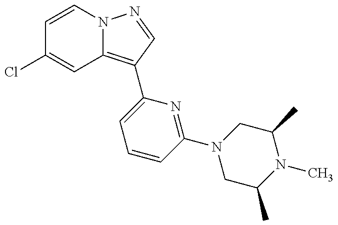 [SMILES: C[C@@H]1CN(c2cccc(-c3cnn4ccc(Cl)cc34)n2)C[C@H](C)N1C] |
| N-(3-(6-(piperidin-3-yl)pyridin-2- yl)pyrazolo[1,5-a]pyridin-5- yl)picolinamide | 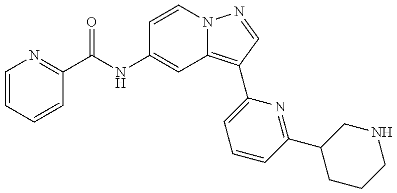 [SMILES: O=C(Nc1ccn2ncc(-c3cccc(C4CCCNC4)n3)c2c1)c1ccccn1] |
| N-(3-(6-(piperidin-3-yl)pyridin-2- yl)pyrazolo[1,5-a]pyridin-5-yl)-2- (pyridin-2-yloxy)acetamide | 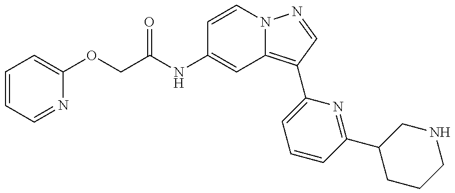 [SMILES: O=C(COc1ccccn1)Nc1ccn2ncc(-c3cccc(C4CCCNC4)n3)c2c1] |
| 3-cyano-N-(3-(6-(piperidin-3- yl)pyridin-2-yl)pyrazolo[1,5-a]pyridin- 5-yl)propanamide | 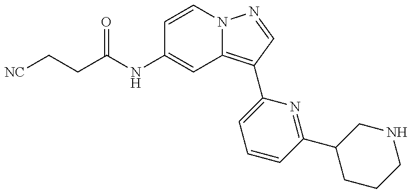 [SMILES: N#CCCC(=O)Nc1ccn2ncc(-c3cccc(C4CCCNC4)n3)c2c1] |
| 4-cyano-N-(3-(6-(piperidin-3- yl)pyridin-2-yl)pyrazolo[1,5-a]pyridin- 5-yl)butanamide | 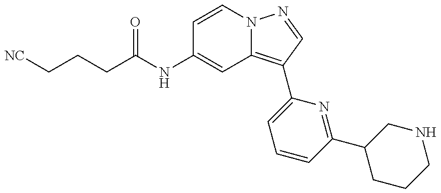 [SMILES: N#CCCCC(=O)Nc1ccn2ncc(-c3cccc(C4CCCNC4)n3)c2c1] |
| N-(3-(6-(piperidin-3-yl)pyridin-2- yl)pyrazolo[1,5-a]pyridin-5-yl)-2- (pyridin-4-yloxy)acetamide | 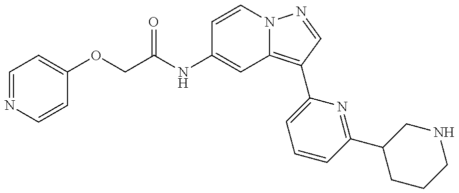 [SMILES: O=C(COc1ccncc1)Nc1ccn2ncc(-c3cccc(C4CCCNC4)n3)c2c1] |
| 2-cyano-N-(3-(6-(piperidin-3- yl)pyridin-2-yl)pyrazolo[1,5-a]pyridin- 5-yl)acetamide | 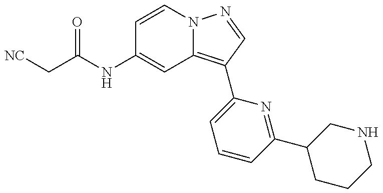 [SMILES: N#CCC(=O)Nc1ccn2ncc(-c3cccc(C4CCCNC4)n3)c2c1] |
| N-(3-(6-(piperidin-3-yl)pyridin-2- yl)pyrazolo[1,5-a]pyridin-5-yl)-2- (pyridin-3-yl)acetamide | 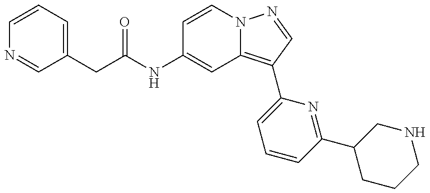 [SMILES: O=C(Cc1cccnc1)Nc1ccn2ncc(-c3cccc(C4CCCNC4)n3)c2c1] |
| N-(3-(6-(piperidin-3-yl)pyridin-2- yl)pyrazolo[1,5-a]pyridin-5-yl)-2- (pyridin-4-ylamino)acetamide | 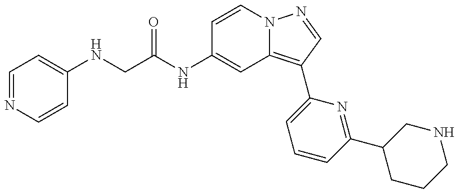 [SMILES: O=C(CNc1ccncc1)Nc1ccn2ncc(-c3cccc(C4CCCNC4)n3)c2c1] |
| N-(3-(6-(piperidin-3-yl)pyridin-2- yl)pyrazolo[1,5-a]pyridin-5-yl)-2- (pyridin-3-ylamino)acetamide | 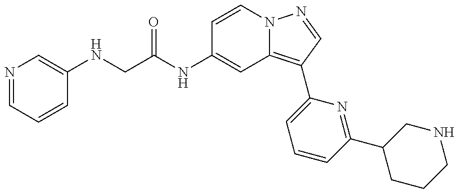 [SMILES: O=C(CNc1cccnc1)Nc1ccn2ncc(-c3cccc(C4CCCNC4)n3)c2c1] |
| N-(3-(6-(piperidin-3-yl)pyridin-2- yl)pyrazolo[1,5-a]pyridin-5-yl)-2- (pyridin-2-yl)acetamide | 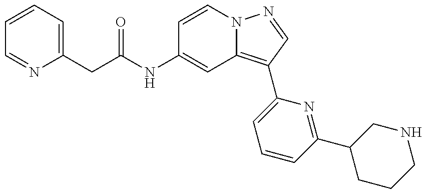 [SMILES: O=C(Cc1ccccn1)Nc1ccn2ncc(-c3cccc(C4CCCNC4)n3)c2c1] |

### Pharmaceutical Compositions

The compounds of Formula I may be formulated into pharmaceutical compositions.

In another aspect, the invention provides pharmaceutical compositions comprising LRRK2 kinase inhibitors according to the invention, or salts of the pharmaceutical compositions together with a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain embodiments, compounds of the invention are administered intravenously in a hospital setting. In certain other embodiments, administration may preferably be by the oral route.

The characteristics of the carrier will depend on the route of administration. As used herein, the term “pharmaceutically acceptable” means a non-toxic material that is compatible with a biological system such as a cell, a cell culture, a tissue, or an organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, A. Gennaro, Mack Publishing Co., Easton, Pa., 1990.

As used herein, the term “pharmaceutically acceptable salts” refers to salts that retain the desired biological activity of the above-identified compounds and exhibit minimal or no undesired toxicological effects. Examples of such salts include, without limitation, acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR⁺Z⁻, wherein R is hydrogen, alkyl, or benzyl, and Z is a counterion, including chloride, bromide, iodide, —O— alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. A dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods described herein.

### Methods of Use

In another aspect, the present disclosure generally relates to methods for treating a disease or disorder associated with LRRK2 kinase activity. These methods comprise administering to a subject in need thereof a therapeutically effective amount of an LRRK2 inhibitor.

In some embodiments, the LRRK2 inhibitor is a compound of Formula (I), or a pharmaceutically acceptable salt, prodrug, solvate, hydrate, isomer, deuterated form, or tautomer thereof. In particular embodiments, the LRRK2 inhibitor comprises a compound selected from Table 1 as described herein.

In another aspect, the present disclosure provides a compound obtainable by, or obtained by, a method for preparing a compound as described herein (e.g., a method comprising one or more steps described in the Examples).

In another aspect, the present disclosure provides a pharmaceutical composition comprising a compound of Formula (I) or a pharmaceutically acceptable salt, prodrug, solvate, hydrate, isomer, deuterated form, or tautomer thereof, and a pharmaceutically acceptable diluent or carrier.

In another aspect, the present disclosure provides an intermediate as described herein, being suitable for use in a method for preparing a compound as described herein (e.g., the intermediate is selected from the intermediates described in the Examples).

In another aspect, the present disclosure provides a method of inhibiting LRRK2 kinase activity, which comprises administering to a patient in need thereof a therapeutically effective amount of a compound of Formula (I) or a pharmaceutically acceptable salt, prodrug, solvate, hydrate, isomer, deuterated form, or tautomer thereof.

In another aspect, the present disclosure provides a method of treating Parkinson's disease, comprising administering to a patient in need thereof a therapeutically effective amount of a compound of Formula (I) or a pharmaceutically acceptable salt, prodrug, solvate, hydrate, isomer, deuterated form, or tautomer thereof.

The compositions and methods provided herein may be used for the treatment of a wide variety of diseases or disorders associated with LRRK2 kinase activity. More particularly, diseases or disorders that may be treated by the compositions and methods of the invention include, without limitation, Parkinson's disease, Alzheimer's disease, multiple sclerosis, HIV-induced dementia, amyotrophic lateral sclerosis, ischemic stroke, traumatic brain injury, and spinal cord injury.

The concentration and route of administration to the patient will vary depending on the disease or disorder to be treated. The compounds, pharmaceutically acceptable salts thereof, or pharmaceutical compositions comprising such compounds and salts, may be co-administered with other compounds, e.g., those increasing dopamine levels in the brain, or used in combination with other treatments, such as surgical intervention, either as an adjuvant prior to surgery or post-operatively.

The present disclosure provides methods of treating, preventing, or ameliorating a disease or disorder in which LRRK2 kinase plays a role by administering to a patient in need thereof a therapeutically effective amount of an LRRK2 inhibitor. The methods of the present disclosure can be used in the treatment of a variety of LRRK2-dependent diseases and disorders.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### GENERAL REACTION SCHEMES, INTERMEDIATES AND EXAMPLES

The compounds of the present invention may be prepared using commercially available reagents and intermediates in the synthetic methods and reaction schemes described herein, or may be prepared using other reagents and conventional methods well known to those skilled in the art. Abbreviations for some of the reagents as described herein may include the following:

| | |
|---|---|
| ACN | Acetonitrile |
| AcOH | Acetic Acid |
| aq | Aqueous |
| ATP | Adenosine triphosphate |
| DCM | Dichloromethane |
| DIEA | Diisopropylethylamine |
| DMF | Dimethylformanide |
| DMSO | Dimethylsulfoxide |
| DTT | Dithiothreitol |
| EDCI | N-(3-Dimethylaminopropyl)-N′- |
| | ethylcarbodiimide |
| EDTA | Ethylenediaminetetraacetic acid |
| EGTA | Egtazic acid |
| ESI | Electrospray ionization |
| Et₃N | Triethylamine |
| EtOAc | Ethyl Acetate |
| EtOH | Ethanol |
| FA | Formic Acid |
| ¹H-NMR | Proton nuclear magnetic resonance |
| HOBt | 1-Hydroxybenzotriazole |
| HPLC | High performance liquid chromatography |
| hr, hrs | hours |
| LCMS | Liquid chromatography-mass spectrometry |
| MeOH | Methanol |
| MS | Mass spectrometry |
| MsCl | Methanesulfonyl chloride |
| m/z | Mass to charge ratio |
| NBS | N-Bromosuccinimide |
| NCS | N-Chlorosuccinimide |
| NIS | N-Iodosuccinimide |
| NMI | N-Methyl imidazole |
| NMP | N-Methyl-2-pyrrolidinone |
| Pd(dppf)Cl₂ | Bis(diphenylphosphino)ferrocene)palladium(II) |
| | dichloride |
| | 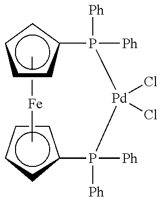 |
| Pd/C | Palladium on Carbon |
| PE | Petroleum ether |
| PFA | Paraformaldehyde |
| psi | Pounds per square inch |
| Rac | Racemic, racemate |
| rt | Room temperature |
| RuPhos PdG3 | (2-Dicyclohexylphosphino-2′,6′-diisopropoxy- |
| | 1,1′-biphenyl)[2-(2′-amino-1,1′- |
| | biphenyl)]palladium(II)methanesulfonate |
| | 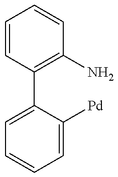 |
| | 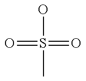 |
| | 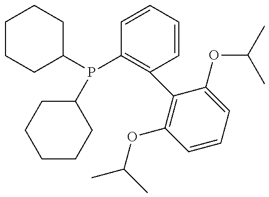 [SMILES: CC(C)Oc1cccc(OC(C)C)c1-c1ccccc1P(C1CCCCC1)C1CCCCC1] |
| SFC | Supercritical fluid chromatograph |
| TCFH | Chloro-N,N,N′,N′-tetramethylformamidinium |
| | hexafluorophosphate |
| TEA | Triethylamine |
| TFA | Trifluoroacetic acid |
| THF | Tetrahydrofuran |
| TMS | Trimethylsilyl |
| t_{R} | Retention time |
| Tris | Tris(hydroxymethyl)aminomethane |
| TR-FRET | Time-resolved fluorescence energy transfer |

### Intermediate A

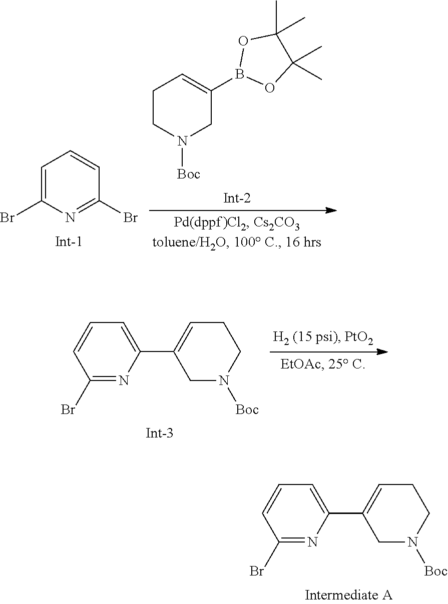 [SMILES: Brc1cccc(Br)n1 ; CC(C)(C)OC(=O)N1CCC=C(B2OC(C)(C)C(C)(C)O2)C1 ; CC(C)(C)OC(=O)N1CCC=C(c2cccc(Br)n2)C1 ; CC(C)(C)OC(=O)N1CCC=C(c2cccc(Br)n2)C1]

Step A: A mixture of 2,6-dibromopyridine (Int-1) (4.6 g, 19.4 mmol), tert-butyl 5-(4,4,5,5-tetramethyl-1,3,2-dioxaborolan-2-yl)-3,6-dihydropyridine-1(2H)-carboxylate (Int-2) (5 g, 16.17 mmol), Cs₂CO₃ (10.54 g, 32.34 mmol), and Pd(dppf)Cl₂ (2.37 g, 3.23 mmol) in toluene (50 mL) and H₂O (13 mL) is degassed and purged with N₂ three times. The mixture is stirred at 100° C. for 16 hours under N₂. The reaction mixture is extracted with EtOAc three times. The combined organic layers are washed with brine three times, dried over anhydrous Na₂SO₄, filtered and concentrated under reduced pressure to give a residue which is purified by flash silica gel chromatography (eluent of 0-5% ethyl acetate/petroleum ether gradient) to yield the title compound tert-butyl 5-(6-bromo-2-pyridyl)-3,6-dihydro-2H-pyridine-1-carboxylate (Int-3) (3.32 g, 60.5% yield) as yellow oil.

¹H NMR (400 MHz, DMSO-d₆) 5=7.76-7.70 (m, 1H), 7.64 (br d, J=7.6 Hz, 1H), 7.52 (d, J=7.6 Hz, 1H), 6.84 (br s, 1H), 4.26 (br s, 2H), 3.46 (br t, J=5.6 Hz, 2H), 2.30 (br d, J=3.6 Hz, 2H), 1.43 (s, 9H).

Step B: To a solution of tert-butyl 5-(6-bromo-2-pyridyl)-3,6-dihydro-2H-pyridine-1-carboxylate (Int-3) (1 g, 2.95 mmol) in EtOAc (10 mL) is added PtO₂ (66.9 mg, 294.8 μmol). The mixture is placed under 15 psi of H₂ at 25° C. for 16 hr. The reaction mixture is filtered, and the filtrate is concentrated under vacuum to yield the title compound tert-butyl 3-(6-bromo-2-pyridyl)piperidine-1-carboxylate (Intermediate A) (950 mg) as a white solid. LCMS (ESI MS) m/z=341.0 [M+H]+ 1.

### Intermediate B

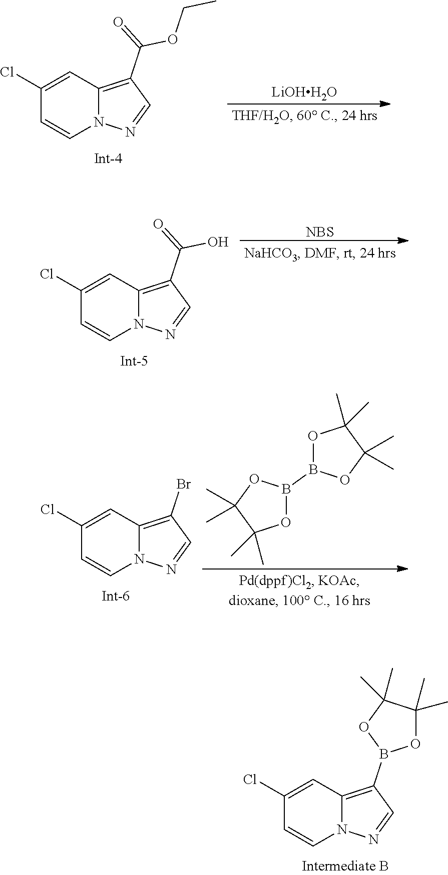

Step A: To a solution of ethyl 5-chloropyrazolo[1,5-a]pyridine-3-carboxylate (Int-4) (800 mg, 3.56 mmol) in H₂O (2 mL) and THF (2 mL) is added LiOH·H₂O (597.8 mg, 14.24 mmol). The mixture is stirred at 60° C. for 24 hr. The reaction mixture is quenched with HCl (1M) and extracted with EtOAc (3×). The combined organic layers are washed with brine twice, dried over anhydrous Na₂SO₄, filtered and concentrated under reduced pressure to give the title compound 5-chloropyrazolo[1,5-a]pyridine-3-carboxylic acid (Int-6) (700 mg) as a white solid. LCMS (ESI MS) m/z=197.1 [M+H]+ 1. The crude product is used in the next step without additional purification.

Step B: To a solution of 5-chloropyrazolo[1,5-a]pyridine-3-carboxylic acid (Int-5) (400 mg, 2.03 mmol) in DMF (1 mL) is added NBS (398 mg, 2.24 mmol) and NaHCO₃ (256.4 mg, 3.05 mmol, 119 μL). The mixture is stirred at 25° C. for 24 hr. The reaction mixture is quenched with H₂O and extracted with EtOAc three times. The combined organic layers are washed with brine twice, dried over anhydrous Na₂SO₄, filtered and concentrated under reduced pressure to give the title compound 3-bromo-5-chloro-pyrazolo[1,5-a]pyridine (Int-6) (300 mg, 1.30 mmol, 63% yield) as a yellow solid. LCMS (ESI MS) m/z=233.0 [M+H]+ 1. The crude product is used directly in the next step without additional purification.

Step C: To a solution of 3-bromo-5-chloropyrazolo[1,5-a]pyridine (Int-6) (300 mg, 1.30 mmol) and 4,4,5,5-tetramethyl-2-(4,4,5,5-tetramethyl-1,3,2-dioxaborolan-2-yl)-1,3,2-dioxaborolane (263.3 mg, 1.04 mmol) in dioxane (3 mL) are added Pd(dppf)Cl₂ (211.7 mg, 259.2 μmol) and potassium acetate (254.4 mg, 2.59 mmol). The mixture is stirred at 100° C. for 16 hrs under a N₂ atmosphere. The reaction mixture is quenched with H₂O and extracted with EtOAc three times. The combined organic layers are washed with brine twice, dried over anhydrous Na₂SO₄, filtered and concentrated under reduced pressure to give a residue which is purified by flash silica gel chromatography (eluent of 0-100% DCM/petroleum ether gradient) to yield the title compound 5-chloro-3-(4,4,5,5-tetramethyl-1,3,2-dioxaborolan-2-yl)pyrazolo[1,5-a]pyridine (Intermediate B) (100 mg) as a white solid. LCMS (ESI MS) m/z=279.1 [M+H]+ 1.

### Intermediate C

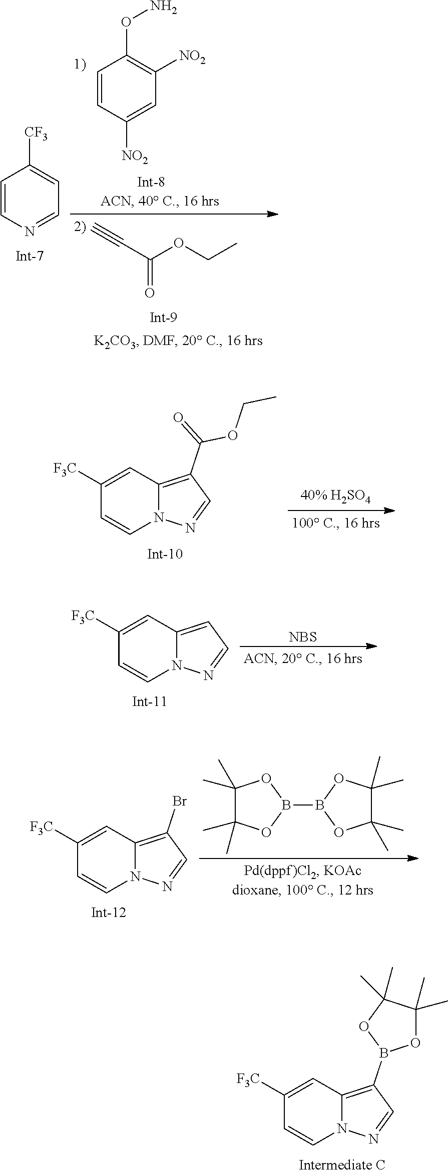

Step A: A solution of 4-trifluoromethylpyridine (Int-7) (3 g, 20.39 mmol) and O-(2,4-dinitrophenyl)hydroxylamine (Int-8) (4.06 g, 20.39 mmol) in ACN (40 mL) is stirred at 40° C. for 16 hr. The solution is concentrated under reduced pressure. The residue is dissolved in DMF (40 mL), and then K₂CO₃ (5.64 g, 40.79 mmol) and ethyl propiolate (Int-9) (2.00 g, 20.39 mmol, 2.0 mL) are added. The mixture is stirred at 20° C. for 16 hr. The reaction mixture is diluted with H₂O and extracted with EtOAc three times. The combined organic layers are washed with brine, dried over Na₂SO₄, filtered and concentrated under reduced pressure to give a residue which is purified by flash silica gel chromatography (eluent of 10% ethyl acetate/petroleum ether gradient) to yield the title compound ethyl 5-(trifluoromethyl)pyrazolo[1,5-a]pyridine-3-carboxylate (Int-10) (1.3 g) as a yellow solid. LCMS (ESI MS) m/z=259.0 [M+H]+ 1.

¹H NMR (400 MHz, DMSO-d₆) δ=9.13 (d, J=7.2 Hz, 1H), 8.64 (s, 1H), 8.32-8.37 (m, 1H), 7.44 (dd, J=7.2, 2.1 Hz, 1H), 4.35 (q, J=7.2 Hz, 2H), 1.35 (t, J=7.2 Hz, 3H).

Step B: A mixture of ethyl 5-(trifluoromethyl)pyrazolo[1,5-a]pyridine-3-carboxylate (Int-10) (1.3 g, 5.03 mmol) in H₂SO₄ (10 mL, 40% aq.) is stirred at 100° C. for 16 hr. The reaction mixture is adjusted to pH=8 using 2N NaOH (aq), and extracted with EtOAc three times. The combined organic layers are washed with brine, dried over Na₂SO₄, filtered and concentrated under reduced pressure to give the title compound 5-(trifluoromethyl)pyrazolo[1,5-a]pyridine crude product (Int-11) (550 mg, crude) as a brown oil. LCMS (ESI MS) m/z=187.0 [M+H]+ 1.

¹H NMR (400 MHz, DMSO-d₆) δ=8.90 (dd, J=7.2, 0.8 Hz, 1H), 8.24-8.28 (m, 1H), 8.19 (d, J=2.4 Hz, 1H), 7.10 (dd, J=7.2, 2.1 Hz, 1H), 6.90 (d, J=2.0 Hz, 1H).

Step C: To a solution of 5-(trifluoromethyl)pyrazolo[1,5-a]pyridine (Int-11) (180 mg, 967.05 μmol) in ACN (4 mL) is added NBS (189 mg, 1.06 mmol). The mixture is stirred at 25° C. for 16 hr. The reaction mixture is concentrated under reduced pressure to give a residue which is purified by flash silica gel chromatography (eluent of 3% ethyl acetate/petroleum ether gradient) to yield the title compound 3-bromo-5-(trifluoromethyl)pyrazolo[1,5-a]pyridine (Int-12) (180 mg) as a yellow solid. LCMS (ESI MS) m/z=264.9 [M+H]+ 1.

Step D: A mixture of 3-bromo-5-(trifluoromethyl)pyrazolo[1,5-a]pyridine (Int-12) (200 mg, 754.6 μmol), 4,4,5,5-tetramethyl-2-(4,4,5,5-tetramethyl-1,3,2-dioxaborolan-2-yl)-1,3,2-dioxaborolane (287 mg, 1.13 mmol), Pd(dppf)Cl₂ (123 mg, 150.9 μmol), and KOAc (148 mg, 1.51 mmol) in dioxane (2 mL) is purged with N₂ three times, and then the mixture is stirred at 100° C. for 12 hr under N₂. The reaction mixture is concentrated under reduced pressure, and the residue is diluted with water and extracted with ethyl acetate three times. The combined organic layers are washed with brine, dried over anhydrous Na₂SO₄, filtered and concentrated under reduced pressure to give a residue which is purified by flash silica gel chromatography (eluent of 0-7% ethyl acetate/petroleum ether gradient) to yield the title compound 3-(4,4,5,5-tetramethyl-1,3,2-dioxaborolan-2-yl)-5-(trifluoromethyl)pyrazolo[1,5-a]pyridine (Intermediate C) (230 mg, crude) as a yellow solid. LCMS (ESI MS) m/z=312.9 [M+H]+1.

### Intermediate D

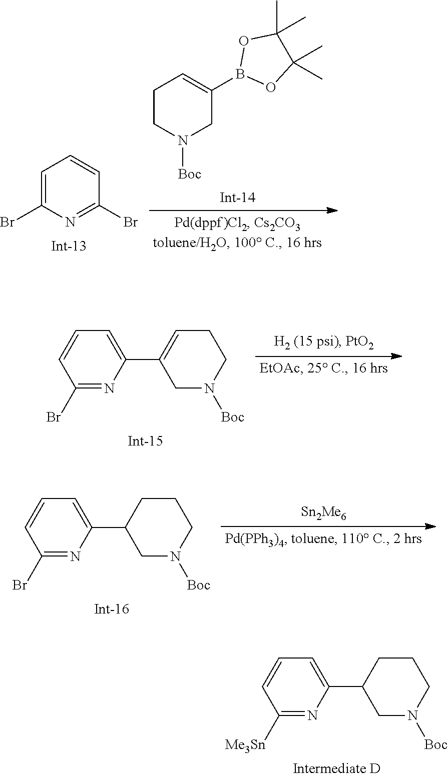

Step A: A mixture of 2,6-dibromopyridine (Int-13) (9.2 g, 19.40 mmol), tert-butyl 5-(4,4,5,5-tetramethyl-1,3,2-dioxaborolan-2-yl)-3,6-dihydropyridine-1(2H)-carboxylate (Int-14) (10 g, 32.34 mmol), Cs₂CO₃ (21.05 g, 64.68 mmol), and Pd(dppf)Cl₂ (2.36 g, 2.34 mmol) in toluene (100 mL) and H₂O (20 mL) is degassed and purged with N₂ three times. The mixture is stirred at 100° C. for 16 hr under a N₂ atmosphere. The mixture is diluted with H₂O and extracted with EtOAc three times. The combined organic layers are washed with brine, dried over anhydrous Na₂SO₄, filtered and concentrated under reduced pressure to give a crude product which is purified by flash silica gel chromatography (eluent of 0-5% ethyl acetate/petroleum ether gradient). The title compound tert-butyl 5-(6-bromo-2-pyridyl)-3,6-dihydro-2H-pyridine-1-carboxylate (Int-15) (6.3 g, 46%) is obtained as a pink solid.

¹H NMR (400 MHz, DMSO-d₆) δ=7.76-7.70 (m, 1H), 7.64 (d, J=8.0 Hz, 1H), 7.51 (d, J=7.6 Hz, 1H), 6.84 (br t, J=4.0 Hz, 1H), 4.25 (br s, 2H), 3.46 (br t, J=5.6 Hz, 2H), 2.30 (br d, J=3.6 Hz, 2H), 1.43 (s, 9H).

Step B: To a solution of tert-butyl 6-bromo-5′,6′-dihydro-[2,3′-bipyridine]-1′(2′H)-carboxylate (Int-15) (6.3 g, 18.57 mmol) in EtOAc (60 mL) is added PtO₂ (600 mg, 2.64 mmol). The mixture is stirred at 25° C. under H₂ (15 psi) for 16 hrs. The reaction mixture is filtered, and the filtrate is concentrated under vacuum to yield the title compound tert-butyl 3-(6-bromo-2-pyridyl)piperidine-1-carboxylate (Int-16) (4.4 g, crude) as a white solid.

¹H NMR (400 MHz, methanol-d₄) δ=7.65-7.58 (m, 1H), 7.42 (d, J=8.0 Hz, 1H), 7.30 (d, J=7.6 Hz, 1H), 4.26-3.97 (m, 2H), 3.18-2.97 (m, 1H), 2.96-2.68 (m, 2H), 2.07-1.97 (m, 1H), 1.86-1.73 (m, 2H), 1.63-1.52 (m, 1H), 1.46 (s, 9H).

Step C: A mixture of tert-butyl 3-(6-bromopyridin-2-yl)piperidine-1-carboxylate (Int-16) (3 g, 7.30 mmol), 1,1,1,2,2,2-hexamethyldistannane (10.36 g, 31.62 mmol, 6.56 mL), and Pd(PPh₃)₄ (843 mg, 729.69 μmol) in toluene (50 mL) is degassed and purged with N₂ three times. The mixture is stirred at 110° C. for 2 hr under a N₂ atmosphere. The mixture is quenched with saturated aqueous KF (30 mL) and stirred at 25° C. for 1 hr. Then the mixture is extracted with ethyl acetate twice. The combined organic layers are washed with brine, dried over anhydrous Na₂SO₄ and evaporated under vacuum to give a residue. The residue is purified by prep-HPLC (column: Xtimate C18, 150×40 mm×10 um; mobile phase: [water (NH₄HCO₃)-ACN]; gradient: 70%-100% B over 32 min) to afford the title compound tert-butyl 3-(6-(trimethylstannyl)pyridin-2-yl)piperidine-1-carboxylate (Intermediate D) (1.6 g, 51% yield) as a yellow oil. LCMS (ESI MS) m/z=427.1 [M+H]+ 1.

### Example 1

### 3-[6-(4-piperidyl)-2-pyridyl]pyrazolo[1,5-a]pyridine

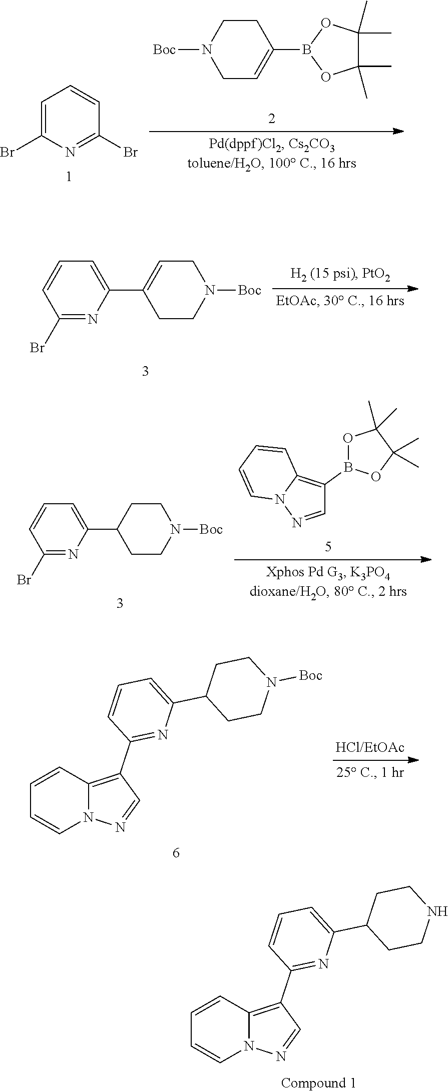

Step A: A mixture of 2,6-dibromopyridine 2 (5 g, 16.17 mmol), tert-butyl 4-(4,4,5,5-tetramethyl-1,3,2-dioxaborolan-2-yl)-3,6-dihydropyridine-1(2H)-carboxylate 1 (4.21 g, 17.79 mmol), Cs₂CO₃ (10.54 g, 32.34 mmol) and Pd(dppf)Cl₂ (2.37 g, 3.23 mmol) in H₂O (5 mL) and toluene (50 mL) is degassed and purged with N₂ three times. The mixture is stirred at 100° C. for 16 hr under N₂. The reaction is concentrated under reduced pressure to give a residue which is purified by flash silica gel chromatography (eluent of 0-20% ethyl acetate/petroleum ether gradient) to give the title compound tert-butyl 4-(6-bromo-2-pyridyl)-3,6-dihydro-2H-pyridine-1-carboxylate 3 (1.8 g, 32.8% yield) as a yellow oil. LCMS (ESI MS) m/z=339.0 [M+H]+ 1.

¹H NMR (400 MHz, methanol-d₄) δ=7.60-7.65 (m, 1H), 7.49 (d, J=7.8 Hz, 1H), 7.41 (d, J=7.8 Hz, 1H), 6.70 (br s, 1H), 4.12 (br d, J=7.2 Hz, 2H), 3.63 (br t, J=5.2 Hz, 2H), 2.56-2.62 (m, 2H), 1.49 (s, 9H).

Step B: To a solution of tert-butyl 6-bromo-3′,6′-dihydro-[2,4′-bipyridine]-1′(2′H)-carboxylate 3 (1.8 g, 5.31 mmol) in EtOAc (20 mL) is added PtO₂ (241 mg, 1.06 mmol) under a N₂ atmosphere. The suspension is degassed and purged with H₂ three times. The mixture is stirred under H₂ (15 psi) at 30° C. for 16 hrs. The reaction mixture is filtered, and the filtrate is concentrated under reduced pressure to give the title compound tert-butyl 4-(6-bromo-2-pyridyl)piperidine-1-carboxylate 4 (1.3 g) as a colorless oil. LCMS (ESI MS) m/z=341.0 [M+H]+ 1.

Step C: A mixture of 3-(4,4,5,5-tetramethyl-1,3,2-dioxaborolan-2-yl)pyrazolo[1,5-a]pyridine 5 (236 mg, 967 μmol), tert-butyl 4-(6-bromopyridin-2-yl)piperidine-1-carboxylate 4 (0.3 g, 879.1 μmol), K₃PO₄ (373 mg, 1.76 mmol), and XPhos Pd G3 (74.4 mg, 87.9 μmol) in dioxane (2 mL) and H₂O (0.2 mL) is degassed and purged with N₂ three times. The mixture is stirred at 80° C. for 2 hr under N₂. The reaction mixture is concentrated under reduced pressure to give a residue which is purified by flash silica gel chromatography (eluent of 10-20% ethyl acetate/petroleum ether gradient) to give the title compound tert-butyl 4-(6-pyrazolo[1,5-a]pyridin-3-yl-2-pyridyl)piperidine-1-carboxylate 6 (180 mg, 40.7% yield) as a yellow solid. LCMS (ESI MS) m/z=379.2 [M+H]+ 1.

¹H NMR (400 MHz, DMSO-d₆) δ=8.76 (d, J=7.2 Hz, 1H), 8.67 (s, 1H), 8.56 (d, J=8.8 Hz, 1H), 7.69-7.74 (m, 2H), 7.36-7.43 (m, 1H), 7.08 (dd, J=5.6, 2.8 Hz, 1H), 7.01 (t, J=6.4 Hz, 1H), 4.10 (br d, J=10.4 Hz, 2H), 2.84-2.99 (m, 3H), 1.92 (br d, J=11.6 Hz, 2H), 1.69 (qd, J=12.4, 4.1 Hz, 2H), 1.44 (s, 9H).

Step D: To tert-butyl 4-(6-(pyrazolo[1,5-a]pyridin-3-yl)pyridin-2-yl)piperidine-1-carboxylate 6 (180 mg, 475.6 μmol) is added HCl/EtOAc (5 mL/5 mL). The mixture is stirred at 25° C. for 1 hr. The reaction mixture is concentrated under reduced pressure to give a residue which is purified by prep-HPLC (column: Welch Xtimate C18, 150×30 mm×5 um; mobile phase: [water (FA)-ACN]; B %: 0%-24%, 25 min.) to yield the title compound 3-[6-(4-piperidyl)-2-pyridyl]pyrazolo[1,5-a]pyridine (Compound 1) (44 mg, 32.6% yield) as a white solid. LCMS (ESI MS) m/z=279.1 [M+H]+ 1.

¹H NMR (400 MHz, methanol-d₄) δ=8.64 (d, J=9.2 Hz, 1H), 8.59 (d, J=7.2 Hz, 1H), 8.56 (s, 1H), 8.52 (s, 1H), 7.72-7.77 (m, 1H), 7.66-7.70 (m, 1H), 7.37-7.43 (m, 1H), 7.11 (d, J=7.6 Hz, 1H), 7.01 (td, J=6.8, 1.1 Hz, 1H), 3.52-3.60 (m, 2H), 3.17-3.24 (m, 2H), 3.09-3.16 (m, 1H), 2.18-2.25 (m, 4H).

### Example 2

### 3-[6-(1-methyl-4-piperidyl)-2-pyridyl]pyrazolo[1,5-a]pyridine

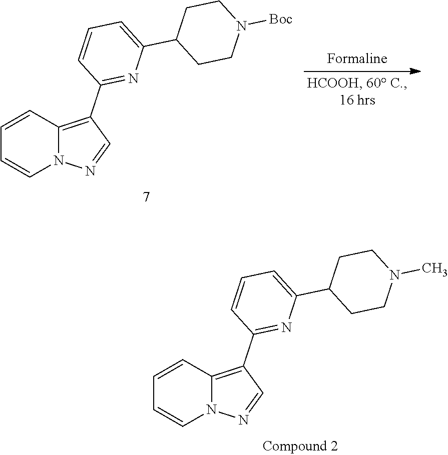 [SMILES: CC(C)(C)OC(=O)N1CCC(c2cccc(-c3cnn4ccccc34)n2)CC1 ; CN1CCC(c2cccc(-c3cnn4ccccc34)n2)CC1]

Step A: To a solution of tert-butyl 4-(6-(pyrazolo[1,5-a]pyridin-3-yl)pyridin-2-yl)piperidine-1-carboxylate 7 (146 mg, 385.8 μmol) in HCOOH (1.5 mL) is added Formaline (1.64 g, 20.15 mmol, 1.5 mL, 37% purity). The mixture is stirred at 60° C. for 16 hrs. The reaction mixture is concentrated under reduced pressure to give a residue which is purified by prep-HPLC (column: Welch Xtimate C18, 150×30 mm×5 um; mobile phase: [water (FA)-ACN]; gradient: 0%-26% B over 25 min) to yield the title compound 3-[6-(1-methyl-4-piperidyl)-2-pyridyl]pyrazolo[1,5-a]pyridine (Compound 2) (48.9 mg 42.9% yield) as a white solid. LCMS (ESI MS) m/z=293.1 [M+H]+ 1.

¹H NMR (400 MHz, methanol-d₄) δ=8.65 (d, J=9.2 Hz, 1H), 8.59 (d, J=7.2 Hz, 1H), 8.54 (s, 1H), 8.51 (s, 1H), 7.71-7.76 (m, 1H), 7.66-7.69 (m, 1H), 7.40 (ddd, J=9.2, 6.8, 1.0 Hz, 1H), 7.11 (d, J=7.6 Hz, 1H), 7.01 (td, J=6.8, 1.3 Hz, 1H), 3.54 (br d, J=12.4 Hz, 2H), 2.98-3.09 (m, 3H), 2.82 (s, 3H), 2.20-2.31 (m, 4H).

### Example 3

### 3-(6-pyrrolidin-3-yl-2-pyridyl)pyrazolo[1,5-a]pyridine

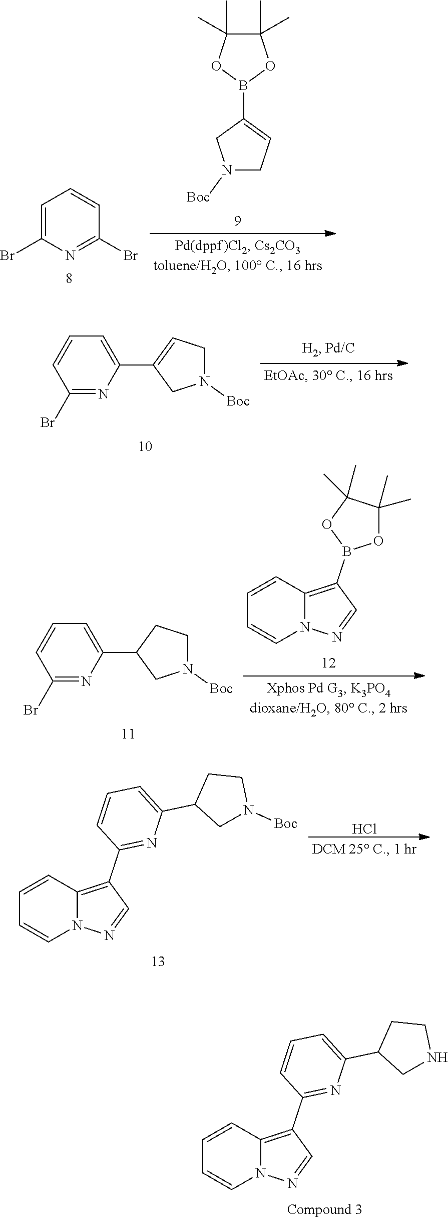

Step A: A mixture tert-butyl 3-(4,4,5,5-tetramethyl-1,3,2-dioxaborolan-2-yl)-2,5-dihydro-1H-pyrrole-1-carboxylate 9 (3 g, 10.16 mmol), 2,6-dibromopyridine 8 (2.89 g, 12.20 mmol), Pd(dppf)Cl₂ (1.12 g, 1.52 mmol), and Cs₂CO₃ (6.62 g, 20.33 mmol) in toluene (100 mL) and H₂O (20 mL) is degassed and purged with N₂ three times. The mixture is stirred at 80° C. for 10 hr under a N₂ atmosphere. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-17% ethyl acetate/petroleum ether gradient) to yield the title compound tert-butyl 3-(6-bromo-2-pyridyl)-2,5-dihydropyrrole-1-carboxylate 10 (1.45 g, 43.4% yield) as a yellow solid. LCMS (ESI MS) m/z=325.0 [M+H]+ 1.

Step B: To a solution of tert-butyl 3-(6-bromopyridin-2-yl)-2,5-dihydro-1H-pyrrole-1-carboxylate 10 (1.7 g, 5.23 mmol) in EtOAc (20 mL) is added PtO₂ (237.4 mg, 1.05 mmol) under a N₂ atmosphere. The suspension is degassed and purged with H₂ three times. The mixture is stirred under H₂ (20 psi) at 30° C. for 16 hr. The reaction mixture is filtered, and the filtrate is concentrated under reduced pressure to give the title compound tert-butyl 3-(6-bromo-2-pyridyl)pyrrolidine-1-carboxylate 11 (1.6 g, 48.6% yield) as a white solid. LCMS (ESI MS) m/z=327.0 [M+H]+ 1.

Step C: A mixture of 3-(4,4,5,5-tetramethyl-1,3,2-dioxaborolan-2-yl)pyrazolo[1,5-a]pyridine 2 (201.4 mg, 825.1 μmol), 11 (300 mg, 916.8 μmol), XPhos Pd G3 (77.6 mg, 91.7 μmol) and K₃PO₄ (389.2 mg, 1.8 mmol) in dioxane (8 mL) and H₂O (2 mL) is degassed and purged with N₂ three times. The mixture is stirred at 80° C. for 2 hr under N₂. The reaction mixture is concentrated under reduced pressure to give a residue which is purified by flash silica gel chromatography (eluent of 0-20% ethyl acetate/petroleum ether gradient) to give the title compound tert-butyl 3-(6-pyrazolo[1,5-a]pyridin-3-yl-2-pyridyl)pyrrolidine-1-carboxylate 13 (120 mg, 7.5% yield) as a yellow solid. LCMS (ESI MS) m/z=365.2 [M+H]+ 1.

Step D: To a solution of tert-butyl 3-(6-(pyrazolo[1,5-a]pyridin-3-yl)pyridin-2-yl)pyrrolidine-1-carboxylate 13 (120 mg, 329.3 μmol) in DCM (1 mL) is added HCl/EtOAc (3 mL). The mixture is stirred at 25° C. for 1 hr. The reaction mixture is concentrated under reduced pressure to give a residue which is purified by prep-HPLC (column: Welch Xtimate C18, 150×30 mm×5 um; mobile phase: [water(FA)-ACN]; gradient: 0%-26% B over 25 min) to yield the title compound 3-(6-pyrrolidin-3-yl-2-pyridyl)pyrazolo[1,5-a]pyridine (Compound 3) (13 mg, 14.8% yield) as a yellow solid. LCMS (ESI MS) m/z=265.1 [M+H]+ 1.

¹H NMR (400 MHz, DMSO-d₆) δ=8.77 (d, J=7.2 Hz, 1H), 8.70 (s, 1H), 8.56 (d, J=8.8 Hz, 1H), 8.43 (br s, 1H), 7.72-7.79 (m, 2H), 7.39-7.47 (m, 1H), 7.15 (dd, J=5.6, 2.8 Hz, 1H), 7.02 (td, J=6.8, 1.1 Hz, 1H), 3.56-3.59 (m, 2H), 3.34 (br d, J=3.2 Hz, 1H), 3.13-3.25 (m, 2H), 2.27-2.41 (m, 1H), 2.04-2.19 (m, 1H).

### Example 4

### Tert-butyl 3-(6-(5-chloropyrazolo[1,5-a]pyridin-3-yl)pyridin-2-yl)pyrrolidine-1-carboxylate

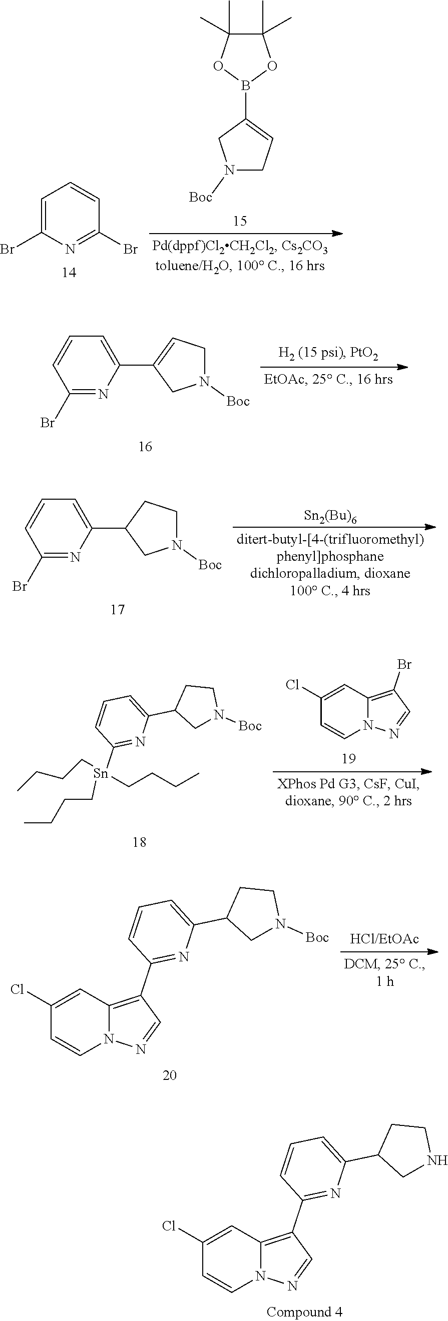

Step A: A mixture of 2,6-dibromopyridine 14 (802.5 mg, 3.4 mmol), tert-butyl 3-(4,4,5,5-tetramethyl-1,3,2-dioxaborolan-2-yl)-2,5-dihydro-1H-pyrrole-1-carboxylate 15 (500 mg, 1.7 mmol), Cs₂CO₃ (1.1 g, 3.4 mmol), Pd(dppf)Cl₂·CH₂Cl₂ (276.6 mg, 338.8 μmol) in toluene (10 mL) and H₂O (3 mL) is purged with N₂ three times. The mixture is stirred at 80° C. for 2 hr under N₂. The reaction is diluted with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-3% ethyl acetate/petroleum ether gradient) to yield the title compound tert-butyl 3-(6-bromopyridin-2-yl)-2,5-dihydro-1H-pyrrole-1-carboxylate 1 (150 mg, 27%) as a white solid. LCMS (ESI MS) m/z=325.0 [M+H]+ 1.

Step B: A mixture of tert-butyl 3-(6-bromopyridin-2-yl)-2,5-dihydro-1H-pyrrole-1-carboxylate 16 (400 mg, 1.23 mmol) and PtO₂ (27.9 mg, 123.0 μmol) in EtOAc (5 mL) is degassed and purged with H₂ three times. The mixture is stirred at 25° C. for 48 hr under a H₂ (15 psi). The reaction mixture is filtered, and the filtrate is concentrated under reduced pressure to give a crude product. The crude product is purified by prep-HPLC (neutral condition; column: Xtimate C18, 150×40 mm×10 um; mobile phase: [water (NH₄HCO₃)-ACN]; gradient: 38%-78% B over 32 min) to yield the title compound tert-butyl 3-(6-bromopyridin-2-yl)pyrrolidine-1-carboxylate 17 (200 mg, 49%) as a white solid. LCMS (ESI MS) m/z=271.0 [M−55]+1.

Step C: A mixture of tert-butyl 3-(6-bromopyridin-2-yl)pyrrolidine-1-carboxylate 17 (100 mg, 305.6 μmol), tributyl(tributylstannyl)stannane (354.5 mg, 611.2 μmol, 306 μL), and di-tert-butyl-[4-(trifluoromethyl)phenyl]phosphane dichloropalladium (23.1 mg, 30.6 μmol) in dioxane (2 mL) is degassed and purged with N₂ three times. The mixture is stirred at 100° C. for 4 hr under a N₂ atmosphere. The mixture is diluted with H₂O and extracted with ethyl acetate three times. The combined organic layers are washed with brine, dried over anhydrous Na₂SO₄, filtered and concentrated under reduced pressure to give a residue. The residue is purified by flash silica gel chromatography (eluent of 0-30% ethyl acetate/petroleum ether gradient) to afford the title compound tert-butyl 3-(6-(tributylstannyl)pyridin-2-yl)pyrrolidine-1-carboxylate 18 (50 mg, 30% yield) as a white solid. LCMS (ESI MS) m/z=539.3 [M+H]+ 1.

Step D: A mixture of 3-bromo-5-chloropyrazolo[1,5-a]pyridine 19 (15 mg, 64.8 μmol), tert-butyl 3-(6-(tributylstannyl)pyridin-2-yl)pyrrolidine-1-carboxylate 18 (48.7 mg, 90.7 μmol), CsF (39.3 mg, 259.1 μmol, 10 μL), CuI (24.6 mg, 129.6 μmol), and XPhos Pd G3 (10.9 mg, 12.96 μmol) in dioxane (2 mL) is degassed and purged with N₂ three times. The mixture is stirred at 90° C. for 2 hr under N₂. The reaction mixture is extracted with ethyl acetate three times. The combined organic layers are dried over anhydrous Na₂SO₄, filtered and concentrated under reduced pressure to give the title compound tert-butyl 3-(6-(5-chloropyrazolo[1,5-a]pyridin-3-yl)pyridin-2-yl)pyrrolidine-1-carboxylate 20 (30 mg, crude) as a white solid. LCMS (ESI MS) m/z=399.1 [M+H]+ 1.

Step E: To a solution of tert-butyl 3-(6-(5-chloropyrazolo[1,5-a]pyridin-3-yl)pyridin-2-yl)pyrrolidine-1-carboxylate 20 (20 mg, 50.1 μmol) in DCM (1 mL) is added HCl/EtOAc (1 mL, 4M). The mixture is stirred at 25° C. for 1 h. The reaction mixture is concentrated under vacuum to give the crude product. The crude product is purified by prep-HPLC (FA condition; column: Xtimate C18, 150×40 mm×10 um; mobile phase: [water(FA)-ACN]; gradient: 0%-32% B over 25 min) affording the title compound tert-butyl 3-(6-(5-chloropyrazolo[1,5-a]pyridin-3-yl)pyridin-2-yl)pyrrolidine-1-carboxylate (Compound 4) (2 mg, 12%) as a white solid. LCMS (ESI MS) m/z=299.1 [M+H]+ 1.

¹H NMR (400 MHz, methanol-d₄) δ=7.23-7.31 (m, 2H), 7.18 (s, 1H), 6.44-6.50 (m, 1H), 6.36-6.42 (m, 1H), 5.88 (br d, J=7.50 Hz, 1H), 5.68 (br d, J=6.63 Hz, 1H), 2.43-2.53 (m, 2H), 2.24-2.36 (m, 2H), 2.12-2.20 (m, 1H), 1.25 (br dd, J=11.82, 5.32 Hz, 1H), 1.00-1.12 (m, 1H).

### Example 5

### Rac-5-chloro-3-[6-(3-piperidyl)-2-pyridyl]pyrazolo[1,5-a]pyridine

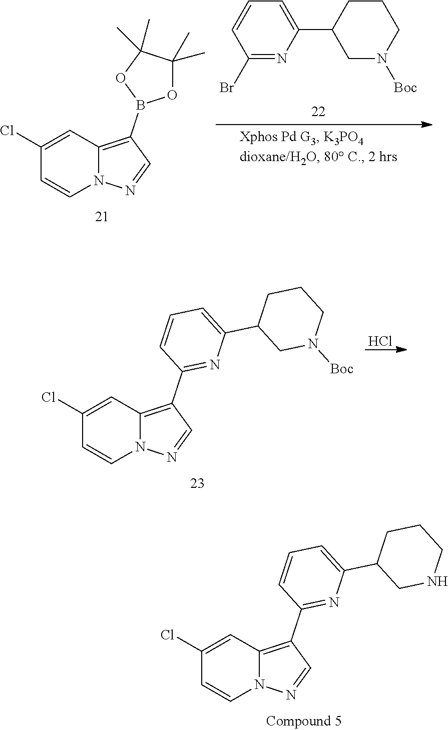

Step A: To a solution of 5-chloro-3-(4,4,5,5-tetramethyl-1,3,2-dioxaborolan-2-yl)pyrazolo[1,5-a]pyridine 21 (80 mg, 287.2 μmol) and tert-butyl 3-(6-bromopyridin-2-yl)piperidine-1-carboxylate 22 (147 mg, 430.8 μmol) in H₂O (2 mL) and toluene (3 mL) are added Pd(dppf)Cl₂ (23.4 mg, 28.7 μmol) and Cs₂CO₃ (93.6 mg, 287.2 μmol). The mixture is stirred at 60° C. for 12 hr under N₂. The reaction mixture is quenched with H₂O and extracted with EtOAc three times. The combined organic layers are washed with brine twice, dried over anhydrous Na₂SO₄, filtered and concentrated under reduced pressure to give a residue which is purified by flash silica gel chromatography (eluent of 0-20% ethyl acetate/petroleum ether gradient) to yield the title compound tert-butyl-3-[6-(5-chloropyrazolo[1,5-a]pyridin-3-yl)-2-pyridyl]piperidine-1-carboxylate 23 (30 mg, 72.6 μmol, 25.3% yield) as a yellow solid. LCMS (ESI MS) m/z=413.1 [M+H]+ 1.

Step B: To a solution of tert-butyl 3-(6-(5-chloropyrazolo[1,5-a]pyridin-3-yl)pyridin-2-yl)piperidine-1-carboxylate 23 (36 mg, 87.2 μmol) in DCM (1 mL) is added HCl (12 M, 29 μL). The mixture is stirred at 25° C. for 1 hr. The reaction mixture is concentrated under reduced pressure to give a residue which is purified by prep-HPLC (FA condition column: Welch Xtimate C18, 150×30 mm×5 um; mobile phase: [water (FA)-ACN]; gradient: 0%-32% B over 25 min) to yield the title compound Rac-5-chloro-3-[6-(3-piperidyl)-2-pyridyl]pyrazolo[1,5-a]pyridine (Compound 5) (31.7 mg, 84.8 μmol, 97.3% yield) as a white solid. LCMS (ESI MS) m/z=313.1 [M+H]+ 1.

¹H NMR (400 MHz, DMSO-d₆) δ=8.83 (d, J=7.6 Hz, 1H), 8.77 (s, 1H), 8.63 (d, J=2.0 Hz, 1H), 7.74-7.79 (m, 2H), 7.15 (dd, J=5.6, 3.6 Hz, 1H), 7.07-7.12 (m, 1H), 3.17 (br d, J=12.8 Hz, 2H), 2.99-3.09 (m, 2H), 2.95 (s, 1H), 2.71 (br d, J=2.8 Hz, 1H), 2.68-2.69 (m, 1H), 2.08 (br d, J=12.0 Hz, 1H), 1.69-1.86 (m, 3H).

### Examples 6 and 7

### 5-chloro-3-[6-[(3R)-3-piperidyl]-2-pyridyl]pyrazolo[1,5-a]pyridine and 5-chloro-3-[6-[(3S)-3-piperidyl]-2-pyridyl]pyrazolo[1,5-a]pyridine

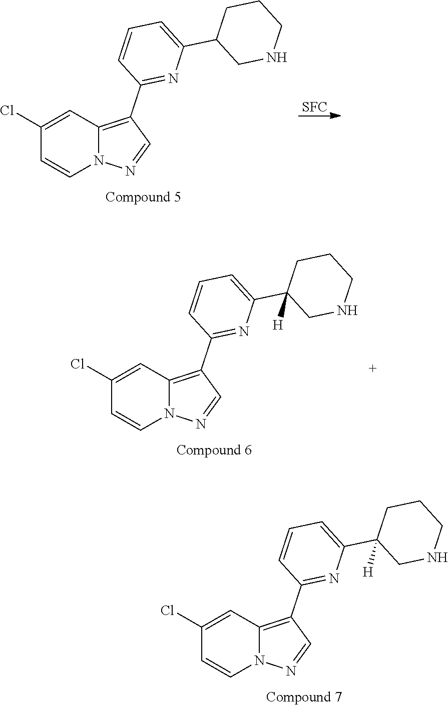

Step A: Rac-5-chloro-3-[6-(3-piperidyl)-2-pyridyl]pyrazolo[1,5-a]pyridine (Compound 5) (100 mg) is separated by SFC (column: DAICEL CHIRALPAK IG (250 mm×30 mm, 10 um); mobile phase: [CO₂-EtOH(0.1% NH₃H₂O)]; B %: 35%, isocratic elution mode) to yield the title compounds Compound 6 (20.3 mg, 32%) as a white solid and Compound 7 (21.8 mg, 33% yield) as a white solid. Stereochemistry not assigned.

For Compound 6: LCMS (ESI MS) m/z=313.2 [M+H]+ 1. ¹H NMR (400 MHz, methanol-d₄) δ=8.61 (d, J=2.4 Hz, 1H), 8.47 (d, J=7.2 Hz, 1H), 8.43 (s, 1H), 7.59-7.66 (m, 1H), 7.51-7.56 (m, 1H), 7.00 (d, J=7.6 Hz, 1H), 6.89 (dd, J=7.6, 2.4 Hz, 1H), 3.27 (br s, 1H), 3.03-3.10 (m, 1H), 2.86-2.96 (m, 2H), 2.65 (td, J=12.4, 2.8 Hz, 1H), 2.03-2.10 (m, 1H), 1.75-1.85 (m, 2H), 1.59-1.71 (m, 1H). [α]_{D}=+10 (c=0.001, MeOH). SFC t_{R}=1.628 min.

For Compound 7: LCMS (ESI MS) m/z=313.1 [M+H]+ 1. ¹H NMR (400 MHz, methanol-d₄) δ=8.75 (d, J=2.4 Hz, 1H), 8.59 (d, J=7.2 Hz, 1H), 8.49-8.55 (m, 1H), 7.69-7.78 (m, 1H), 7.59-7.68 (m, 1H), 7.11 (d, J=7.6 Hz, 1H), 7.01 (dd, J=7.6, 2.4 Hz, 1H), 3.38 (br s, 1H), 3.09-3.20 (m, 1H), 2.92-3.04 (m, 2H), 2.72 (br t, J=12.0 Hz, 1H), 2.11-2.24 (m, 1H), 1.84-1.93 (m, 2H), 1.69-1.80 (m, 1H). [α]_{D}=−10 (c=0.001, MeOH). SFC t_{R}=1.792 min.

### Example 8

### 5-bromo-3-[6-(3-piperidyl)-2-pyridyl]pyrazolo[1,5-a]pyridine

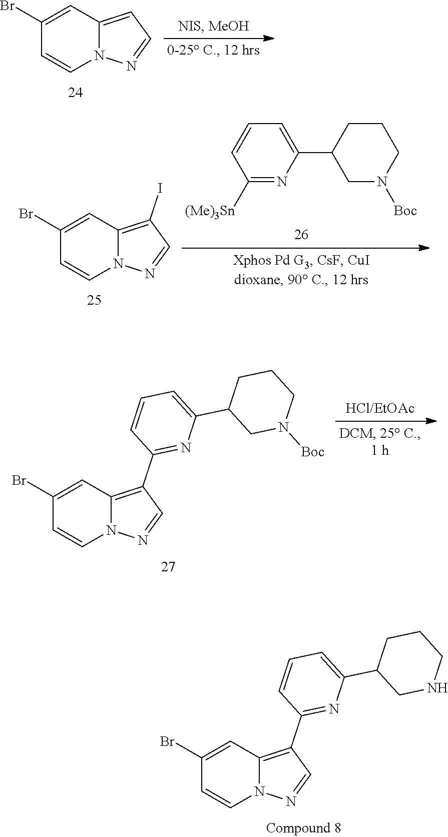

Step A: To a solution of 5-bromopyrazolo[1,5-a]pyridine 24 (500 mg, 2.54 mmol) in MeOH (5 mL) is added N-iodosuccinimide (NIS) (570.9 mg, 2.54 mmol) at 0° C. The reaction temperature is allowed to slowly warm to 25° C., and the reaction solution is then stirred at 25° C. for 12 hrs. The reaction mixture is concentrated under reduced pressure to give the title compound 5-bromo-3-iodo-pyrazolo[1,5-a]pyridine 25 (480 mg) as a white solid. LCMS (ESI MS) m/z=324.7 [M+H]+ 1.

Step B: To a solution of 5-bromo-3-iodopyrazolo[1,5-a]pyridine 25 (100 mg, 309.7 μmol) and tert-butyl 3-(6-(trimethylstannyl)pyridin-2-yl)piperidine-1-carboxylate 26 (144.8 mg, 340.6 μmol) in dioxane (2 mL) are added Xphos Pd G3 (35.8 mg, 30.97 μmol), CsF (188.2 mg, 1.24 mmol, 45.7 μL) and CuI (117.9 mg, 619.3 μmol). The mixture is stirred at 90° C. for 12 hr under N₂. The reaction mixture is concentrated under reduced pressure to give a residue which is purified by flash silica gel chromatography (eluent of 0-30% DCM/MeOH gradient) to yield the title compound tert-butyl 3-[6-(5-bromopyrazolo[1,5-a]pyridin-3-yl)-2-pyridyl]piperidine-1-carboxylate 27 (20 mg) as a yellow solid. LCMS (ESI MS) m/z=456.9 [M+H]+1.

Step C: To tert-butyl 3-(6-(5-bromopyrazolo[1,5-a]pyridin-3-yl)pyridin-2-yl)piperidine-1-carboxylate 27 (20 mg, 43.73 μmol) is added HCl/DCM (6.38 mg, 174.9 μmol). The mixture is stirred at 25° C. for 1 hr. The reaction mixture is concentrated under reduced pressure to give a residue which is purified by prep-HPLC (FA condition, column: Welch Xtimate C18, 150×30 mm×5 um; mobile phase: [water (FA)-ACN]; gradient: 0%-36% B over 25 min) to yield the title compound 5-bromo-3-[6-(3-piperidyl)-2-pyridyl]pyrazolo[1,5-a]pyridine (Compound 8) (2.1 mg, 5.29 μmol, 12.1% yield) as a white solid. LCMS (ESI MS) m/z=357.0 [M+H]+ 1.

¹H NMR (400 MHz, DMSO-d₆) δ=8.69-8.83 (m, 3H), 7.75-7.87 (m, 2H), 7.12-7.27 (m, 2H), 3.51-3.60 (m, 1H), 3.04-3.25 (m, 3H), 2.83-2.95 (m, 1H), 2.06-2.17 (m, 1H), 1.91-1.99 (m, 1H), 1.76-1.90 (m, 2H).

### Example 9

### 5-methyl-3-[6-(3-piperidyl)-2-pyridyl]pyrazolo[1,5-a]pyridine

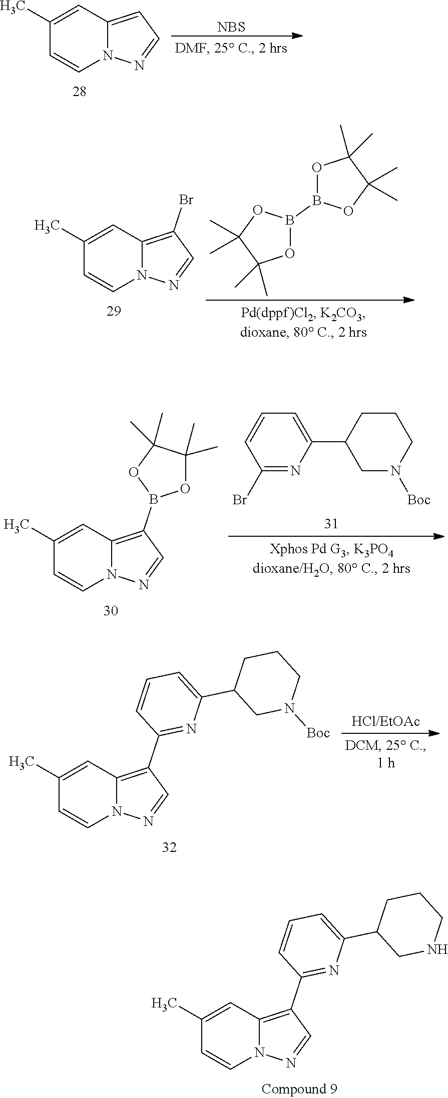

Step A: To a solution of 5-methylpyrazolo[1,5-a]pyridine 28 (500 mg, 3.78 mmol) in DMF (6 mL) is added NBS (673 mg, 3.78 mmol). The mixture is stirred at 25° C. for 16 hrs. The reaction mixture is quenched with H₂O, and the mixture is extracted with EtOAc three times. The combined organic layers are washed with brine, dried over Na₂SO₄, filtered and concentrated under reduced pressure to give a residue which is purified by flash silica gel chromatography (eluent of 0-30% ethyl acetate/petroleum ether gradient) to yield the title compound 9 (600 mg) as a white solid. LCMS (ESI MS) m/z=213.0 [M+H]+ 1.

Step B: A mixture of 3-bromo-5-methylpyrazolo[1,5-a]pyridine 29 (250 mg, 1.18 mmol), 4,4,4′,4′,5,5,5′,5′-octamethyl-2,2′-bi(1,3,2-dioxaborolane) (601 mg, 2.37 mmol), K₂CO₃ (245 mg, 1.78 mmol), and Pd(dppf)Cl₂·CH₂Cl₂ (96 mg, 118.45 μmol) in dioxane (5 mL) is purged with N₂ three times, and then the mixture is stirred at 80° C. for 2 hr under N₂. The reaction mixture is quenched with H₂O and extracted with EtOAc three times. The combined organic layers re washed with brine, dried over Na₂SO₄, filtered and concentrated under reduced pressure to give a residue which is purified by flash silica gel chromatography (eluent of 20% ethyl acetate/petroleum ether gradient) to yield the title compound 5-methyl-3-(4,4,5,5-tetramethyl-1,3,2-dioxaborolan-2-yl) pyrazolo[1,5-a]pyridine 30 (200 mg) as a white solid. LCMS (ESI MS) m/z=258.9 [M+H]+ 1.

Step C: A mixture of 5-methyl-3-(4,4,5,5-tetramethyl-1,3,2-dioxaborolan-2-yl)pyrazolo[1,5-a]pyridine 30 (70 mg, 271.19 μmol), tert-butyl 3-(6-bromopyridin-2-yl)piperidine-1-carboxylate 31 (185 mg, 542.38 μmol), K₃PO₄ (115 mg, 542.4 μmol), and XantPhos Pd G3 (25 mg, 27.12 μmol) in dioxane (1 mL) and H₂O (0.3 mL) is purged with N₂ three times, and then the mixture is stirred at 60° C. for 12 hr under N₂. The reaction mixture is diluted with H₂O, and the mixture is extracted with EtOAc three times. The combined organic layers are washed with brine, dried over Na₂SO₄, filtered and concentrated under reduced pressure to give a residue which is purified by flash silica gel chromatography (eluent of 20% ethyl acetate/petroleum ether gradient) to yield the title compound tert-butyl 3-[6-(5-methylpyrazolo[1,5-a]pyridin-3-yl)-2-pyridyl]piperidine-1-carboxylate 2 (80 mg) as a white solid. LCMS (ESI MS) m/z=393.3 [M+H]+ 1.

Step D: To a solution of tert-butyl 3-(6-(5-methylpyrazolo[1,5-a]pyridin-3-yl)pyridin-2-yl) piperidine-1-carboxylate 32 (50 mg, 127.4 μmol) in DCM (0.5 mL) is added HCl/EtOAc (4 M, 0.5 mL). The mixture is stirred at 25° C. for 1 hr. The reaction mixture is concentrated under vacuum to give the crude product which is purified by prep-HPLC (FA condition; column: Welch Xtimate C18, 150×30 mm×5 um; mobile phase: [water (FA)-ACN]; gradient: 0%-30% B over 25 min) to yield the title compound 5-methyl-3-[6-(3-piperidyl)-2-pyridyl]pyrazolo[1,5-a]pyridine (Compound 9) (18 mg, 47%) as a white solid. LCMS (ESI MS) m/z=293.2 [M+H]+ 1.

¹H NMR (400 MHz, methanol-d₄) δ=8.61 (br d, J=9.01 Hz, 1H), 8.55 (d, J=6.88 Hz, 1H), 8.41 (d, J=3.25 Hz, 1H), 7.41-7.49 (m, 1H), 7.31-7.40 (m, 1H), 7.05 (dd, J=8.38, 3.25 Hz, 1H), 6.98 (t, J=6.82 Hz, 1H), 3.36 (br d, J=10.26 Hz, 1H), 3.18 (br d, J=12.63 Hz, 1H), 2.89-3.08 (m, 2H), 2.70-2.81 (m, 1H), 2.04-2.15 (m, 1H), 1.66-1.93 (m, 3H).

### Example 10

### 3-[6-(3-piperidyl)-2-pyridyl]-5-(trifluoromethyl)pyrazolo[1,5-a]pyridine

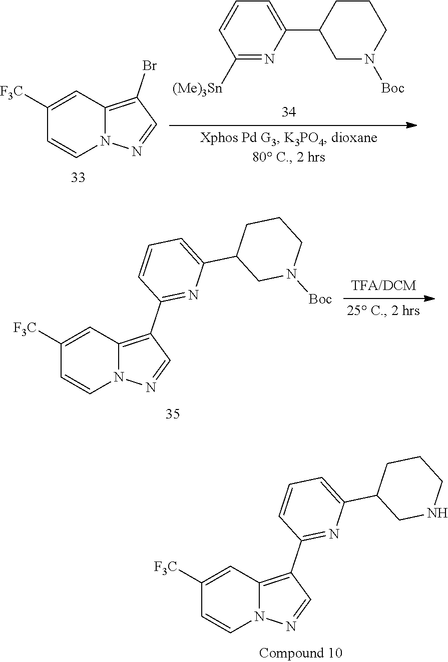

Step A: A mixture of 3-bromo-5-(trifluoromethyl)pyrazolo[1,5-a]pyridine 33 (60 mg, 226.4 μmol), tert-butyl 3-(6-(trimethylstannyl)pyridin-2-yl)piperidine-1-carboxylate 34 (115.5 mg, 271.7 μmol), XPhos Pd G3 (19.1 mg, 22.6 μmol), and K₃PO₄ (96.1 mg, 452.8 μmol) in dioxane (2 mL) is purged with N₂ three times. The mixture is stirred at 80° C. for 2 hours under N₂. The reaction mixture is concentrated directly to yield the title compound tert-butyl 3-[6-[5-(trifluoromethyl)pyrazolo[1,5-a]pyridin-3-yl]-2-pyridyl]piperidine-1-carboxylate 35 (101.0 mg, crude) as a brown solid. LCMS (ESI MS) m/z=447.2 [M+H]+ 1.

Step B: To a solution of tert-butyl 3-(6-(5-(trifluoromethyl)pyrazolo[1,5-a]pyridin-3-yl)pyridin-2-yl)piperidine-1-carboxylate 35 (100 mg, 224 μmol) in DCM (2 mL) is added trifluoroacetic acid (TFA) (0.4 mL). The mixture is stirred at 20° C. for 2 hrs. The reaction mixture is extracted with H₂O three times. The combined water layers are concentrated under reduced pressure to give a residue which w is as purified by prep-HPLC (column: Xtimate C18, 150×40 mm×10 um; mobile phase: [water(FA)-ACN]; gradient: 0%-36% B over 25 min] to yield the title compound 3-[6-(3-piperidyl)-2-pyridyl]-5-(trifluoromethyl)pyrazolo[1,5-a]pyridine (Compound 10) (37.2 mg, 47%) as a white solid. LCMS (ESI MS) m/z=347.1 [M+H]+ 1.

¹H NMR (400 MHz, methanol-d₄) δ=9.01 (s, 1H), 8.80 (d, J=7.2 Hz, 1H), 8.69 (s, 1H), 8.53 (s, 1H), 7.76-7.87 (m, 2H), 7.21 (ddd, J=14.0, 7.3, 1.6 Hz, 2H), 3.65-3.72 (m, 1H), 3.48 (br d, J=12.4 Hz, 1H), 3.33-3.40 (m, 1H), 3.28 (br d, J=3.6 Hz, 1H), 3.02 (td, J=12.4, 3.4 Hz, 1H), 2.23-2.32 (m, 1H), 2.07-2.15 (m, 1H), 1.91-2.02 (m, 2H).

### Example 11

### 5-methoxy-3-[6-(3-piperidyl)-2-pyridyl]pyrazolo[1,5-a]pyridine

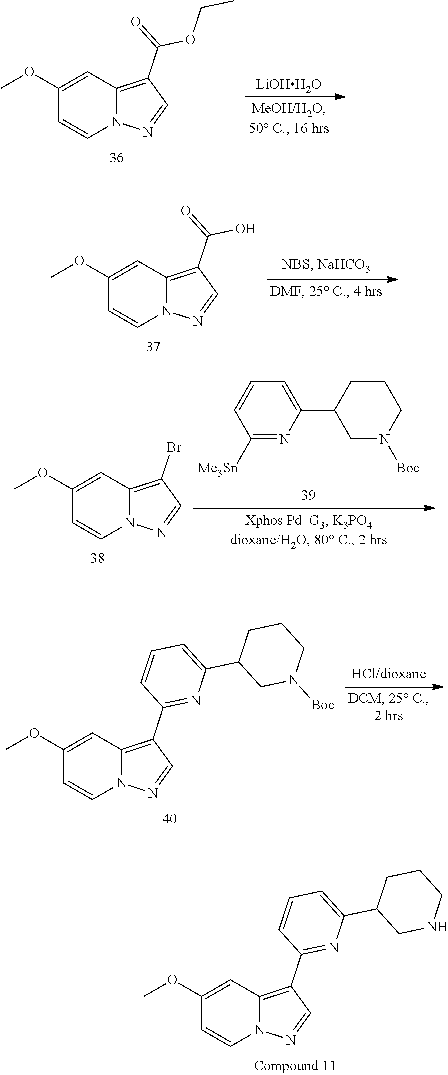

Step A: To a solution of ethyl 5-methoxypyrazolo[1,5-a]pyridine-3-carboxylate 36 (0.5 g, 2.27 mmol) in MeOH (2.5 mL) and H₂O (2.5 mL) is added LiOH·H₂O (476 mg, 11.35 mmol). The mixture is stirred at 50° C. for 16 hrs. The resulting mixture is then adjusted to pH˜5 by aq. HCl (1 M), and the reaction mixture is filtered. The filter cake is dried under reduced pressure to yield the title compound 5-methoxypyrazolo[1,5-a]pyridine-3-carboxylic acid 37 (390 mg, 89%) as a white solid.

¹H NMR (400 MHz, DMSO-d₆) δ=8.68 (d, J=7.6 Hz, 1H), 8.26 (s, 1H), 7.33 (d, J=2.8 Hz, 1H), 6.78 (dd, J=2.8, 7.6 Hz, 1H), 3.90 (s, 3H).

Step B: To a solution of 3-bromo-5-methoxypyrazolo[1,5-a]pyridine 37 (740 mg, 3.85 mmol) in DMF (8 mL) is added NaHCO₃ (970.4 mg, 11.55 mmol) and N-bromosuccinimide (NBS) (685.3 mg, 3.85 mmol) at 25° C. The mixture is stirred at 25° C. for 4 hrs. The mixture is poured into water and extracted with EtOAc. The combined organic layers are washed with brine three times, dried over Na₂SO₄, filtered and concentrated under reduced pressure to give a residue which is purified by flash silica gel chromatography (eluent of 20-40% EtOAc/PE) to yield the title compound 3-bromo-5-methoxy-pyrazolo[1,5-a]pyridine 38 (753 mg, 86%) as a white solid.

¹H NMR (400 MHz, DMSO-d₆) δ=8.58 (d, J=7.6 Hz, 1H), 8.03 (s, 1H), 6.79 (d, J=2.8 Hz, 1H), 6.64 (dd, J=2.8, 7.6 Hz, 1H), 3.88 (s, 3H).

Step C: A mixture of tert-butyl 3-(6-(trimethylstannyl)pyridin-2-yl)piperidine-1-carboxylate 3 (187.2 mg, 440.4 μmol), 3-bromo-5-methoxypyrazolo[1,5-a]pyridine 38 (0.1 g, 440.4 μmol), XPhos Pd G3 (37 mg, 44.0 μmol), and K₃PO₄ (187 mg, 880.8 μmol) in dioxane (2 mL) and H₂O (0.2 mL) is degassed and purged with N₂ three times. The mixture is stirred at 80° C. for 2 hours under N₂. The reaction mixture is concentrated under reduced pressure to give a residue which is purified by flash silica gel chromatography (eluent of 20-30% ethyl acetate/petroleum ether gradient) to yield the title compound tert-butyl 3-[6-(5-methoxypyrazolo[1,5-a]pyridin-3-yl)-2-pyridyl]piperidine-1-carboxylate 40 (80 mg, 44%) as a white solid.

¹H NMR (400 MHz, methanol-d₄) δ=8.41-8.38 (m, 2H), 8.11 (d, J=2.0 Hz, 1H), 7.72-7.65 (m, 1H), 7.62-7.55 (m, 1H), 7.05 (d, J=7.6 Hz, 1H), 6.67 (dd, J=2.8, 7.6 Hz, 1H), 4.51-4.30 (m, 1H), 4.18-4.11 (m, 1H), 3.97 (s, 3H), 2.93-2.79 (m, 2H), 2.18 (br d, J=12.8 Hz, 1H), 2.00-1.90 (m, 1H), 1.87-1.80 (m, 1H), 1.76-1.53 (m, 2H), 1.47 (s, 9H).

Step D: To a solution of tert-butyl 3-(6-(5-methoxypyrazolo[1,5-a]pyridin-3-yl)pyridin-2-yl)piperidine-1-carboxylate 40 (0.07 g, 171.36 μmol, 1 eq) in DCM (1 mL) is added HCl/dioxane (1 mL, 4M). The mixture is stirred at 25° C. for 2 hrs. The reaction mixture is partitioned between DCM and H₂O. The organic phase is discarded, and the water phase is lyophilized to yield the title compound 5-methoxy-3-[6-(3-piperidyl)-2-pyridyl]pyrazolo[1,5-a]pyridine (Compound 11) (33.7 mg, 68%) as a white solid. LCMS (ESI MS) m/z=308.8 [M+H]+ 1.

¹H NMR (400 MHz, methanol-d₄) δ=8.69 (s, 1H), 8.61 (d, J=7.6 Hz, 1H), 8.40 (t, J=8.0 Hz, 1H), 8.15 (d, J=8.0 Hz, 1H), 7.66 (d, J=7.6 Hz, 1H), 7.48 (d, J=2.4 Hz, 1H), 6.88 (dd, J=2.8, 7.6 Hz, 1H), 4.03 (s, 3H), 3.80-3.70 (m, 2H), 3.52 (br d, J=11.6 Hz, 1H), 3.47-3.39 (m, 1H), 3.19-3.08 (m, 1H), 2.34-2.26 (m, 1H), 2.19-2.08 (m, 1H), 2.06-1.96 (m, 2H).

### Example 12

### 3-(6-(piperidin-3-yl)pyridin-2-yl)pyrazolo[1,5-a]pyridin-5-ol

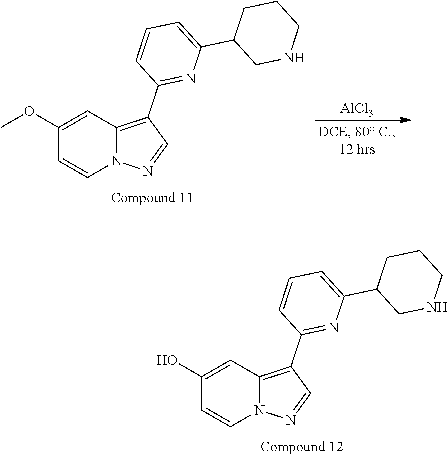

Step A: To a solution of 5-methoxy-3-(6-piperidin-3-yl)pyridine-2-yl)pyrazolo[1,5-a]pyridine Compound 11 (30 mg, 97.28 μmol) in DCE (2 mL) is added AlCl₃ (130 mg, 972.84 μmol). The mixture is stirred at 80° C. for 12 hr. The mixture is quenched with water at 0° C. and filtered. The filtrate is extracted with DCM three times. The aqueous phase is concentrated under reduced pressure to give a residue which is purified by prep-HPLC (column: Xtimate C18, 150×40 mm×10 um; mobile phase: [water (HCl)-ACN]; gradient: 0%-24% B over 36 min) to yield the title compound 3-(6-(piperidin-3-yl)pyridin-2-yl)pyrazolo[1,5-a]pyridin-5-ol (Compound 12) (6 mg, 20%) as a brown solid. LCMS (ESI MS) m/z=295.0 [M+H]+ 1.

¹H NMR (400 MHz, methanol-d₄) δ=8.51-8.66 (m, 2H), 8.11 (br t, J=6.94 Hz, 1H), 7.87 (br d, J=7.50 Hz, 1H), 7.58 (br s, 1H), 7.39 (br d, J=6.63 Hz, 1H), 6.77 (br s, 1H), 3.70 (br d, J=8.50 Hz, 1H), 3.49 (br s, 3H), 3.16 (br d, J=2.88 Hz, 1H), 2.25-2.34 (m, 1H), 2.09-2.19 (m, 1H), 1.95-2.04 (m, 2H).

### Example 13

### 5-isopropoxy-3-[6-(3-piperidyl)-2-pyridyl]pyrazolo[1,5-a]pyridine

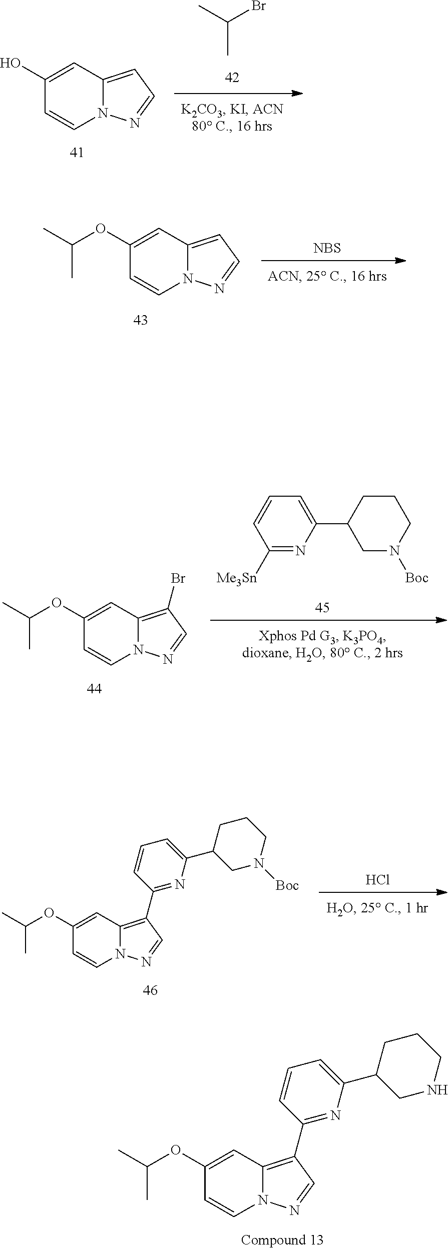

Step A: To a solution of 2-bromopropane 42 (252.1 mg, 2.05 mmol, 193 μL) and pyrazolo[1,5-a]pyridin-5-ol 41 (250 mg, 1.86 mmol) in ACN (1 mL) are added K₂CO₃ (515.1 mg, 3.73 mmol) and KI (61.8 mg, 372.8 μmol). The mixture is stirred at 80° C. for 16 hr. The reaction mixture is concentrated directly to give a crude product which is purified by flash silica gel chromatography (eluent of 0-15% ethyl acetate/petroleum ether gradient) to yield the title compound 5-isopropoxypyrazolo[1,5-a]pyridine 43 (140 mg, crude) as a white solid.

Step B: To a solution of 5-isopropoxypyrazolo[1,5-a]pyridine 43 (100 mg, 567.5 μmol) in ACN (3 mL) is added NBS (151.5 mg, 851.2 μmol). The mixture is stirred at 25° C. for 16 hr. The reaction mixture is concentrated to give a crude product which is purified by flash silica gel chromatography (eluent of 0-4% ethyl acetate/petroleum ether gradient) to yield the title compound 3-bromo-5-isopropoxy-pyrazolo[1,5-a]pyridine 44 (120 mg, crude) as colorless oil.

¹H NMR (400 MHz, DMSO-d₆) δ=8.56 (d, J=7.6 Hz, 1H), 8.00 (s, 1H), 6.76 (d, J=2.4 Hz, 1H), 6.59 (dd, J=2.8, 7.6 Hz, 1H), 4.78 (td, J=6.0, 12.0 Hz, 1H), 1.31 (d, J=6.0 Hz, 6H).

Step C: A mixture of tert-butyl 3-(6-(trimethylstannyl)pyridin-2-yl)piperidine-1-carboxylate 45 (50.0 mg, 117.61 μmol), 3-bromo-5-isopropoxypyrazolo[1,5-a]pyridine 44 (30.0 mg, 117.6 μmol), XPhos Pd G3 (9.9 mg, 11.76 μmol), and K₃PO₄ (49.9 mg, 235.2 μmol) in dioxane (1 mL) and H₂O (0.25 mL) is purged with N₂ three times, and then the mixture is stirred at 80° C. for 2 hrs under N₂. The reaction mixture is concentrated to give a crude product which is purified by flash silica gel chromatography (eluent of 0-10% ethyl acetate/petroleum ether gradient) to yield the title compound tert-butyl 3-[6-(5-isopropoxypyrazolo[1,5-a]pyridin-3-yl)-2-pyridyl]piperidine-1-carboxylate 46 (70 mg) as yellow oil.

Step D: To a solution of tert-butyl 3-(6-(5-isopropoxypyrazolo[1,5-a]pyridin-3-yl)pyridin-2-yl)piperidine-1-carboxylate 46 (60 mg, 137.4 μmol) in H₂O (1 mL) is added HCl (12 M, 171 μL). The mixture is stirred at 25° C. for 1 hr. The reaction mixture is concentrated to give the crude product which is purified by prep-HPLC (column: Xtimate C18, 150×40 mm×10 um; mobile phase: [water (HCl)-ACN]; gradient: 0%-38% B over 36 min) to yield the title compound 5-isopropoxy-3-[6-(3-piperidyl)-2-pyridyl]pyrazolo[1,5-a]pyridine (Compound 13) (19.2 mg, 40%) as a yellow solid. LCMS (ESI MS) m/z=337.2 [M+H]+ 1.

¹H NMR (400 MHz, methanol-d₄) δ=8.64-8.54 (m, 2H), 8.29-8.20 (m, 1H), 8.00 (d, J=8.0 Hz, 1H), 7.59-7.47 (m, 2H), 6.82 (dd, J=2.4, 7.6 Hz, 1H), 4.89-4.88 (m, 1H), 3.72 (br dd, J=1.2, 12.0 Hz, 1H), 3.64-3.56 (m, 1H), 3.51 (br d, J=12.4 Hz, 1H), 3.42-3.36 (m, 1H), 3.16-3.03 (m, 1H), 2.30 (br dd, J=2.4, 6.0 Hz, 1H), 2.19-2.10 (m, 1H), 2.06-1.96 (m, 2H), 1.44 (dd, J=1.2, 6.0 Hz, 6H).

### Example 14

### 5-(cyclopropylmethoxy)-3-[6-(3-piperidyl)-2-pyridyl]pyrazolo[1,5-a]pyridine

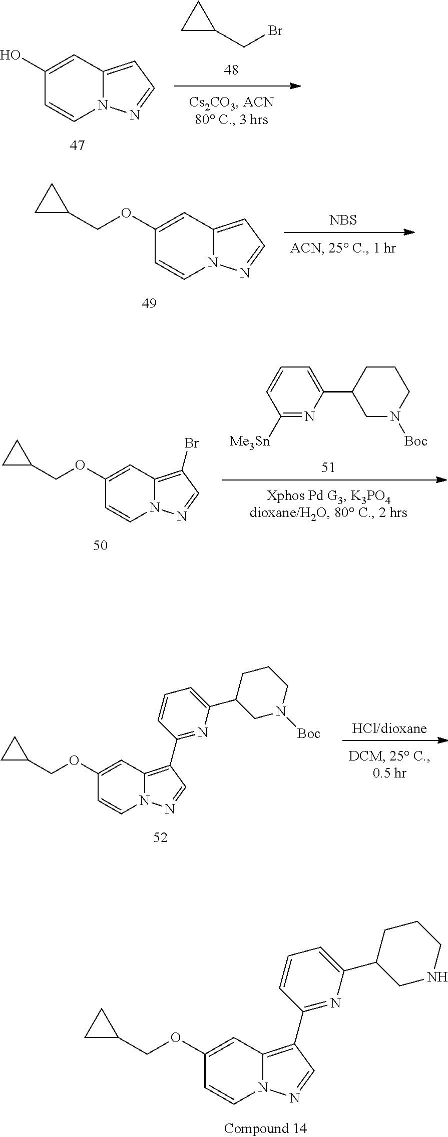

Step A: To a solution of (bromomethyl)cyclopropane 48 (251 mg, 1.86 mmol) and pyrazolo[1,5-a]pyridin-5-ol 47 (0.25 g, 1.86 mmol) in ACN (5 mL) is added Cs₂CO₃ (1.2 g, 3.73 mmol). The mixture is stirred at 80° C. for 3 hrs. The reaction mixture is concentrated under reduced pressure to give a residue which is purified by flash silica gel chromatography (eluent of 0-30% ethyl acetate/petroleum ether gradient) to yield the title compound 5-(cyclopropylmethoxy)pyrazolo[1,5-a]pyridine 49 (341 mg, 81%) as a white solid. LCMS (ESI MS) m/z=189.2 [M+H]+ 1.

Step B: To a solution of 5-(cyclopropylmethoxy)pyrazolo[1,5-a]pyridine 49 (280 mg, 1.49 mmol) in ACN (5 mL) is added NBS (238 mg, 1.34 mmol). The mixture is stirred at 25° C. for 1 hr. The reaction mixture is concentrated under reduced pressure to give a residue which is purified by flash silica gel chromatography (eluent of 0-10% ethyl acetate/petroleum ether gradient) to yield the title compound 3-bromo-5-(cyclopropylmethoxy)pyrazolo[1,5-a]pyridine 50 (283 mg, 71%) as a white solid.

¹H NMR (400 MHz, methanol-d₄) δ=8.35 (d, J=7.6 Hz, 1H), 7.84 (s, 1H), 6.73 (d, J=2.4 Hz, 1H), 6.63 (dd, J=2.8, 7.6 Hz, 1H), 3.93 (d, J=7.2 Hz, 2H), 1.38-1.26 (m, 1H), 0.71-0.63 (m, 2H), 0.44-0.38 (m, 2H).

Step C: A mixture of tert-butyl 3-(6-(trimethylstannyl)pyridin-2-yl)piperidine-1-carboxylate 51 (238.7 mg, 561.54 μmol), 3-bromo-5-(cyclopropylmethoxy)pyrazolo[1,5-a]pyridine 50 (150 mg, 561.5 μmol, 1 eq), XPhos Pd G3 (47.5 mg, 56.15 μmol), and K₃PO₄ (238.4 mg, 1.12 mmol) in dioxane (4 mL) and H₂O (0.1 mL) is purged with N₂ three times. The mixture is stirred at 80° C. for 2 hr under N₂. The reaction mixture is concentrated under reduced pressure to give a residue which is purified by prep-HPLC (column: Xtimate C18, 150×40 mm×10 um; mobile phase: [water (NH₃H₂O+NH₄HCO₃)-ACN]; gradient: 54%-94% B over 32 min) to yield the title compound tert-butyl 3-[6-[5-(cyclopropylmethoxy)pyrazolo[1,5-a]pyridin-3-yl]-2-pyridyl]piperidine-1-carboxylate 52 (70 mg, 27.8%) as a white solid.

¹H NMR (400 MHz, methanol-d₄) δ=8.41-8.38 (m, 2H), 8.10 (d, J=2.8 Hz, 1H), 7.71-7.64 (m, 1H), 7.60-7.57 (m, 1H), 7.04 (d, J=7.2 Hz, 1H), 6.68 (dd, J=2.8, 7.6 Hz, 1H), 4.17 (br d, J=11.6 Hz, 1H), 3.99 (d, J=7.2 Hz, 2H), 2.93-2.79 (m, 2H), 2.17 (br d, J=13.2 Hz, 1H), 2.01-1.79 (m, 2H), 1.71-1.58 (m, 1H), 1.48 (s, 9H), 1.42-1.26 (m, 2H), 0.72-0.62 (m, 2H), 0.43-0.36 (m, 2H).

Step D: To a solution of tert-butyl 3-(6-(5-(cyclopropylmethoxy)pyrazolo[1,5-a]pyridin-3-yl)pyridin-2-yl)piperidine-1-carboxylate 2 (40.0 mg, 89.18 μmol) in DCM (0.5 mL) is added HCl/dioxane (4 M, 0.5 mL). The mixture is stirred at 25° C. for 0.5 hr. The reaction mixture is partitioned between DCM and H₂O. The organic phase is discarded, and the aqueous phase is lyophilized to give the title compound 5-(cyclopropylmethoxy)-3-[6-(3-piperidyl)-2-pyridyl]pyrazolo[1,5-a]pyridine (Compound 14) (17.7 mg, 56%) as an off-white solid. LCMS (ESI MS) m/z=349.2 [M+H]+ 1.

¹H NMR (400 MHz, methanol-d₄) δ=8.68 (s, 1H), 8.61 (d, J=7.6 Hz, 1H), 8.39 (t, J=8.0 Hz, 1H), 8.13 (d, J=8.0 Hz, 1H), 7.65 (d, J=7.6 Hz, 1H), 7.43 (d, J=2.0 Hz, 1H), 6.89 (dd, J=2.4, 7.6 Hz, 1H), 4.07 (d, J=7.2 Hz, 2H), 3.77-3.69 (m, 2H), 3.52 (br d, J=12.4 Hz, 1H), 3.47-3.39 (m, 1H), 3.18-3.07 (m, 1H), 2.30 (br d, J=9.2 Hz, 1H), 2.19-2.11 (m, 1H), 2.08-1.93 (m, 2H), 1.41-1.28 (m, 1H), 0.74-0.65 (m, 2H), 0.44 (q, J=4.8 Hz, 2H).

### Example 15

### 5-(oxetan-3-ylmethoxy)-3-[6-(3-piperidyl)-2-pyridyl]pyrazolo[1,5-a]pyridine

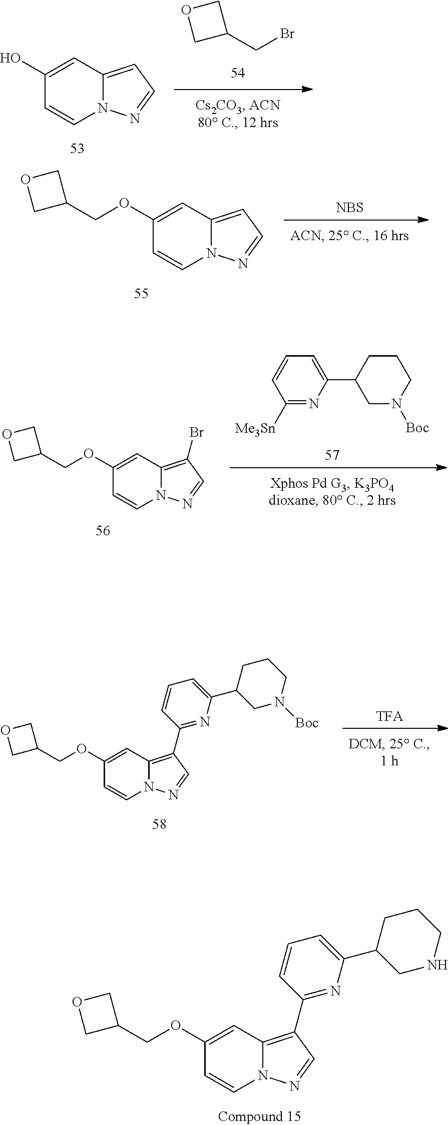

Step A: To a solution of pyrazolo[1,5-a]pyridin-5-ol 53 (300 mg, 2.24 mmol) and 3-(bromomethyl)oxetane 54 (405 mg, 2.68 mmol) in ACN (6 mL) is added Cs₂CO₃ (1.4 g, 4.47 mmol). The mixture is stirred at 80° C. for 12 hrs. The reaction mixture is quenched with H₂O, and the mixture is extracted with EtOAc three times. The combined organic layers are washed with brine, dried over anhydrous Na₂SO₄, filtered and concentrated under reduced pressure to give the title compound 5-(oxetan-3-ylmethoxy)pyrazolo[1,5-a]pyridine 55 (400 mg, crude) as a white solid. LCMS (ESI MS) m/z=205.2.

Step B: To a solution of 5-(oxetan-3-ylmethoxy)pyrazolo[1,5-a]pyridine 55 (380 mg, 1.86 mmol) in ACN (5 mL) is added NBS (264 mg, 1.49 mmol). The mixture is stirred at 25° C. for 1 hr. The reaction is quenched with water and extracted with EtOAc three times. The combined organic layers are dried over anhydrous Na₂SO₄, filtered and concentrated under reduced pressure to give a residue which is purified by flash silica gel chromatography (eluent of 0-25% ethyl acetate/petroleum ether gradient) to yield the title compound 3-bromo-5-(oxetan-3-ylmethoxy)pyrazolo[1,5-a]pyridine 56 (250 mg) as a white solid. LCMS (ESI MS) m/z=285.0 [M+H]+ 1.

Step C: A mixture of 3-bromo-5-(oxetan-3-ylmethoxy)pyrazolo[1,5-a]pyridine 6 (60 mg, 141.13 μmol), tert-butyl 3-(6-(trimethylstannyl)pyridin-2-yl)piperidine-1-carboxylate 57 (47 mg, 169.35 μmol), XPhos Pd G3 (11 mg, 14.11 μmol), and K₃PO₄ (59 mg, 282.2 μmol) in dioxane (3 mL) and H₂O (1 mL) is purged with N₂ three times. The mixture is stirred at 80° C. for 2 hr under N₂. The reaction is filtered, and the filtrate is concentrated under vacuum to give the title compound tert-butyl 3-[6-[5-(oxetan-3-ylmethoxy)pyrazolo[1,5-a]pyridin-3-yl]-2-pyridyl]piperidine-1-carboxylate 8 (120 mg, crude) as a brown oil. LCMS (ESI MS) m/z=465.2 [M+H]+ 1.

Step D: To a solution of tert-butyl 3-(6-(5-(oxetan-3-ylmethoxy)pyrazolo[1,5-a]pyridin-3-yl)pyridin-2-yl)piperidine-1-carboxylate 58 (50 mg, 107.6 μmol) in DCM (1 mL) is added TFA (0.5 mL). The mixture is stirred at 25° C. for 1 hr. The reaction mixture is concentrated under vacuum to give the crude product which is purified by prep-HPLC (column: Xtimate C18, 150×40 mm×10 um; mobile phase: [water (TFA)-ACN]; gradient: 0%-34% B over 36 min) to yield the title compound 5-(oxetan-3-ylmethoxy)-3-[6-(3-piperidyl)-2-pyridyl]pyrazolo[1,5-a]pyridine (Compound 15) (8 mg, 17%) as a white solid. LCMS (ESI MS) m/z=365.0 [M+H]+ 1.

¹H NMR (400 MHz, methanol-d₄) δ=8.49 (s, 1H), 8.47-8.48 (m, 1H), 8.47 (s, 1H), 8.46-8.47 (m, 1H), 7.94 (s, 1H), 7.75-7.83 (m, 1H), 7.70 (d, J=8.00 Hz, 1H), 7.16 (d, J=7.63 Hz, 1H), 6.78 (dd, J=7.38, 2.00 Hz, 1H), 4.96 (s, 2H), 4.64-4.69 (m, 2H), 4.43 (d, J=6.13 Hz, 2H), 3.70 (br d, J=10.76 Hz, 1H), 3.51-3.62 (m, 2H), 3.38-3.48 (m, 2H), 3.04-3.15 (m, 1H), 2.30 (br d, J=11.88 Hz, 1H), 2.07-2.17 (m, 2H), 1.98 (br t, J=12.01 Hz, 1H).

### Example 16

### 4-[3-[6-(3-piperidyl)-2-pyridyl]pyrazolo[1,5-a]pyridin-5-yl]morpholine

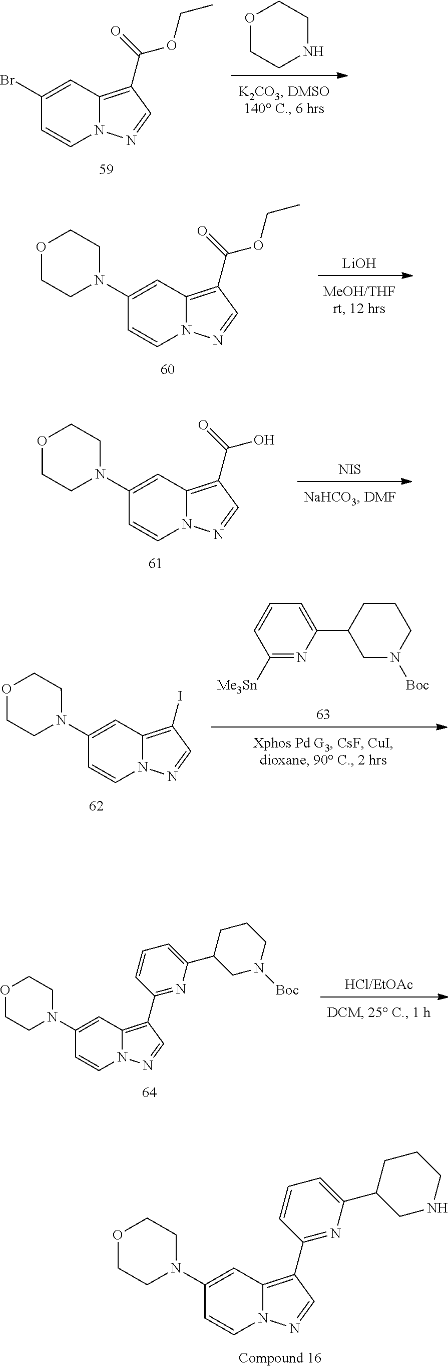

Step A: To a solution of ethyl 5-bromopyrazolo[1,5-a]pyridine-3-carboxylate 59 (900 mg, 3.34 mmol) and morpholine (874.1 mg, 10.0 mmol, 883 μL) in DMSO (5 mL) is added K₂CO₃ (924.5 mg, 6.69 mmol). The mixture is stirred at 140° C. for 6 hrs. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-20% ethyl acetate/petroleum ether gradient) to yield the title compound ethyl 5-morpholinopyrazolo[1,5-a]pyridine-3-carboxylate 60 (600 mg) as a yellow solid. LCMS (ESI MS) m/z=276.0 [M+H]+ 1.

Step B: To a solution of ethyl 5-morpholinopyrazolo[1,5-a]pyridine-3-carboxylate 60 (500 mg, 1.82 mmol) in THF (3 mL) and H₂O (3 mL) is added LiOH·H₂O (304.9 mg, 7.26 mmol). The mixture is stirred at 50° C. for 12 hrs. The reaction is quenched with HCl (1M) and extracted with EtOAc three times. The combined organic layers are washed with HCl (1M) twice, dried over anhydrous Na₂SO₄, filtered and concentrated under reduced pressure to give the title compound 5-morpholinopyrazolo[1,5-a]pyridine-3-carboxylic acid 61 (500 mg) as a yellow solid. LCMS (ESI MS) m/z=247.9 [M+H]+ 1.

Step C: To a solution of 5-morpholinopyrazolo[1,5-a]pyridine-3-carboxylic acid 61 (180 mg, 728 μmol) in DMF (2 mL) are added NIS (327.6 mg, 1.46 mmol) and NaHCO₃ (244.6 mg, 2.91 mmol, 113 μL). The mixture is stirred at 25° C. for 12 hr. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-30% ethyl acetate/petroleum ether gradient) to yield the title compound 4-(3-iodopyrazolo[1,5-a]pyridin-5-yl)morpholine 2 (80 mg) as a yellow oil. LCMS (ESI MS) m/z=330.1 [M+H]+ 1.

Step D: To a solution of 4-(3-iodopyrazolo[1,5-a]pyridin-5-yl)morpholine 62 (20 mg, 60.8 μmol) and tert-butyl 3-(6-(trimethylstannyl)pyridin-2-yl)piperidine-1-carboxylate 6 (51.7 mg, 121.5 μmol) in dioxane (1 mL) are added copper iodide (23.1 mg, 121.5 μmol), CsF (36.9 mg, 243 μmol, 9 μL) and Xphos Pd G3 (10.3 mg, 12.2 μmol). The mixture is stirred at 90° C. for 2 hr under N₂. The reaction mixture is filtered, and the filtrate is concentrated under reduced pressure to give the title compound tert-butyl 3-[6-(5-morpholinopyrazolo[1,5-a]pyridin-3-yl)-2-pyridyl]piperidine-1-carboxylate 64 (15 mg) as a white solid. LCMS (ESI MS) m/z=464.2 [M+H]+ 1.

Step E: To a solution of tert-butyl 3-(6-(5-morpholinopyrazolo[1,5-a]pyridin-3-yl)pyridin-2-yl)piperidine-1-carboxylate 64 (12 mg, 25.9 μmol) in DCM (1 mL) is added HCl/EtOAc (4 M, 6.5 μL). The mixture is stirred at 25° C. for 1 hr. The reaction mixture is concentrated under reduced pressure to give a residue which is purified by prep-HPLC (FA condition; column: Welch Xtimate C18, 150×40 mm×10 um; mobile phase: [water (FA)-ACN]; gradient: 0%-32% B over 25 min) to yield the title compound 4-[3-[6-(3-piperidyl)-2-pyridyl]pyrazolo[1,5-a]pyridin-5-yl]morpholine (Compound 16) (2.7 mg) as a white solid. LCMS (ESI MS) m/z=364.0 [M+H]+ 1.

¹H NMR (400 MHz, methanol-d₄) δ=8.34-8.44 (m, 2H), 7.71-7.84 (m, 2H), 7.64 (br d, J=7.6 Hz, 1H), 7.10 (d, J=7.6 Hz, 1H), 6.92 (dd, J=7.6, 2.8 Hz, 1H), 3.82-3.99 (m, 4H), 3.67 (br d, J=10.4 Hz, 1H), 3.49 (br d, J=13.2 Hz, 1H), 3.38-3.45 (m, 1H), 3.34-3.38 (m, 4H), 3.19-3.29 (m, 1H), 2.96-3.09 (m, 1H), 2.22-2.32 (m, 1H), 1.89-2.14 (m, 3H).

### Example 17

### 3-[6-(3-piperidyl)-2-pyridyl]pyrazolo[1,5-a]pyridine-5-carbonitrile

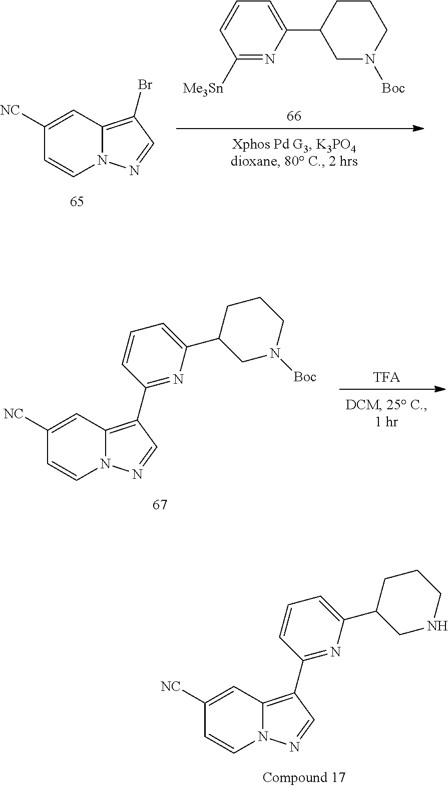

Step A: A mixture of tert-butyl 3-(6-(trimethylstannyl)pyridin-2-yl)piperidine-1-carboxylate 66 (50 mg, 117.6 μmol), 3-bromopyrazolo[1,5-a]pyridine-5-carbonitrile 65 (26.1 mg, 117.6 μmol), XPhos Pd G3 (9.9 mg, 11.8 μmol), and K₃PO₄ (49.9 mg, 235.2 μmol) in dioxane (1 mL) and H₂O (0.25 mL) is purged with N₂ three times. The mixture is stirred at 80° C. for 2 hr under N₂. The reaction mixture is concentrated directly to yield the title compound tert-butyl 3-[6-(5-cyanopyrazolo[1,5-a]pyridin-3-yl)-2-pyridyl]piperidine-1-carboxylate 67 (47.4 mg, crude) as a black solid.

Step B: To a solution of tert-butyl 3-(6-(5-cyanopyrazolo[1,5-a]pyridin-3-yl)pyridin-2-yl)piperidine-1-carboxylate 67 (47 mg, 116.5 μmol) in DCM (5 mL) is added TFA (0.5 mL). The mixture is stirred at 25° C. for 1 hr. The reaction mixture is concentrated to give the crude product which is purified by prep-HPLC (column: Xtimate C18, 150×40 mm×10 um; mobile phase: [water (FA)-ACN]; gradient: 0%-32% B over 25 min) to yield the title compound 3-[6-(3-piperidyl)-2-pyridyl]pyrazolo[1,5-a]pyridine-5-carbonitrile (Compound 17) (5.8 mg, 16%) as a greenish-white solid. LCMS (ESI MS) m/z=304.3 [M+H]+ 1.

¹H NMR (400 MHz, methanol-d₄) δ=9.01 (d, J=0.8 Hz, 1H), 8.77 (dd, J=0.8, 7.2 Hz, 1H), 8.69 (s, 1H), 8.54 (s, 1H), 7.86-7.78 (m, 2H), 7.25 (dd, J=1.2, 7.2 Hz, 1H), 7.17 (dd, J=2.0, 7.2 Hz, 1H), 3.70 (td, J=1.6, 12.0 Hz, 1H), 3.49-3.36 (m, 3H), 3.14-3.0 (m, 1H), 2.26 (br d, J=8.8 Hz, 1H), 2.13-2.07 (m, 1H), 2.00-1.91 (m, 2H).

### Example 18

### 5-cyclopropyl-3-[6-(3-piperidyl)-2-pyridyl]pyrazolo[1,5-a]pyridine

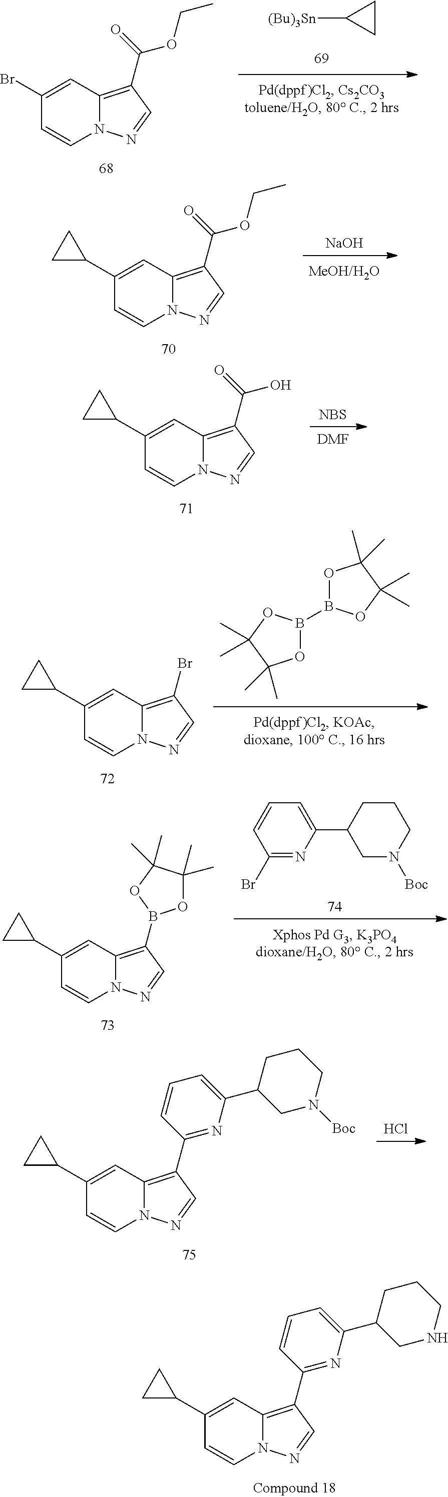

Step A: To a solution of tributyl(cyclopropyl)stannane 69 (922.9 mg, 2.79 mmol) and ethyl 5-bromopyrazolo[1,5-a]pyridine-3-carboxylate 68 (500 mg, 1.86 mmol) in toluene (3 mL) and H₂O (1 mL) are added Pd(dppf)Cl₂ (151.7 mg, 185.8 μmol) and Cs₂CO₃ (1.21 g, 3.7 mmol). The mixture is stirred at 80° C. for 2 hr under N₂. The reaction mixture is concentrated under reduced pressure to give a residue which is purified by silica gel chromatography (eluent of 0-20% ethyl acetate/petroleum ether gradient) to yield the title compound 5-cyclopropylpyrazolo[1,5-a]pyridine-3-carboxylate 70 (200 mg, crude) as a white solid. LCMS (ESI MS) m/z=231.1 [M+H]+ 1.

Step B: To a solution of ethyl 5-cyclopropylpyrazolo[1,5-a]pyridine-3-carboxylate 70 (200 mg, 868.6 μmol) in THF (1 mL) and H₂O (1 mL) is added LiOH·H₂O (145.8 mg, 3.47 mmol). The mixture is stirred at 60° C. for 36 hr. The reaction is quenched with HCl (1M) and extracted with EtOAc three times. The combined organic layers are washed with brine twice, dried over anhydrous Na₂SO₄, filtered and concentrated under reduced pressure to give the title compound 5-cyclopropylpyrazolo[1,5-a]pyridine-3-carboxylic acid 71 (180 mg) as a black oil. LCMS (ESI MS) m/z=203.2 [M+H]+ 1. The crude product is used in the next step without additional purification.

Step C: To a solution of 5-cyclopropylpyrazolo[1,5-a]pyridine-3-carboxylic acid 71 (180 mg, 890.2 μmol) in DMF (2 mL) are added NBS (174 mg, 979.2 μmol) and NaHCO₃ (112 mg, 1.34 mmol, 52 μL). The mixture is stirred at 25° C. for 12 hr. The reaction is quenched with H₂O and extracted with EtOAc three times. The combined organic layers are washed with brine twice, dried over anhydrous Na₂SO₄, filtered and concentrated under reduced pressure to give a residue which is purified by flash silica gel chromatography eluent of 0-10% DCM/petroleum ether gradient) to yield the title compound 3-bromo-5-cyclopropylpyrazolo[1,5-a]pyridine 72 (120 mg) as a yellow oil. LCMS (ESI MS) m/z=239.0 [M+H]+ 1.

Step D: To a solution of 3-bromo-5-cyclopropylpyrazolo[1,5-a]pyridine 72 (140 mg, 492.8 μmol) and 4,4,4′,4′,5,5,5′,5′-octamethyl-2,2′-bi(1,3,2-dioxaborolane) (187 mg, 739 μmol) in dioxane (3 mL) are added Pd(dppf)Cl₂ (40.3 mg, 49.3 μmol) and K₂CO₃ (102.2 mg, 739.2 μmol). The mixture is stirred at 80° C. for 2 hr under N₂. The reaction is quenched with H₂O and extracted with EtOAc three times. The combined organic layers are washed with brine twice, dried over anhydrous Na₂SO₄, filtered and concentrated under reduced pressure to give a residue which is purified by flash silica gel chromatography (eluent of 0-20% DCM/petroleum ether gradient) to yield the title compound 5-cyclopropyl-3-(4,4,5,5-tetramethyl-1,3,2-dioxaborolan-2-yl)pyrazolo[1,5-a]pyridine 73 (120 mg, 85.7% yield) as a white solid. LCMS (ESI MS) m/z=285.1 [M+H]+ 1.

Step E: To a solution of tert-butyl 3-(6-(trimethylstannyl)pyridin-2-yl)piperidine-1-carboxylate 74 (70 mg, 246.3 μmol) and 5-cyclopropyl-3-(4,4,5,5-tetramethyl-1,3,2-dioxaborolan-2-yl)pyrazolo[1,5-a]pyridine 73 (168.1 mg, 492.7 μmol) in H₂O (0.2 mL) and dioxane (0.8 mL) are added K₃PO₄ (104.6 mg, 492.7 μmol) and Xphos Pd G3 (41.7 mg, 49.3 μmol). The mixture is stirred at 60° C. for 12 hr under a N₂ atmosphere. The reaction is quenched with H₂O and extracted with EtOAc three times. The combined organic layers are washed with brine twice, dried over anhydrous Na₂SO₄, filtered and concentrated under reduced pressure to give a residue which is purified by flash silica gel chromatography (eluent of 0-20% ethyl acetate/petroleum ether gradient) to yield the title compound tert-butyl-3-[6-(5-cyclopropylpyrazolo[1,5-a]pyridin-3-yl)-2-pyridyl]piperidine-1-carboxylate 75 (14 mg, 13.6% yield) as a yellow solid. LCMS (ESI MS) m/z=419.3 [M+H]+ 1.

Step F: To a solution of tert-butyl 3-(6-(5-cyclopropylpyrazolo[1,5-a]pyridin-3-yl)pyridin-2-yl)piperidine-1-carboxylate 75 (14 mg, 33.45 μmol) is added HCl/dioxane (4 M, 33.5 μL). The mixture is stirred at 25° C. for 1 hr. The reaction mixture is concentrated under reduced pressure give a residue which is purified by prep-HPLC (FA condition, column: Welch Xtimate C18, 150×30 mm×5 um; mobile phase: [water(FA)-ACN]; gradient: 0%-34% B over 25 min) to yield the title compound 5-cyclopropyl-3-[6-(3-piperidyl)-2-pyridyl]pyrazolo[1,5-a]pyridine (Compound 18) (11.8 mg, 94.9% yield) as a white solid. LCMS (ESI MS) m/z=319.2 [M+H]+ 1.

¹H NMR (400 MHz, DMSO-d₆) δ=8.56-8.65 (m, 2H), 8.32 (s, 2H), 7.65-7.78 (m, 2H), 7.00-7.11 (m, 1H), 6.77 (br d, J=7.6 Hz, 1H), 3.10-3.15 (m, 2H), 2.97 (br dd, J=7.6, 4.8 Hz, 3H), 2.69-2.77 (m, 1H), 2.02-2.16 (m, 3H), 1.76-1.86 (m, 2H), 1.09-1.15 (m, 2H), 0.81-0.88 (m, 2H).

### Example 19

### 3-[6-(3-piperidyl)-2-pyridyl]pyrazolo[1,5-a]pyridin-5-amine

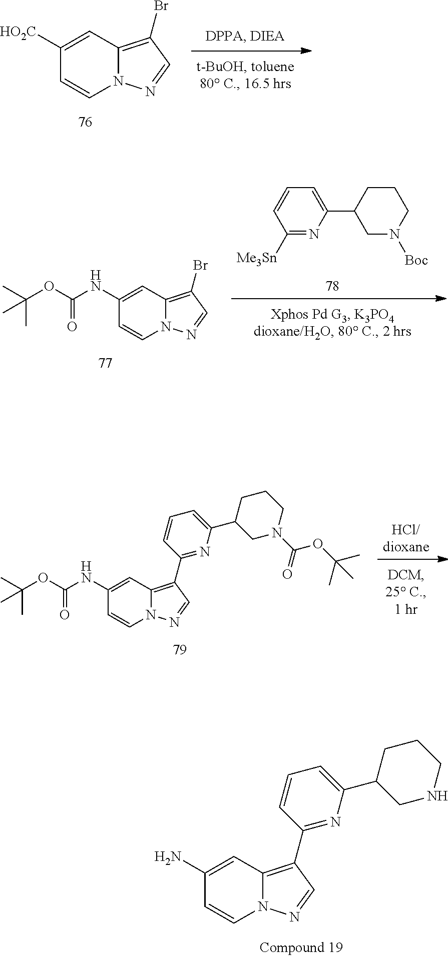

Step A: To a solution of 3-bromopyrazolo[1,5-a]pyridine-5-carboxylic acid 76 (1 g, 4.15 mmol) in t-BuOH (1 mL) and toluene (4 mL) is added dropwise N,N-diisopropylethylamine (DIEA) (536 mg, 4.15 mmol, 722.6 μL) at 25° C. After addition, the mixture is stirred at this temperature for 30 min, and then diphenylphosphoryl azide (DPPA) (1.37 g, 4.98 mmol, 1.1 mL) is added dropwise at 25° C. The resulting mixture is stirred at 80° C. for 16 hrs. The reaction mixture is concentrated under reduced pressure to give a crude product which is purified by flash silica gel chromatography (eluent of 20-40% ethyl acetate/petroleum ether gradient) to yield the title compound tert-butyl N-(3-bromopyrazolo[1,5-a]pyridin-5-yl)carbamate 77 (0.9 g, 53%) as a yellow solid.

¹H NMR (400 MHz, methanol-d₄) δ=8.37 (d, J=7.6 Hz, 1H), 7.86 (s, 1H), 7.78 (d, J=2.0 Hz, 1H), 6.88 (dd, J=2.4, 7.6 Hz, 1H), 1.55 (s, 9H).

Step B: A mixture of tert-butyl 3-(6-(trimethylstannyl)pyridin-2-yl)piperidine-1-carboxylate 78 (89.8 mg, 211.4 μmol), tert-butyl (3-bromopyrazolo[1,5-a]pyridin-5-yl)carbamate 77 (60 mg, 192.2 μmol), XPhos Pd G3 (16.2 mg, 19.2 μmol), and K₃PO₄ (81.6 mg, 384.4 μmol) in dioxane (2 mL) and H₂O (0.1 mL) is degassed and purged with N₂ three times. The mixture is stirred at 80° C. for 2 hr under a N₂ atmosphere. The reaction mixture is concentrated under reduced pressure to give a residue which is purified by flash silica gel chromatography (eluent of 30-40% ethyl acetate/petroleum ether gradient) to yield the title compound tert-butyl 3-[6-[5-(tert-butoxycarbonylamino)pyrazolo[1,5-a]pyridin-3-yl]-2-pyridyl]piperidine-1-carboxylate 79 (50 mg, 52%) as a white solid. LCMS (ESI MS) m/z=494.6 [M+H]+ 1.

Step C: To a solution of tert-butyl 3-(6-(5-((tert-butoxycarbonyl)amino)pyrazolo[1,5-a]pyridin-3-yl)pyridin-2-yl)piperidine-1-carboxylate 79 (40 mg, 80.2 μmol) in DCM (1 mL) is added HCl/dioxane (1 mL, 4M). The mixture is stirred at 25° C. for 1 hr. The reaction mixture is concentrated under reduced pressure to give a residue which is purified by prep-HPLC (column: Xtimate C18, 150×40 mm×10 um; mobile phase: [water (NH₃H₂O+NH₄HCO₃)-ACN]; gradient: 10%-50% B over 32 min) to yield the title compound 3-[6-(3-piperidyl)-2-pyridyl]pyrazolo[1,5-a]pyridin-5-amine (Compound 19) (7.9 mg, 25%) as a white solid. LCMS (ESI MS) m/z=294.1 [M+H]+ 1.

¹H NMR (400 MHz, methanol-d₄) δ=8.27 (s, 1H), 8.22 (d, J=7.6 Hz, 1H), 7.69-7.64 (m, 1H), 7.53 (s, 1H), 7.50 (br d, J=3.2 Hz, 1H), 7.00 (d, J=7.6 Hz, 1H), 6.52 (dd, J=2.4, 7.6 Hz, 1H), 3.55-3.44 (m, 1H), 3.35 (br d, J=1.6 Hz, 1H), 3.29-3.26 (m, 1H), 3.19-3.07 (m, 1H), 3.05-2.96 (m, 1H), 2.22-2.13 (m, 1H), 2.02-1.93 (m, 2H), 1.91-1.81 (m, 1H).

### Example 20

### N-methyl-3-[6-(3-piperidyl)-2-pyridyl]pyrazolo[1,5-a]pyridin-5-amine

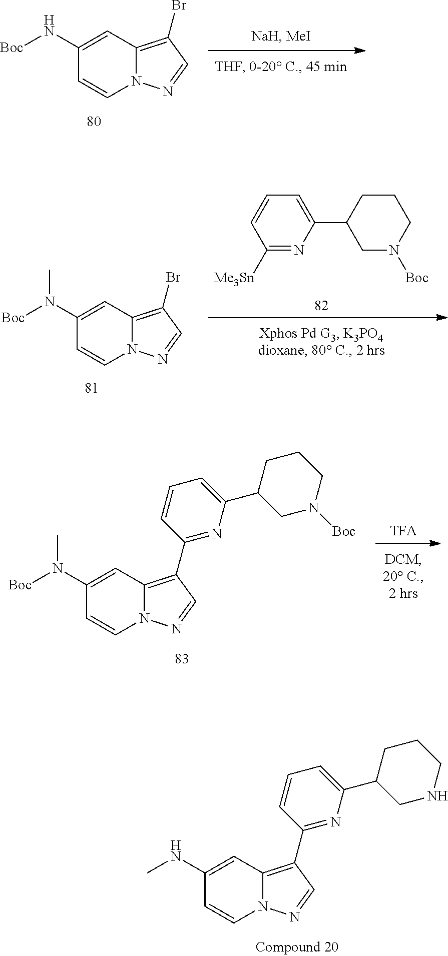

Step A: To a solution of tert-butyl (3-bromopyrazolo[1,5-a]pyridin-5-yl)carbamate 80 (200 mg, 640.7 μmol) in THF (3 mL) is added NaH (51 mg, 1.28 mmol, 60% purity) at 0° C. After addition, the mixture is stirred at this temperature for 15 min, and then Mel (109.1 mg, 768.8 μmol, 47 μL) is added dropwise at 0° C. The resulting mixture is stirred at 20° C. for 30 min. The reaction mixture is quenched by addition 1N NH₄Cl (aq) and extracted with EtOAc three times. The combined organic layers are washed with brine, dried over Na₂SO₄, filtered and concentrated under reduced pressure to give a residue. The residue is purified by flash silica gel chromatography (eluent of 0-10% ethyl acetate/petroleum ether gradient) to yield the title compound tert-butyl N-(3-bromopyrazolo[1,5-a]pyridin-5-yl)-N-methyl-carbamate 81 (200 mg, 90%) as a colorless oil. LCMS (ESI MS) m/z=326.0 [M+H]+ 1.

¹H NMR (400 MHz, DMSO-d₆) δ=8.66 (d, J=7.6 Hz, 1H), 8.12 (s, 1H), 7.38 (d, J=2.4 Hz, 1H), 7.03 (dd, J=7.6, 2.1 Hz, 1H), 3.29 (s, 3H), 1.45 (s, 9H).

Step B: A mixture of tert-butyl (3-bromopyrazolo[1,5-a]pyridin-5-yl)(methyl)carbamate 81 (60 mg, 183.9 μmol), tert-butyl 3-(6-(trimethylstannyl)pyridin-2-yl)piperidine-1-carboxylate 82 (93.8 mg, 220.7 μmol), XPhos Pd G3 (15.5 mg, 18.39 μmol), and K₃PO₄ (78 mg, 367.9 μmol) in dioxane (2 mL) is purged with N₂ three times. The mixture is stirred at 80° C. for 2 hr under N₂. The reaction mixture is concentrated directly to yield the title compound tert-butyl 3-[6-[5-[tert-butoxycarbonyl(methyl)amino]pyrazolo[1,5-a]pyridin-3-yl]-2-pyridyl]piperidine-1-carboxylate 8 (93 mg, crude) as a brown solid. LCMS (ESI MS) m/z=508.2 [M+H]+ 1.

Step C: To a solution of tert-butyl 3-(6-(5-((tert-butoxycarbonyl)(methyl)amino)-pyrazolo[1,5-a]pyridin-3-yl)pyridin-2-yl)piperidine-1-carboxylate 8 (90 mg, 177.3 μmol) in DCM (2 mL) is added TFA (0.4 mL). The mixture is stirred at 20° C. for 2 hrs. The reaction mixture is extracted with H₂O three times. The combined water layers are concentrated under reduced pressure to give a residue which is purified by prep-HPLC (column: Xtimate C18, 150×40 mm×10 um; mobile phase: [water (FA)-ACN]; gradient: 0%-28% B over 25 min) to yield the title compound N-methyl-3-[6-(3-piperidyl)-2-pyridyl]pyrazolo[1,5-a]pyridin-5-amine (Compound 20) (33.1 mg, 58%, FA) as a yellow solid. LCMS (ESI MS) m/z=308.1 [M+H]+ 1.

¹H NMR (400 MHz, methanol-d₄) δ=8.53 (s, 1H), 8.28 (s, 1H), 8.19 (d, J=7.6 Hz, 1H), 7.67-7.72 (m, 1H), 7.57 (d, J=7.6 Hz, 1H), 7.30 (d, J=2.4 Hz, 1H), 7.04 (d, J=7.4 Hz, 1H), 6.48 (dd, J=7.6, 2.6 Hz, 1H), 3.66 (dd, J=12.4, 3.8 Hz, 1H), 3.41-3.49 (m, 2H), 3.23 (tt, J=10.8, 3.7 Hz, 1H), 3.02 (td, J=12.4, 3.3 Hz, 1H), 2.93 (s, 3H), 2.20-2.29 (m, 1H), 2.00-2.11 (m, 2H), 1.90-1.99 (m, 1H).

### Example 21

### N-ethyl-3-[6-(3-piperidyl)-2-pyridyl]pyrazolo[1,5-a]pyridin-5-amine

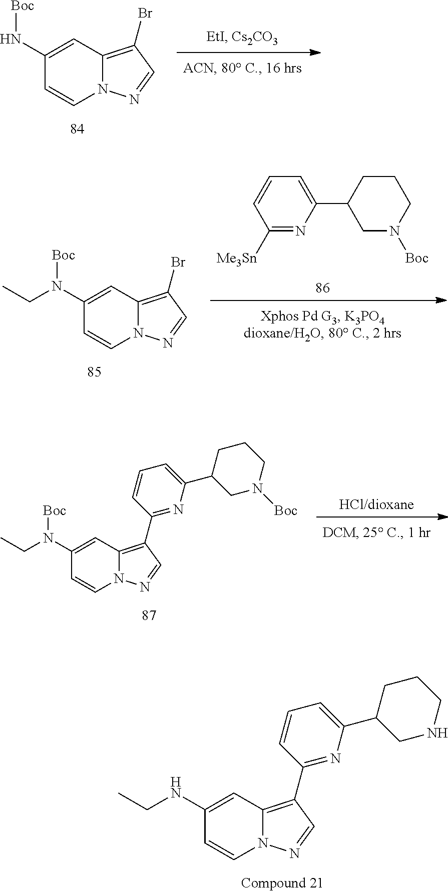

Step A: To a solution of EtI (199.9 mg, 1.28 mmol) and tert-butyl (3-bromopyrazolo[1,5-a]pyridin-5-yl)carbamate 84 (0.1 g, 320.3 μmol) in ACN (3 mL) is added Cs₂CO₃ (208 mg, 640.7 μmol). The mixture is stirred at 80° C. for 16 hr. The reaction mixture is concentrated under reduced pressure to give a residue which is purified by flash silica gel chromatography (eluent of 0-20% ethyl acetate/petroleum ether gradient) to yield the title compound tert-butyl N-(3-bromopyrazolo[1,5-a]pyridin-5-yl)-N-ethyl-carbamate 85 (140 mg, crude) as a white solid.

¹H NMR (400 MHz, methanol-d₄) δ=8.49 (d, J=7.6 Hz, 1H), 7.96 (s, 1H), 7.37 (d, J=2.0 Hz, 1H), 6.89 (dd, J=2.4, 7.6 Hz, 1H), 3.78 (q, J=7.2 Hz, 2H), 1.48 (s, 9H), 1.21 (t, J=7.2 Hz, 3H).

Step B: A mixture of tert-butyl 3-(6-(trimethylstannyl)pyridin-2-yl)piperidine-1-carboxylate 8 (175 mg, 411.5 μmol), tert-butyl (3-bromopyrazolo[1,5-a]pyridin-5-yl)(ethyl)carbamate 85 (140 mg, 411.5 μmol), K₃PO₄ (175 mg, 823.0 μmol), and XPhos Pd G3 (35 mg, 41.15 μmol) in dioxane (1 mL) and H₂O (0.2 mL) is purged with N₂ three times, and then the mixture is stirred at 80° C. for 2 hr under N₂. The reaction mixture is concentrated under reduced pressure to give a residue which is purified by flash silica gel chromatography (eluent of 0-30% ethyl acetate/petroleum ether gradient) to yield the title compound tert-butyl 3-[6-[5-[tert-butoxycarbonyl(ethyl)amino]pyrazolo[1,5-a]pyridin-3-yl]-2-pyridyl]piperidine-1-carboxylate 87 (0.14 g) as a white solid.

¹H NMR (400 MHz, methanol-d₄) δ=8.58 (d, J=1.6 Hz, 1H), 8.53 (d, J=7.6 Hz, 1H), 8.50 (s, 1H), 7.73-7.68 (m, 1H), 7.66-7.63 (m, 1H), 7.08 (d, J=7.6 Hz, 1H), 6.97 (dd, J=2.4, 7.6 Hz, 1H), 4.40-4.26 (m, 1H), 4.19-4.12 (m, 1H), 3.82 (q, J=7.2 Hz, 2H), 2.94-2.80 (m, 2H), 2.19-2.10 (m, 1H), 1.98-1.77 (m, 3H), 1.69-1.57 (m, 2H), 1.51-1.46 (m, 18H), 1.24 (t, J=7.2 Hz, 2H).

Step C: To a solution of tert-butyl 3-(6-(5-((tert-butoxycarbonyl)(ethyl)amino)-pyrazolo[1,5-a]pyridin-3-yl)pyridin-2-yl)piperidine-1-carboxylate 87 (0.07 g, 134.19 μmol) in DCM (3 mL) is added HCl/dioxane (4 M, 34 μL). The mixture is stirred at 25° C. for 1 hr. The reaction mixture is partitioned between DCM and H₂O. The organic phase is discarded, and the aqueous phase is lyophilized to yield the title compound N-ethyl-3-[6-(3-piperidyl)-2-pyridyl]pyrazolo[1,5-a]pyridin-5-amine (Compound 21) (33.7 mg, 78%) as a white solid. LCMS (ESI MS) m/z=321.8 [M+H]+ 1.

¹H NMR (400 MHz, methanol-d₄) δ=8.56 (s, 1H), 8.34 (d, J=7.6 Hz, 1H), 8.26 (br t, J=8.4 Hz, 1H), 7.99 (d, J=8.4 Hz, 1H), 7.46 (d, J=8.0 Hz, 1H), 6.89 (br s, 1H), 6.65 (d, J=7.6 Hz, 1H), 3.71 (br d, J=12.0 Hz, 1H), 3.66-3.57 (m, 1H), 3.51 (br d, J=12.0 Hz, 1H), 3.45-3.37 (m, 1H), 3.30-3.27 (m, 2H), 3.15-3.07 (m, 1H), 2.33-2.26 (m, 1H), 2.18-2.12 (m, 1H), 2.06-1.93 (m, 2H), 1.34 (t, J=7.2 Hz, 3H).

### Example 22

### 3-(methylsulfonamido)-N-(3-(6-(piperidin-3-yl)pyridin-2-yl)pyrazolo[1,5-a]pyridin-5-yl)propanamide

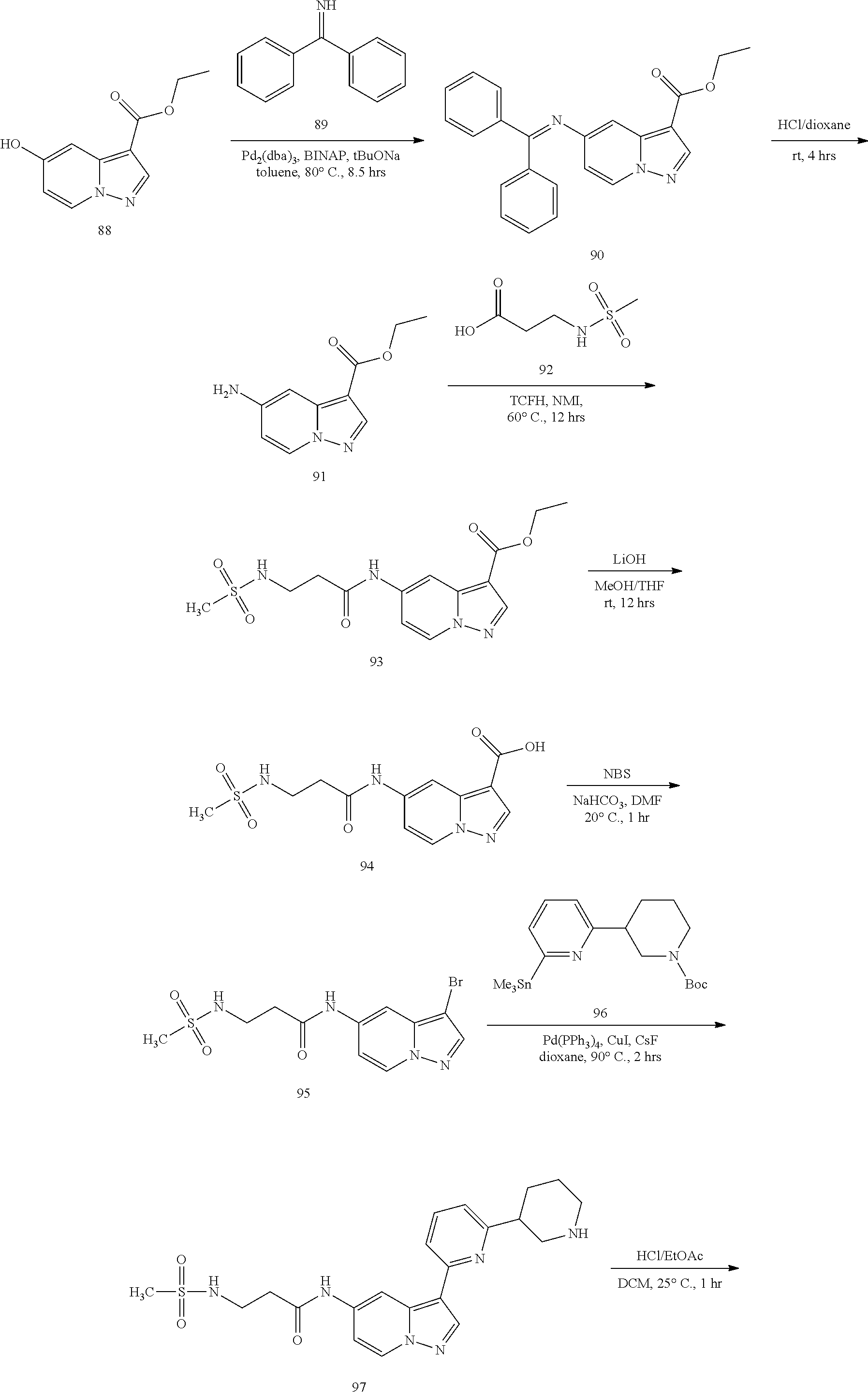

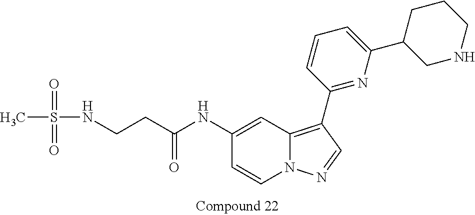 [SMILES: CS(=O)(=O)NCCC(=O)Nc1ccn2ncc(-c3cccc(C4CCCNC4)n3)c2c1]

Step A: To a solution of ethyl 5-bromopyrazolo[1,5-a]pyridine-3-carboxylate 88 (2 g, 7.43 mmol), diphenylmethanimine 89 (1.7 g, 9.66 mmol, 1.6 mL) in toluene (20 mL) are added BINAP (2.3 g, 3.72 mmol, 0.5 eq), Pd₂(dba)₃ (1.3 g, 1.49 mmol, 0.2 eq) and t-BuONa (2.1 g, 22.3 mmol). The mixture is stirred at 80° C. for 8.5 hr. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-5% ethyl acetate/petroleum ether gradient) to yield the title compound ethyl 5-(benzhydrylideneamino)pyrazolo[1,5-a]pyridine-3-carboxylate 90 (700 mg, 1.89 mmol, 25.5% yield) as a yellow oil. LCMS (ESI MS) m/z=370.1 [M+H]+ 1.

Step B: To a solution of ethyl 5-((diphenylmethylene)amino)pyrazolo[1,5-a]pyridine-3-carboxylate 90 (600 mg, 1.62 mmol) in DCM (5 mL) is added HCl/dioxane (5 mL). The mixture is stirred at 25° C. for 4 hrs. The reaction wa is s quenched with water and extracted with EtOAc three times. The combined organic layers are washed with brine twice, dried over anhydrous Na₂SO₄, filtered and concentrated under reduced pressure to give the title compound ethyl 5-aminopyrazolo[1,5-a]pyridine-3-carboxylate 91 (400 mg) as a white solid. LCMS (ESI MS) m/z=206.2 [M+H]+ 1.

Step C: To a solution of ethyl 5-aminopyrazolo[1,5-a]pyridine-3-carboxylate 9 (400 mg, 1.95 mmol) and 3-(methylsulfonamido)propanoic acid 2 (391 mg, 2.34 mmol) in DMF (10 mL) are added TCFH (1.1 g, 3.90 mmol) and NMI (480 mg, 5.85 mmol, 466 μL). The mixture is stirred at 50° C. for 12 hrs. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-80% ethyl acetate/petroleum ether gradient) to yield the title compound ethyl 5-[3-(methanesulfonamido)propanoylamino]pyrazolo[1,5-a]pyridine-3-carboxylate 93 (400 mg, 1.13 mmol, 57.9% yield) as a yellow solid. LCMS (ESI MS) m/z=355.1 [M+H]+ 1.

Step D: To a solution of ethyl 5-(3-(methylsulfonamido)propanamido)pyrazolo[1,5-a]pyridine-3-carboxylate 3 (400 mg, 1.13 mmol) in MeOH (1 mL) is added LiOH·H₂O (236.8 mg, 5.64 mmol) in H₂O (1 mL). The mixture is stirred at 25° C. for 12 hrs. The reaction is quenched with water and extracted with EtOAc three times. The combined organic layers are washed with brine twice, dried over anhydrous Na₂SO₄, filtered and concentrated under reduced pressure to give the title compound 5-[3-(methanesulfonamido)propanoylamino]pyrazolo[1,5-a]pyridine-3-carboxylic acid 94 (350 mg) as a white solid. LCMS (ESI MS) m/z=206.2 [M+H]+ 1.

Step E: To a solution of 5-(3-(methylsulfonamido)propanamido)pyrazolo[1,5-a]pyridine-3-carboxylic acid 94 (340 mg, 1.04 mmol) in DMF (4 mL) are added NaHCO₃ (131 mg, 1.56 mmol, 61 μL) and NBS (185.4 mg, 1.04 mmol). The mixture is stirred at 25° C. for 1 hr. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-80% ethyl acetate/petroleum ether gradient) to yield the title compound 5-[3-(methanesulfonamido)propanoylamino]pyrazolo[1,5-a]pyridine-3-carboxylic acid 9 (150 mg, 415.3 μmol, 39.9% yield) as a white solid. LCMS (ESI MS) m/z=355.1 [M+H]+ 1.

¹H NMR (400 MHz, DMSO-d₆) δ=10.43 (s, 1H), 8.65 (d, J=7.50 Hz, 1H), 7.96-8.24 (m, 2H), 7.14 (t, J=5.82 Hz, 1H), 6.94 (dd, J=7.50, 2.25 Hz, 1H), 3.34 (s, 2H), 2.93 (s, 3H), 2.63 (t, J=6.88 Hz, 2H).

Step F: A mixture of N-(3-bromopyrazolo[1,5-a]pyridin-5-yl)-3-(methylsulfonamido)-propanamide 95 (50 mg, 138.4 μmol), tert-butyl 3-(6-(trimethylstannyl)pyridin-2-yl)piperidine-1-carboxylate 96 (117.7 mg, 276.8 μmol), XPhos Pd G₃ (23.4 mg, 27.68 μmol), CuI (52.7 mg, 276.8 μmol), and CsF (84.1 mg, 553.7 μmol, 20.4 μL) in dioxane (3 mL) is purged with N₂ three times. The mixture is stirred at 90° C. for 2 hr under N₂. The reaction mixture is concentrated under vacuum to give the title compound tert-butyl 3-[6-[5-[3-(methanesulfonamido)propanoylamino]-pyrazolo[1,5-a]pyridin-3-yl]-2-pyridyl]piperidine-1-carboxylate 97 (80 mg) as a white solid. LCMS (ESI MS) m/z=443.2 [M+H]+ 1.

Step G: To a solution of tert-butyl 3-(6-(5-(3-(methylsulfonamido)propanamido)-pyrazolo[1,5-a]pyridin-3-yl)pyridin-2-yl)piperidine-1-carboxylate 9 (40 mg, 73.7 μmol) in DCM (1 mL) is added TFA (1 mL). The mixture is stirred at 25° C. for 1 hr. The reaction mixture is concentrated under vacuum to give the crude product which is purified by prep-HPLC (FA condition; column: Welch Xtimate C18, 150×40 mm×10 um; mobile phase: [water (FA)-ACN]; gradient:0%-30% B over 25 min) to yield the title compound 3-(methylsulfonamido)-N-(3-(6-(piperidin-3-yl)pyridin-2-yl)pyrazolo[1,5-a]pyridin-5-yl)propanamide (Compound 22) (6 mg, 11.5 μmol, 15.6% yield) as a white solid. LCMS (ESI MS) m/z=443.2 [M+H]+ 1.

¹H NMR (400 MHz, methanol-d₄) δ=9.49 (s, 1H), 8.47 (br s, 2H), 7.70-7.79 (m, 1H), 7.63 (d, J=7.88 Hz, 1H), 7.12 (d, J=7.50 Hz, 1H), 6.82 (br d, J=0.63 Hz, 1H), 3.77-3.89 (m, 1H), 3.65 (br dd, J=12.38, 3.13 Hz, 1H), 3.53 (t, J=6.32 Hz, 2H), 3.40-3.49 (m, 2H), 3.27 (br t, J=10.76 Hz, 1H), 3.04 (s, 3H), 2.77 (t, J=6.32 Hz, 2H), 2.03-2.18 (m, 3H), 1.97 (br d, J=11.76 Hz, 1H).

### Example 23

### 3-(methanesulfonamido)-N-[3-[6-(3-piperidyl)-2-pyridyl]pyrazolo[1,5-a]pyridin-6-yl]propanamide

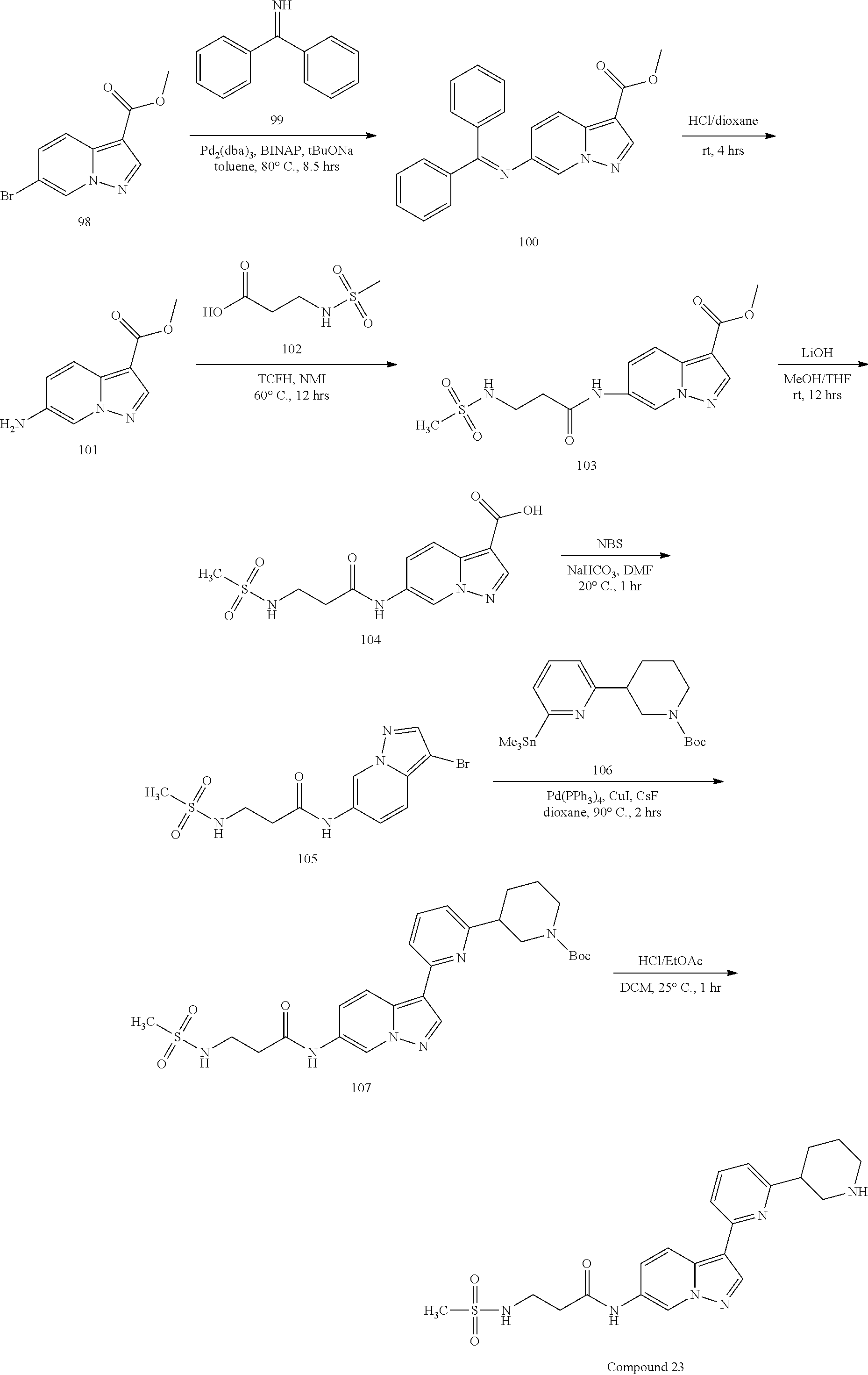

Step A: A mixture of methyl 6-bromopyrazolo[1,5-a]pyridine-3-carboxylate 98 (1 g, 3.92 mmol), diphenylmethanimine 99 (1.4 g, 7.84 mmol, 1.32 mL), Pd₂(dba)₃ (718 mg, 784.1 μmol), BINAP (1.2 g, 1.96 mmol), and t-BuONa (1.1 g, 11.76 mmol) in toluene (20 mL) is purged with N₂ three times. The mixture is stirred at 80° C. for 8.5 hr under N₂. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-5% ethyl acetate/petroleum ether gradient) to yield the title compound methyl 6-(benzhydrylideneamino)pyrazolo[1,5-a]pyridine-3-carboxylate 100 (400 mg) as a yellow oil. LCMS (ESI MS) m/z=356.2 [M+H]+ 1.

Step B: To a solution of methyl 6-((diphenylmethylene)amino)pyrazolo[1,5-a]pyridine-3-carboxylate 100 (350 mg, 984.8 μmol) in DCM (2 mL) is added HCl/dioxane (2 mL). The mixture is stirred at 25° C. for 4 hr. The reaction is quenched with water and extracted with EtOAc three times. The combined organic layers are washed with brine twice, dried over anhydrous Na₂SO₄, filtered and concentrated under reduced pressure to give a residue which is used without purification to give the title compound methyl 6-aminopyrazolo[1,5-a]pyridine-3-carboxylate 101 (200 mg, crude) as a white solid. LCMS (ESI MS) m/z=192.2 [M+H]+ 1.

Step C: To a solution of methyl 6-aminopyrazolo[1,5-a]pyridine-3-carboxylate 101 (200 mg, 878.5 μmol) and 3-(methylsulfonamido)propanoic acid 102 (146 mg, 878.55 μmol) in DMF (3 mL) are added TCFH (493 mg, 1.76 mmol) and NMI (216.4 mg, 2.64 mmol, 210 μL). The mixture is stirred at 25° C. for 12 hr. The mixture is stirred at 60° C. for 12 hrs. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-80% ethyl acetate/petroleum ether gradient) to yield the title compound methyl 6-[3-(methanesulfonamido) propanoylamino]pyrazolo[1,5-a]pyridine-3-carboxylate 103 (200 mg, 53.5% yield, 80% purity) as a white solid. LCMS (ESI MS) m/z=340.9 [M+H]+ 1.

Step D: To a solution of methyl 6-(3-(methylsulfonamido)propanamido)pyrazolo[1,5-a]pyridine-3-carboxylate 103 (200 mg, 587.6 μmol) in MeOH (2 mL) is added LiOH·H₂O (123 mg, 2.94 mmol, 5 eq) in H₂O (2 mL). The mixture is stirred at 25° C. for 12 hr. The reaction is quenched with water and extracted with EtOAc three times. The combined organic layers are washed with brine twice, dried over anhydrous Na₂SO₄, filtered and concentrated under reduced pressure to give the title compound 6-[3-(methanesulfonamido)propanoylamino]pyrazolo[1,5-a]pyridine-3-carboxylic acid 104 (170 mg, 86.9% yield) as a white solid. LCMS (ESI MS) m/z=327.0 [M+H]+ 1.

Step E: To a solution of 6-(3-(methylsulfonamido)propanamido)pyrazolo[1,5-a]pyridine-3-carboxylic acid 104 (140 mg, 429 μmol) in DMF (3 mL) are added NaHCO₃ (54 mg, 643.5 μmol, 25.0 μL) and NBS (99 mg, 557.7 μmol). The mixture is stirred at 20° C. for 1 hr. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-80% ethyl acetate/petroleum ether gradient) to yield the title compound N-(3-bromopyrazolo[1,5-a]pyridin-6-yl)-3-(methanesulfonamido)propanamide 105 (150 mg, 96.8% yield) as a white solid. LCMS (ESI MS) m/z=362.9 [M+H]+ 1.

¹H NMR (400 MHz, methanol-d₄) δ=7.58-7.72 (m, 1H), 7.44 (d, J=7.75 Hz, 1H), 7.32 (d, J=7.50 Hz, 1H), 4.04-4.17 (m, 2H), 3.76 (br s, 2H), 2.83-3.02 (m, 4H), 1.98-2.07 (m, 1H).

Step F: A mixture of N-(3-bromopyrazolo[1,5-a]pyridin-6-yl)-3-(methylsulfonamido)-propanamide 105 (20 mg, 7.4 μmol), tert-butyl 3-(6-(trimethylstannyl)pyridin-2-yl)piperidine-1-carboxylate 106 (21 mg, 8.8 μmol), K₃PO₄ (3 mg, 14.7 μmol), and XPhos Pd G₃ (1.3 mg, 1.5 μmol) in dioxane (1 mL) and H₂O (0.2 mL) is purged with N₂ three times. The mixture is stirred at 90° C. for 2 hr under N₂. The reaction mixture is concentrated under vacuum to give the title compound tert-butyl 3-[6-[6-[3-(methanesulfonamido)propanoylamino]pyrazolo [1,5-a]pyridin-3-yl]-2-pyridyl]piperidine-1-carboxylate 107 (80 mg, crude) as a white solid. LCMS (ESI MS) m/z=543.2 [M+H]+ 1.

Step G: To a solution of tert-butyl 3-(6-(6-(3-(methylsulfonamido)-propanamido)pyrazolo[1,5-a]pyridin-3-yl)pyridin-2-yl)piperidine-1-carboxylate 107 (15 mg, 27.6 μmol) in DCM (0.5 mL) is added TFA (0.5 mL). The mixture is stirred at 25° C. for 1 hr. The reaction mixture is concentrated under vacuum to give the crude product which is purified by prep-HPLC (FA condition; column: Welch Xtimate C18, 150×40 mm×10 um; mobile phase: [water(FA)-ACN]; gradient: 0%-26% B over 25 min) to give the title compound 3-(methanesulfonamido)-N-[3-[6-(3-piperidyl)-2-pyridyl]pyrazolo[1,5-a]pyridin-6-yl]propanamide (Compound 23) (1 mg, 7.6% yield) as a white solid. LCMS (ESI MS) m/z=442.8 [M+H]+ 1.

¹H NMR (400 MHz, methanol-d₄) δ=9.40 (s, 1H), 8.46-8.54 (m, 2H), 7.77-7.84 (m, 1H), 7.67-7.75 (m, 1H), 7.38-7.46 (m, 1H), 7.17 (d, J=7.38 Hz, 1H), 3.63-3.72 (m, 1H), 3.42-3.54 (m, 5H), 3.10-3.19 (m, 1H), 3.00 (s, 3H), 2.73 (t, J=6.50 Hz, 2H), 2.25 (br dd, J=10.82, 2.81 Hz, 1H), 2.04-2.13 (m, 1H), 1.88-2.02 (m, 2H).

### Example 24

### N-[2-(methanesulfonamido)ethyl]-3-[6-(3-piperidyl)-2-pyridyl]pyrazolo[1,5-a]pyridine-6-carboxamide

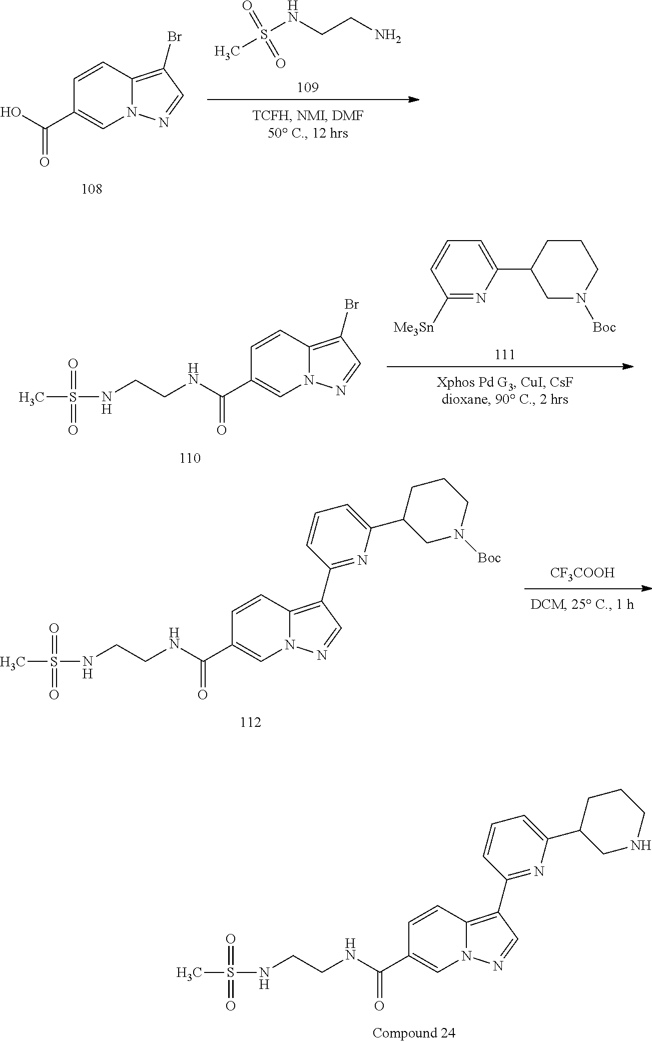

Step A: To a solution of 3-bromopyrazolo[1,5-a]pyridine-6-carboxylic acid 108 (200 mg, 829.7 μmol) and 109 (229.3 mg, 1.66 mmol) in DMF (5 mL) are added TCFH (465.6 mg, 1.66 mmol) and NMI (204.4 mg, 2.49 mmol, 198.4 μL). The mixture is stirred at 50° C. for 12 hrs. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-4% MeOH/DCM gradient) to yield the title compound 3-bromo-N-[2-(methanesulfonamido)ethyl]pyrazolo[1,5-a]pyridine-6-carboxamide 110 (100 mg, 33% yield) as a white solid. LCMS (ESI MS) m/z=362.8 [M+H]+ 1.

Step B: To a solution of 3-bromo-N-(2-(methylsulfonamido)ethyl)pyrazolo[1,5-a]pyridine-6-carboxamide 110 (40 mg, 110.74 μmol) and tert-butyl 3-(6-(trimethylstannyl)pyridin-2-yl)piperidine-1-carboxylate 111 (94.16 mg, 221.48 μmol) in dioxane (4 mL) are added CuI (42.2 mg, 221.5 μmol), CsF (67.3 mg, 442.5 μmol, 16.4 μL) and XPhos Pd G₃ (18.8 mg, 22.2 μmol). The mixture is stirred at 100° C. for 2 hr under a N₂ atmosphere. The reaction mixture is filtered and concentrated under reduced pressure to give the title compound 3-[6-[6-[2-(methanesulfonamido)ethylcarbamoyl]pyrazolo[1,5-a]pyridin-3-yl]-2-pyridyl]piperidine-1-carboxylate 112 (15 mg, crude) as a yellow solid. LCMS (ESI MS) m/z=543.1 [M+H]+ 1.

Step C: To a solution of tert-butyl 3-(6-(6-((2-(methylsulfonamido)ethyl)-carbamoyl)pyrazolo[1,5-a]pyridin-3-yl)pyridin-2-yl)piperidine-1-carboxylate 112 (15 mg, 27.6 μmol, 1 eq) in DCM (1 mL) is added CF₃COOH (9.5 mg, 82.9 μmol, 6.2 μL, 3 eq.). The mixture is stirred at 25° C. for 1 hr. The reaction mixture is concentrated under reduced pressure to give a residue which is purified by prep-HPLC (FA condition; column: Welch Xtimate C18, 150×40 mm×10 um; mobile phase: [water (FA)-ACN]; gradient: 0%-28% B over 25 min) to yield the title compound N-[2-(methanesulfonamido)ethyl]-3-[6-(3-piperidyl)-2-pyridyl]pyrazolo[1,5-a]pyridine-6-carboxamide (Compound 24) (3.3 mg, 24% yield) as a white solid. LCMS (ESI MS) m/z=443.1 [M+H]+ 1.

¹H NMR (400 MHz, methanol-d₄) δ=9.18 (s, 1H), 8.67 (s, 1H), 8.57 (d, J=9.6 Hz, 1H), 7.71-7.86 (m, 3H), 7.21 (d, J=7.6 Hz, 1H), 3.69 (br d, J=12.4 Hz, 1H), 3.54-3.62 (m, 2H), 3.42-3.52 (m, 2H), 3.35-3.39 (m, 2H), 3.23-3.30 (m, 1H), 3.10-3.19 (m, 1H), 2.99 (s, 3H), 2.25 (br s, 1H), 1.88-2.14 (m, 3H).

### Example 25

### N-[2-oxo-2-[3-(6-pyrazolo[1,5-a]pyridin-3-yl-2-pyridyl)-1-piperidyl]ethyl]methanesulfonamide

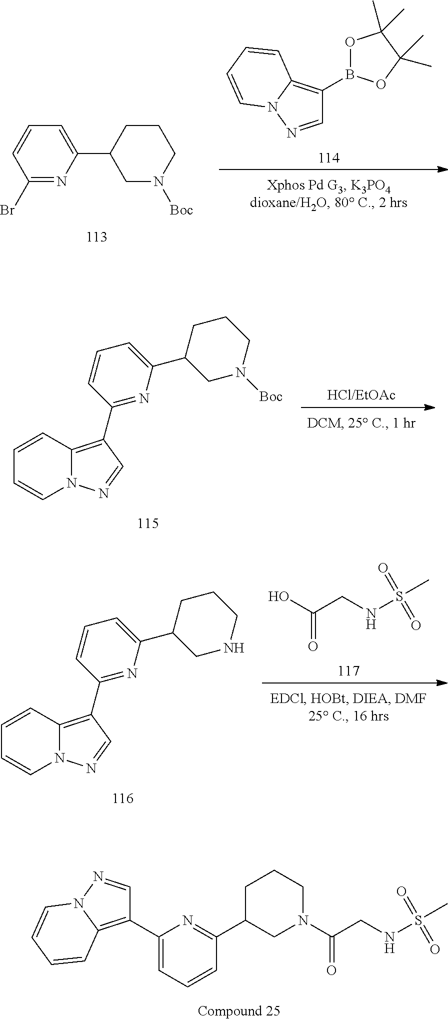

Step A: A mixture of 3-(4,4,5,5-tetramethyl-1,3,2-dioxaborolan-2-yl)pyrazolo[1,5-a]pyridine 114 (679.5 mg, 2.78 mmol), tert-butyl 3-(6-bromopyridin-2-yl)piperidine-1-carboxylate 113 (950 mg, 2.78 mmol), XPhos Pd G3 (235.6 mg, 278.4 μmol), and K₃PO₄ (1.18 g, 5.57 mmol) in dioxane (10 mL) and H₂O (2.5 mL) is degassed and purged with N₂ three times. The mixture is stirred at 80° C. for 2 hr under N₂. The reaction mixture is concentrated to give a residue which is purified by flash silica gel chromatography (ISCO®; 4 g SepaFlash® Silica Flash Column, eluent of 0-11% ethyl acetate/petroleum ether gradient @25 mL/min) to yield the title compound tert-butyl 3-(6-pyrazolo[1,5-a]pyridin-3-yl-2-pyridyl)piperidine-1-carboxylate 115 (230 mg, 16.3% yield) as a white solid. LCMS (ESI MS) m/z=379.2 [M+H]+ 1.

Step B: A solution of tert-butyl 3-(6-(pyrazolo[1,5-a]pyridin-3-yl)pyridin-2-yl)piperidine-1-carboxylate 115 (230 mg, 607.7 μmol) in 4M HCl/EtOAc (1 mL) and DCM (0.5 mL) is stirred at 25° C. for 1 hr. The reaction mixture is concentrated under vacuum to yield the title compound 3-[6-(3-piperidyl)-2-pyridyl]pyrazolo[1,5-a]pyridine 116 (150 mg) as a yellow solid. LCMS (ESI MS) m/z=279.1 [M+H]+ 1.

Step C: To a solution of (methylsulfonyl)glycine 117 (33 mg, 215.6 μmol) and 3-(6-(piperidin-3-yl)pyridin-2-yl)pyrazolo[1,5-a]pyridine 116 (50 mg, 179.6 μmol) in DMF (2 mL) are added HOBt (36.4 mg, 269.4 μmol), DIEA (69.6 mg, 538.9 μmol, 94 μL) and EDCI (51.6 mg, 269.4 μmol). The mixture is stirred at 25° C. for 16 hrs. The reaction mixture is filtered and concentrated. The residue is purified by prep-HPLC (column: Welch Xtimate C₁₈, 150×30 mm×5 um; mobile phase: [water(FA)-ACN]; gradient: 10%-50% B over 25 min) to give the title compound N-[2-oxo-2-[3-(6-pyrazolo[1,5-a]pyridin-3-yl-2-pyridyl)-1-piperidyl]ethyl]methane-sulfonamide (Compound 25) (14 mg, 18.3% yield) as a white solid. LCMS (ESI MS) m/z=414.1 [M+H]+ 1.

¹H NMR (400 MHz, methanol-d₄) δ=8.64 (d, J=8.8 Hz, 1H), 8.58 (d, J=6.8 Hz, 1H), 8.51 (d, J=1.2 Hz, 1H), 7.73 (q, J=8.0 Hz, 1H), 7.69-7.63 (m, 1H), 7.48-7.37 (m, 1H), 7.13 (dd, J=18.4, 7.2 Hz, 1H), 7.04-6.96 (m, 1H), 4.81-4.48 (m, 1H), 4.22-3.90 (m, 3H), 3.47 (dd, J=13.6, 11.2 Hz, 1H), 3.25-3.16 (m, 1H), 3.12-3.03 (m, 1H), 2.99 (d, J=17.2 Hz, 3H), 2.94-2.80 (m, 1H), 2.25-2.12 (m, 1H), 2.09-1.86 (m, 2H), 1.83-1.58 (m, 1H).

### Example 26

### 3-[6-(1-methylsulfonyl-3-piperidyl)-2-pyridyl]pyrazolo[1,5-a]pyridine

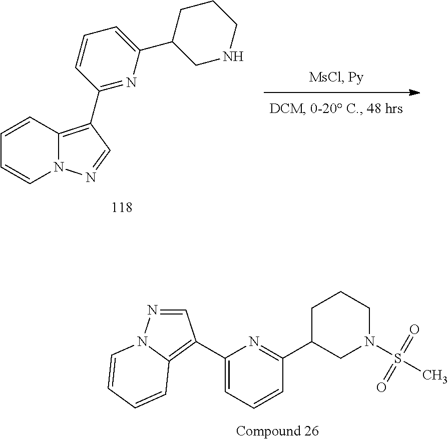 [SMILES: c1cc(-c2cnn3ccccc23)nc(C2CCCNC2)c1 ; "MsCl, Py" ; CS(=O)(=O)N1CCCC(c2cccc(-c3cnn4ccccc34)n2)C1]

Step A: To a solution of 3-(6-(piperidin-3-yl)pyridin-2-yl)pyrazolo[1,5-a]pyridine 118 (70 mg, 251.5 μmol) in DCM (3 mL) are added MsCl (0.19 g, 1.66 mmol, 128.4 μL) and pyridine (99. mg, 1.26 mmol, 101 μL). The mixture is stirred at 20° C. for 48 hr. The reaction mixture is quenched with sat. NaHCO₃ at 0° C. and extracted with CH₂Cl₂ three times and NH₄Cl three times. The combined organic layers are concentrated to give a residue which is purified by prep-HPLC (column: Welch Xtimate C18, 150×30 mm×5 um; mobile phase: [water(FA)-ACN]; gradient: 20%-60% B over 25 min) to afford the title compound 3-[6-(1-methylsulfonyl-3-piperidyl)-2-pyridyl]pyrazolo[1,5-a]pyridine (Compound 26) (5.6 mg, 4.7% yield) as a white solid. LCMS (ESI MS) m/z=357.1 [M+H]+ 1.

¹H NMR (400 MHz, DMSO-d₆) δ=8.76 (d, J=7.0 Hz, 1H), 8.69 (s, 1H), 8.58 (d, J=8.8 Hz, 1H), 7.78-7.73 (m, 2H), 7.48-7.38 (m, 1H), 7.16 (dd, J=5.6, 2.8 Hz, 1H), 7.05-6.97 (m, 1H), 3.84 (br d, J=10.4 Hz, 1H), 3.63 (br d, J=12.4 Hz, 1H), 3.06-2.94 (m, 2H), 2.91 (s, 3H), 2.81 (dt, J=11.6, 2.4 Hz, 1H), 2.13-2.03 (m, 1H), 1.90 (td, J=12.8, 2.8 Hz, 1H), 1.84-1.61 (m, 2H).

### Example 27

### 3-(4-methoxy-6-(piperidin-3-yl)pyridin-2-yl)pyrazolo[1,5-a]pyridine

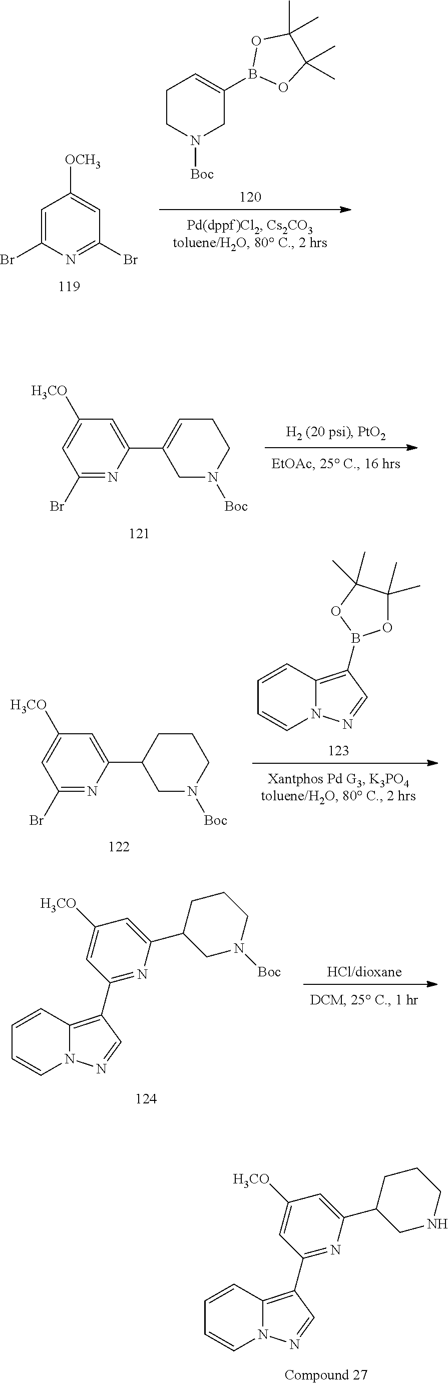

Step A: A mixture of 2,6-dibromo-4-methoxypyridine 119 (863.2 mg, 3.23 mmol), tert-butyl 5-(4,4,5,5-tetramethyl-1,3,2-dioxaborolan-2-yl)-3,6-dihydropyridine-1(2H)-carboxylate 120 (500 mg, 1.62 mmol), Pd(dppf)Cl₂ (264.1 mg, 323.4 μmol), and Cs₂CO₃ (1.05 g, 3.23 mmol) in toluene (5 mL) and H₂O (1 mL) is degassed and purged with N₂ three times. The mixture is stirred at 80° C. for 2 hours under a N₂ atmosphere. The reaction mixture is concentrated under reduced pressure to remove solvent. The residue is diluted with water and extracted with ethyl acetate three times. The combined organic layers are washed with brine twice, dried over Na₂SO₄, filtered and concentrated under reduced pressure to give a residue. The residue is purified by flash silica gel chromatography (eluent of 0-23% ethyl acetate/petroleum ether gradient) to afford the title compound tert-butyl 6-bromo-4-methoxy-5′,6′-dihydro-[2,3′-bipyridine]-1′(2′H)-carboxylate 121 (320 mg, crude) as a colorless oil. LCMS (ESI MS) m/z=313.0 [M+H]+ 1.

Step B: To a solution of tert-butyl 6-bromo-4-methoxy-5′,6′-dihydro-[2,3′-bipyridine]-1′(2′H)-carboxylate 121 (270 mg, 731.2 μmol) in EtOAc (5 mL) is added PtO₂ (16.6 mg, 73.1 μmol) under N₂. The suspension is degassed under vacuum and purged with H₂ several times. The mixture is stirred under H₂ (20 psi) at 25° C. for 16 hrs. The reaction mixture is filtered and concentrated under reduced pressure to remove solvent. The residue is purified by flash silica gel chromatography (eluent of 0-13% ethyl acetate/petroleum ether gradient) to afford the title compound tert-butyl 3-(6-bromo-4-methoxypyridin-2-yl)piperidine-1-carboxylate 122 (100 mg, crude) as a colorless oil. LCMS (ESI MS) m/z=373.0 [M+H]+ 1.

Step C: A mixture of tert-butyl 3-(6-bromo-4-methoxypyridin-2-yl)piperidine-1-carboxylate 122 (80 mg, 215.5 μmol), 3-(4,4,5,5-tetramethyl-1,3,2-dioxaborolan-2-yl)pyrazolo[1,5-a]pyridine 123 (52.6 mg, 215.5 μmol), K₃PO₄ (91.4 mg, 430.9 μmol), and XantPhos Pd G3 (40.8 mg, 43.1 μmol) in dioxane (3 mL) and H₂O (0.7 mL) is degassed and purged with N₂ three times. The mixture is stirred at 80° C. for 2 hours under a N₂ atmosphere. The reaction mixture is concentrated under reduced pressure to remove solvent. The residue is diluted with water and extracted with ethyl acetate three times. The combined organic layers are washed with brine twice, dried over Na₂SO₄, filtered and concentrated under reduced pressure to give a residue. The residue is purified by flash silica gel chromatography (eluent of 0-23% ethyl acetate/petroleum ether gradient) to afford the title compound tert-butyl 3-(4-methoxy-6-(pyrazolo[1,5-a]pyridin-3-yl)pyridin-2-yl)piperidine-1-carboxylate 124 (88 mg, crude) as a colorless oil. LCMS (ESI MS) m/z=409.3 [M+H]+ 1.

Step D: To a solution of tert-butyl 3-(4-methoxy-6-(pyrazolo[1,5-a]pyridin-3-yl)pyridin-2-yl)piperidine-1-carboxylate 124 (78 mg, 190.9 μmol) in DCM (0.5 mL) is added HCl/dioxane (4 M, 0.5 mL). The mixture is stirred at 25° C. for 1 hr. The reaction mixture is concentrated under reduced pressure to remove solvent. The resulting residue is washed with acetonitrile twice and concentrated under reduced pressure to afford the title compound 3-(4-methoxy-6-(piperidin-3-yl)pyridin-2-yl)pyrazolo[1,5-a]pyridine (Compound 27) (26 mg, 35%) as a white solid. LCMS (ESI MS) m/z=309.2 [M+H]+ 1.

¹H NMR (400 MHz, DMSO-d₆) δ=9.16-9.50 (m, 2H), 8.77-8.86 (m, 2H), 8.43 (br d, J=7.00 Hz, 1H), 7.49 (br t, J=7.32 Hz, 1H), 7.43 (br s, 1H), 7.10 (br t, J=6.19 Hz, 1H), 6.83-7.05 (m, 1H), 4.00 (br s, 3H), 3.51-3.57 (m, 2H), 3.31 (br d, J=10.13 Hz, 2H), 2.94 (br d, J=11.01 Hz, 1H), 2.12 (br d, J=11.38 Hz, 1H), 1.92 (br s, 3H).

### Example 28

### 3-(4-chloro-6-(piperidin-3-yl)pyridin-2-yl)pyrazolo[1,5-a]pyridine

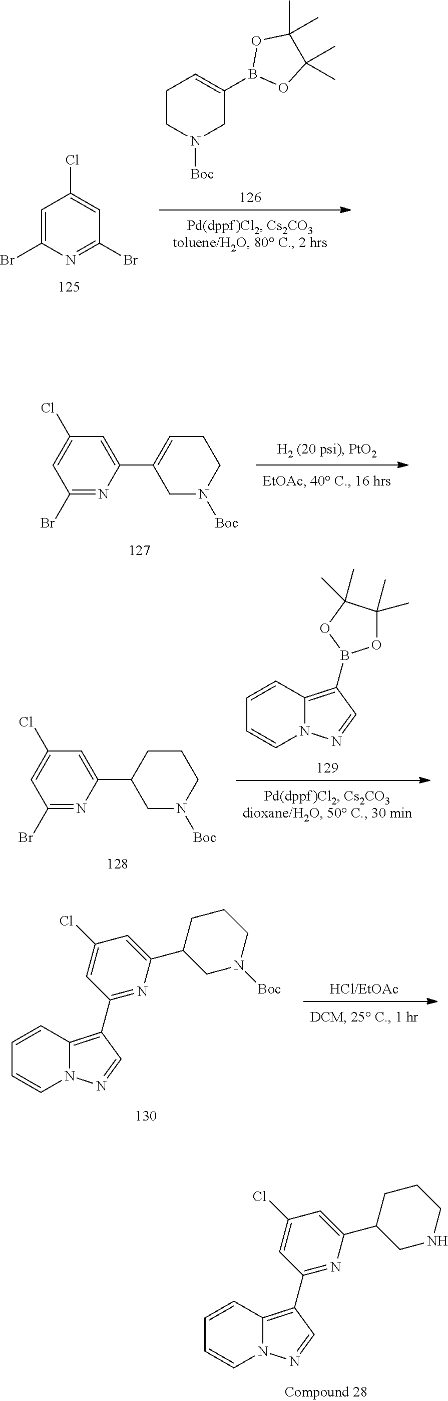

Step A: A mixture of 2,6-dibromo-4-chloropyridine 125 (527 mg, 1.94 mmol), tert-butyl 5-(4,4,5,5-tetramethyl-1,3,2-dioxaborolan-2-yl)-3,6-dihydropyridine-1(2H)-carboxylate 126 (300 mg, 970.2 μmol), Cs₂CO₃ (632 mg, 1.94 mmol), and Pd(dppf)Cl₂·CH₂Cl₂ (158 mg, 194.04 μmol) in toluene (4 mL) and H₂O (1 mL) is degassed and purged with N₂ three times. The mixture is stirred at 80° C. for 2 hr under a N₂ atmosphere. The reaction mixture is diluted with water and extracted with ethyl acetate three times. The combined organic layers are dried over Na₂SO₄, filtered and concentrated under reduced pressure to give a crude product which is purified by flash silica gel chromatography (eluent of 0-15% ethyl acetate/etroleum ether gradient) to yield the title compound tert-butyl 5-(6-bromo-4-chloro-2-pyridyl)-3,6-dihydro-2H-pyridine-1-carboxylate 127 (270 mg, 74.5% yield) as a colorless oil. LCMS (ESI MS) m/z=318.7 [M−56]+1.

Step B: A mixture of tert-butyl 6-bromo-4-chloro-5′,6′-dihydro-[2,3′-bipyridine]-1′(2′H)-carboxylate 127 (250 mg, 669 μmol) and PtO₂ (46 mg, 200.7 μmol) in EtOAc (5 mL) is degassed and purged with H₂ three times. The mixture is stirred at 40° C. for 16 hr under a 20 psi H₂ atmosphere. The reaction mixture is filtered, and the filtrate is concentrated to give the title compound tert-butyl 3-(6-bromo-4-chloro-2-pyridyl)piperidine-1-carboxylate 128 (140 mg) as a yellow oil. LCMS (ESI MS) m/z=376.9 [M+H]+ 1.

Step C: A mixture of tert-butyl 3-(6-bromo-4-chloropyridin-2-yl)piperidine-1-carboxylate 128 (100 mg, 266.3 μmol), 3-(4,4,5,5-tetramethyl-1,3,2-dioxaborolan-2-yl)pyrazolo[1,5-a]pyridine 129 (50 mg, 204.8 μmol), Pd(dppf)Cl₂·CH₂Cl₂ (33 mg, 41 μmol), and Cs₂CO₃ (135 mg, 409.7 μmol) in dioxane (2 mL) and H₂O (0.5 mL) is degassed and purged with N₂ three times. The mixture is stirred at 50° C. for 0.5 hr under a N₂ atmosphere. The reaction mixture is diluted with water and extracted with ethyl acetate three times. The combined organic layers are dried over Na₂SO₄, filtered and concentrated under reduced pressure to give a crude product which is purified by flash silica gel chromatography (eluent of 0-10% ethyl acetate/petroleum ether gradient) to yield the title compound tert-butyl 3-(4-chloro-6-pyrazolo[1,5-a]pyridin-3-yl-2-pyridyl)piperidine-1-carboxylate 130 (27 mg, 31.9% yield) as a yellow oil. LCMS (ESI MS) m/z=413.2 [M+H]+ 1.

Step D: To a solution of tert-butyl 3-(4-chloro-6-(pyrazolo[1,5-a]pyridin-3-yl)pyridin-2-yl)piperidine-1-carboxylate 130 (11 mg, 26.6 μmol) in DCM (0.5 mL) is added HCl/EtOAc (0.5 mL/0.5 mL). The mixture is stirred at 25° C. for 1 hr. The reaction mixture is filtered and concentrated under vacuum to give the crude product which is purified by prep-HPLC (column: Welch Xtimate C18, 150×40 mm×10 um; mobile phase: [water (FA)-ACN]; gradient: 0%-36% B over 25 min) to yield the title compound 3-(4-chloro-6-(piperidin-3-yl)pyridin-2-yl)pyrazolo[1,5-a]pyridine (Compound 28) (6.8 mg, 74.5%) as a white solid. LCMS (ESI MS) m/z=313.1 [M+H]+ 1.

¹H NMR (400 MHz, methanol-d₄) δ=8.61 (br d, J=6.88 Hz, 1H), 8.50-8.57 (m, 2H), 7.78 (d, J=1.50 Hz, 1H), 7.42-7.48 (m, 1H), 7.22 (s, 1H), 6.99-7.07 (m, 1H), 3.67 (br d, J=11.38 Hz, 1H), 3.38-3.49 (m, 2H), 3.22-3.29 (m, 1H), 3.06-3.16 (m, 1H), 2.23 (br d, J=8.00 Hz, 1H), 2.02-2.13 (m, 1H), 1.90-2.01 (m, 2H).

### Example 29

### 3-[5-chloro-6-(3-piperidyl)-2-pyridyl]pyrazolo[1,5-a]pyridine

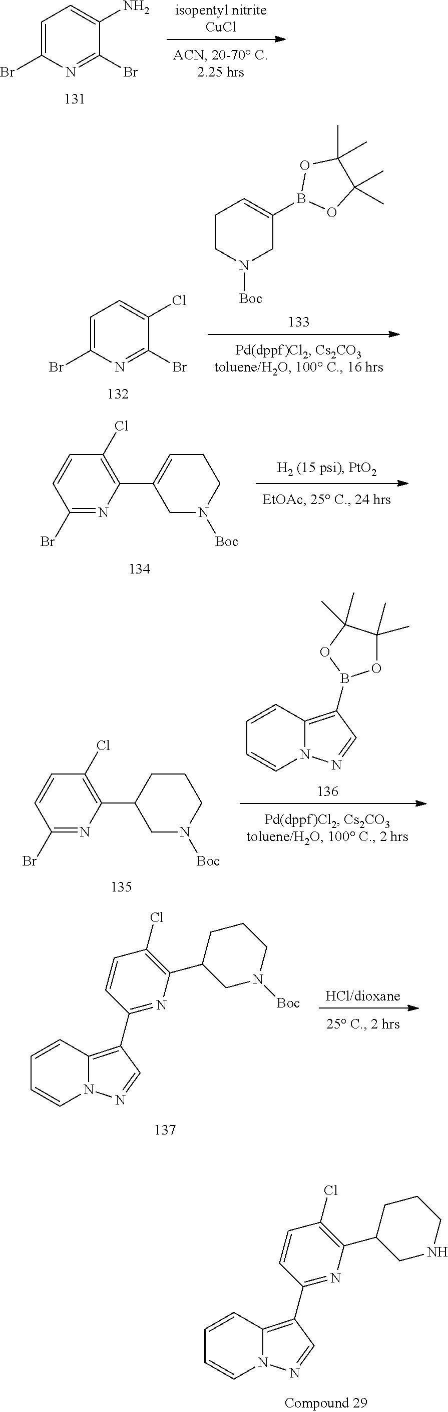

Step A: A mixture of 2,6-dibromopyridin-3-amine 131 (3 g, 11.91 mmol) and CuCl (1.77 g, 17.86 mmol) in ACN (40 mL) is stirred for 15 min at 20° C. Then isopentyl nitrite (2.79 g, 23.82 mmol) is added. The mixture is stirred for 2 hr at 70° C. The reaction mixture is concentrated directly. The residue is purified by flash silica gel chromatography (eluent of 5% ethyl acetate/petroleum ether gradient) to yield the title compound 2, 6-dibromo-3-chloropyridine 132 (2.38 g) as a white solid. LCMS (ESI MS) m/z=269.8 [M+H]+ 1.

Step B: A mixture of 2,6-dibromo-3-chloropyridine 132 (2.38 g, 8.77 mmol), tert-butyl 5-(4,4,5,5-tetramethyl-1,3,2-dioxaborolan-2-yl)-3,6-dihydropyridine-1(2H)-carboxylate 133 (1.63 g, 5.26 mmol), Pd(dppf)Cl₂ (1.28 g, 1.75 mmol), and Cs₂CO₃ (5.72 g, 17.54 mmol) in toluene (40 mL) and H₂O (10 mL) is purged with N₂ three times, and then the mixture is stirred at 100° C. for 16 hr under N₂. The reaction mixture is diluted with H₂O and extracted with EtOAc three times. The combined organic layers are washed with brine, dried, filtered and concentrated under reduced pressure to give a residue. The residue is purified by flash silica gel chromatography (eluent of 7% ethyl acetate/petroleum ether gradient) to yield the title compound tert-butyl 5-(6-bromo-3-chloro-2-pyridyl)-3,6-dihydro-2H-pyridine-1-carboxylate 134 (240 mg) as a yellow solid. LCMS (ESI MS) m/z=373.0 [M+H]+ 1.

Step C: To a solution of tert-butyl 6-bromo-3-chloro-5′,6′-dihydro-[2,3′-bipyridine]-1′(2′H)-carboxylate 134 (150 mg, 401.42 μmol) in EtOAc (3 mL) is added PtO₂ (18.2 mg, 80.28 μmol) under N₂. The suspension is purged with H₂ three times. The mixture is stirred under H₂ (15 psi) at 25° C. for 24 hr. The reaction mixture is concentrated directly. The residue is purified by flash silica gel chromatography (eluent of 4% ethyl acetate/petroleum ether gradient) to yield the title compound tert-butyl 3-(6-bromo-3-chloro-2-pyridyl)piperidine-1-carboxylate 135 (35 mg) as a white solid. LCMS (ESI MS) m/z=375.0 [M+H]+ 1.

Step D: A mixture of tert-butyl 3-(6-bromo-3-chloropyridin-2-yl)piperidine-1-carboxylate 135 (35 mg, 93.16 μmol), 3-(4,4,5,5-tetramethyl-1,3,2-dioxaborolan-2-yl)pyrazolo[1,5-a]pyridine 136 (22.7 mg, 93.16 μmol), Pd(dppf)Cl₂ (13.6 mg, 18.63 μmol), and Cs₂CO₃ (60.7 mg, 186.32 μmol) in toluene (1 mL) and H₂O (0.25 mL) is purged with N₂ three times, and then the mixture is stirred at 100° C. for 2 hr under N₂. The reaction mixture is diluted with H₂O and extracted with EtOAc three times. The combined organic layers are washed with brine, dried, filtered and concentrated under reduced pressure to give the title compound tert-butyl 3-(3-chloro-6-pyrazolo[1,5-a]pyridin-3-yl-2-pyridyl)piperidine-1-carboxylate 137 (38 mg) as a brown oil. LCMS (ESI MS) m/z=413.1 [M+H]+ 1.

Step E: A solution of tert-butyl 3-(3-chloro-6-(pyrazolo[1,5-a]pyridin-3-yl)pyridin-2-yl)piperidine-1-carboxylate 137 (35 mg, 84.76 μmol) in HCl/dioxane (4 M, 2 mL) is stirred at 25° C. for 2 hr. The reaction mixture is concentrated directly. The residue is purified by prep-HPLC (column: Xtimate C18, 150×40 mm×10 um; mobile phase: [water (FA)-ACN]; gradient: 0%-34% B over 25 min) to yield the title compound 3-[5-chloro-6-(3-piperidyl)-2-pyridyl]pyrazolo[1,5-a]pyridine (Compound 29) (8.8 mg, 28%, FA) as a white solid. LCMS (ESI MS) m/z=313.1 [M+H]+1.

¹H NMR (400 MHz, methanol-d₄) δ=8.54-8.63 (m, 2H), 8.50 (s, 1H), 8.44 (d, J=9.2 Hz, 1H), 7.76-7.82 (m, 1H), 7.69 (d, J=8.4 Hz, 1H), 7.41-7.47 (m, 1H), 7.02 (t, J=6.4 Hz, 1H), 3.69-3.78 (m, 1H), 3.57-3.63 (m, 1H), 3.54 (d, J=11.6 Hz, 1H), 3.44-3.50 (m, 1H), 3.10-3.20 (m, 1H), 2.19-2.27 (m, 1H), 2.05-2.13 (m, 1H), 1.86-2.01 (m, 2H).

### Example 30

### 3-(3-chloro-6-(piperidin-3-yl)pyridin-2-yl)pyrazolo[1,5-a]pyridine

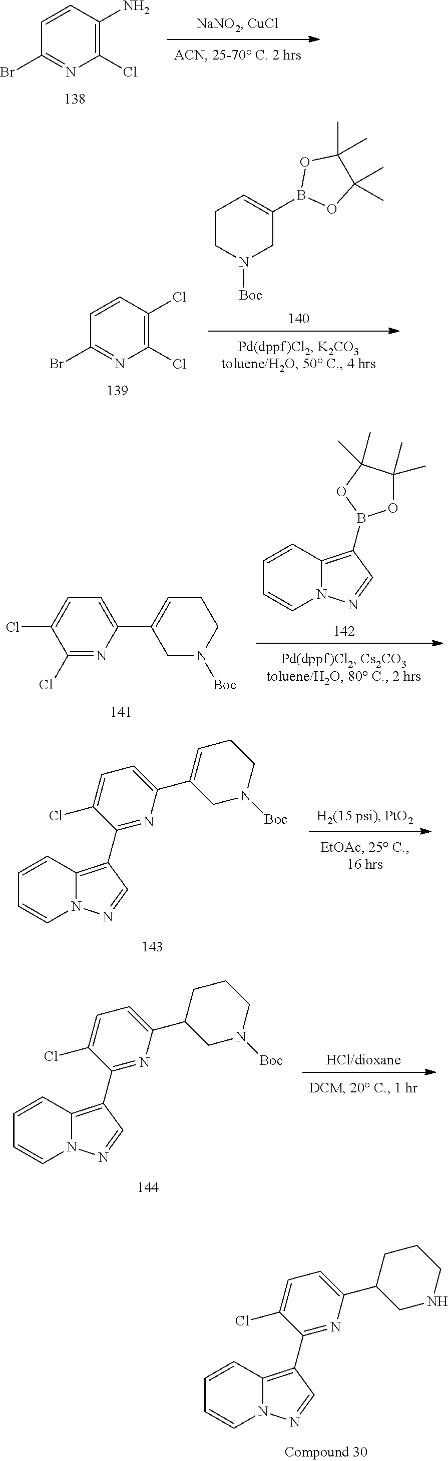

Step A: A mixture of 6-bromo-2-chloropyridin-3-amine 138 (2 g, 9.64 mmol) and CuCl (1.43 g, 14.46 mmol) in ACN (20 mL) is stirred for 15 min at 25° C. Then isopentyl nitrite (2.26 g, 19.28 mmol) is added, and the mixture is stirred at 70° C. for 2 hr. The mixture is filtered, and the filtrate is evaporated under vacuum to give a residue. The residue is purified by flash silica gel chromatography (eluent of 0-10% ethyl acetate/petroleum ether gradient) to yield the title compound 6-bromo-2,3-dichloropyridine 139 (0.5 g) as a yellow solid. LCMS (ESI MS) m/z=227.7 [M+3H].

Step B: A mixture of 6-bromo-2,3-dichloropyridine 139 (470 mg, 2.07 mmol), tert-butyl 5-(4,4,5,5-tetramethyl-1,3,2-dioxaborolan-2-yl)-3,6-dihydropyridine-1(2H)-carboxylate 140 (640.5 mg, 2.07 mmol), Pd(dppf)Cl₂·CH₂Cl₂ (338.3 mg, 414.30 μmol), and K₂CO₃ (572.5 mg, 4.14 mmol) in toluene (5 mL) and H₂O (1 mL) is purged with N₂ three times, and then the mixture is stirred at 50° C. for 4 hr under N₂. The reaction mixture is concentrated under reduced pressure to remove solvent. The residue is diluted with H₂O and extracted with ethyl acetate three times. The combined organic layers are washed with brine twice, dried over Na₂SO₄, filtered and concentrated under reduced pressure to give a residue. The residue is purified by flash silica gel chromatography (eluent of 0-13% ethyl acetate/petroleum ether gradient) to yield the title compound tert-butyl 5,6-dichloro-5′,6′-dihydro-[2,3′-bipyridine]-1′(2′H)-carboxylate 141 (215 mg) as a colorless oil. LCMS (ESI MS) m/z=273.1 [M+H]+ 1.

Step C: A mixture of tert-butyl 5,6-dichloro-5′,6′-dihydro-[2,3′-bipyridine]-1′(2′H)-carboxylate 141 (170 mg, 516.37 μmol), 3-(4,4,5,5-tetramethyl-1,3,2-dioxaborolan-2-yl)pyrazolo[1,5-a]pyridine 142 (100.8 mg, 413.10 μmol), Pd(dppf)Cl₂·CH₂Cl₂ (84.3 mg, 103.27 μmol), and Cs₂CO₃ (336.4 mg, 1.03 mmol) in H₂O (0.7 mL) and dioxane (3 mL) is purged with N₂ three times, and then the mixture is stirred at 80° C. for 2 hours under N₂. The reaction mixture is concentrated under reduced pressure to remove solvent. The residue is diluted with H₂O and extracted with ethyl acetate three times. The combined organic layers are washed with brine twice, dried over Na₂SO₄, filtered and concentrated under reduced pressure to give a residue. The residue is purified by flash silica gel chromatography (eluent of 0-15% ethyl acetate/petroleum ether gradient) to yield the title compound tert-butyl 5-chloro-6-(pyrazolo[1,5-a]97yridine-3-yl)-5′,6′-dihydro-[2,3′-bipyridine]-1′(2′H)-carboxylate 143 (60 mg) is obtained as a colorless oil. LCMS (ESI MS) m/z=411.1 [M+H]+ 1.

Step D: To a solution of tert-butyl 5-chloro-6-(pyrazolo[1,5-a]pyridin-3-yl)-5′,6′-dihydro-[2,3′-bipyridine]-1′(2′H)-carboxylate 143 (80 mg, 194.70 μmol) in EtOAc (8 mL) is added PtO₂ (4.4 mg, 19.47 μmol) under N₂. The suspension is degassed under vacuum and purged with H₂ several times. The mixture is stirred under H₂ (15 psi) at 25° C. for 16 hr. The reaction mixture is filtered and concentrated under reduced pressure to remove solvent. The residue is purified by flash silica gel chromatography (eluent of 0-18% ethyl acetate/petroleum ether gradient) to yield the title compound tert-butyl 3-(5-chloro-6-(pyrazolo[1,5-a]97yridine-3-yl)97yridine-2-yl)piperidine-1-carboxylate 144 (46 mg) is obtained as a yellow oil. LCMS (ESI MS) m/z=413.2 [M+H]+ 1.

Step E: To a solution of tert-butyl 3-(5-chloro-6-(pyrazolo[1,5-a]pyridin-3-yl)pyridin-2-yl) piperidine-1-carboxylate 144 (40 mg, 96.87 μmol) in DCM (0.3 mL) is added HCl/dioxane (4 M, 0.3 mL). The mixture is stirred at 20° C. for 1 h. The reaction mixture is concentrated under reduced pressure to give a residue. The residue is purified by prep-HPLC (column: Xtimate C18, 150×40 mm×10 um; mobile phase: [water (FA)-ACN]; gradient: 0%-32% B over 25 min) to yield the title compound 3-(3-chloro-6-(piperidin-3-yl)pyridin-2-yl)pyrazolo[1,5-a]pyridine (Compound 30) (10 mg, 32%) as a brown solid. LCMS (ESI MS) m/z=313.1 [M+H]+ 1.

¹H NMR (400 MHz, methanol-d₄) δ=8.62 (s, 1H), 8.52 (d, J=7.00 Hz, 1H), 8.33 (d, J=9.01 Hz, 1H), 7.72 (d, J=8.25 Hz, 1H), 7.25-7.36 (m, 1H), 7.03 (d, J=8.25 Hz, 1H), 6.94 (td, J=6.85, 1.06 Hz, 1H), 3.17 (br s, 1H), 2.85-3.03 (m, 2H), 2.82 (d, J=11.63 Hz, 1H), 2.57 (s, 1H), 2.03 (br d, J=9.38 Hz, 1H), 1.67-1.81 (m, 2H), 1.50-1.66 (m, 1H).

### Example 31

### 3-[4-fluoro-6-(3-piperidyl)-2-pyridyl]pyrazolo[1,5-a]pyridine

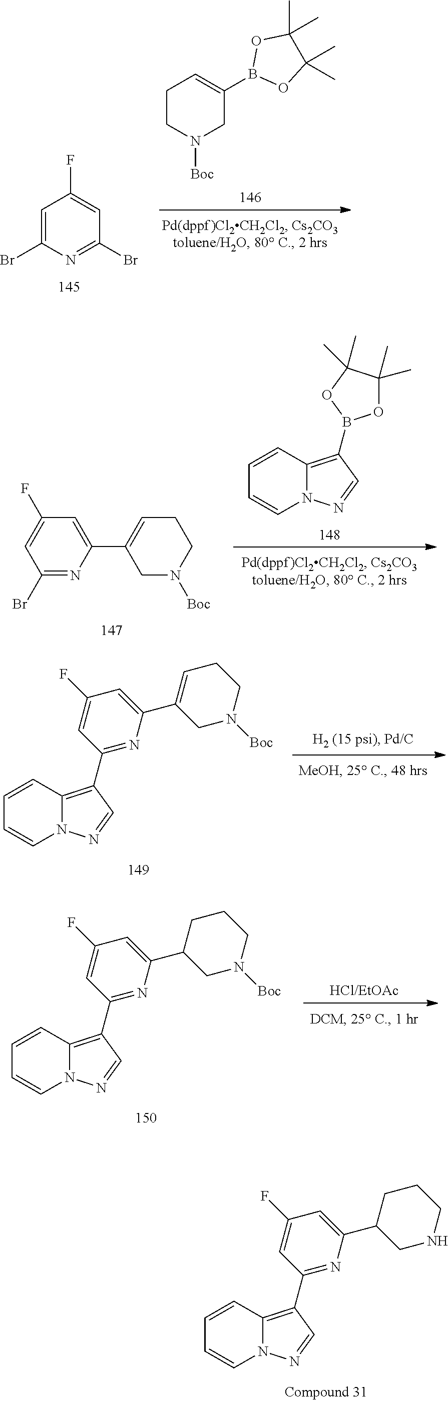

Step A: A mixture of tert-butyl 5-(4,4,5,5-tetramethyl-1,3,2-dioxaborolan-2-yl)-3,6-dihydropyridine-1(2H)-carboxylate 146 (400 mg, 1.29 mmol), 2,6-dibromo-4-fluoropyridine 145 (659 mg, 2.59 mmol), Cs₂CO₃ (842 mg, 2.59 mmol), and Pd(dppf)Cl₂·CH₂Cl₂ (211 mg, 258.73 μmol) in toluene (5 mL) and H₂O (1 mL) is purged with N₂ three times, and then the mixture is stirred at 80° C. for 2 hr under N₂. The reaction is diluted with H₂O and extracted with EtOAc three times. The combined organic layers are dried over anhydrous Na₂SO₄, filtered and concentrated under reduced pressure to give a residue which is purified by flash silica gel chromatography (eluent of 0-15% ethyl acetate/petroleum ether gradient) to yield the title compound tert-butyl 5-(6-bromo-4-fluoro-2-pyridyl)-3,6-dihydro-2H-pyridine-1-carboxylate 147 (400 mg) as a white solid. LCMS (ESI MS) m/z=300.9 [M+H]+ 1.

Step B: A mixture of tert-butyl 6-bromo-4-fluoro-5′,6′-dihydro-[2,3′-bipyridine]-1′(2′H)-carboxylate 147 (280 mg, 783.84 μmol), 3-(4,4,5,5-tetramethyl-1,3,2-dioxaborolan-2-yl)pyrazolo[1,5-a]pyridine 148 (287 mg, 1.18 mmol), Pd(dppf)Cl₂·CH₂Cl₂ (128 mg, 156.77 μmol), and Cs₂CO₃ (510 mg, 1.57 mmol) in toluene (5 mL) and H₂O (1 mL) is purged with N₂ three times. The mixture is stirred at 80° C. for 2 hr under N₂. The reaction is diluted with H₂O and extracted with EtOAc three times. The combined organic layers are dried over anhydrous Na₂SO₄, filtered and concentrated under reduced pressure to give a residue which is purified by flash silica gel chromatography (eluent of 0-15% ethyl acetate/petroleum ether gradient) to yield the title compound tert-butyl 5-(4-fluoro-6-pyrazolo [1,5-a]pyridin-3-yl-2-pyridyl)-3,6-dihydro-2H-pyridine-1-carboxylate 149 (200 mg) as a white solid. LCMS (ESI MS) m/z=395.1 [M+H]+ 1.

Step C: A mixture of tert-butyl 4-fluoro-6-(pyrazolo[1,5-a]pyridin-3-yl)-5′,6′-dihydro-[2,3′-bipyridine]-1′(2′H)-carboxylate 149 (60 mg, 152.11 μmol) and Pd/C (32 mg, 30.42 μmol, 10%) in MeOH (2 mL) is purged with H₂ three times, and then the mixture is stirred at 25° C. for 48 hr under a H₂ (15 psi) atmosphere. The reaction is filtered, and the filtrate is concentrated under vacuum to give the title compound tert-butyl 3-(4-fluoro-6-pyrazolo[1,5-a]pyridin-3-yl-2-pyridyl)piperidine-1-carboxylate 150 (30 mg) as a brown oil. LCMS (ESI MS) m/z=397.2 [M+H]+ 1.

Step D: To a solution of tert-butyl 3-(4-fluoro-6-(pyrazolo[1,5-a]pyridin-3-yl)pyridin-2-yl)piperidine-1-carboxylate 150 (30 mg, 75.67 μmol) in DCM (0.5 mL) is added HCl/EtOAc (4 M, 0.5 mL). The mixture is stirred at 25° C. for 1 hr. The reaction mixture is concentrated under vacuum to give the crude product which is purified by prep-HPLC (column: Xtimate C18, 150×40 mm×10 um; mobile phase: [water (FA)-ACN]; gradient: 0%-32% B over 25 min) to yield the title compound 3-[4-fluoro-6-(3-piperidyl)-2-pyridyl]pyrazolo[1,5-a]pyridine (Compound 31) (16.0 mg, 25%) as a white solid. LCMS (ESI MS) m/z=297.0 [M+H]+ 1.

¹H NMR (400 MHz, methanol-d₄) δ=8.60 (d, J=7.00 Hz, 2H), 8.51-8.55 (m, 2H), 7.42-7.52 (m, 2H), 6.95-7.08 (m, 2H), 3.71 (br d, J=10.76 Hz, 1H), 3.39-3.51 (m, 2H), 3.25-3.31 (m, 1H), 3.11 (td, J=12.16, 3.31 Hz, 1H), 2.21-2.28 (m, 1H), 2.04-2.13 (m, 1H), 1.92-2.00 (m, 2H).

### Example 32

### 3-[3-fluoro-6-(3-piperidyl)-2-pyridyl]pyrazolo[1,5-a]pyridine

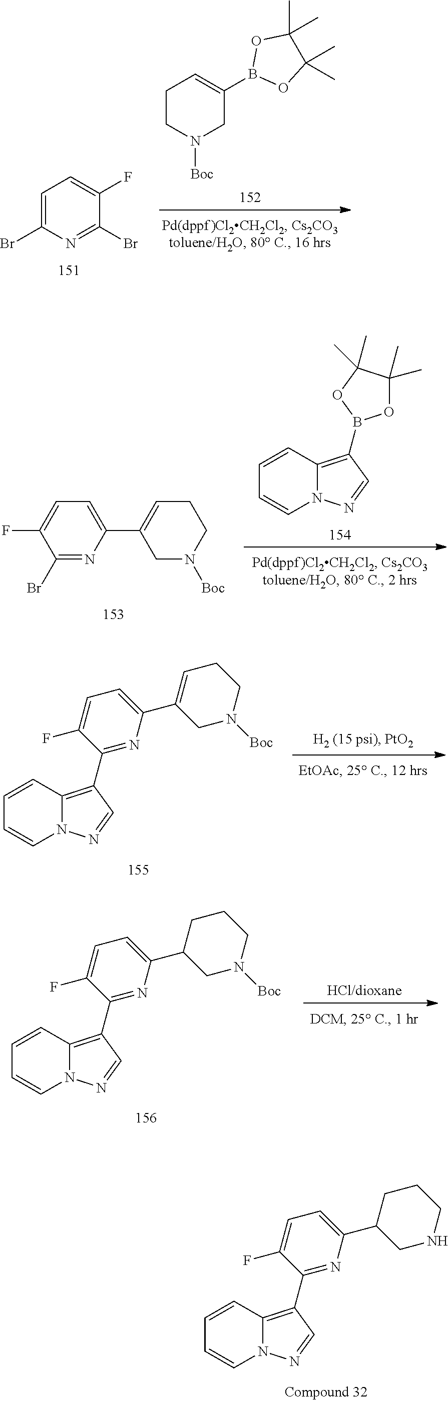

Step A: A mixture of 2,6-dibromo-3-fluoropyridine 151 (866 mg, 3.0 mmol), tert-butyl 5-(4,4,5,5-tetramethyl-1,3,2-dioxaborolan-2-yl)-3,6-dihydropyridine-1(2H)-carboxylate 152 (700 mg, 2.26 mmol), Pd(dppf)Cl₂·CH₂Cl₂ (166 mg, 226.4 μmol), and Cs₂CO₃ (1.48 g, 4.53 mmol) in toluene (20 mL) and H₂O (5 mL) is degassed and purged with N₂ three times. The mixture is stirred at 80° C. for 16 hours under a N₂ atmosphere. The reaction mixture is quenched with H₂O, and the mixture is extracted with EtOAc three times. The combined organic layers are washed with brine, dried over Na₂SO₄, filtered and concentrated under reduced pressure to give a residue which is purified by flash silica gel chromatography (eluent of 9-10% ethyl acetate/petroleum ether gradient) to yield the title compound tert-butyl 5-(6-bromo-5-fluoro-2-pyridyl)-3,6-dihydro-2H-pyridine-1-carboxylate 153 (330 mg) as a white solid. LCMS (ESI MS) m/z=357.0 [M+H]+ 1.

¹H NMR (400 MHz, DMSO-d₆) δ=7.83-7.88 (m, 1H), 7.72 (br dd, J=8.4, 3.1 Hz, 1H), 6.80 (br s, 1H), 4.25 (br s, 2H), 3.46 (br t, J=5.2 Hz, 2H), 2.30 (br d, J=2.8 Hz, 2H), 1.42 (s, 9H).

Step B: A mixture of tert-butyl 6-bromo-5-fluoro-5′,6′-dihydro-[2,3′-bipyridine]-1′(2′H)-carboxylate 153 (300 mg, 739 μmol), 3-(4,4,5,5-tetramethyl-1,3,2-dioxaborolan-2-yl)pyrazolo[1,5-a]pyridine 154 (198 mg, 812.9 μmol), Cs₂CO₃ (482 mg, 1.48 mmol), and Pd(dppf)Cl₂·CH₂Cl₂ (60 mg, 73.9 μmol) in toluene (4 mL) and H₂O (1 mL) is degassed and purged with N₂ three times. The mixture is stirred at 80° C. for 2 hr under N₂. The reaction is quenched with water and extracted with EtOAc three times. The combined organic layers are dried over anhydrous Na₂SO₄, filtered and concentrated under reduced pressure to give a residue which is purified by flash silica gel chromatography (eluent of 0-15% ethyl acetate/petroleum ether gradient) to yield the title compound tert-butyl 5-(5-fluoro-6-pyrazolo[1,5-a]pyridin-3-yl-2-pyridyl)-3,6-dihydro-2H-pyridine-1-carboxylate 15 (290 mg) as a white solid. LCMS (ESI MS) m/z=395.4 [M+H]+ 1.

Step C: A mixture of tert-butyl 5-fluoro-6-(pyrazolo[1,5-a]pyridin-3-yl)-5′,6′-dihydro-[2,3′-bipyridine]-1′(2′H)-carboxylate 155 (200 mg, 507 μmol) and PtO₂ (35 mg, 152.1 μmol) in EtOAc (5 mL) is purged with H₂ three times. The mixture is stirred at 25° C. for 12 hr under a H₂ (15 psi) atmosphere. The reaction is filtered to remove insoluble matter, and the filtrate is concentrated under vacuum to give the title compound tert-butyl 3-(5-fluoro-6-pyrazolo[1,5-a]pyridin-3-yl-2-pyridyl)piperidine-1-carboxylate 15 (120 mg, crude) as a brown oil. LCMS (ESI MS) m/z=397.6 [M+H]+ 1.

Step D: To a solution of tert-butyl 3-(5-fluoro-6-(pyrazolo[1,5-a]pyridin-3-yl)pyridin-2-yl)piperidine-1-carboxylate 156 (100 mg, 252.2 μmol) in DCM (2 mL) is added HCl/dioxane (4 M, 504 μL). The mixture is stirred at 25° C. for 1 hr. The reaction mixture is concentrated under vacuum to give the crude product which is purified by prep-HPLC (column: Xtimate C18, 150×40 mm×10 um; mobile phase: [water (NH₃H₂O+NH₄HCO₃)-ACN]; gradient: 12%-52% B over 32 min) to yield the title compound 3-[3-fluoro-6-(3-piperidyl)-2-pyridyl]pyrazolo[1,5-a]pyridine (Compound 32) (44.8 mg, 58%) as a white solid. LCMS (ESI MS) m/z=297.2 [M+H]+1.

¹H NMR (400 MHz, methanol-d₄) δ=8.61 (br d, J=9.01 Hz, 1H), 8.55 (d, J=6.88 Hz, 1H), 8.41 (d, J=3.25 Hz, 1H), 7.41-7.49 (m, 1H), 7.31-7.40 (m, 1H), 7.05 (dd, J=8.38, 3.25 Hz, 1H), 6.98 (t, J=6.82 Hz, 1H), 3.36 (br d, J=10.26 Hz, 1H), 3.18 (br d, J=12.63 Hz, 1H), 2.89-3.08 (m, 2H), 2.70-2.81 (m, 1H), 2.04-2.15 (m, 1H), 1.66-1.93 (m, 3H).

### Example 33

### 3-[5-fluoro-6-(3-piperidyl)-2-pyridyl]pyrazolo[1,5-a]pyridine

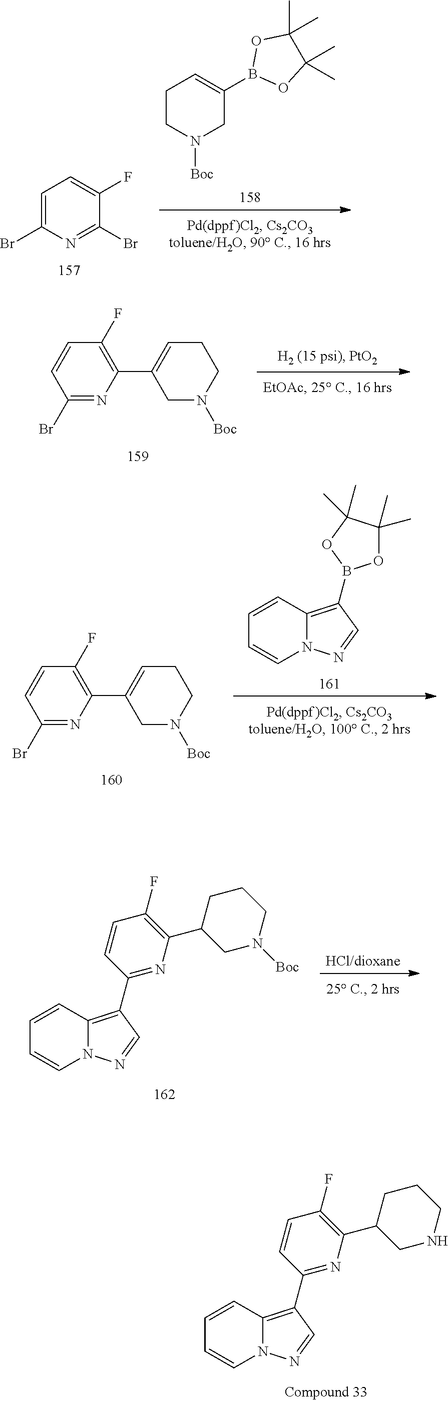

Step A: A mixture of 2,6-dibromo-3-fluoropyridine 157 (741.8 mg, 2.91 mmol), tert-butyl 5-(4,4,5,5-tetramethyl-1,3,2-dioxaborolan-2-yl)-3,6-dihydropyridine-1(2H)-carboxylate 158 (600 mg, 1.94 mmol), Pd(dppf)Cl₂ (141.9 mg, 194.04 μmol), and Cs₂CO₃ (1.26 g, 3.88 mmol) in toluene (12 mL) and H₂O (3 mL) is purged with N₂ three times, and then the mixture is stirred at 90° C. for 16 hr under N₂. The reaction mixture is diluted with H₂O and extracted with EtOAc three times. The combined organic layers are washed with brine, dried over Na₂SO₄, filtered and concentrated under reduced pressure to give a residue. The residue is purified by flash silica gel chromatography (eluent of 0-10% ethyl acetate/petroleum ether gradient) to yield the title compound tert-butyl 5-(6-bromo-3-fluoro-2-pyridyl)-3,6-dihydro-2H-pyridine-1-carboxylate 159 (150 mg) as a white solid. LCMS (ESI MS) m/z=357.0 [M+H]+ 1.

Step B: To a solution of tert-butyl 6-bromo-3-fluoro-5′,6′-dihydro-[2,3′-bipyridine]-1′(2′H)-carboxylate 159 (190 mg, 531.89 μmol) in EtOAc (5 mL) is added PtO₂ (24.1 mg, 106.38 μmol) under N₂. The suspension is purged with H₂ three times. The mixture is stirred under H₂ (15 psi) at 25° C. for 16 hr. The reaction mixture is concentrated to yield the title compound tert-butyl 3-(6-bromo-3-fluoro-2-pyridyl)piperidine-1-carboxylate 160 (170 mg, crude) as a white solid. LCMS (ESI MS) m/z=359.0 [M+H]+ 1.

Step C: A mixture of tert-butyl 3-(6-bromo-3-fluoropyridin-2-yl)piperidine-1-carboxylate 160 (150 mg, 417.56 μmol), 3-(4,4,5,5-tetramethyl-1,3,2-dioxaborolan-2-yl)pyrazolo[1,5-a]pyridine 161 (101.9 mg, 417.56 μmol), Pd(dppf)Cl₂ (61.1 mg, 83.51 μmol), and Cs₂CO₃ (272.1 mg, 835.11 μmol) in toluene (2 mL) and H₂O (0.5 mL) is purged with N₂ three times. The mixture is stirred at 100° C. for 2 hr under N₂. The reaction mixture is diluted with H₂O and extracted with EtOAc three times. The combined organic layers are washed with brine, dried, filtered and concentrated under reduced pressure to give a residue. The residue is purified by flash silica gel chromatography (eluent of 0-20% ethyl acetate/petroleum ether gradient) to yield the title compound tert-butyl 3-(3-fluoro-6-pyrazolo[1,5-a]pyridin-3-yl-2-pyridyl)piperidine-1-carboxylate 162 (130 mg) as a white solid. LCMS (ESI MS) m/z=397.2 [M+H]+ 1.

Step D: A solution of tert-butyl 3-(3-fluoro-6-(pyrazolo[1,5-a]pyridin-3-yl)pyridin-2-yl)piperidine-1-carboxylate 162 (110 mg, 277.46 μmol) in HCl/dioxane (4 M, 2 mL) is stirred at 25° C. for 2 hr. The reaction mixture is concentrated directly. The residue is purified by prep-HPLC (column: Xtimate C18, 150×40 mm×10 um; mobile phase: [water (FA)-ACN]; gradient: 0%-32% B over 25 min) to yield the title compound 3-[5-fluoro-6-(3-piperidyl)-2-pyridyl]pyrazolo[1,5-a]pyridine (Compound 33) (5 mg, 5%, FA) as a white solid. LCMS (ESI MS) m/z=297.1 [M+H]+ 1.

¹H NMR (400 MHz, methanol-d₄) δ=8.54-8.59 (m, 2H), 8.46 (s, 1H), 8.43 (d, J=9.2 Hz, 1H), 7.72 (dd, J=8.8, 3.6 Hz, 1H), 7.56 (dd, J=9.6, 8.8 Hz, 1H), 7.40 (ddd, J=8.8, 6.8, 0.8 Hz, 1H), 6.99 (td, J=6.8, 1.1 Hz, 1H), 3.57-3.65 (m, 2H), 3.54 (d, J=12.8 Hz, 1H), 3.43-3.50 (m, 1H), 3.15 (td, J=12.4, 3.6 Hz, 1H), 2.14-2.24 (m, 1H), 2.02-2.12 (m, 1H), 1.91-2.01 (m, 2H).

### Example 34

### 5-chloro-3-[4-chloro-6-(3-piperidyl)-2-pyridyl]pyrazolo[1,5-a]pyridine

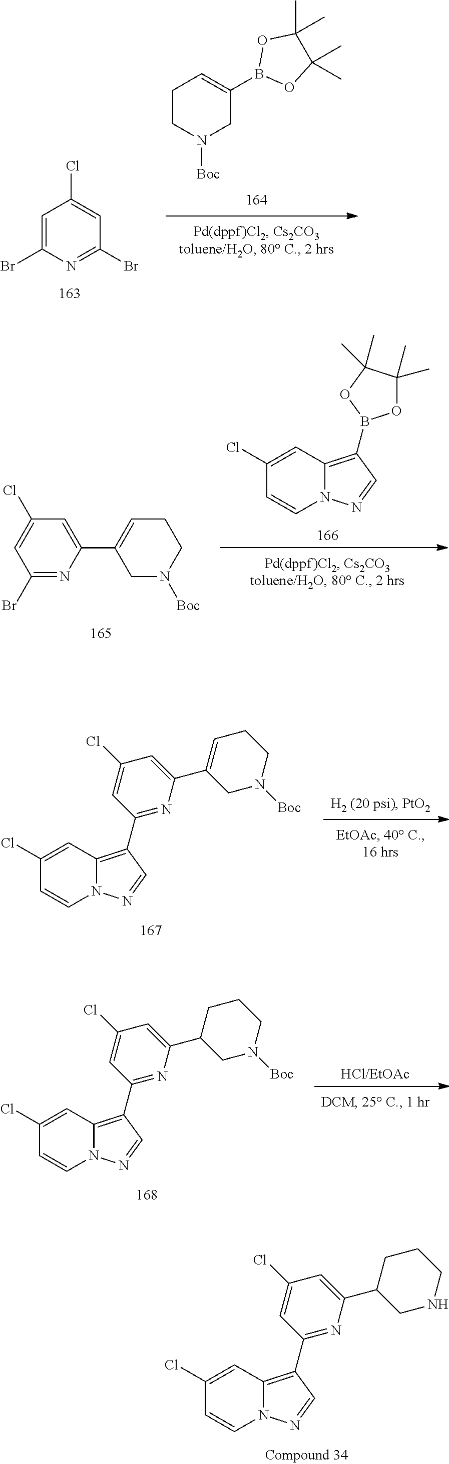

Step A: A mixture of 2,6-dibromo-4-chloropyridine 163 (658 mg, 2.43 mmol), tert-butyl 5-(4,4,5,5-tetramethyl-1,3,2-dioxaborolan-2-yl)-3,6-dihydropyridine-1(2H)-carboxylate 164 (500 mg, 1.62 mmol), Pd(dppf)Cl₂·CH₂Cl₂ (264 mg, 323.41 μmol), and Cs₂CO₃ (1 g, 3.23 mmol) in toluene (5 mL) and H₂O (1 mL) is purged with N₂ three times, and then the mixture is stirred at 80° C. for 2 hr under N₂. The reaction mixture is diluted with H₂O, and the mixture is extracted with EtOAc three times. The combined organic layers are washed with brine, dried over Na₂SO₄, filtered and concentrated under reduced pressure to give a residue which is purified by flash silica gel chromatography (eluent of 0-30% ethyl acetate/petroleum ether gradient) to yield the title compound tert-butyl 5-(6-bromo-5-fluoro-2-pyridyl)-3,6-dihydro-2H-pyridine-1-carboxylate 165 (330 mg) as a yellow oil. LCMS (ESI MS) m/z=318.9 [M+H]+ 1.

Step B: A mixture of tert-butyl 6-bromo-4-chloro-5′,6′-dihydro-[2,3′-bipyridine]-1′(2′H)-carboxylate 165 (60 mg, 159.71 μmol), 5-chloro-3-(4,4,5,5-tetramethyl-1,3,2-dioxaborolan-2-yl)pyrazolo[1,5-a]pyridine 166 (53 mg, 191.65 μmol), Cs₂CO₃ (104 mg, 319.41 μmol), and Pd(dppf)Cl₂·CH₂Cl₂ (26 mg, 31.9 μmol) in toluene (3 mL) and H₂O (1 mL) is purged with N₂ three times, and then the mixture is stirred at 80° C. for 2 hr under N₂. The reaction is diluted with H₂O and extracted with EtOAc three times. The combined organic layers are dried over anhydrous Na₂SO₄, filtered and concentrated under reduced pressure to give a residue which is purified by flash silica gel chromatography (eluent of 0-10% ethyl acetate/petroleum ether gradient) to yield the title compound tert-butyl 3-[4-chloro-6-(5-chloropyrazolo[1,5-a]pyridin-3-yl)-2-pyridyl]piperidine-1-carboxylate 167 (290 mg) as a white solid. LCMS (ESI MS) m/z=444.9 [M+H]+ 1.

Step C: To a solution of tert-butyl 4-chloro-6-(5-chloropyrazolo[1,5-a]pyridin-3-yl)-5′,6′-dihydro-[2,3′-bipyridine]-1′(2′H)-carboxylate 167 (60 mg, 134.73 μmol) and PtO₂ (3 mg, 13.47 μmol) in EtOAc (3 mL) is purged with H₂ three times, and then the mixture is stirred at 40° C. for 16 hr under a H₂ atmosphere (20 psi). The reaction is filtered to remove the insolubles and concentrated under vacuum to give the title compound tert-butyl 3-[4-chloro-6-(5-chloropyrazolo[1,5-a]105yridine-3-yl)-2-pyridyl]piperidine-1-carboxylate 168 (50 mg, crude) as a brown oil. LCMS (ESI MS) m/z=447.0 [M+H]+ 1.

Step D: To a solution of tert-butyl 3-(4-chloro-6-(5-chloropyrazolo[1,5-a]pyridin-3-yl)pyridin-2-yl)piperidine-1-carboxylate 168 (50 mg, 111.77 μmol) in DCM (1 mL) is added HCl/dioxane (4 M, 504 μL). The mixture is stirred at 25° C. for 1 hr. The reaction mixture is concentrated under vacuum to give the crude product which is purified by prep-HPLC (column: Xtimate C18, 150×40 mm×10 um; mobile phase: [water (FA)-ACN]; gradient: 0%-38% B over 25 min) to yield the title compound 5-chloro-3-[4-chloro-6-(3-piperidyl)-2-pyridyl]pyrazolo[1,5-a]pyridine (Compound 34) (2.0 mg, 5%) as a white solid. LCMS (ESI MS) m/z=347.1 [M+H]+ 1.

¹H NMR (400 MHz, methanol-d₄) δ=8.62 (s, 1H), 8.60 (s, 1H), 8.57 (s, 1H), 8.55-8.57 (m, 1H), 7.79 (s, 1H), 7.25 (s, 1H), 7.04 (br d, J=7.25 Hz, 1H), 3.68 (br d, J=11.26 Hz, 1H), 3.45 (br d, J=12.51 Hz, 1H), 3.22-3.31 (m, 2H), 2.99-3.10 (m, 1H), 2.26 (br d, J=6.88 Hz, 1H), 2.10 (br d, J=6.38 Hz, 1H), 1.95 (br t, J=9.57 Hz, 2H).

### Example 35

### 5-chloro-3-[4-fluoro-6-(3-piperidyl)-2-pyridyl] pyrazolo 1,5-a]pyridine

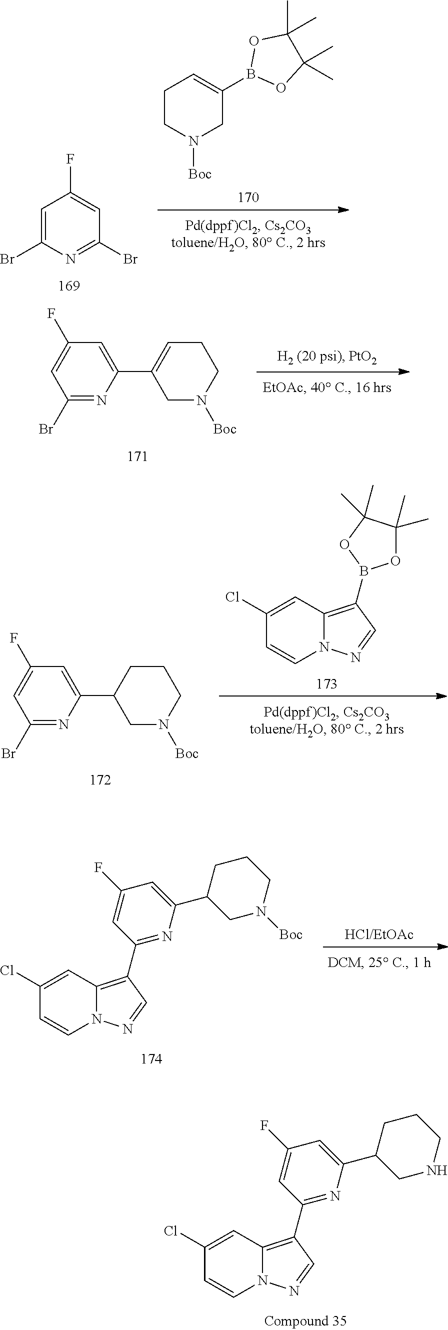

Step A: A mixture of tert-butyl 5-(4,4,5,5-tetramethyl-1,3,2-dioxaborolan-2-yl)-3,6-dihydropyridine-1(2H)-carboxylate 170 (400 mg, 1.29 mmol), 2,6-dibromo-4-fluoropyridine 169 (659 mg, 2.59 mmol), Cs₂CO₃ (842 mg, 2.59 mmol), and Pd(dppf)Cl₂·CH₂Cl₂ (211 mg, 258.73 μmol) in toluene (5 mL) and H₂O (1 mL) is purged with N₂ three times, and then the mixture is stirred at 80° C. for 2 hr under N₂. The reaction is diluted with H₂O and extracted with EtOAc three times. The combined organic layers are dried over anhydrous Na₂SO₄, filtered and concentrated under reduced pressure to give a residue which is purified by flash silica gel chromatography (eluent of 0-15% ethyl acetate/petroleum ether gradient) to yield the title compound tert-butyl 5-(6-bromo-4-fluoro-2-pyridyl)-3,6-dihydro-2H-pyridine-1-carboxylate 171 (400 mg) as a white solid. LCMS (ESI MS) m/z=300.9 [M+H]+ 1.

Step B: A mixture of tert-butyl 6-bromo-4-fluoro-5′,6′-dihydro-[2,3′-bipyridine]-1′(2′H)-carboxylate 171 (300 mg, 839.82 μmol), and PtO₂ (19.07 mg, 83.98 μmol) in EtOAc (5 mL) is purged with H₂ three times, and then the mixture is stirred at 40° C. for 16 hr under H₂ (20 psi). The reaction mixture is filtered, and the filtrate is concentrated under reduced pressure to give a crude product which is purified by flash silica gel chromatography (eluent of 0-15% ethyl acetate/petroleum ether gradient) to yield the title compound tert-butyl 3-(6-bromo-4-fluoro-2-pyridyl) piperidine-1-carboxylate 172 (150 mg) as a white solid. LCMS (ESI MS) m/z=305.0 [M+H]+ 1.

Step C: A mixture of tert-butyl 3-(6-bromo-4-fluoropyridin-2-yl)piperidine-1-carboxylate 172 (25 mg, 69.6 μmol), 5-chloro-3-(4,4,5,5-tetramethyl-1,3,2-dioxaborolan-2-yl)pyrazolo[1,5-a]pyridine 173 (29 mg, 104.39 μmol), Cs₂CO₃ (45 mg, 139.19 μmol), and Pd(dppf)Cl₂·CH₂Cl₂ (11 mg, 13.92 μmol) in toluene (1 mL) and H₂O (0.3 mL) is purged with N₂ three times and then stirred at 80° C. for 2 hr under N₂. The reaction is diluted with H₂O and extracted with EtOAc three times. The combined organic layers are dried over anhydrous Na₂SO₄, filtered and concentrated under reduced pressure to give a residue which is purified by flash silica gel chromatography (eluent of 0-30% ethyl acetate/petroleum ether gradient) to yield the title compound tert-butyl 3-[6-(5-chloropyrazolo[1,5-a]pyridin-3-yl)-4-fluoro-2-pyridyl]piperidine-1-carboxylate 174 (25 mg) as a white solid. LCMS (ESI MS) m/z=431.2 [M+H]+1.

Step D: To a solution of tert-butyl 3-(6-(5-chloropyrazolo[1,5-a]pyridin-3-yl)-4-fluoropyridin-2-yl) piperidine-1-carboxylate 174 (40 mg, 92.83 μmol) in DCM (1 mL) is added HCl/dioxane (4 M, 0.5 mL). The mixture is stirred at 25° C. for 1 hr. The reaction mixture is concentrated under vacuum to give the crude product which is purified by prep-HPLC (column: Xtimate C18, 150×40 mm×10 um; mobile phase: [water (FA)-ACN]; gradient: 0%-34% B over 25 min) to yield the title compound 5-chloro-3-[4-fluoro-6-(3-piperidyl)-2-pyridyl] pyrazolo 1,5-a]pyridine (Compound 35) (6.0 mg, 18%) as a white solid. LCMS (ESI MS) m/z=331.1 [M+H]+1.

¹H NMR (400 MHz, methanol-d₄) δ=8.61 (s, 1H), 8.60 (s, 1H), 8.58 (s, 1H), 8.56 (d, J=2.25 Hz, 1H), 7.49-7.57 (m, 1H), 6.99-7.06 (m, 2H), 3.73 (br d, J=11.01 Hz, 1H), 3.49 (br d, J=11.88 Hz, 1H), 3.33-3.42 (m, 2H), 3.08 (td, J=12.44, 3.38 Hz, 1H), 2.25-2.31 (m, 1H), 2.09-2.19 (m, 1H), 1.92-2.03 (m, 2H).

### Example 36

### 3-(4-chloro-6-(piperidin-3-yl)pyridin-2-yl)-5-(trifluoromethyl)pyrazolo[1,5-a]pyridine

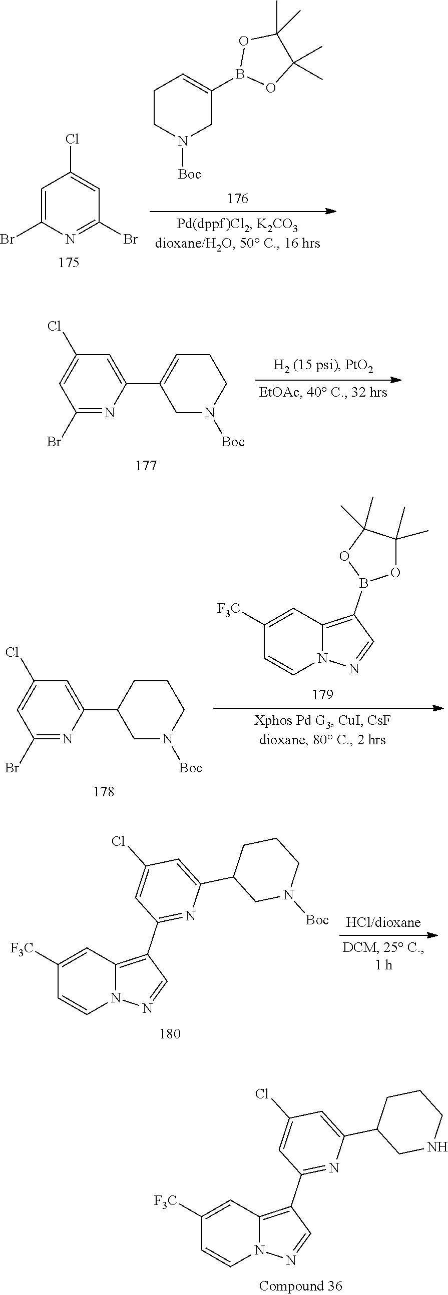

Step A: A mixture of 2,6-dibromo-4-chloropyridine 175 (500 mg, 1.84 mmol), tert-butyl 6-bromo-4-chloro-5′,6′-dihydro-[2,3′-bipyridine]-1′(2′H)-carboxylate 176 (399 mg, 1.29 mmol), Pd(dppf)Cl₂ (301 mg, 368.5 μmol), and K₂CO₃ (509 mg, 3.69 mmol) in dioxane (5 mL) and H₂O (1 mL) is purged with N₂ three times, and then the mixture is stirred at 50° C. for 16 hr under N₂. The reaction mixture is concentrated under reduced pressure to remove solvent. The residue is diluted with water and extracted with ethyl acetate three times. The combined organic layers are washed with brine, dried over anhydrous Na₂SO₄, filtered and concentrated under reduced pressure to give a residue which is purified by flash silica gel chromatography (eluent of 0-7% ethyl acetate/petroleum ether gradient) to yield the title compound tert-butyl 6-bromo-4-chloro-5′,6′-dihydro-[2,3′-bipyridine]-1′(2′H)-carboxylate 177 (270 mg, crude) as a colorless oil. LCMS (ESI MS) m/z=318.9 [M+3]+1.

Step B: To a solution of tert-butyl 6-bromo-4-chloro-5′,6′-dihydro-[2,3′-bipyridine]-1′(2′H)-carboxylate 177 (200 mg, 535.2 μmol) in EtOAc (10 mL) is added PtO₂ (12 mg, 53.52 μmol) under a H₂ (15 psi) atmosphere. The mixture is stirred at 40° C. for 32 hr. The reaction mixture is filtered, and the filtrate is concentrated under reduced pressure. The residue is purified by flash silica gel chromatography (eluent of 0-5% ethyl acetate/petroleum ether gradient) to yield the title compound tert-butyl 3-(6-bromo-4-chloropyridin-2-yl)piperidine-1-carboxylate 178 (130 mg, crude) as a colorless oil. LCMS (ESI MS) m/z=320.7 [M+H]+ 1.

Step C: A mixture of 3-(4,4,5,5-tetramethyl-1,3,2-dioxaborolan-2-yl)-5-(trifluoromethyl)pyrazolo[1,5-a]pyridine 179 (66 mg, 212.94 μmol), tert-butyl 3-(6-bromo-4-chloropyridin-2-yl)piperidine-1-carboxylate 178 (80 mg, 212.9 μmol), CsF (129 mg, 851.77 μmol), CuI (81 mg, 425.89 μmol), and XPhos Pd G3 (36 mg, 42.59 μmol) in dioxane (1 mL) is purged with N₂ three times, and then the mixture is stirred at 80° C. for 2 hr under N₂. The reaction mixture is filtered and concentrated under reduced pressure to give a residue which is purified by flash silica gel chromatography (eluent of 0-50% ethyl acetate/petroleum ether gradient) to yield the title compound tert-butyl 3-(4-chloro-6-(5-(trifluoromethyl)pyrazolo[1,5-a]pyridin-3-yl)pyridin-2-yl)piperidine-1-carboxylate 180 (70 mg, crude) as a brown oil. LCMS (ESI MS) m/z=481.0 [M+H]+ 1.

Step D: To a solution tert-butyl 3-(4-chloro-6-(5-(trifluoromethyl)pyrazolo[1,5-a]pyridin-3-yl) pyridin-2-yl)piperidine-1-carboxylate of 180 (50 mg, 103.97 μmol) in DCM (0.5 mL) is added HCl/dioxane (4 M, 26 μL). The mixture is stirred at 25° C. for 1 hr. The reaction mixture is concentrated under reduced pressure, and the residue is purified by prep-HPLC (column: Xtimate C18, 150×40 mm×10 um; mobile phase: [water (FA)-ACN]; gradient: 0%-40% B over 25 min) to give the title compound 3-(4-chloro-6-(piperidin-3-yl)pyridin-2-yl)-5-(trifluoromethyl)pyrazolo[1,5-a]pyridine (Compound 36) (3.5 mg, FA salt) as a white solid. LCMS (ESI MS) m/z=380.9 [M+H]+ 1.

¹H NMR (400 MHz, DMSO-d₆) δ=9.07 (br s, 1H), 9.01-9.05 (m, 1H), 9.00 (s, 1H), 8.31-8.40 (m, 1H), 8.03 (d, J=1.63 Hz, 1H), 7.31 (s, 2H), 3.05-3.15 (m, 2H), 2.93-3.05 (m, 2H), 2.89 (br dd, J=11.57, 4.69 Hz, 1H), 2.04-2.11 (m, 1H), 1.77 (br d, J=10.76 Hz, 2H), 1.58-1.69 (m, 1H).

### Example 37

### 3-(6-piperazin-1-yl-2-pyridyl)pyrazolo[1,5-a]pyridine

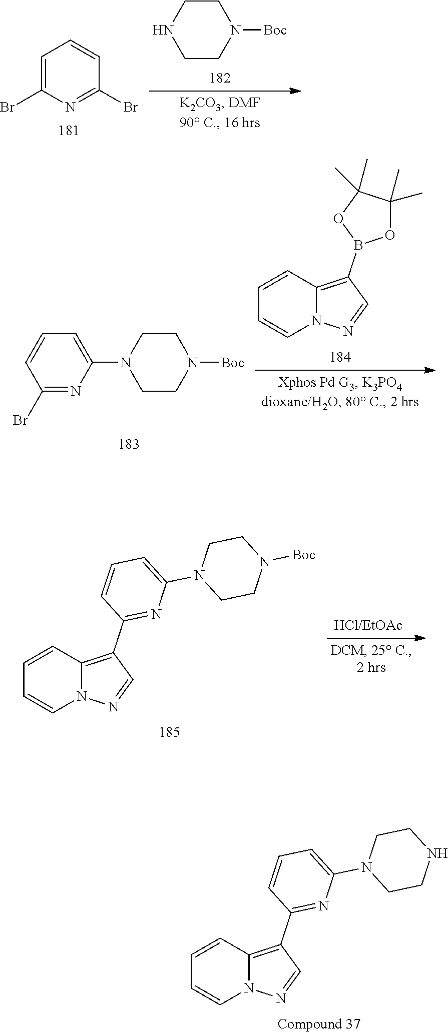 [SMILES: Brc1cccc(Br)n1 ; CC(C)(C)OC(=O)N1CCNCC1 ; CC(C)(C)OC(=O)N1CCN(c2cccc(Br)n2)CC1 ; CC1(C)OB(c2cnn3ccccc23)OC1(C)C ; CC(C)(C)OC(=O)N1CCN(c2cccc(-c3cnn4ccccc34)n2)CC1 ; c1cc(-c2cnn3ccccc23)nc(N2CCNCC2)c1]

Step A: To a solution of tert-butyl piperazine-1-carboxylate 182 (1 g, 5.37 mmol) and 2,6-dibromopyridine 181 (1.91 g, 8.05 mmol) in DMF (20 mL) is added K₂CO₃ (1.48 g, 10.74 mmol). The mixture is stirred at 90° C. for 16 hrs. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-3% ethyl acetate/petroleum ether gradient) to yield the title compound tert-butyl 4-(6-bromo-2-pyridyl)piperazine-1-carboxylate 183 (1.7 g, 84.2% yield) as a white solid. LCMS (ESI MS) m/z=342.0 [M+H]+ 1.

Step B: A mixture of tert-butyl 4-(6-bromopyridin-2-yl)piperazine-1-carboxylate 183 (1 g, 2.92 mmol), 3-(4,4,5,5-tetramethyl-1,3,2-dioxaborolan-2-yl)pyrazolo[1,5-a]pyridine 184 (855.9 mg, 3.51 mmol), XPhos Pd G3 (247.3 mg, 292.2 μmol), and K₃PO₄ (1.24 g, 5.84 mmol) in dioxane (20 mL) and H₂O (5 mL) is degassed and purged with N₂ three times. The mixture is stirred at 80° C. for 2 hr under N₂. The reaction mixture is concentrated under reduced pressure to give a residue which is purified by flash silica gel chromatography (eluent of 0-21% ethyl acetate/petroleum ether gradient) to yield the title compound tert-butyl 4-(6-pyrazolo[1,5-a]pyridin-3-yl-2-pyridyl)piperazine-1-carboxylate 185 (1 g, 84.8% yield) as a yellow solid. LCMS (ESI MS) m/z=380.2 [M+H]+ 1.

Step C: To a solution of tert-butyl 4-(6-(pyrazolo[1,5-a]pyridin-3-yl)pyridin-2-yl)-piperazine-1-carboxylate 185 (700 mg, 1.84 mmol) in DCM (5 mL) is added HCl/EtOAc (10 mL). The mixture is stirred at 25° C. for 2 hr. The reaction mixture is extracted with H₂O three times. The combined water layers are lyophilized to give the title compound 3-(6-piperazin-1-yl-2-pyridyl)pyrazolo[1,5-a]pyridine (Compound 37) (500 mg, 96.8% yield) as a yellow solid. LCMS (ESI MS) m/z=280.1 [M+H]+ 1.

¹H NMR (400 MHz, methanol-d₄) δ=8.72 (d, J=7.2 Hz, 1H), 8.59 (s, 1H), 8.16 (d, J=9.2 Hz, 1H), 8.07 (dd, J=8.8, 7.8 Hz, 1H), 7.55 (ddd, J=8.8, 6.9, 0.9 Hz, 1H), 7.39 (d, J=7.6 Hz, 1H), 7.11-7.18 (m, 2H), 4.01-4.05 (m, 4H), 3.46-3.50 (m, 4H).

### Example 38

### N-methyl-3-(6-piperazin-1-yl-2-pyridyl)pyrazolo[1,5-a]pyridin-5-amine

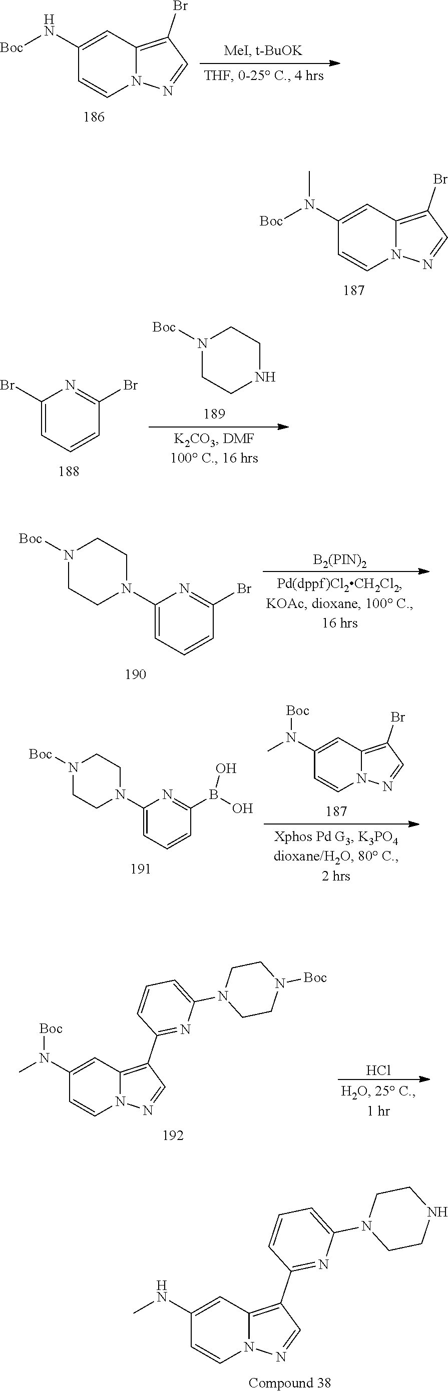

Step A: To a solution of tert-butyl (3-bromopyrazolo[1,5-a]pyridin-5-yl)carbamate 186 (750.0 mg, 2.40 mmol) in THF (10 mL) is added dropwise t-BuOK (1.08 g, 9.61 mmol) at 0° C. After addition, the mixture is stirred at this temperature for 0.5 hr, and then Mel (1.36 g, 9.61 mmol) is added dropwise at 0° C. The resulting mixture is stirred at 25° C. for 3.5 hrs. The reaction is quenched with aq. NH₃·H₂O (5 mL) drop-wise. The resulting mixture is quenched with water and extracted with EtOAc twice. The combined organic phase is washed with brine, dried over anhydrous Na₂SO₄, filtered and concentrated under reduced pressure to give a residue which is purified by flash silica gel chromatography (eluent of 0-30% ethyl acetate/petroleum ether gradient) to yield the title compound tert-butyl N-(3-bromopyrazolo[1,5-a]pyridin-5-yl)-N-methyl-carbamate 187 (680 mg) as a yellow oil.

¹H NMR (400 MHz, methanol-d₄) δ=8.44 (d, J=7.6 Hz, 1H), 7.93 (s, 1H), 7.35 (d, J=2.4 Hz, 1H), 6.99 (dd, J=2.4, 7.6 Hz, 1H), 3.32 (s, 3H), 1.49 (s, 9H).

Step B: A mixture of 2,6-dibromopyridine 188 (953.9 mg, 4.03 mmol), tert-butyl piperazine-1-carboxylate 1_8 (500 mg, 2.68 mmol) and K₂CO₃ (742.0 mg, 5.37 mmol) in DMF (15 mL) is stirred at 100° C. for 16 hrs. The reaction mixture is diluted with H₂O, and the mixture is extracted with EtOAc three times. The combined organic layers are washed with brine, dried over Na₂SO₄, filtered and concentrated under reduced pressure to give a residue which is purified by flash silica gel chromatography (eluent of 0-8% ethyl acetate/petroleum ether gradient) to yield the title compound tert-butyl 4-(6-bromo-2-pyridyl)piperazine-1-carboxylate 190 (650 mg) as a white solid. LCMS (ESI MS) m/z=342.0 [M+H]+ 1.

Step C: A mixture of 4,4,5,5-tetramethyl-2-(4,4,5,5-tetramethyl-1,3,2-dioxaborolan-2-yl)-1,3,2-dioxaborolane (486.1 mg, 1.91 mmol), tert-butyl 4-(6-bromopyridin-2-yl)piperazine-1-carboxylate 190 (400 mg, 1.06 mmol), Pd(dppf)Cl₂·CH₂Cl₂ (86.8 mg, 106.36 μmol), and KOAc (156.5 mg, 1.60 mmol) in dioxane (10 mL) is purged with N₂ three times, and then the mixture is stirred at 80° C. for 16 hr under N₂. The reaction mixture is concentrated directly to give the crude title compound tert-butyl4-[6-(4,4,5,5-tetramethyl-1,3,2-dioxaborolan-2-yl)-2-pyridyl]-piperazine-1-carboxylate 191 (414 mg) as a brown solid. LCMS (ESI MS) m/z=308.1 [M+H]+ 1.

Step D: A mixture of tert-butyl (3-bromopyrazolo[1,5-a]pyridin-5-yl)(methyl)carbamate 187 (80 mg, 233 μmol), (6-(4-(tert-butoxycarbonyl)piperazin-1-yl)pyridin-2-yl)boronic acid 191 (272 mg, 699 μmol), K₃PO₄ (98.9 mg, 466 μmol), and XPhos Pd G3 (19.7 mg, 23.3 μmol) in dioxane (2 mL) and H₂O (0.5 mL) is purged with N₂ three times, and then the mixture is stirred at 80° C. for 2 hr under N₂. The reaction mixture is concentrated directly to yield the title compound tert-butyl4-[6-[5-[tert-butoxycarbonyl(methyl)amino]pyrazolo[1,5-a]pyridin-3-yl]-2-pyridyl-]piperazine-1-carboxylate 192 (90 mg, crude) as a black solid.

Step E: To a solution of tert-butyl 4-(6-(5-((tert-butoxycarbonyl)(methyl)amino)-pyrazolo[1,5-a]pyridin-3-yl)pyridin-2-yl)piperazine-1-carboxylate 192 (80 mg, 157.3 μmol) in H₂O (0.5 mL) is added HCl (12 M, 13 μL). The mixture is stirred at 25° C. for 1 hr. The reaction mixture is concentrated directly to give a residue which is purified by prep-HPLC (column: Nano C18, 100×40 mm 10 um; mobile phase: [water (HCl)-ACN]; gradient: 0%-26% B over 36 min) to yield the title compound N-methyl-3-(6-piperazin-1-yl-2-pyridyl)pyrazolo[1,5-a]pyridin-5-amine (Compound 38) (12.6 mg, 25% yield) as a yellow solid. LCMS (ESI MS) m/z=309.1 [M+H]+ 1.

¹H NMR (400 MHz, methanol-d₄) δ=8.42 (s, 1H), 8.31 (d, J=7.6 Hz, 1H), 7.95 (t, J=8.0 Hz, 1H), 7.31 (d, J=7.6 Hz, 1H), 7.00-6.88 (m, 2H), 6.63 (d, J=7.6 Hz, 1H), 3.99-3.95 (m, 4H), 3.47-3.43 (m, 4H), 2.92 (s, 3H).

### Example 39

### 3-(4-chloro-6-piperazin-1-yl-2-pyridyl)-N-methyl-pyrazolo[1,5-a]pyridin-5-amine

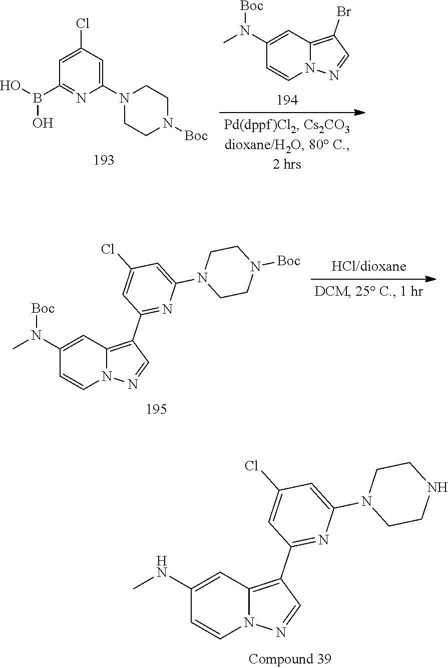 [SMILES: CC(C)(C)OC(=O)N1CCN(c2cc(Cl)cc(B(O)O)n2)CC1 ; CN(C(=O)OC(C)(C)C)c1ccn2ncc(Br)c2c1 ; CN(C(=O)OC(C)(C)C)c1ccn2ncc(-c3cc(Cl)cc(N4CCN(C(=O)OC(C)(C)C)CC4)n3)c2c1 ; CNc1ccn2ncc(-c3cc(Cl)cc(N4CCNCC4)n3)c2c1]

Step A: A mixture of (6-(4-(tert-butoxycarbonyl)piperazin-1-yl)-4-chloropyridin-2-yl)boronic acid 193 (126 mg, 367.8 μmol), tert-butyl (3-bromopyrazolo[1,5-a]pyridin-5-yl)(methyl)carbamate 194 (60 mg, 183.9 μmol), Pd(dppf)Cl₂ (30 mg, 36.8 μmol), and Cs₂CO₃ (72 mg, 220.7 μmol) in dioxane (2 mL) and H₂O (0.2 mL) is purged with N₂ three times, and then the mixture is stirred at 80° C. for 2 hr under N₂. The reaction is quenched with water and extracted with EtOAc three times. The combined organic layers are dried over anhydrous Na₂SO₄, filtered and concentrated under reduced pressure to give a residue which is purified by flash silica gel chromatography (eluent of 0-50% ethyl acetate/petroleum ether gradient) to yield the title compound tert-butyl 4-[6-[5-[tert-butoxycarbonyl(methyl)amino]pyrazolo[1,5-a]pyridin-3-yl]-4-chloro-2-pyridyl]piperazine-1-carboxylate 195 (40 mg) as a yellow oil. LCMS (ESI MS) m/z=543.4 [M+H]+ 1.

Step B: To a solution of tert-butyl 4-(6-(5-((tert-butoxycarbonyl)(methyl)amino)-pyrazolo[1,5-a]pyridin-3-yl)-4-chloropyridin-2-yl)piperazine-1-carboxylate 195 (20 mg, 36.8 μmol) in DCM (2 mL) is added HCl/dioxane (4 M, 1 mL). The mixture is stirred at 25° C. for 1 hr. The reaction mixture is concentrated under vacuum to give the crude product which is purified by prep-HPLC (column: Xtimate C18, 150×40 mm×10 um; mobile phase: [water (FA)-ACN]; gradient: 0%-34% B over 25 min]; gradient: 12%-52% B over 32 min) to yield the title compound 3-(4-chloro-6-piperazin-1-yl-2-pyridyl)-N-methyl-pyrazolo[1,5-a]pyridin-5-amine (Compound 39) (5.6 mg, FA salt) as a white solid. LCMS (ESI MS) m/z=343 [M+H]+ 1.

¹H NMR (400 MHz, methanol-d₄) δ=8.51 (br s, 1H), 8.25 (s, 1H), 8.13-8.19 (m, 1H), 7.20 (br s, 1H), 7.12 (s, 1H), 6.64 (s, 1H), 6.42-6.49 (m, 1H), 3.86-3.90 (m, 4H), 3.28 (br s, 4H), 2.87 (s, 3H).

### Example 40

### 3-(6-piperazin-1-yl-2-pyridyl)-5-(trifluoromethyl)pyrazolo[1,5-a]pyridine

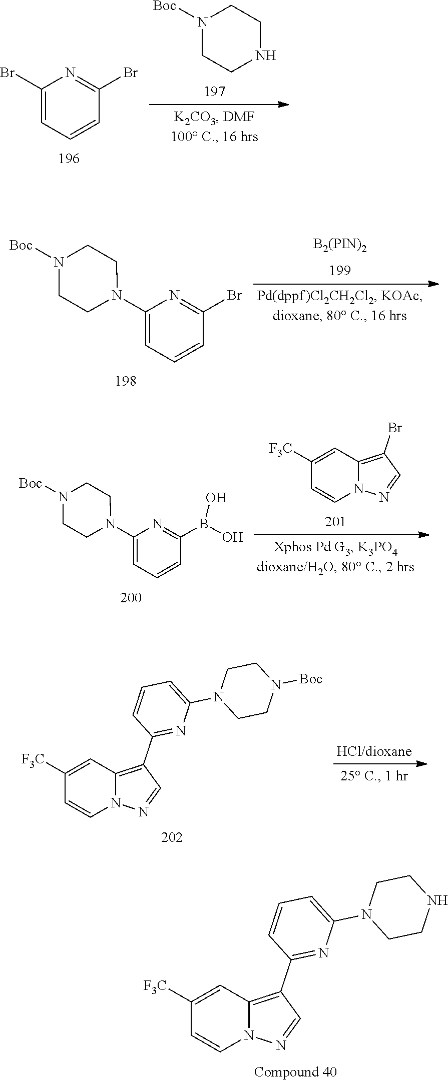

Step A: A mixture of 2,6-dibromopyridine 196 (954 mg, 4.03 mmol), tert-butyl piperazine-1-carboxylate 197 (500 mg, 2.68 mmol) and K₂CO₃ (742.0 mg, 5.37 mmol) in DMF (15 mL) is stirred at 100° C. for 16 hr. The reaction mixture is diluted with H₂O, and the mixture is extracted with EtOAc three times. The combined organic layers are washed with brine, dried over Na₂SO₄, filtered and concentrated under reduced pressure to give a residue which is purified by flash silica gel chromatography (eluent of 0-8% ethyl acetate/petroleum ether gradient) to yield the title compound tert-butyl 4-(6-bromo-2-pyridyl)piperazine-1-carboxylate 198 (650 mg) as a white solid. LCMS (ESI MS) m/z=342.0 [M+H]+ 1.

Step B: A mixture of 4,4,5,5-tetramethyl-2-(4,4,5,5-tetramethyl-1,3,2-dioxaborolan-2-yl)-1,3,2-dioxaborolane 199 (486.1 mg, 1.91 mmol), 195 (400 mg, 1.06 mmol), Pd(dppf)Cl₂·CH₂Cl₂ (86.8 mg, 106.36 μmol), and KOAc (156.5 mg, 1.60 mmol) in dioxane (10 mL) is purged with N₂ three times, and then the mixture is stirred at 80° C. for 16 hr under N₂. The reaction mixture is concentrated directly to give the title compound tert-butyl 4-[6-(4,4,5,5-tetramethyl-1,3,2-dioxaborolan-2-yl)-2-pyridyl]piperazine-1-carboxylate 200 (414 mg, crude) as a brown solid. LCMS (ESI MS) m/z=308.1 [M+H]+ 1.

Step C: A mixture of 3-bromo-5-(trifluoromethyl)pyrazolo[1,5-a]pyridine 201 (100 mg, 369.77 μmol), tert-butyl 4-[6-(4,4,5,5-tetramethyl-1,3,2-dioxaborolan-2-yl)-2-pyridyl]piperazine-1-carboxylate 200 (287.9 mg, 739.5 μmol), XPhos Pd G3 (31 mg, 36.98 μmol), and K₃PO₄ (156.9 mg, 739.5 μmol) in dioxane (4 mL) and H₂O (1 mL) is purged with N₂ three times, and then the mixture is stirred at 80° C. for 2 hr under N₂. The reaction mixture is concentrated under reduced pressure to give the title compound tert-butyl 4-[6-[5-(trifluoromethyl)pyrazolo[1,5-a]pyridin-3-yl]-2-pyridyl]piperazine-1-carboxylate 202 (165 mg, crude) as a brown solid. LCMS (ESI MS) m/z=448.1 [M+H]+ 1.

Step D: A solution of tert-butyl 4-[6-[5-(trifluoromethyl)pyrazolo[1,5-a]pyridin-3-yl]-2-pyridyl]piperazine-1-carboxylate 202 (100 mg, 223.5 μmol) in HCl/dioxane (4 M, 2 mL) is stirred at 25° C. for 2 hr. The reaction mixture is concentrated to give a residue which is purified by prep-HPLC (column: Xtimate C18, 150×40 mm×10 um; mobile phase: [water (FA)-ACN]; gradient: 0%-34% B over 25 min) to yield the title compound 3-(6-piperazin-1-yl-2-pyridyl)-5-(trifluoromethyl)pyrazolo[1,5-a]pyridine (Compound 40) (32.0 mg, 36%, FA salt) as a white solid. LCMS (ESI MS) m/z=348.1 [M+H]+ 1.

¹H NMR (400 MHz, methanol-d₄) δ=8.84 (br s, 1H), 8.71 (d, J=7.2 Hz, 1H), 8.56 (d, J=0.8 Hz, 1H), 8.53 (s, 1H), 7.65 (t, J=8.0 Hz, 1H), 7.21-7.26 (m, 1H), 7.11 (br d, J=7.2 Hz, 1H), 6.79 (d, J=8.4 Hz, 1H), 3.85-3.89 (m, 4H), 3.32-3.36 (m, 4H).

### Example 41

### 3-(4-chloro-6-piperazin-1-yl-2-pyridyl)-5-(trifluoromethyl) pyrazolo[1,5-a]pyridine

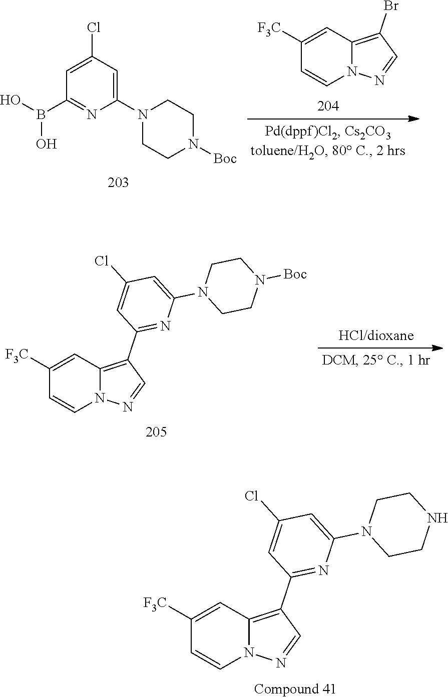 [SMILES: CC(C)(C)OC(=O)N1CCN(c2cc(Cl)cc(B(O)O)n2)CC1 ; FC(F)(F)c1ccn2ncc(Br)c2c1 ; CC(C)(C)OC(=O)N1CCN(c2cc(Cl)cc(-c3cnn4ccc(C(F)(F)F)cc34)n2)CC1 ; FC(F)(F)c1ccn2ncc(-c3cc(Cl)cc(N4CCNCC4)n3)c2c1]

Step A: A mixture of (6-(4-(tert-butoxycarbonyl)piperazin-1-yl)-4-chloropyridin-2-yl)boronic acid 203 (60 mg, 226.4 μmol), 3-bromo-5-(trifluoromethyl)pyrazolo[1,5-a]pyridine 204 (116 mg, 339.6 μmol), Cs₂CO₃ (147 mg, 452.8 μmol), and Pd(dppf)Cl₂ (36.9 mg, 45.3 μmol) in toluene (2 mL) and H₂O (0.5 mL) is purged with N₂ three times, and then the mixture is stirred at 80° C. for 2 hr under N₂. The reaction is diluted with water and extracted with EtOAc three times. The combined organic layers are dried over anhydrous Na₂SO₄, filtered and concentrated under reduced pressure to give a residue which is purified by flash silica gel chromatography (eluent of 0-5% ethyl acetate/petroleum ether gradient) to yield the title compound tert-butyl 4-[4-chloro-6-[5-(trifluoromethyl) pyrazolo[1,5-a]pyridin-3-yl]-2-pyridyl]piperazine-1-carboxylate 205 (40 mg) as a white solid. LCMS (ESI MS) m/z=482.0 [M+H]+1.

Step B: To a solution of tert-butyl 4-(4-chloro-6-(5-(trifluoromethyl)pyrazolo[1,5-a]pyridin-3-yl) pyridin-2-yl)piperazine-1-carboxylate 205 (10 mg, 20.76 μmol) in DCM (1 mL) is added HCl/dioxane (4M, 0.5 mL). The mixture is stirred at 25° C. for 1 hr. The reaction mixture is concentrated under vacuum to give the crude product which is purified by prep-HPLC (column: Xtimate C18, 150×40 mm×10 um; mobile phase: [water (FA)-ACN]; gradient: 0%-38% B over 25 min) to yield the title compound 3-(4-chloro-6-piperazin-1-yl-2-pyridyl)-5-(trifluoromethyl) pyrazolo[1,5-a]pyridine (Compound 41) (2.0 mg, FA salt) as a white solid. LCMS (ESI MS) m/z=382.1 [M+H]+ 1.

¹H NMR (400 MHz, DMSO-d₆) δ=8.99 (d, J=7.25 Hz, 1H), 8.91 (s, 1H), 8.88 (s, 1H), 8.21 (s, 1H), 7.38 (d, J=1.38 Hz, 1H), 7.27 (dd, J=7.32, 2.06 Hz, 1H), 6.83 (d, J=1.13 Hz, 1H), 3.61 (br s, 4H), 2.92 (br s, 4H).

### Example 42

### 5-chloro-3-(6-piperazin-1-yl-2-pyridyl)pyrazolo[1,5-a]pyridine

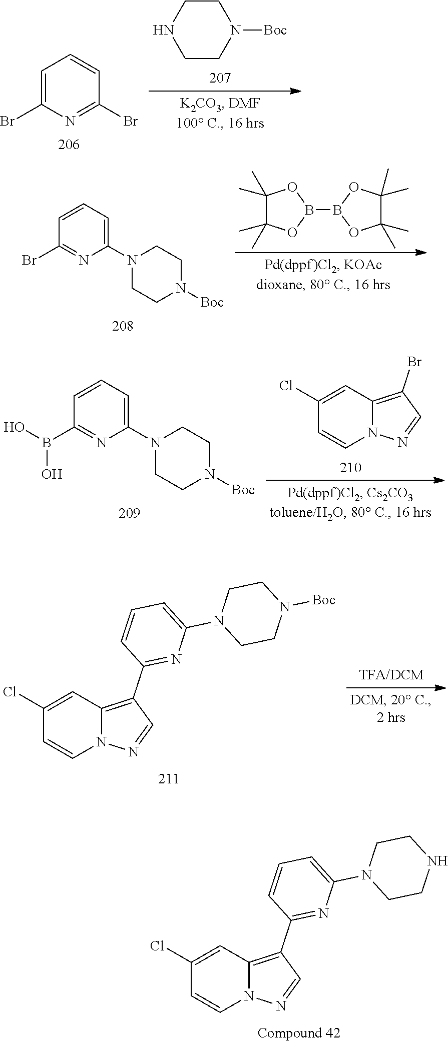

Step A: A mixture of 2,6-dibromopyridine 206 (954 mg, 4.03 mmol), tert-butyl piperazine-1-carboxylate 207 (500 mg, 2.68 mmol) and K₂CO₃ (742 mg, 5.37 mmol) in DMF (15 mL) is stirred at 100° C. for 16 hrs. Water is added to the reaction mixture, and the mixture is extracted with EtOAc three times. The combined organic layers are washed with brine, dried, filtered and concentrated under reduced pressure to give a residue. The residue is purified by flash silica gel chromatography (eluent of 0-8% ethyl acetate/petroleum ether gradient) to yield the title compound tert-butyl 4-(6-bromo-2-pyridyl)piperazine-1-carboxylate 208 (650 mg, 65%) as a white solid. LCMS (ESI MS) m/z=341.9 [M+H]+ 1.

Step B: A mixture of tert-butyl 4-(6-bromopyridin-2-yl)piperazine-1-carboxylate 208 (150 mg, 438.3 μmol), 4,4,5,5-tetramethyl-2-(4,4,5,5-tetramethyl-1,3,2-dioxaborolan-2-yl)-1,3,2-dioxaborolane (200 mg, 788.9 μmol), Pd(dppf)Cl₂·CH₂Cl₂ (35.7 mg, 43.8 μmol), and KOAc (64.5 mg, 657.4 μmol) in dioxane (4 mL) is degassed and purged with N₂ three times. The mixture is stirred at 80° C. for 16 hr under a N₂ atmosphere. The reaction mixture is concentrated directly to yield the title compound [6-(4-tert-butoxycarbonylpiperazin-1-yl)-2-pyridyl]boronic acid 209 (134.6 mg, crude) as a brown solid. LCMS (ESI MS) m/z=308.1 [M+H]+ 1.

Step C: A mixture of (6-(4-(tert-butoxycarbonyl)piperazin-1-yl)pyridin-2-yl)boronic acid 20 (130 mg, 423.2 μmol), 3-bromo-5-chloropyrazolo[1,5-a]pyridine 210 (107.7 mg, 465.6 μmol), Pd(dppf)Cl₂ (30.9 mg, 42.3 μmol), and Cs₂CO₃ (275.8 mg, 846.49 μmol) in toluene (4 mL) and H₂O (1 mL) is degassed and purged with N₂ three times. The mixture is stirred at 80° C. for 16 hr under N₂. The reaction mixture is concentrated under vacuum. The residue is purified by flash silica gel chromatography (eluent of 0-20% ethyl acetate/petroleum ether gradient) to yield the title compound tert-butyl 4-[6-(5-chloropyrazolo[1,5-a]pyridin-3-yl)-2-pyridyl]piperazine-1-carboxylate 211 (90 mg, 20%) as a yellow solid. LCMS (ESI MS) m/z=414.1 [M+H]+ 1.

Step D: To a solution of tert-butyl 4-(6-(5-chloropyrazolo[1,5-a]pyridin-3-yl)pyridin-2-yl)piperazine-1-carboxylate 211 (80 mg, 193.3 μmol) in DCM (2 mL) is added TFA (0.4 mL). The mixture is stirred at 20° C. for 2 hrs. The reaction mixture is extracted with H₂O three times. The combined water layers are concentrated under reduced pressure to give a residue. The residue is purified by prep-HPLC (column: Xtimate C18, 150×40 mm×10 um; mobile phase: [water (FA)-ACN]; gradient: 0%-32% B over 25 min) to yield the title compound 5-chloro-3-(6-piperazin-1-yl-2-pyridyl)pyrazolo[1,5-a]pyridine (Compound 42) (12.8 mg, 21%) as a white solid. LCMS (ESI MS) m/z=314.1 [M+H]+ 1.

¹H NMR (400 MHz, methanol-d₄) δ=8.57 (d, J=7.2 Hz, 1H), 8.50 (s, 1H), 8.46 (d, J=2.4 Hz, 1H), 7.62-7.69 (m, 1H), 7.23 (d, J=7.6 Hz, 1H), 6.97 (dd, J=7.2, 2.4 Hz, 1H), 6.78 (d, J=8.4 Hz, 1H), 3.84-3.90 (m, 4H), 3.33-3.39 (m, 4H).

### Example 43

### 5-chloro-3-(4-chloro-6-piperazin-1-yl-2-pyridyl)pyrazolo[1,5-a]pyridine

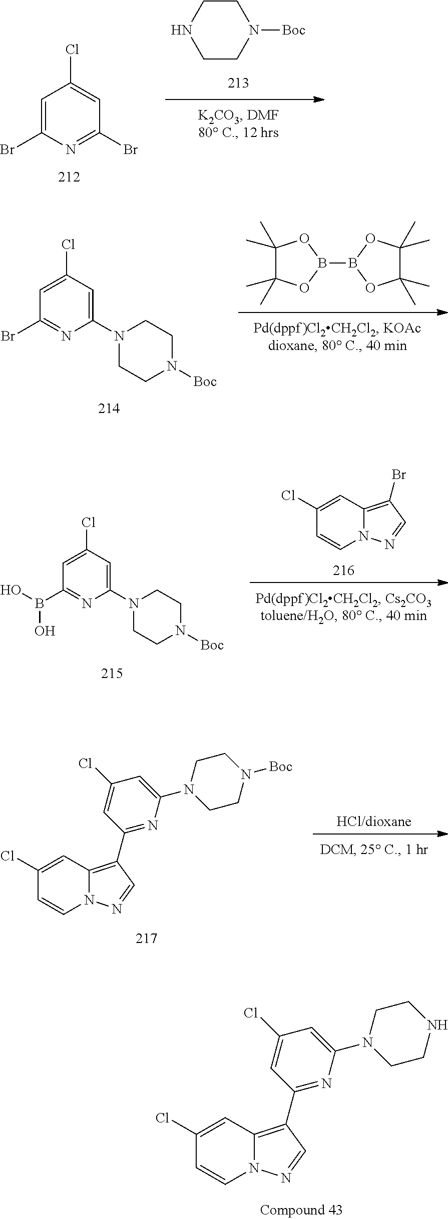

Step A: To a solution of 2,6-dibromo-4-chloropyridine 212 (503 mg, 1.85 mmol) and tert-butyl piperazine-1-carboxylate 213 (230 mg, 1.23 mmol) in DMF (5 mL) is added K₂CO₃ (340 mg, 2.47 mmol). The mixture is stirred at 80° C. for 12 hrs. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-11% ethyl acetate/petroleum ether gradient) to yield the title compound tert-butyl 4-(6-bromo-4-chloro-2-pyridyl)piperazine-1-carboxylate 214 (270 mg) as a white solid. LCMS (ESI MS) m/z=321.9 [M−56]+1.

¹H NMR (400 MHz, DMSO-d₆) δ=6.95 (d, J=1.38 Hz, 2H), 3.48-3.55 (m, 4H), 3.40 (br d, J=5.13 Hz, 4H), 1.42 (s, 9H).

Step B: A mixture of tert-butyl 4-(6-bromo-4-chloropyridin-2-yl)piperazine-1-carboxylate 214 (100 mg, 265.5 μmol), 4,4,5,5-tetramethyl-2-(4,4,5,5-tetramethyl-1,3,2-dioxaborolan-2-yl)-1,3,2-dioxaborolane (135 mg, 530.9 μmol), Pd(dppf)Cl₂·CH₂Cl₂ (44 mg, 53.1 μmol), and KOAc (31 mg, 318.6 μmol) in dioxane (1 mL) is degassed and purged with N₂ three times. The mixture is stirred at 80° C. for 40 min under a N₂ atmosphere. The reaction is filtered to remove the insoluble, and the filtrate is concentrated under vacuum to give the title compound [6-(4-tert-butoxycarbonylpiperazin-1-yl)-4-chloro-2-pyridyl]boronic acid 215 (220 mg, crude) as a brown oil. LCMS (ESI MS) m/z=342.1 [M+H]+ 1.

Step C: A mixture of (6-(4-(tert-butoxycarbonyl)piperazin-1-yl)-4-chloropyridin-2-yl)boronic acid 215 (206 mg, 362.9 μmol), 3-bromo-5-chloropyrazolo[1,5-a]pyridine 216 (70 mg, 302.4 μmol), Cs₂CO₃ (197 mg, 604.8 μmol), and Pd(dppf)Cl₂·CH₂Cl₂ (50 mg, 60.5 μmol) in toluene (2 mL) and H₂O (0.5 mL) is degassed and purged with N₂ three times. The mixture is stirred at 80° C. for 40 min under N₂. The reaction is quenched with water and extracted with EtOAc three times. The combined organic layers are dried over anhydrous Na₂SO₄, filtered and concentrated under reduced pressure to give a residue which is purified by flash silica gel chromatography (eluent of 0-15% ethyl acetate/petroleum ether gradient) to yield the title compound tert-butyl 4-[4-chloro-6-(5-chloropyrazolo[1,5-a]pyridin-3-yl)-2-pyridyl]piperazine-1-carboxylate 217 (45 mg) as a yellow solid. LCMS (ESI MS) m/z=448.1 [M+H]+ 1.

Step D: To a solution of tert-butyl 4-(4-chloro-6-(5-chloropyrazolo[1,5-a]pyridin-3-yl)pyridin-2-yl)piperazine-1-carboxylate 217 (40 mg, 66.9 μmol) in DCM (1 mL) is added HCl/dioxane (4 M, 133 μL). The mixture is stirred at 25° C. for 1 hr. The reaction mixture is concentrated under vacuum to give the crude product which is purified by prep-HPLC (column: Xtimate C18, 150×40 mm×10 um; mobile phase: [water (FA)-ACN]; gradient: 0%-36% B over min) to yield the title compound 5-chloro-3-(4-chloro-6-piperazin-1-yl-2-pyridyl)pyrazolo[1,5-a]pyridine (Compound 43) (20.6 mg, 58%) as a white solid. LCMS (ESI MS) m/z=348.0 [M+H]+ 1.

¹H NMR (400 MHz, DMSO-d₆) δ=8.81 (d, J=7.38 Hz, 1H), 8.76 (s, 1H), 8.41 (br s, 1H), 8.26 (br d, J=8.88 Hz, 1H), 7.29 (s, 1H), 7.08 (dd, J=7.32, 1.94 Hz, 1H), 6.77 (s, 1H), 3.63-3.73 (m, 4H), 2.94 (br s, 4H).

### Example 44

### 5-chloro-3-(4-fluoro-6-piperazin-1-yl-2-pyridyl)pyrazolo[1,5-a]pyridine

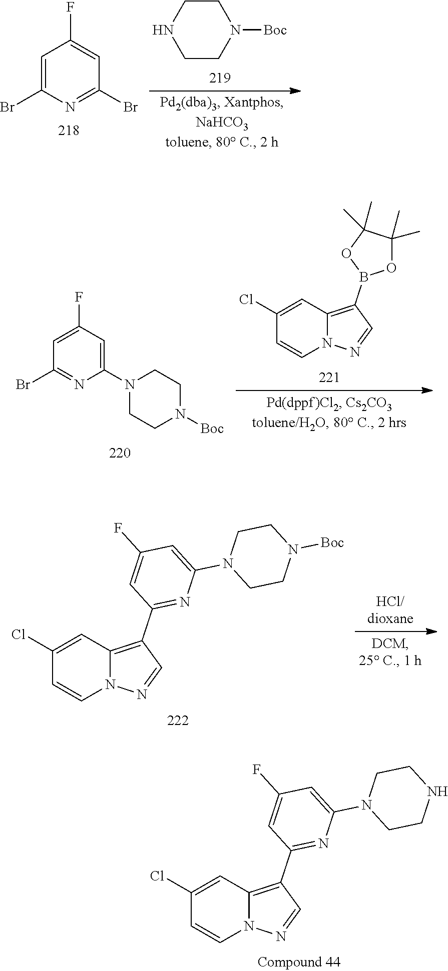 [SMILES: Fc1cc(Br)nc(Br)c1 ; CC(C)(C)OC(=O)N1CCNCC1 ; CC(C)(C)OC(=O)N1CCN(c2cc(F)cc(Br)n2)CC1 ; CC1(C)OB(c2cnn3ccc(Cl)cc23)OC1(C)C ; CC(C)(C)OC(=O)N1CCN(c2cc(F)cc(-c3cnn4ccc(Cl)cc34)n2)CC1 ; Fc1cc(-c2cnn3ccc(Cl)cc23)nc(N2CCNCC2)c1]

Step A: A mixture of 2,6-dibromo-4-fluoropyridine 218 (460 mg, 1.80 mmol), tert-butyl piperazine-1-carboxylate 219 (672.2 mg, 3.61 mmol), Pd₂(dba)₃ (165.2 mg, 180.48 μmol), Xantphos (522.1 mg, 902.38 μmol), and NaHCO₃ (151.6 mg, 1.80 mmol) in toluene (5 mL) is purged with N₂ three times, and then the mixture is stirred at 80° C. for 2 hours under N₂. The reaction mixture is filtered, and the filtrate is concentrated under reduced pressure to give a residue. The residue is purified by flash silica gel chromatography (eluent of 9-10% ethyl acetate/petroleum ether gradient) to give a crude product, which is further purified by prep-HPLC (Neutral condition; column: Xtimate C18, 150×40 mm×10 um; mobile phase: [water (NH₄HCO₃)-ACN]; gradient: 46%-86% B over 32 min) to yield the title compound tert-butyl 4-(6-bromo-4-fluoropyridin-2-yl)piperazine-1-carboxylate 220 (100 mg) as a white solid. LCMS (ESI MS) m/z=304.0 [M−55].

Step B: A mixture of tert-butyl 4-(6-bromo-4-fluoropyridin-2-yl)piperazine-1-carboxylate 220 (20 mg, 55.52 μmol), 5-chloro-3-(4,4,5,5-tetramethyl-1,3,2-dioxaborolan-2-yl)pyrazolo[1,5-a]pyridine 221 (30.9 mg, 111.04 μmol), Cs₂CO₃ (27.1 mg, 83.28 μmol), and Pd(dppf)Cl₂·CH₂Cl₂ (9.1 mg, 11.10 μmol) in toluene (1 mL) and H₂O (1 mL) is purged with N₂ three times, and then the mixture is stirred at 80° C. for 2 hr under N₂. The reaction is diluted with H₂O and extracted with EtOAc three times. The combined organic layers are dried over anhydrous Na₂SO₄, filtered and concentrated under reduced pressure to give a residue which is purified by flash silica gel chromatography (eluent of 15-20% ethyl acetate/petroleum ether gradient) to yield the title compound tert-butyl4-[6-(5-chloropyrazolo[1,5-a]pyridin-3-yl)-4-fluoro-2-pyridyl]-piperazine-1-carboxylate 222 (30 mg) as a yellow solid. LCMS (ESI MS) m/z=432.2 [M+H]+ 1.

Step C: To a solution of tert-butyl 4-(6-(5-chloropyrazolo[1,5-a]pyridin-3-yl)-4-fluoropyridin-2-yl) piperazine-1-carboxylate 222 (30 mg, 69.46 μmol) in DCM (1 mL) is added HCl/dioxane (4 M, 70 μL). The mixture is stirred at 25° C. for 1 hr. The reaction mixture is concentrated under vacuum to give the crude product which is purified by prep-HPLC (HCl condition; column: Xtimate C18 150×40 mm×10 um; mobile phase: [water (HCl)-ACN]; gradient: 0%-40% B over 36 min) to yield the title compound 5-chloro-3-(4-fluoro-6-piperazin-1-yl-2-pyridyl)pyrazolo[1,5-a]pyridine (Compound 44) (14.9 mg, 64%) as a white solid. LCMS (ESI MS) m/z=332.1 [M+H]+ 1.

¹H NMR (400 MHz, methanol-d₄) δ=8.61 (d, J=7.2 Hz, 1H), 8.55 (s, 1H), 8.45 (d, J=1.6 Hz, 1H), 8.42-8.49 (m, 1H), 7.08 (dd, J=9.6, 1.6 Hz, 1H), 7.03 (dd, J=7.6, 2.4 Hz, 1H), 6.59 (dd, J=12.0, 1.6 Hz, 1H), 3.92-3.99 (m, 4H), 3.40-3.44 (m, 4H).

¹⁹F NMR (377 MHz, methanol-d₄) δ=−104.39 (s, 1 F).

### Example 45

### 3-[6-(3,6-Diazabicyclo[3.1.1]heptan-3-yl)-2-pyridyl]pyrazolo[1,5-a]pyridine

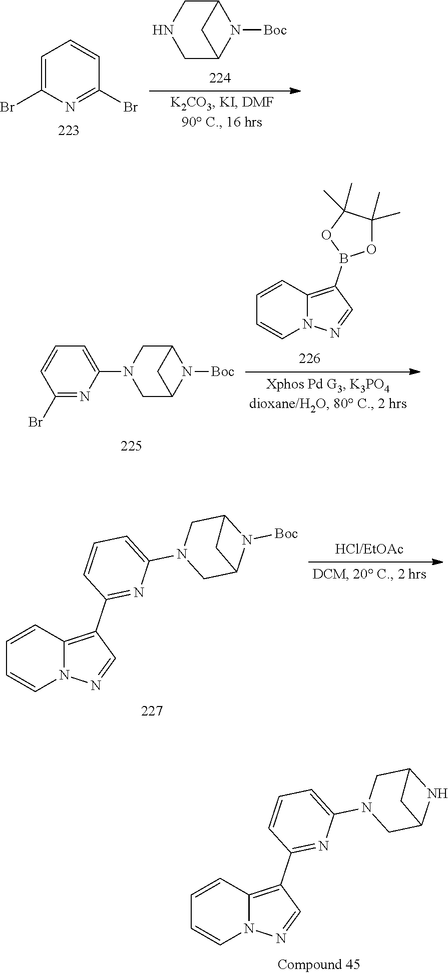 [SMILES: Brc1cccc(Br)n1 ; CC(C)(C)OC(=O)N1C2CNCC1C2 ; CC1(C)OB(c2cnn3ccccc23)OC1(C)C ; CC(C)(C)OC(=O)N1C2CC1CN(c1cccc(Br)n1)C2 ; CC(C)(C)OC(=O)N1C2CC1CN(c1cccc(-c3cnn4ccccc34)n1)C2 ; c1cc(-c2cnn3ccccc23)nc(N2CC3CC(C2)N3)c1]

Step A: To a solution of 2,6-dibromopyridine 223 (716.9 mg, 3.03 mmol) in DMF (10 mL) are added tert-butyl 3,6-diazabicyclo[3.1.1]heptane-6-carboxylate 224 (500 mg, 2.52 mmol), K₂CO₃ (1.05 g, 7.57 mmol) and KI (209.3 mg, 1.26 mmol). The mixture is stirred at 90° C. for 16 hr. The reaction is quenched with water and extracted with EtOAc three times. The combined organic layers are washed with brine twice, dried over anhydrous Na₂SO₄, filtered and concentrated under reduced pressure to give a residue which is purified by flash silica gel chromatography (eluent of 0-10% ethyl acetate/petroleum ether gradient) to yield the title compound tert-butyl 3-(6-bromo-2-pyridyl)-3,6-diazabicyclo[3.1.1]heptane-6-carboxylate 225 (600 mg, 67% yield) as a white solid. LCMS (ESI MS) m/z=354.0 [M+H]+ 1.

¹H NMR (400 MHz, chloroform-d) 5=7.30 (t, J=8.0 Hz, 1H), 6.77 (d, J=7.2 Hz, 1H), 6.41 (d, J=8.4 Hz, 1H), 4.28 (br d, J=5.2 Hz, 2H), 4.10-3.98 (m, 2H), 3.55-3.34 (m, 2H), 2.70-2.61 (m, 1H), 1.47 (d, J=8.4 Hz, 1H), 1.38 (s, 9H).

Step B: A mixture of tert-butyl 3-(6-bromopyridin-2-yl)-3,6-diazabicyclo[3.1.1]heptane-6-carboxylate 225 (217.7 mg, 0.61 mmol), 3-(4,4,5,5-tetramethyl-1,3,2-dioxaborolan-2-yl)pyrazolo[1,5-a]pyridine 226 (150 mg, 0.61 mmol), K₃PO₄ (260.9 mg, 1.23 mmol), and XPhos Pd G₃ (52.0 mg, 61.45 μmol) in dioxane (4 mL) and H₂O (1 mL) is degassed and purged with N₂ three times. The mixture is stirred at 80° C. for 2 hr under a N₂ atmosphere. The reaction mixture is concentrated under reduced pressure to give a residue which is purified by flash silica gel chromatography (eluent of 0-20% ethyl acetate/petroleum ether gradient) to yield the title compound tert-butyl3-(6-pyrazolo[1,5-a]pyridin-3-yl-2-pyridyl)-3,6-diazabicyclo[3.1.1]heptane-6-carboxylate 227 (200 mg, 83.1% yield) as a yellow oil. LCMS (ESI MS) m/z=392.0 [M+H]+ 1.

¹H NMR (400 MHz, chloroform-d) 5=8.60 (d, J=8.8 Hz, 1H), 8.49 (d, J=6.8 Hz, 1H), 8.38 (s, 1H), 7.53 (dd, J=8.4, 7.6 Hz, 1H), 7.26-7.21 (m, 1H), 7.04 (d, J=7.6 Hz, 1H), 6.83 (td, J=6.8, 1.2 Hz, 1H), 6.35 (d, J=8.4 Hz, 1H), 4.36 (br s, 2H), 4.22 (br s, 2H), 3.61 (br d, J=10.8 Hz, 2H), 2.73-2.64 (m, 1H), 1.40-1.35 (m, 10H).

Step C: To a solution of tert-butyl 3-(6-(pyrazolo[1,5-a]pyridin-3-yl)pyridin-2-yl)-3,6-diazabicyclo[3.1.1]heptane-6-carboxylate 227 (50 mg, 0.13 mmol) in DCM (1 mL) is added 4M HCl/EtOAc (1 mL). The mixture is stirred at 20° C. for 2 hr. The reaction mixture is concentrated under reduced pressure to give a residue which is purified by prep-HPLC (column: Welch Xtimate C18, 150×40 mm×10 um; mobile phase: [water (FA)-ACN]; gradient: 18%-56% B over min) to yield the title compound 3-[6-(3,6-Diazabicyclo[3.1.1]heptan-3-yl)-2-pyridyl]pyrazolo[1,5-a]pyridine (Compound 45) (29.1 mg, 77.5% yield) as a white solid. LCMS (ESI MS) m/z=292.1 [M+H]+ 1.

¹H NMR (400 MHz, methanol-d₄) δ=8.64-8.53 (m, 2H), 8.47 (s, 1H), 7.64 (t, J=8.0 Hz, 1H), 7.35 (dd, J=8.4, 7.2 Hz, 1H), 7.22 (d, J=7.6 Hz, 1H), 6.97 (td, J=6.8, 0.8 Hz, 1H), 6.55 (d, J=8.4 Hz, 1H), 4.57 (br d, J=6.0 Hz, 2H), 4.21-4.13 (m, 2H), 4.11-4.04 (m, 2H), 3.08 (dt, J=10.0, 6.4 Hz, 1H), 2.03 (d, J=10.4 Hz, 1H).

### Example 46

### 3-(6-(5-chloropyrazolo[1,5-a]pyridin-3-yl)pyridin-2-yl)-3,6-diazabicyclo[3.1.1]heptane

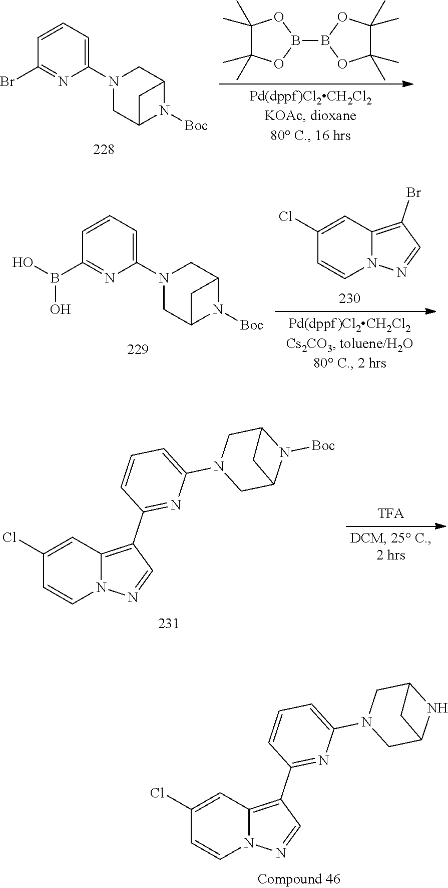

Step A: A mixture of tert-butyl 3-(6-bromopyridin-2-yl)-3,6-diazabicyclo[3.1.1]heptane-6-carboxylate 228 (100 mg, 0.28 mmol), 4,4,5,5-tetramethyl-2-(4,4,5,5-tetramethyl-1,3,2-dioxaborolan-2-yl)-1,3,2-dioxaborolane (143.4 mg, 0.56 mmol), KOAc (41.6 mg, 0.42 mmol), and Pd(dppf)Cl₂·CH₂Cl₂ (23.1 mg, 28.23 μmol) in dioxane (2 mL) is purged with N₂ three times. The mixture is stirred at 80° C. for 16 hr under N₂. The reaction mixture is concentrated under reduced pressure to afford the title compound (6-(6-(tert-butoxycarbonyl)-3,6-diazabicyclo[3.1.1]heptan-3-yl)pyridin-2-yl)boronic acid 229 (90 mg, crude) as a brown oil, which is used in the next step without further purification.

Step B: A mixture of (6-(6-(tert-butoxycarbonyl)-3,6-diazabicyclo[3.1.1]heptan-3-yl)pyridin-2-yl)boronic acid 229 (90 mg, 0.28 mmol), 3-bromo-5-chloropyrazolo[1,5-a]pyridine 230 (65.3 mg, 0.28 mmol), Cs₂CO₃ (183.8 mg, 0.56 mmol), and Pd(dppf)Cl₂·CH₂Cl₂ (46.1 mg, 56.4 μmol) in toluene (4 mL) and H₂O (1 mL) is purged with N₂ three times. The mixture is stirred at 80° C. for 2 hr under N₂. The reaction mixture is concentrated under vacuum to afford the title compound tert-butyl 3-(6-(5-chloropyrazolo[1,5-a]pyridin-3-yl)pyridin-2-yl)-3,6-diazabicyclo-[3.1.1]heptane-6-carboxylate 231 (120 mg, crude) as a brown solid, which is used directly without further purification.

Step C: To a solution of tert-butyl 3-(6-(5-chloropyrazolo[1,5-a]pyridin-3-yl)pyridin-2-yl)-3,6-diazabicyclo[3.1.1]heptane-6-carboxylate 231 (120 mg, 0.28 mmol) in DCM (4 mL) is added TFA (1 mL). The mixture is stirred at 25° C. for 2 hrs. The reaction mixture is diluted with DCM and extracted with H₂O three times. The combined water layers are concentrated under reduced pressure. The residue is purified by prep-HPLC (column: Xtimate C18, 150×40 mm×10 um; mobile phase: [water (FA)-MeOH]; gradient: 12%-52% B over 25 min] to yield the title compound 3-(6-(5-chloropyrazolo[1,5-a]pyridin-3-yl)pyridin-2-yl)-3,6-diazabicyclo[3.1.1]heptane (Compound 46) (12.4 mg, 11%, FA salt) as a white solid. LCMS (ESI MS) m/z=326.1 [M+H]+ 1.

¹H NMR (400 MHz, methanol-d₄) δ=8.62 (d, J=2.0 Hz, 1H), 8.56-8.53 (m, 1H), 8.53-8.51 (m, 1H), 8.49 (s, 1H), 7.65 (t, J=8.0 Hz, 1H), 7.20 (d, J=7.6 Hz, 1H), 6.95 (dd, J=2.4, 7.2 Hz, 1H), 6.57 (d, J=8.4 Hz, 1H), 4.56 (br d, J=6.0 Hz, 2H), 4.19-4.03 (m, 4H), 3.08 (td, J=6.4, 10.4 Hz, 1H), 2.03 (d, J=10.4 Hz, 1H).

### Example 47

### 5-chloro-3-[4-chloro-6-(3,6-diazabicyclo[3.1.1]heptan-3-yl)-2-pyridyl]pyrazolo[1,5-a]pyridine

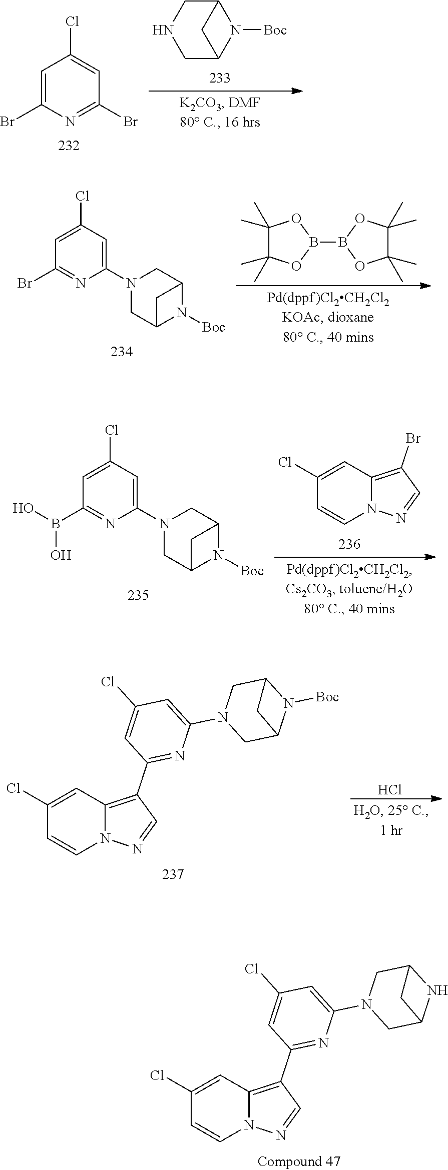

Step A: A mixture of tert-butyl 3,6-diazabicyclo[3.1.1]heptane-6-carboxylate 233 (0.7 g, 3.53 mmol), 2,6-dibromo-4-chloropyridine 232 (1.4 g, 5.30 mmol), and K₂CO₃ (975.9 mg, 7.06 mmol) in DMF (2 mL) is degassed and purged with N₂ three times. The mixture is stirred at 80° C. for 16 hr under a N₂ atmosphere. The reaction mixture is extracted with EtOAc (3×). The combined organic layers are washed with brine three times, dried over Na₂SO₄, filtered and concentrated under reduced pressure to give a crude product which is purified by flash silica gel chromatography (eluent of 0-10% ethyl acetate/petroleum ether gradient) to yield the title compound tert-butyl 3-(6-bromo-4-chloro-2-pyridyl)-3,6-diazabicyclo[3.1.1]heptane-6-carboxylate 234 (600 mg) as an orange solid.

¹H NMR (400 MHz, DMSO-d₆) δ=6.97 (d, J=0.8 Hz, 1H), 6.77 (d, J=0.8 Hz, 1H), 4.18 (br d, J=6.0 Hz, 2H), 4.01-3.76 (m, 2H), 3.39 (br d, J=11.6 Hz, 2H), 2.58-2.53 (m, 1H), 1.45 (d, J=8.8 Hz, 1H), 1.28 (s, 9H).

Step B: A mixture of 4,4,5,5-tetramethyl-2-(4,4,5,5-tetramethyl-1,3,2-dioxaborolan-2-yl)-1,3,2-dioxaborolane (391.9 mg, 1.54 mmol), tert-butyl 3-(6-bromo-4-chloropyridin-2-yl)-3,6-diazabicyclo[3.1.1]heptane-6-carboxylate 234 (200 mg, 514.5 μmol), KOAc (60.6 mg, 617.5 μmol), and Pd(dppf)Cl₂·CH₂Cl₂ (84 mg, 102.91 μmol) in dioxane (2 mL) is degassed and purged with N₂ three times. The mixture is stirred at 80° C. for 40 min under N₂. The reaction mixture is filtered, and the filtrate is concentrated under vacuum to yield the title compound [6-(6-tert-butoxycarbonyl-3,6-diazabicyclo[3.1.1]heptan-3-yl)-4-chloro-2-pyridyl]boronic acid 235 (185 mg, crude) as a black solid.

Step C: A mixture of 3-bromo-5-chloropyrazolo[1,5-a]pyridine 236 (110 mg, 475.2 μmol), (6-(6-(tert-butoxycarbonyl)-3,6-diazabicyclo[3.1.1]heptan-3-yl)-4-chloropyridin-2-yl)boronic acid 235 (184.8 mg, 522.7 μmol), Cs₂CO₃ (309.6 mg, 950.4 μmol), and Pd(dppf)Cl₂·CH₂Cl₂ (77.6 mg, 95 μmol) in toluene (1 mL) and H₂O (0.25 mL) is degassed and purged with N₂ three times. The mixture is stirred at 80° C. for 40 min under N₂. The reaction mixture is filtered, and the filtrate is concentrated to yield the title compound tert-butyl 3-[4-chloro-6-(5-chloropyrazolo[1,5-a]pyridin-3-yl)-2-pyridyl]-3,6-diazabicyclo[3.1.1]heptane-6-carboxylate 237 (185 mg, crude) as a black solid.

Step D: To a solution of tert-butyl 3-(4-chloro-6-(5-chloropyrazolo[1,5-a]pyridin-3-yl)pyridin-2-yl)-3,6-diazabicyclo[3.1.1]heptane-6-carboxylate 237 (180 mg, 391.0 μmol) in H₂O (1 mL) is added HCl (12 M, 32 μL). The mixture is stirred at 25° C. for 1 hr. The reaction mixture is concentrated under vacuum to give the crude product which is purified by prep-HPLC (column: Xtimate C18, 150×40 mm×10 um; mobile phase: [water (HCl)-ACN]; gradient: 4%-44% B over 36 min) to yield the title compound 5-chloro-3-[4-chloro-6-(3,6-diazabicyclo[3.1.1]heptan-3-yl)-2-pyridyl]pyrazolo[1,5-a]pyridine (Compound 47) (5.1 mg, 3%) as a white solid. LCMS (ESI MS) m/z=360.1 [M+H]+ 1.

¹H NMR (400 MHz, methanol-d₄) δ=8.62-8.53 (m, 3H), 7.29 (s, 1H), 7.06-6.94 (m, 1H), 6.63 (s, 1H), 4.64 (br d, J=6.4 Hz, 2H), 4.26-4.02 (m, 4H), 3.18-3.07 (m, 1H), 2.07 (d, J=10.4 Hz, 1H).

### Example 48

### 3-[6-(2,5-diazabicyclo[2.2.2]octan-2-yl)-2-pyridyl]pyrazolo[1,5-a]pyridine

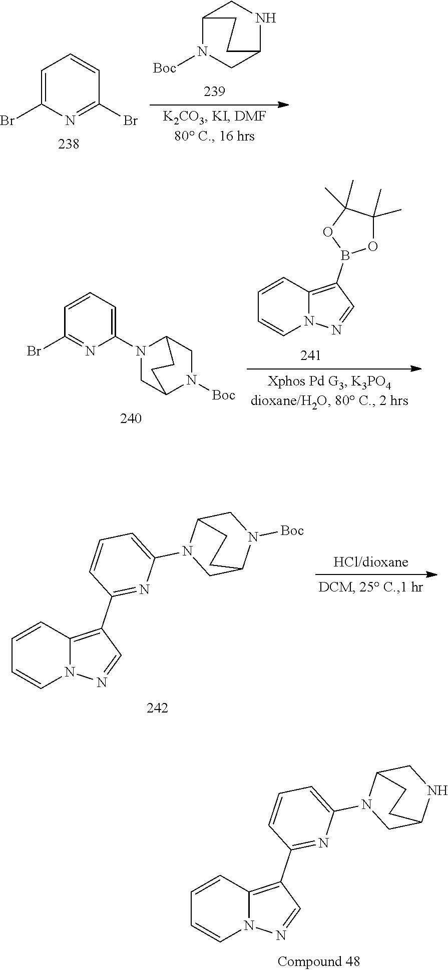 [SMILES: Brc1cccc(Br)n1 ; CC(C)(C)OC(=O)N1CC2CCC1CN2 ; CC(C)(C)OC(=O)N1CC2CCC1CN2c1cccc(Br)n1 ; CC1(C)OB(c2cnn3ccccc23)OC1(C)C ; CC(C)(C)OC(=O)N1CC2CCC1CN2c1cccc(-c2cnn3ccccc23)n1 ; c1cc(-c2cnn3ccccc23)nc(N2CC3CCC2CN3)c1]

Step A: To a solution of tert-butyl 2,5-diazabicyclo[2.2.2]octane-2-carboxylate 239 (89.6 mg, 422.1 μmol) and 2,6-dibromopyridine 238 (0.1 g, 422.1 μmol) in DMF (2 mL) are added KI (35 mg, 211.1 μmol) and K₂CO₃ (175 mg, 1.27 mmol). The mixture is stirred at 80° C. for 16 hr. The reaction mixture is partitioned between EtOAc and H₂O. The organic phase is separated, washed with brine twice, dried over Na₂SO₄, filtered and concentrated under reduced pressure to give a crude product which is purified by flash silica gel chromatography (eluent of 20-30% ethyl acetate/petroleum ether gradient) to yield the title compound tert-butyl 5-(6-bromo-2-pyridyl)-2,5-diazabicyclo[2.2.2]octane-2-carboxylate 240 (0.1 g, 28%) as a yellow oil. LCMS (ESI MS) m/z=311.9 [M−55]+1.

Step B: A mixture of 3-(4,4,5,5-tetramethyl-1,3,2-dioxaborolan-2-yl)pyrazolo[1,5-a]pyridine 241 (25.4 mg, 104.2 μmol), tert-butyl 5-(6-bromopyridin-2-yl)-2,5-diazabicyclo[2.2.2]octane-2-carboxylate 240 (43 mg, 104.2 μmol), K₃PO₄ (44.2 mg, 208.4 μmol), and XPhos Pd G3 (8.8 mg, 10.4 μmol) in dioxane (1 mL) and H₂O (0.1 mL) is purged with N₂ three times. The mixture is stirred at 80° C. for 2 hr under N₂. The reaction mixture is concentrated under reduced pressure to give a residue which is purified by flash silica gel chromatography (eluent of 20-40% ethyl acetate/petroleum ether gradient) to yield the title compound tert-butyl 5-(6-pyrazolo[1,5-a]pyridin-3-yl-2-pyridyl)-2,5-diazabicyclo[2.2.2]octane-2-carboxylate 242 (0.08 g, 80%) as a white solid. LCMS (ESI MS) m/z=406.5 [M+H]+ 1.

Step C: To a solution of tert-butyl 5-(6-(pyrazolo[1,5-a]pyridin-3-yl)pyridin-2-yl)-2,5-diazabicyclo[2.2.2]octane-2-carboxylate 242 (0.04 g, 98.6 μmol) in DCM (1 mL) is added HCl/dioxane (1 mL, 4M). The mixture is stirred at 25° C. for 1 hr. The reaction mixture is partitioned between DCM and H₂O. The organic phase is separated, and the water phase is lyophilized to yield the title compound 3-[6-(2,5-diazabicyclo[2.2.2]octan-2-yl)-2-pyridyl]pyrazolo[1,5-a]pyridine (Compound 48) (15.1 mg, 46%) as a white solid. LCMS (ESI MS) m/z=306.1 [M+H]+ 1.

¹H NMR (400 MHz, methanol-d₄) δ=8.66 (br t, J=6.4 Hz, 1H), 8.56-8.46 (m, 1H), 8.30-8.14 (m, 1H), 7.95-7.84 (m, 1H), 7.47 (br d, J=6.0 Hz, 1H), 7.31-7.18 (m, 1H), 7.07 (br d, J=5.6 Hz, 1H), 6.82 (br d, J=8.8 Hz, 1H), 4.11-3.99 (m, 2H), 3.91 (br dd, J=5.2, 12.4 Hz, 1H), 3.67-3.55 (m, 2H), 2.33-2.04 (m, 5H).

### Example 49

### 3-[6-[(3S,5R)-3,5-dimethylpiperazin-1-yl]-2-pyridyl]pyrazolo[1,5-a]pyridine

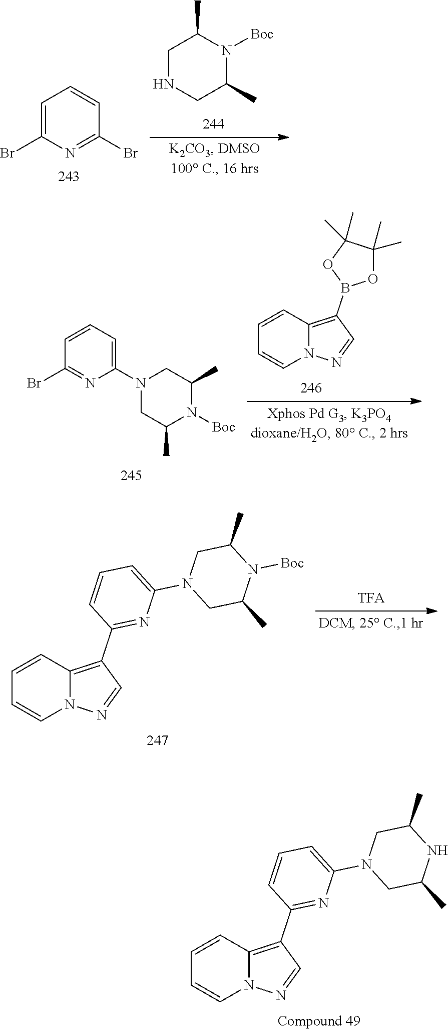

Step A: A mixture of 2,6-dibromopyridine 243 (106 mg, 447.9 μmol), tert-butyl (2S,6R)-2,6-dimethylpiperazine-1-carboxylate 244 (80 mg, 373.3 μmol), and K₂CO₃ (103 mg, 746.6 μmol) in DMSO (2 mL) is degassed and purged with N₂ three times. The mixture is stirred at 100° C. for 16 hr under a N₂ atmosphere. The reaction is quenched with water and extracted with EtOAc three times. The combined organic layers are dried over anhydrous Na₂SO₄, filtered and concentrated under reduced pressure to give a residue which is purified by flash silica gel chromatography (eluent of 0-10% ethyl acetate/petroleum ether gradient) to yield the title compound tert-butyl (2S,6R)-4-(6-bromo-2-pyridyl)-2,6-dimethyl-piperazine-1-carboxylate 245 (113 mg) as a white solid. LCMS (ESI MS) m/z=316.0 [M−55]+1.

¹H NMR (400 MHz, DMSO-d₆) δ=7.43 (dd, J=8.38, 7.50 Hz, 1H), 6.87 (d, J=8.50 Hz, 1H), 6.76 (d, J=7.38 Hz, 1H), 4.07-4.15 (m, 4H), 3.01 (dd, J=13.26, 4.75 Hz, 2H), 1.42 (s, 9H), 1.12 (d, J=6.75 Hz, 6H).

Step B: A mixture of tert-butyl (2S,6R)-4-(6-bromopyridin-2-yl)-2,6-dimethylpiperazine-1-carboxylate 245 (73 mg, 196.6 μmol), 3-(4,4,5,5-tetramethyl-1,3,2-dioxaborolan-2-yl)pyrazolo[1,5-a]pyridine 246 (40 mg, 163.9 μmol), K₃PO₄ (70 mg, 327.7 μmol), and XPhos Pd G3 (14 mg, 16.4 μmol) in dioxane (2 mL) and H₂O (0.5 mL) is purged with N₂ three times. The mixture is stirred at 80° C. for 2 hours under N₂. The reaction is quenched with water and extracted with EtOAc three times. The combined organic layers are dried over anhydrous Na₂SO₄, filtered and concentrated under reduced pressure to give a residue which is purified by flash silica gel chromatography (eluent of 0-25% ethyl acetate/petroleum ether gradient) to yield the title compound tert-butyl (2S,6R)-2,6-dimethyl-4-(6-pyrazolo[1,5-a]pyridin-3-yl-2-pyridyl)-piperazine-1-carboxylate 247 (65 mg) as a yellow oil. LCMS (ESI MS) m/z=408.3 [M+H]+ 1.

Step C: To a solution of tert-butyl (2S,6R)-2,6-dimethyl-4-(6-(pyrazolo[1,5-a]pyridin-3-yl)pyridin-2-yl)piperazine-1-carboxylate 247 (50 mg, 122.7 μmol) in DCM (1.5 mL) is added TFA (112 mg, 981.6 μmol, 75 μL). The mixture is stirred at 25° C. for 1 hr. The reaction mixture is concentrated under reduced pressure to give a residue which is purified by prep-HPLC (column: Xtimate C18, 150×40 mm×10 um; mobile phase: [water (FA)-ACN]; gradient: 0%-30% B over 25 min) to yield the title compound 3-[6-[(3S,5R)-3,5-dimethylpiperazin-1-yl]-2-pyridyl]pyrazolo[1,5-a]pyridine (Compound 49) (25.8 mg, 66%) as a white solid. LCMS (ESI MS) m/z=308.2 [M+H]+ 1.

¹H NMR (400 MHz, methanol-d₄) δ=8.52-8.57 (m, 2H), 8.41-8.45 (m, 1H), 8.32-8.38 (m, 1H), 7.58-7.64 (m, 1H), 7.31-7.39 (m, 1H), 7.14-7.20 (m, 1H), 6.92-6.99 (m, 1H), 6.74 (dd, J=8.32, 2.56 Hz, 1H), 4.56 (br d, J=13.63 Hz, 2H), 3.37-3.48 (m, 2H), 2.90 (dd, J=14.01, 11.38 Hz, 2H), 1.42 (d, J=6.50 Hz, 6H).

### Example 50

### 3-[6-[(3R,5R)-3,5-dimethylpiperazin-1-yl]-2-pyridyl]pyrazolo[1,5-a]pyridine

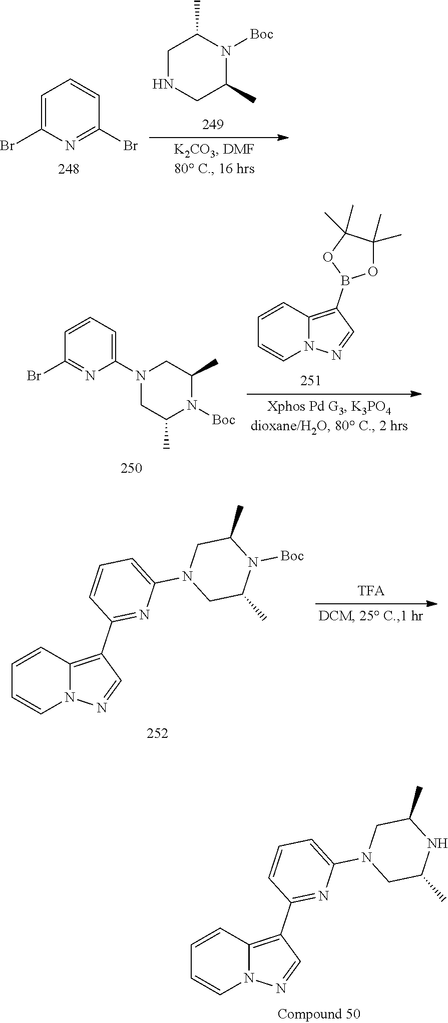

Step A: A mixture of tert-butyl (2S,6S)-2,6-dimethylpiperazine-1-carboxylate 249 (500 mg, 2.33 mmol), 2,6-dibromopyridine 248 (663.2 mg, 2.80 mmol), and K₂CO₃ (644.9 mg, 4.67 mmol) in DMF (6 mL) is degassed and purged with N₂ three times. The mixture is stirred at 80° C. for 16 hours under N₂. The reaction mixture is quenched with H₂O, and the mixture is extracted with EtOAc three times. The combined organic layers are washed with brine three times, dried over anhydrous Na₂SO₄, filtered and concentrated under reduced pressure to give a residue which is purified by flash silica gel chromatography (eluent of 0-2% ethyl acetate/petroleum ether gradient) to yield the title compound tert-butyl (2R,6R)-4-(6-bromo-2-pyridyl)-2,6-dimethyl-piperazine-1-carboxylate 250 (400 mg) as a white solid. LCMS (ESI MS) m/z=372.1 [M+H]+ 1.

Step B: A mixture of 3-(4,4,5,5-tetramethyl-1,3,2-dioxaborolan-2-yl)pyrazolo[1,5-a]pyridine 251 (145 mg, 594.1 μmol), tert-butyl (2R,6R)-4-(6-bromopyridin-2-yl)-2,6-dimethylpiperazine-1-carboxylate 250 (200 mg, 540.1 μmol), K₃PO₄ (229.3 mg, 1.08 mmol), and XPhos Pd G3 (45.7 mg, 54 μmol) in dioxane (1 mL) and H₂O (0.25 mL) is degassed and purged with N₂ three times. The mixture is stirred at 80° C. for 2 hours under N₂. The reaction mixture is concentrated under vacuum to give the crude product which is purified by flash silica gel chromatography (eluent of 0-15% ethyl acetate/petroleum ether gradient) to yield the title compound tert-butyl (2R,6R)-2,6-dimethyl-4-(6-pyrazolo[1,5-a]pyridin-3-yl-2-pyridyl)piperazine-1-carboxylate 252 (120 mg, 50%) as a yellow solid. LCMS (ESI MS) m/z=408.3 [M+H]+ 1.

¹H NMR (400 MHz, methanol-d₄) δ=8.60-8.50 (m, 2H), 8.44 (d, J=4.4 Hz, 1H), 7.61-7.51 (m, 1H), 7.42-7.31 (m, 1H), 7.09 (dd, J=4.4, 7.2 Hz, 1H), 6.96 (dt, J=2.8, 6.8 Hz, 1H), 6.44 (dd, J=4.0, 8.4 Hz, 1H), 4.32-4.20 (m, 2H), 3.98 (br d, J=12.4 Hz, 2H), 3.84-3.74 (m, 2H), 1.52 (d, J=4.4 Hz, 9H), 1.30 (dd, J=4.4, 6.4 Hz, 6H), 1.20 (d, J=4.4 Hz, 2H).

Step C: To a solution of tert-butyl (2R,6R)-2,6-dimethyl-4-(6-(pyrazolo[1,5-a]pyridin-3-yl)pyridin-2-yl)piperazine-1-carboxylate 252 (100 mg, 202 μmol) in DCM (2 mL) is added TFA (0.5 mL). The mixture is stirred at 25° C. for 1 hr. The reaction mixture is concentrated under vacuum to give the crude product which is purified by prep-HPLC (column: Xtimate C18, 150×40 mm×10 um; mobile phase: [water (FA)-ACN]; gradient: 0%-30% B over min) to yield the title compound 3-[6-[(3R,5R)-3,5-dimethylpiperazin-1-yl]-2-pyridyl]pyrazolo[1,5-a]pyridine (Compound 50) (56 mg, 77%) as a yellow solid. LCMS (ESI MS) m/z=308.0 [M+H]+ 1.

¹H NMR (400 MHz, methanol-d₄) δ=8.58 (d, J=6.8 Hz, 1H), 8.52 (s, 1H), 8.47 (s, 1H), 8.42 (d, J=8.8 Hz, 1H), 7.64 (t, J=8.0 Hz, 1H), 7.43-7.34 (m, 1H), 7.21 (d, J=7.6 Hz, 1H), 7.03-6.94 (m, 1H), 6.74 (d, J=8.4 Hz, 1H), 3.96 (dd, J=3.2, 13.6 Hz, 2H), 3.82-3.72 (m, 2H), 3.71-3.63 (m, 2H), 1.44 (d, J=6.6 Hz, 6H).

### Example 51

### 3-[4-chloro-6-[(3S,5R)-3,5-dimethylpiperazin-1-yl]-2-pyridyl]pyrazolo[1,5-a]pyridine

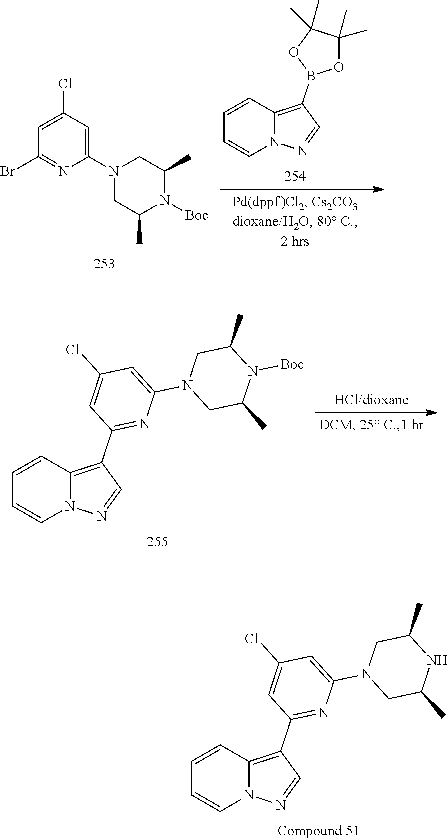

Step A: To a solution of tert-butyl (2S,6R)-4-(6-bromo-4-chloropyridin-2-yl)-2,6-dimethylpiperazine-1-carboxylate 253 (50 mg, 123.54 μmol) in dioxane (1 mL) and H₂O (1 mL) are added 3-(4,4,5,5-tetramethyl-1,3,2-dioxaborolan-2-yl)pyrazolo[1,5-a]pyridine 254 (60.3 mg, 247.1 μmol), Pd(dppf)Cl₂ (20 mg, 24.71 μmol), and Cs₂CO₃ (60 mg, 185.3 μmol). The mixture is stirred at 80° C. for 2 hr under N₂. The reaction mixture is quenched with H₂O, and the mixture is extracted with EtOAc three times. The combined organic layers are washed with brine, dried over Na₂SO₄, filtered and concentrated under reduced pressure to give a residue which is purified by flash silica gel chromatography (eluent of 15% ethyl acetate/petroleum ether gradient) to yield the title compound tert-butyl (2S,6R)-4-(4-chloro-6-pyrazolo[1,5-a]pyridin-3-yl-2-pyridyl)-2,6-dimethyl-piperazine-1-carboxylate crude 255 (30 mg) as a white solid. LCMS (ESI MS) m/z=444.0 [M+H]+ 1.

Step B: To a solution of tert-butyl (2S,6R)-4-(4-chloro-6-(pyrazolo[1,5-a]pyridin-3-yl)pyridin-2-yl)-2,6-dimethylpiperazine-1-carboxylate 255 (30 mg, 67.88 μmol) in DCM (1 mL) is added HCl/dioxane (4 M, 67.9 μL). The mixture is stirred at 25° C. for 1 hr. The reaction mixture is concentrated under reduced pressure to give a residue which is purified by prep-HPLC (HCl condition; column: Xtimate C18, 150×40 mm×10 um; mobile phase: [water (HCl)-ACN]; gradient: 2%-42% B over 36 min) to yield the title compound 3-[4-chloro-6-[(3S,5R)-3,5-dimethylpiperazin-1-yl]-2-pyridyl]pyrazolo[1,5-a]pyridine (Compound 51) (8.3 mg, 34%) as a white solid. LCMS (ESI MS) m/z=342.1 [M+H]+ 1.

¹H NMR (400 MHz, methanol-d₄) δ=8.63 (d, J=7.2 Hz, 1H), 8.54 (s, 1H), 8.40 (d, J=9.2 Hz, 1H), 7.42-7.51 (m, 1H), 7.29 (d, J=1.2 Hz, 1H), 7.04 (td, J=6.8, 1.2 Hz, 1H), 6.88 (d, J=1.2 Hz, 1H), 4.68 (dd, J=14.0, 2.8 Hz, 2H), 3.46-3.57 (m, 2H), 2.96 (dd, J=14.4, 11.6 Hz, 2H), 1.46 (d, J=6.8 Hz, 6H).

### Example 52

### 3-[6-[(3R,5S)-3,5-dimethylpiperazin-1-yl]-4-fluoro-2-pyridyl]pyrazolo[1,5-a]pyridine

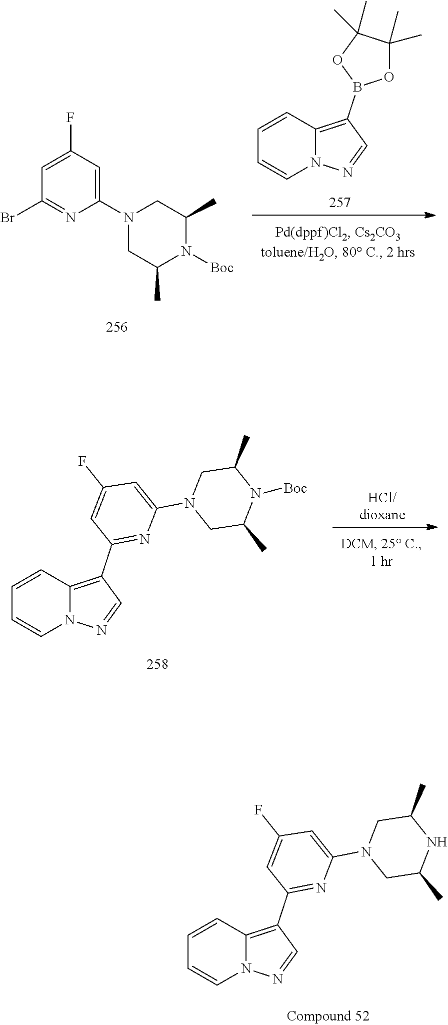 [SMILES: C[C@@H]1CN(c2cc(F)cc(Br)n2)C[C@H](C)N1C(=O)OC(C)(C)C ; CC1(C)OB(c2cnn3ccccc23)OC1(C)C ; C[C@@H]1CN(c2cc(F)cc(-c3cnn4ccccc34)n2)C[C@H](C)N1C(=O)OC(C)(C)C ; C[C@@H]1CN(c2cc(F)cc(-c3cnn4ccccc34)n2)C[C@H](C)N1]

Step A: A mixture of tert-butyl (2S,6R)-4-(6-bromo-4-fluoropyridin-2-yl)-2,6-dimethylpiperazine-1-carboxylate 256 (70 mg, 180.28 μmol), 3-(4,4,5,5-tetramethyl-1,3,2-dioxaborolan-2-yl)pyrazolo[1,5-a]pyridine 257 (88 mg, 360.57 μmol), Pd(dppf)Cl₂ (29 mg, 36.06 μmol), and Cs₂CO₃ (117 mg, 360.57 μmol) in toluene (2 mL) and H₂O (0.5 mL) is purged with N₂ three times. The mixture is stirred at 80° C. for 2 hr under N₂. The reaction mixture is quenched with H₂O, and the mixture is extracted with EtOAc three times. The combined organic layers are dried over anhydrous Na₂SO₄, filtered and concentrated under reduced pressure to give a residue which is purified by flash silica gel chromatography (eluent of 0-32% ethyl acetate/petroleum ether gradient) to yield the title compound tert-butyl (2S,6R)-4-(4-fluoro-6-pyrazolo[1,5-a]pyridin-3-yl-2-pyridyl)-2,6-dimethyl-piperazine-1-carboxylate 258 (60 mg) as a yellow oil. LCMS (ESI MS) m/z=426.5 [M+H]+ 1.

Step B: To a solution of tert-butyl (2S,6R)-4-(4-fluoro-6-(pyrazolo[1,5-a]pyridin-3-yl)pyridin-2-yl)-2,6-dimethylpiperazine-1-carboxylate 258 (40 mg, 94.01 μmol) in DCM (1 mL) is added HCl/dioxane (4 M, 0.5 mL). The mixture is stirred at 25° C. for 1 hr. The reaction mixture is concentrated under vacuum to give the crude product which is purified by prep-HPLC (column: Xtimate C18, 150×40 mm×10 um; mobile phase: [water (FA)-ACN]; gradient: 0%-34% B over 25 min) to yield the title compound 3-[6-[(3R,5S)-3,5-dimethylpiperazin-1-yl]-4-fluoro-2-pyridyl]pyrazolo[1,5-a]pyridine (Compound 52) (9 mg, 25%) as a white solid. LCMS (ESI MS) m/z=326.2 [M+H]+ 1.

¹H NMR (400 MHz, methanol-d₄) δ=8.58 (d, J=7.00 Hz, 1H), 8.51 (s, 1H), 8.48 (s, 1H), 8.37 (d, J=8.88 Hz, 1H), 7.38-7.44 (m, 1H), 6.97-7.03 (m, 2H), 6.54 (br d, J=12.01 Hz, 1H), 4.59 (br dd, J=13.82, 1.81 Hz, 2H), 3.41 (ddd, J=10.19, 6.50, 3.44 Hz, 2H), 2.86-2.97 (m, 2H), 1.41 (d, J=6.50 Hz, 6H).

### Example 53

### 3-[6-[(3S,5R)-3,5-dimethylpiperazin-1-yl]-2-pyridyl]-N-methyl-pyrazolo[1,5-a]pyridin-5-amine

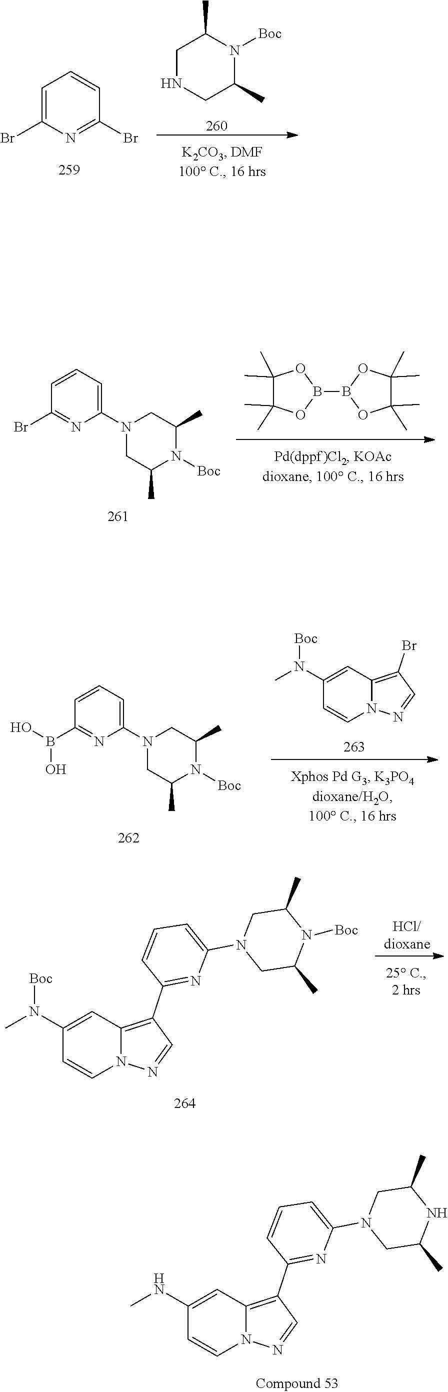

Step A: To a solution of 2,6-dibromopyridine 259 (2.21 g, 9.33 mmol) and tert-butyl (2S,6R)-2,6-dimethylpiperazine-1-carboxylate 260 (1 g, 4.67 mmol) in DMF (30 mL) is added K₂CO₃ (1.29 g, 9.33 mmol). The mixture is stirred at 100° C. for 16 hr. The reaction mixture is diluted with H₂O, and the mixture is extracted with EtOAc three times. The combined organic layers are washed with brine, dried over anhydrous Na₂SO₄, filtered and concentrated under reduced pressure to give a residue which is purified by flash silica gel chromatography (eluent of 0-10% ethyl acetate/petroleum ether gradient) to yield the title compound tert-butyl (2S,6R)-4-(6-bromo-2-pyridyl)-2,6-dimethyl-piperazine-1-carboxylate 261 (630 mg) as a white solid. LCMS (ESI MS) m/z=370.1 [M+H]+ 1.

Step B: A mixture of tert-butyl (2S,6R)-4-(6-bromo-2-pyridyl)-2,6-dimethyl-piperazine-1-carboxylate 261 (580 mg, 1.57 mmol), 4,4,5,5-tetramethyl-2-(4,4,5,5-tetramethyl-1,3,2-dioxaborolan-2-yl)-1,3,2-dioxaborolane (715.9 mg, 2.82 mmol), Pd(dppf)Cl₂ (127.9 mg, 156.64 μmol), and KOAc (230.5 mg, 2.35 mmol) in dioxane (10 mL) is purged with N₂ three times. The mixture is then stirred at 100° C. for 16 hr under N₂. The reaction mixture is concentrated under vacuum to give the title compound [6-[(3S,5R)-4-tert-butoxycarbonyl-3,5-dimethyl-piperazin-1-yl]-2-pyridyl]boronic acid 6 (400 mg, crude) as a white solid. LCMS (ESI MS) m/z=336.2 [M+H]+ 1.

¹H NMR (400 MHz, methanol-d₄) δ=7.90 (dd, J=8.8, 7.1 Hz, 1H), 7.13 (d, J=8.8 Hz, 1H), 7.03 (d, J=6.8 Hz, 1H), 4.32-4.41 (m, 2H), 4.03 (br d, J=14.2 Hz, 2H), 3.49 (dd, J=13.6, 4.6 Hz, 2H), 1.50 (s, 9H), 1.31 (s, 3H), 1.29 (s, 3H).

Step C: A mixture of [6-[(3S,5R)-4-tert-butoxycarbonyl-3,5-dimethyl-piperazin-1-yl]-2-pyridyl]boronic acid 262 (92.4 mg, 275.9 μmol), tert-butyl (3-bromopyrazolo[1,5-a]pyridin-5-yl)(methyl)carbamate 263 (60 mg, 183.9 μmol), XPhos Pd G3 (31.1 mg, 36.79 μmol), and K₃PO₄ (78.0 mg, 367.8 μmol) in dioxane (1 mL) and H₂O (0.25 mL) is purged with N₂ three times, and then the mixture is stirred at 100° C. for 16 hr under N₂. The reaction mixture is diluted with H₂O, and the mixture is extracted with EtOAc three times. The combined organic layers are washed with brine, dried over anhydrous Na₂SO₄, filtered and concentrated under reduced pressure to give a residue. The residue is purified by flash silica gel chromatography (eluent of 25% ethyl acetate/petroleum ether gradient) to yield the title compound tert-butyl (2S,6R)-4-[6-[5-[tert-butoxycarbonyl(methyl)amino]pyrazolo [1,5-a]pyridin-3-yl]-2-pyridyl]-2,6-dimethyl-piperazine-1-carboxylate 24 (60 mg) as a white solid. LCMS (EB9118-326-P1B1). LCMS (ESI MS) m/z=537.3 [M+H]+ 1.

Step D: A solution of tert-butyl (2S,6R)-4-[6-[5-[tert-butoxycarbonyl(methyl)amino]-pyrazolo [1,5-a]pyridin-3-yl]-2-pyridyl]-2,6-dimethyl-piperazine-1-carboxylate 24 (50 mg, 93.17 μmol) in HCl/dioxane (4 M, 2 mL) is stirred at 25° C. for 2 hr. The reaction mixture is concentrated under vacuum to give a residue which is purified by prep-HPLC (column: Xtimate C18, 150×40 mm×10 um; mobile phase: [water (FA)-ACN]; gradient: 0%-30% B over 25 min) to yield the title compound 3-[6-[(3S,5R)-3,5-dimethylpiperazin-1-yl]-2-pyridyl]-N-methyl-pyrazolo[1,5-a]pyridin-5-amine (Compound 53) (5.0 mg, 15%) as a brown solid. LCMS (ESI MS) m/z=337.2 [M+H]+ 1.

¹H NMR (400 MHz, methanol-d₄) δ=8.51 (s, 1H), 8.19-8.24 (m, 1H), 8.11-8.16 (m, 1H), 7.52-7.60 (m, 1H), 7.16 (br d, J=2.4 Hz, 1H), 7.08-7.12 (m, 1H), 6.64 (br d, J=8.4 Hz, 1H), 6.42 (dd, J=7.6, 2.6 Hz, 1H), 4.55 (br dd, J=13.6, 2.7 Hz, 2H), 3.43 (ddd, J=10.8, 6.6, 3.3 Hz, 2H), 2.86-2.89 (m, 3H), 2.84 (br d, J=2.4 Hz, 1H), 2.81 (s, 1H), 1.40 (d, J=6.4 Hz, 6H).

### Example 54

### 3-[4-chloro-6-[(3S,5R)-3,5-dimethylpiperazin-1-yl]-2-pyridyl]-N-methyl-pyrazolo[1,5-a]pyridin-5-amine

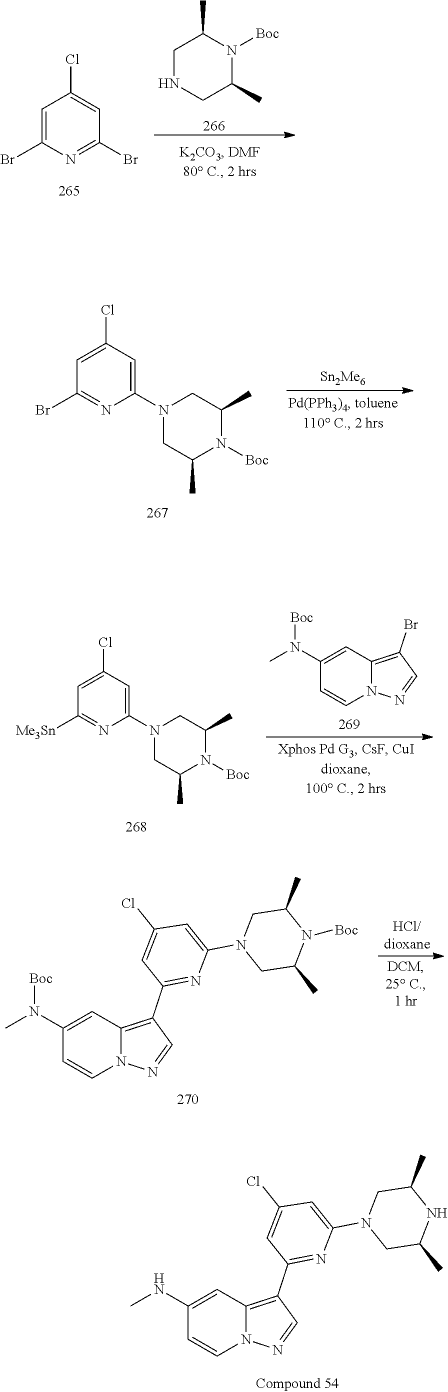

Step A: A mixture of 2,6-dibromo-4-chloropyridine 265 (500 mg, 1.84 mmol), tert-butyl (2S,6R)-2,6-dimethylpiperazine-1-carboxylate 266 (592 mg, 2.76 mmol), and K₂CO₃ (509 mg, 3.69 mmol) in DMF (10 mL) is stirred at 80° C. for 2 hours. The reaction mixture is quenched with water and extracted with EtOAc three times. The combined organic layers are dried over anhydrous Na₂SO₄, filtered and concentrated under reduced pressure to give a residue which is purified by flash silica gel chromatography (eluent of 0-5% ethyl acetate/petroleum ether gradient) to give the title compound tert-butyl (2S,6R)-4-(6-bromo-4-chloro-2-pyridyl)-2,6-dimethyl-piperazine-1-carboxylate 267 (300 mg) as a white solid. LCMS (ESI MS) m/z=405.9 [M+H]+ 1.

Step B: A mixture of tert-butyl (2S,6R)-4-(6-bromo-4-chloropyridin-2-yl)-2,6-dimethylpiperazine-1-carboxylate 267 (200 mg, 494.16 μmol), Sn₂Me₆ (1.13 g, 3.45 mmol, 715.19 μL), and Pd(PPh₃)₄ (114 mg, 98.8 μmol) in toluene (4 mL) is purged with N₂ three times. The mixture is stirred at 110° C. for 2 hr under N₂. The reaction is quenched with sat. KF and extracted with EtOAc three times. The combined organic layers are dried over anhydrous Na₂SO₄, filtered and concentrated under reduced pressure to give the title compound tert-butyl (2S,6R)-4-(4-chloro-6-(trimethylstannyl)pyridin-2-yl)-2,6-dimethylpiperazine-1-carboxylate 268 (160 mg) as a black solid. LCMS (ESI MS) m/z=489.1 [M+H]+ 1.

Step C: To a solution of tert-butyl (2S,6R)-4-(4-chloro-6-(trimethylstannyl)pyridin-2-yl)-2,6-dimethylpiperazine-1-carboxylate 268 (157 mg, 321.9 μmol) and tert-butyl (3-bromopyrazolo[1,5-a]pyridin-5-yl)(methyl)carbamate 269 (70 mg, 214.6 μmol) in dioxane (3 mL) are added XPhos Pd G3 (36 mg, 42.9 μmol), CuI (82 mg, 429.2 μmol), and CsF (49 mg, 321.9 μmol). The mixture is stirred at 100° C. for 2 hr under N₂. The reaction mixture is filtered and concentrated under reduced pressure to give a residue which is purified by flash silica gel chromatography (eluent of 0-10% ethyl acetate/petroleum ether gradient) to give the title compound tert-butyl (2S,6R)-4-[6-[5-[tert-butoxycarbonyl(methyl)amino]pyrazolo[1,5-a]pyridin-3-yl]-4-chloro-2-pyridyl]-2,6-dimethyl-piperazine-1-carboxylate 270 (30 mg) as a white solid. LCMS (ESI MS) m/z=571.3 [M+H]+ 1.

Step D: To a solution of tert-butyl (2S,6R)-4-(6-(5-((tert-butoxycarbonyl)(methyl)amino)pyrazolo[1,5-a]pyridin-3-yl)-4-chloropyridin-2-yl)-2,6-dimethyl-piperazine-1-carboxylate 270 (30 mg, 52.5 μmol) in DCM (1 mL) is added HCl/dioxane (4 M, 169 μL). The mixture is stirred at 25° C. for 1 hr. The reaction mixture is concentrated under vacuum to give the crude product which is purified by prep-HPLC (HCl condition; column: Xtimate C18, 150×40 mm×10 um; mobile phase: [water (HCl)-ACN]; gradient: 2%-42% B over 36 min) to yield the title compound 3-[4-chloro-6-[(3S,5R)-3,5-dimethylpiperazin-1-yl]-2-pyridyl]-N-methyl-pyrazolo[1,5-a]pyridin-5-amine (Compound 54) (5.0 mg, 24%) as an off-white solid. LCMS (ESI MS) m/z=371.1 [M+H]+ 1.

¹H NMR (400 MHz, methanol-d₄) δ=8.30-8.38 (m, 1H), 8.33 (s, 1H), 8.18-8.24 (m, 1H), 8.22 (d, J=7.6 Hz, 1H), 7.19 (s, 2H), 6.78 (s, 1H), 6.52 (d, J=7.2 Hz, 1H), 4.67 (br dd, J=13.6, 2.0 Hz, 2H), 3.48-3.54 (m, 2H), 2.90-2.97 (m, 5H), 1.45 (d, J=6.4 Hz, 6H).

### Example 55

### 3-[6-[(3S,5R)-3,5-dimethylpiperazin-1-yl]-2-pyridyl]-5-(trifluoromethyl)pyrazolo[1,5-a]pyridine

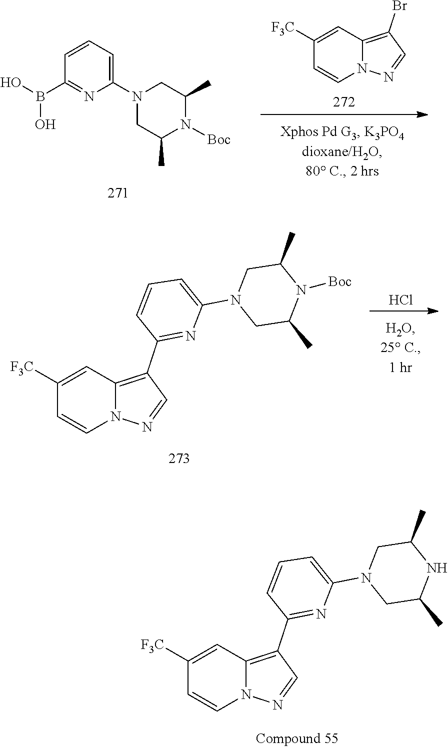 [SMILES: C[C@@H]1CN(c2cccc(B(O)O)n2)C[C@H](C)N1C(=O)OC(C)(C)C ; FC(F)(F)c1ccn2ncc(Br)c2c1 ; C[C@@H]1CN(c2cccc(-c3cnn4ccc(C(F)(F)F)cc34)n2)C[C@H](C)N1C(=O)OC(C)(C)C ; Cl ; C[C@@H]1CN(c2cccc(-c3cnn4ccc(C(F)(F)F)cc34)n2)C[C@H](C)N1]

Step A: A mixture of 3-bromo-5-(trifluoromethyl)pyrazolo[1,5-a]pyridine 272 (200 mg, 754.6 μmol), (6-((3S,5R)-4-(tert-butoxycarbonyl)-3,5-dimethylpiperazin-1-yl)pyridin-2-yl)boronic acid 271 (303.5 mg, 905.5 μmol), XPhos Pd G3 (63.8 mg, 75.4 μmol, 0.1 eq), and K₃PO₄ (320.3 mg, 1.51 mmol) in dioxane (5 mL) and H₂O (1.25 mL) is purged with N₂ three times. The mixture is stirred at 80° C. for 2 hr under N₂. The reaction mixture is concentrated under reduced pressure to give a crude product which is purified by flash silica gel chromatography (eluent of 0-11% ethyl acetate/petroleum ether gradient) to yield the title compound tert-butyl (2S,6R)-2,6-dimethyl-4-[6-[5-(trifluoromethyl)pyrazolo[1,5-a]pyridin-3-yl]-2-pyridyl]piperazine-1-carboxylate 273 (110 mg, crude) as an off-white solid.

Step B: To a solution of tert-butyl (2S,6R)-2,6-dimethyl-4-(6-(5-(trifluoromethyl)-pyrazolo [1,5-a]pyridin-3-yl)pyridin-2-yl)piperazine-1-carboxylate 273 (70 mg, 147.21 μmol) in H₂O (1 mL) is added aqueous HCl (12 M, aq. 184 μL). The mixture is stirred at 25° C. for 1 hr. The reaction mixture is concentrated directly to give a crude product which is purified by prep-HPLC (column: Xtimate C18, 150×40 mm×10 um; mobile phase: [water (HCl)-ACN]; gradient: 4%-44% B over 36 min) to yield the title compound 3-[6-[(3S,5R)-3,5-dimethylpiperazin-1-yl]-2-pyridyl]-5-(trifluoromethyl)pyrazolo[1,5-a]pyridine (Compound 55) (12.5 mg, 22% yield) as a white solid. LCMS (ESI MS) m/z=376.2 [M+H]+ 1.

¹H NMR (400 MHz, methanol-d₄) δ=8.93 (s, 1H), 8.76 (d, J=7.2 Hz, 1H), 8.62 (s, 1H), 7.69 (t, J=8.0 Hz, 1H), 7.31 (d, J=7.6 Hz, 1H), 7.15 (dd, J=1.6, 7.2 Hz, 1H), 6.87 (d, J=8.4 Hz, 1H), 4.61 (dd, J=2.4, 13.6 Hz, 2H), 3.52 (ddd, J=3.2, 6.8, 10.4 Hz, 2H), 2.92 (dd, J=11.6, 13.6 Hz, 2H), 1.46 (d, J=6.4 Hz, 6H).

### Example 56

### 3-[4-chloro-6-[(3S,5R)-3,5-dimethylpiperazin-1-yl]-2-pyridyl]-5-(trifluoromethyl)pyrazolo[1,5-a]pyridine

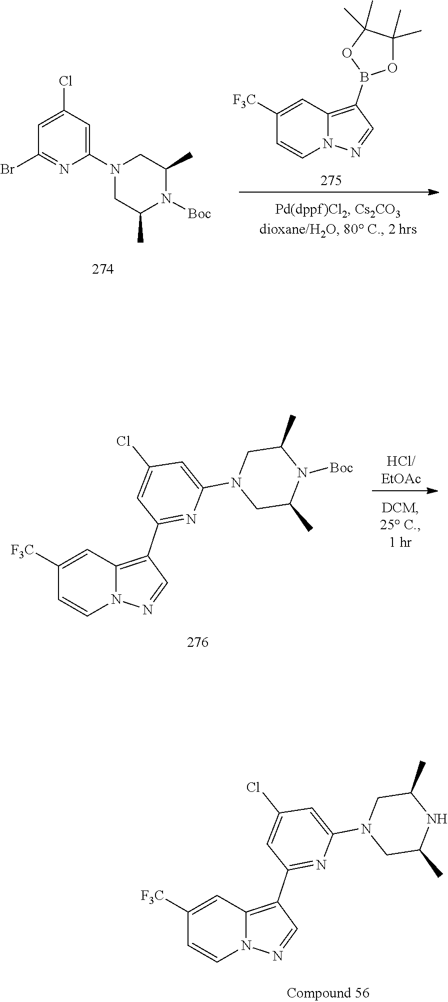 [SMILES: C[C@@H]1CN(c2cc(Cl)cc(Br)n2)C[C@H](C)N1C(=O)OC(C)(C)C ; CC1(C)OB(c2cnn3ccc(C(F)(F)F)cc23)OC1(C)C ; C[C@@H]1CN(c2cc(Cl)cc(-c3cnn4ccc(C(F)(F)F)cc34)n2)C[C@H](C)N1C(=O)OC(C)(C)C ; C[C@@H]1CN(c2cc(Cl)cc(-c3cnn4ccc(C(F)(F)F)cc34)n2)C[C@H](C)N1]

Step A: A mixture of tert-butyl (2S,6R)-4-(6-bromo-4-chloropyridin-2-yl)-2,6-dimethylpiperazine-1-carboxylate 274 (50 mg, 123.5 μmol), 3-(4,4,5,5-tetramethyl-1,3,2-dioxaborolan-2-yl)-5-(trifluoromethyl)pyrazolo[1,5-a]pyridine 275 (77 mg, 247.08 μmol), Pd(dppf)Cl₂ (20 mg, 24.71 μmol), and Cs₂CO₃ (60 mg, 185.31 μmol) in dioxane (2 mL) and H₂O (0.3 mL) is purged with N₂ three times, and the mixture is stirred at 80° C. for 2 hr under N₂. The reaction mixture is quenched with H₂O, and the mixture is extracted with EtOAc three times. The combined organic layers are dried over anhydrous Na₂SO₄, filtered and concentrated under reduced pressure to give a residue which is purified by flash silica gel chromatography (eluent of 0-8% ethyl acetate/petroleum ether gradient) to yield the title compound tert-butyl (2S,6R)-4-[4-chloro-6-[5-(trifluoromethyl)pyrazolo [1,5-a]pyridin-3-yl]-2-pyridyl]-2,6-dimethyl-piperazine-1-carboxylate 276 (33 mg) as a yellow solid. LCMS (ESI MS) m/z=510.2 [M+1]+1.

Step B: To a solution of tert-butyl (2S,6R)-4-[4-chloro-6-[5-(trifluoromethyl)pyrazolo [1,5-a]pyridin-3-yl]-2-pyridyl]-2,6-dimethyl-piperazine-1-carboxylate 276 in DCM (1 mL) is added HCl/dioxane (4 M, 98 μL). The mixture is stirred at 25° C. for 1 hr. The reaction mixture is concentrated under vacuum to give the crude product which is purified by prep-HPLC (column: Xtimate C18, 150×40 mm×10 um; mobile phase: [water (FA)-ACN]; gradient: 2%-42% B over 25 min) to yield the title compound 3-[4-chloro-6-[(3S,5R)-3,5-dimethylpiperazin-1-yl]-2-pyridyl]-5-(trifluoromethyl)pyrazolo[1,5-a]pyridine (Compound 56) (4.5 mg, 21%) as a white solid. LCMS (ESI MS) m/z=410.0 [M+H]+ 1.

¹H NMR (400 MHz, methanol-d₄) δ=8.87 (s, 1H), 8.76 (d, J=7.38 Hz, 1H), 8.62 (s, 1H), 7.28 (s, 1H), 7.16 (dd, J=7.25, 1.75 Hz, 1H), 6.84 (s, 1H), 4.51 (br d, J=2.25 Hz, 1H), 4.48 (br d, J=2.38 Hz, 1H), 3.33-3.39 (m, 2H), 2.78-2.85 (m, 2H), 1.38 (d, J=6.38 Hz, 6H).

### Example 57

### 5-chloro-3-[6-[(3R,5S)-3,5-dimethylpiperazin-1-yl]-2-pyridyl]pyrazolo[1,5-a]pyridine

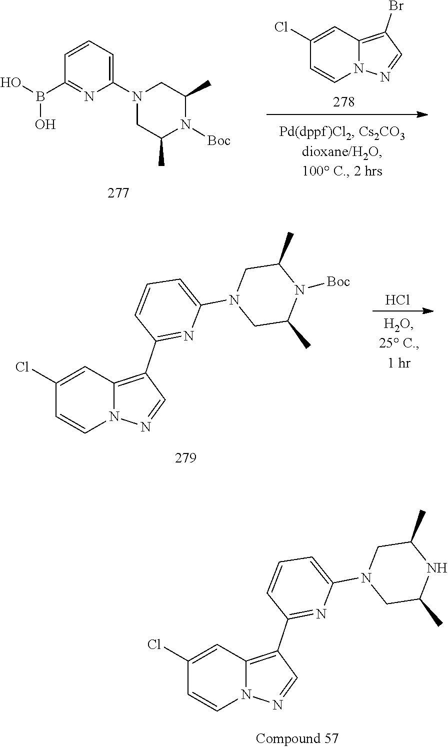 [SMILES: C[C@@H]1CN(c2cccc(B(O)O)n2)C[C@H](C)N1C(=O)OC(C)(C)C ; Clc1ccn2ncc(Br)c2c1 ; C[C@@H]1CN(c2cccc(-c3cnn4ccc(Cl)cc34)n2)C[C@H](C)N1C(=O)OC(C)(C)C ; Cl ; C[C@@H]1CN(c2cccc(-c3cnn4ccc(Cl)cc34)n2)C[C@H](C)N1]

Step A: A mixture of 3-bromo-5-chloropyrazolo[1,5-a]pyridine 278 (165.7 mg, 716 μmol), (6-((3S,5R)-4-(tert-butoxycarbonyl)-3,5-dimethylpiperazin-1-yl)pyridin-2-yl)boronic acid 277 (200 mg, 596.6 μmol), Pd(dppf)Cl₂ (44 mg, 59.7 μmol), and Cs₂CO₃ (389 mg, 1.19 mmol) in dioxane (5 mL) and H₂O (1.25 mL) is purged with N₂ three times. The mixture is stirred at 100° C. for 2 hr under N₂. The reaction mixture is concentrated under reduced pressure to give a crude product which is purified by flash silica gel chromatography (eluent of 0-11% ethyl acetate/petroleum ether gradient) to yield the title compound tert-butyl (2S,6R)-4-[6-(5-chloropyrazolo[1,5-a]pyridin-3-yl)-2-pyridyl]-2,6-dimethyl-piperazine-1-carboxylate 279 (13.4 mg, crude) as a yellow solid.

Step B: To a solution of tert-butyl (2S,6R)-4-(6-(5-chloropyrazolo[1,5-a]pyridin-3-yl)pyridin-2-yl)-2,6-dimethylpiperazine-1-carboxylate 279 (13 mg, 29.41 μmol) in H₂O (1 mL) is added aqueous HCl (12 M, 73 μL). The mixture is stirred at 25° C. for 1 hr. The reaction mixture is concentrated under reduced pressure to give the crude product which is purified by prep-HPLC (column: Xtimate C18, 150×40 mm×10 um; mobile phase: [water (HCl)-ACN]; gradient: 0%-40% B over 36 min) to yield the title compound 5-chloro-3-[6-[(3R,5S)-3,5-dimethylpiperazin-1-yl]-2-pyridyl]pyrazolo[1,5-a]pyridine (Compound 57) (9.7 mg, 94% yield) as a white solid. LCMS (ESI MS) m/z=342.2 [M+H]+ 1.

¹H NMR (400 MHz, methanol-d₄) δ=8.67-8.41 (m, 3H), 7.75-7.60 (m, 1H), 7.32-7.17 (m, 1H), 7.08-6.93 (m, 1H), 6.89-6.74 (m, 1H), 4.62 (br d, J=13.2 Hz, 2H), 3.60-3.42 (m, 2H), 3.04-2.86 (m, 2H), 1.53-1.41 (m, 6H).

### Example 58

### 5-chloro-3-[6-[(3R,5S)-3,5-dimethylpiperazin-1-yl]-4-fluoro-2-pyridyl]pyrazolo[1,5-a]pyridine

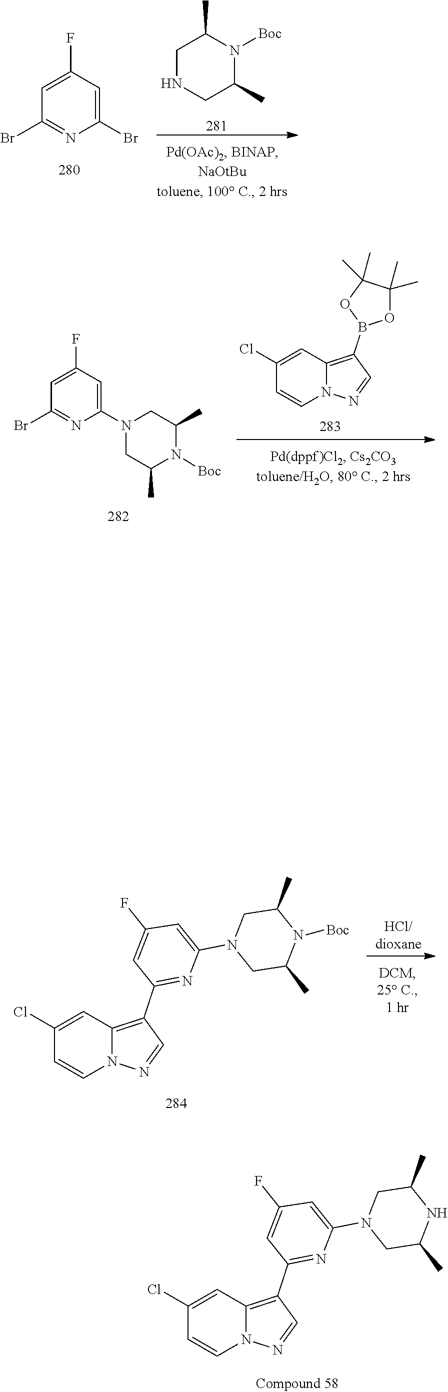 [SMILES: Fc1cc(Br)nc(Br)c1 ; C[C@@H]1CNC[C@H](C)N1C(=O)OC(C)(C)C ; C[C@@H]1CN(c2cc(F)cc(Br)n2)C[C@H](C)N1C(=O)OC(C)(C)C ; CC1(C)OB(c2cnn3ccc(Cl)cc23)OC1(C)C ; C[C@@H]1CN(c2cc(F)cc(-c3cnn4ccc(Cl)cc34)n2)C[C@H](C)N1C(=O)OC(C)(C)C ; C[C@@H]1CN(c2cc(F)cc(-c3cnn4ccc(Cl)cc34)n2)C[C@H](C)N1]

Step A: A mixture of tert-butyl (2S,6R)-2,6-dimethylpiperazine-1-carboxylate 281 (100 mg, 466.6 μmol), 2,6-dibromo-4-fluoropyridine 280 (238 mg, 933.3 μmol), Pd(OAc)₂ (21 mg, 93.33 μmol), BINAP (58 mg, 93.3 μmol), and t-BuONa (89 mg, 933.26 μmol) in toluene (2 mL) is purged with N₂ three times. The mixture is stirred at 100° C. for 2 hr under N₂. The reaction mixture is quenched with H₂O, and the mixture is extracted with EtOAc three times. The combined organic layers are dried over anhydrous Na₂SO₄, filtered and concentrated under reduced pressure to give a residue which is purified by flash silica gel chromatography (eluent of 0-8% ethyl acetate/petroleum ether gradient) to yield the title compound tert-butyl (2S,6R)-4-(6-bromo-4-fluoro-2-pyridyl)-2,6-dimethyl-piperazine-1-carboxylate 282 (480 mg) as a yellow solid. LCMS (ESI MS) m/z=331.9 [M−55]+1.

Step B: A mixture of tert-butyl (2S,6R)-4-(6-bromo-4-fluoropyridin-2-yl)-2,6-dimethylpiperazine-1-carboxylate 282 (33 mg, 84.99 μmol), 5-chloro-3-(4,4,5,5-tetramethyl-1,3,2-dioxaborolan-2-yl)pyrazolo[1,5-a]pyridine 283 (36 mg, 127.5 μmol), Pd(dppf)Cl₂ (14 mg, 17.00 μmol), and Cs₂CO₃ (55 mg, 169.98 μmol) in toluene (2 mL) and H₂O (0.5 mL) is purged with N₂ three times. The mixture is stirred at 80° C. for 2 hr under N₂. The reaction is quenched with water and extracted with EtOAc three times. The combined organic layers are dried over anhydrous Na₂SO₄, filtered and concentrated under reduced pressure to give a residue which is purified by flash silica gel chromatography (eluent of 0-18% ethyl acetate/petroleum ether gradient) to yield the title compound tert-butyl (2S,6R)-4-[6-(5-chloropyrazolo[1,5-a]pyridin-3-yl)-4-fluoro-2-pyridyl]-2,6-dimethyl-piperazine-1-carboxylate 284 (30 mg) as a white solid. LCMS (ESI MS) m/z=462.0 [M+H]+ 1.

Step C: To a solution of tert-butyl (2S,6R)-4-(6-(5-chloropyrazolo[1,5-a]pyridin-3-yl)-4-fluoropyridin-2-yl)-2,6-dimethylpiperazine-1-carboxylate 284 (25 mg, 54.35 μmol) in DCM (1 mL) is added HCl/dioxane (4 M, 108 μL). The mixture is stirred at 25° C. for 1 hr. The reaction is concentrated under vacuum to give the crude product which is purified by prep-HPLC (column: Xtimate C18, 150×40 mm×10 um; mobile phase: [water (FA)-ACN]; gradient: 0%-36% B over 25 min) to yield the title compound 5-chloro-3-[6-[(3R,5S)-3,5-dimethylpiperazin-1-yl]-4-fluoro-2-pyridyl]pyrazolo[1,5-a]pyridine (Compound 58) (12.5 mg, 56%) as a white solid. LCMS (ESI MS) m/z=360.2 [M+H]+ 1.

¹H NMR (400 MHz, methanol-d₄) δ=8.57 (br d, J=7.38 Hz, 1H), 8.51 (br s, 2H), 8.46 (br s, 1H), 6.99 (br s, 2H), 6.55 (br d, J=11.88 Hz, 1H), 4.54 (br d, J=13.88 Hz, 2H), 3.36-3.44 (m, 2H), 2.90 (br t, J=12.51 Hz, 2H), 1.42 (br d, J=6.13 Hz, 6H).

### Example 59

### 5-chloro-3-[4-chloro-6-[(3S,5R)-3,5-dimethylpiperazin-1-yl]-2-pyridyl]pyrazolo[1,5-a]pyridine

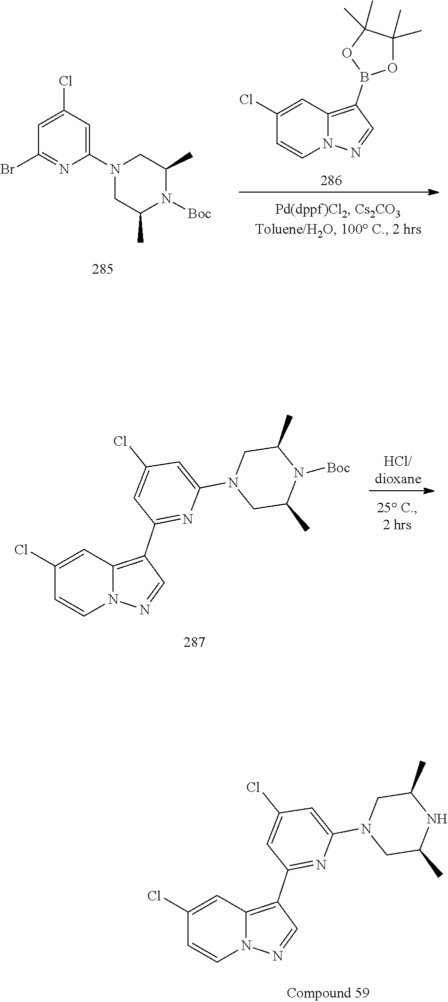 [SMILES: C[C@@H]1CN(c2cc(Cl)cc(Br)n2)C[C@H](C)N1C(=O)OC(C)(C)C ; CC1(C)OB(c2cnn3ccc(Cl)cc23)OC1(C)C ; C[C@@H]1CN(c2cc(Cl)cc(-c3cnn4ccc(Cl)cc34)n2)C[C@H](C)N1C(=O)OC(C)(C)C ; C[C@@H]1CN(c2cc(Cl)cc(-c3cnn4ccc(Cl)cc34)n2)C[C@H](C)N1]

Step A: A mixture of tert-butyl (2S,6R)-4-(6-bromo-4-chloropyridin-2-yl)-2,6-dimethylpiperazine-1-carboxylate 285 (50 mg, 123.54 μmol), 3-(4,4,5,5-tetramethyl-1,3,2-dioxaborolan-2-yl)-5-chloropyrazolo[1,5-a]pyridine 286 (34.4 mg, 123.5 μmol), Pd(dppf)Cl₂ (20.1 mg, 24.71 μmol), and Cs₂CO₃ (80.5 mg, 247.08 μmol) in toluene (1 mL) and H₂O (0.25 mL) is purged with N₂ three times, and then the mixture is stirred at 100° C. for 2 hr under N₂. The reaction mixture is concentrated under reduced pressure to give the title compound 28Z (58.8 mg, crude) as a brown solid. LCMS (ESI MS) m/z=476.1 [M+H]+ 1.

Step B: A solution of tert-butyl (2S,6R)-4-[4-chloro-6-(5-chloropyrazolo[1,5-a]pyridin-3-yl)-2-pyridyl]-2,6-dimethyl-piperazine-1-carboxylate 287 (50 mg, 104.9 μmol) in HCl/dioxane (4 M, 2 mL) is stirred at 25° C. for 2 hr. The reaction mixture is concentrated, and the residue is purified by prep-HPLC (column: Xtimate C18, 150×40 mm×10 um; mobile phase: [water (NH₃H₂O+NH₄HCO₃)-ACN]; gradient: 42%-82% B over 32 min) to yield the title compound 5-chloro-3-[4-chloro-6-[(3S,5R)-3,5-dimethylpiperazin-1-yl]-2-pyridyl]pyrazolo[1,5-a]pyridine (Compound 59) (1.5 mg, 3%) as a white solid. LCMS (ESI MS) m/z=376.1 [M+H]+ 1.

¹H NMR (400 MHz, methanol-d₄) δ=8.56 (s, 1H), 8.54 (s, 1H), 8.49 (s, 1H), 7.12 (s, 1H), 6.95-7.01 (m, 1H), 6.66 (s, 1H), 4.28 (dd, J=12.4, 2.1 Hz, 2H), 2.88-3.02 (m, 2H), 2.55 (t, J=11.8 Hz, 2H), 1.22 (d, J=6.4 Hz, 6H).

### Example 60

### 5-chloro-3-[5-chloro-6-[(3S,5R)-3,5-dimethylpiperazin-1-yl]-2-pyridyl]pyrazolo[1,5-a]pyridine

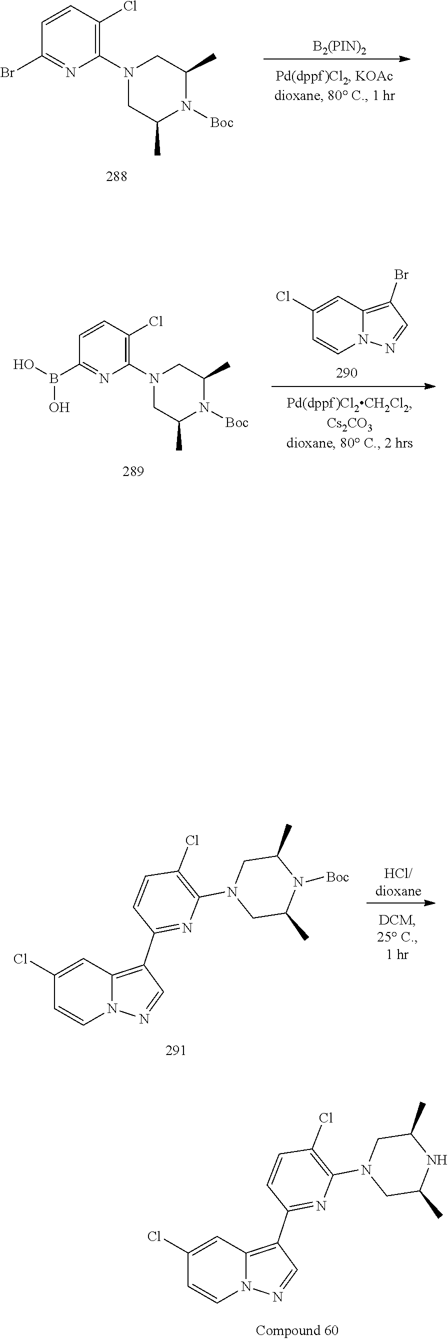 [SMILES: C[C@@H]1CN(c2nc(Br)ccc2Cl)C[C@H](C)N1C(=O)OC(C)(C)C ; "B2(PIN)2" ; C[C@@H]1CN(c2nc(B(O)O)ccc2Cl)C[C@H](C)N1C(=O)OC(C)(C)C ; Clc1ccn2ncc(Br)c2c1 ; C[C@@H]1CN(c2nc(-c3cnn4ccc(Cl)cc34)ccc2Cl)C[C@H](C)N1C(=O)OC(C)(C)C ; C[C@@H]1CN(c2nc(-c3cnn4ccc(Cl)cc34)ccc2Cl)C[C@H](C)N1]

Step A: A mixture of tert-butyl (2S,6R)-4-(6-bromo-3-chloropyridin-2-yl)-2,6-dimethylpiperazine-1-carboxylate 288 (100 mg, 247.08 μmol), 4,4,5,5-tetramethyl-2-(4,4,5,5-tetramethyl-1,3,2-dioxaborolan-2-yl)-1,3,2-dioxaborolane (126 mg, 494.16 μmol), KOAc (49 mg, 494.16 μmol), and Pd(dppf)Cl₂ (36 mg, 49.42 μmol) in dioxane (2 mL) is purged with N₂ three times. The mixture is stirred at 80° C. for 1 hr under N₂. The reaction mixture is filtered and concentrated under reduced pressure to yield the title compound [6-[(3S,5R)-4-tert-butoxycarbonyl-3,5-dimethyl-piperazin-1-yl]-5-chloro-2-pyridyl]boronic acid 289 (200 mg, crude) as a brown oil. LCMS (ESI MS) m/z=370.1 [M+H]+ 1.

Step B: A mixture of 3-bromo-5-chloropyrazolo[1,5-a]pyridine 290 (56 mg, 243.47 μmol), (6-((3S,5R)-4-(tert-butoxycarbonyl)-3,5-dimethylpiperazin-1-yl)-5-chloropyridin-2-yl)boronic acid 289 (90 mg, 243.47 μmol), Pd(dppf)Cl₂·CH₂Cl₂ (40 mg, 48.69 μmol), and Cs₂CO₃ (159 mg, 486.95 μmol) in dioxane (2 mL) is purged with N₂ three times. The mixture is stirred at 80° C. for 2 hr under N₂. The reaction mixture is concentrated under reduced pressure to give a residue which is purified by flash silica gel chromatography (eluent of 0-20% ethyl acetate/petroleum ether gradient) to yield the title compound (2S,6R)-4-[3-chloro-6-(5-chloropyrazolo[1,5-a]pyridin-3-yl)-2-pyridyl]-2,6-dimethyl-piperazine-1-carboxylate 291 (70 mg, 60%) as a white solid.

¹H NMR (400 MHz, methanol-d₄) δ=8.82 (d, J=7.6 Hz, 1H), 8.76 (s, 1H), 8.54 (d, J=2.0 Hz, 1H), 7.85 (d, J=8.4 Hz, 1H), 7.59 (d, J=8.4 Hz, 1H), 7.09 (dd, J=2.4, 7.6 Hz, 1H), 4.20 (br dd, J=4.4, 6.0 Hz, 2H), 3.68 (br d, J=12.0 Hz, 2H), 2.99 (br dd, J=4.0, 12.0 Hz, 2H), 1.45 (s, 9H), 1.38 (d, J=6.8 Hz, 6H).

Step C: To a solution of tert-butyl (2S,6R)-4-(3-chloro-6-(5-chloropyrazolo[1,5-a]pyridin-3-yl)pyridin-2-yl)-2,6-dimethylpiperazine-1-carboxylate 291 (60 mg, 125.95 μmol) in DCM (1 mL) is added HCl/dioxane (4 M, 6.0 mL). The mixture is stirred at 25° C. for 1 hr. The reaction mixture is partitioned between DCM and H₂O. The organic phase is discarded, and the aqueous phase is lyophilized to give the title compound 5-chloro-3-[5-chloro-6-[(3S,5R)-3,5-dimethylpiperazin-1-yl]-2-pyridyl]pyrazolo[1,5-a]pyridine (Compound 60) (28.3 mg, 56%) as a white solid. LCMS (ESI MS) m/z=376.1 [M+H]+ 1.

¹H NMR (400 MHz, methanol-d₄) δ=8.59 (d, J=7.6 Hz, 1H), 8.54 (s, 2H), 7.77 (d, J=8.0 Hz, 1H), 7.50 (d, J=8.0 Hz, 1H), 7.01 (dd, J=2.0, 7.2 Hz, 1H), 4.12 (br d, J=13.2 Hz, 2H), 3.69-3.60 (m, 2H), 3.02 (dd, J=11.6, 13.2 Hz, 2H), 1.46 (d, J=6.8 Hz, 6H).

### Example 61

### 5-chloro-3-[6-[(3S,5R)-3,5-dimethylpiperazin-1-yl]-5-fluoro-2-pyridyl]pyrazolo[1,5-a]pyridine

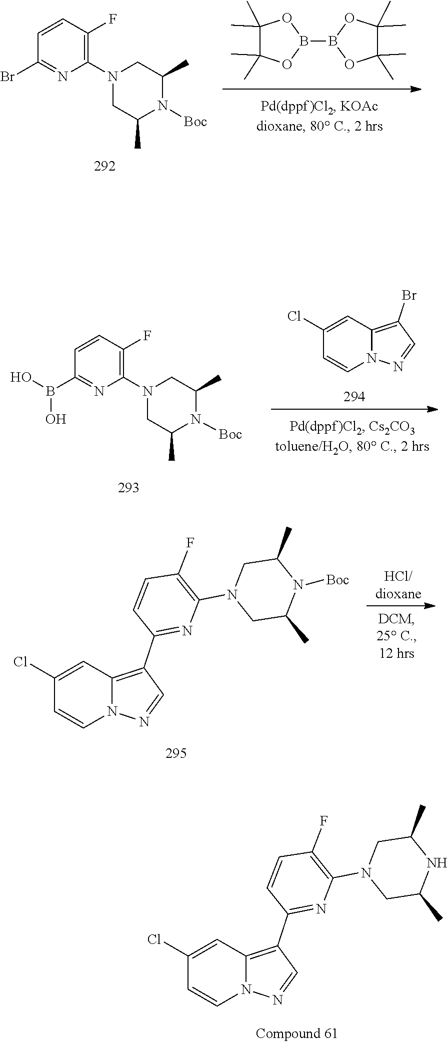

Step A: A mixture of tert-butyl (2S,6R)-4-(6-bromo-3-fluoropyridin-2-yl)-2,6-dimethylpiperazine-1-carboxylate 292 (261 mg, 1.03 mmol), 4,4,4′,4′,5,5,5′,5′-octamethyl-2,2′-bi(1,3,2-dioxaborolane) (200 mg, 515.10 μmol), KOAc (101 mg, 1.03 mmol), and Pd(dppf)Cl₂·CH₂Cl₂ (84 mg, 103.02 μmol) in dioxane (5 mL) is purged with N₂ three times. The mixture is stirred at 80° C. for 2 hr under N₂. The reaction mixture is quenched with H₂O, and the mixture is extracted with EtOAc three times. The combined organic layers are dried over anhydrous Na₂SO₄, filtered and concentrated under reduced pressure to give the title compound [6-[(3S,5R)-4-tert-butoxycarbonyl-3,5-dimethyl-piperazin-1-yl]-5-fluoro-2-pyridyl]boronic acid 293 (170 mg, crude) as a white solid. LCMS (ESI MS) m/z=354.1 [M+H]+ 1.

Step B: A mixture of (6-((3S,5R)-4-(tert-butoxycarbonyl)-3,5-dimethylpiperazin-1-yl)-5-fluoropyridin-2-yl)boronic acid 2_9 (60 mg, 259.21 μmol), 3-bromo-5-chloropyrazolo[1,5-a]pyridine 294 (137 mg, 388.8 μmol), Cs₂CO₃ (168 mg, 518.4 μmol), and Pd(dppf)Cl₂·CH₂Cl₂ (42 mg, 51.84 μmol) in toluene (3 mL) and H₂O (1 mL) is purged with N₂ three times. The mixture is stirred at 80° C. for 2 hr under N₂. The reaction is quenched with water and extracted with EtOAc three times. The combined organic layers are dried over anhydrous Na₂SO₄, filtered and concentrated under reduced pressure to give a residue which is purified by flash silica gel chromatography (eluent of 0-25% ethyl acetate/petroleum ether gradient) to yield the title compound tert-butyl (2S,6R)-4-[6-(5-chloropyrazolo[1,5-a]pyridin-3-yl)-3-fluoro-2-pyridyl]-2,6-dimethyl-piperazine-1-carboxylate 295 (50 mg) as a white solid. LCMS (ESI MS) m/z=460.0 [M+H]+ 1.

Step C: To a solution of tert-butyl (2S,6R)-4-(6-(5-chloropyrazolo[1,5-a]pyridin-3-yl)-3-fluoropyridin-2-yl)-2,6-dimethylpiperazine-1-carboxylate 295 (40 mg, 86.97 μmol) in DCM (1 mL) is added HCl/dioxane (4 M, 1 mL). The mixture is stirred at 25° C. for 12 hrs. The reaction mixture is partitioned between DCM and H₂O. The organic phase is discarded, and the aqueous phase is lyophilized to give the title compound 5-chloro-3-[6-[(3S,5R)-3,5-dimethylpiperazin-1-yl]-5-fluoro-2-pyridyl]pyrazolo[1,5-a]pyridine (Compound 61) (19.0 mg, 58%) as a white solid. LCMS (ESI MS) m/z=360.1 [M+H]+ 1.

¹H NMR (400 MHz, DMSO-d₆) δ=8.68 (d, J=7.38 Hz, 1H), 8.60 (s, 1H), 8.34 (d, J=2.13 Hz, 1H), 7.57 (dd, J=12.63, 8.25 Hz, 1H), 7.42 (dd, J=8.25, 2.38 Hz, 1H), 7.03 (dd, J=7.44, 2.19 Hz, 1H), 4.10 (br d, J=12.13 Hz, 2H), 3.45 (ddd, J=10.35, 6.79, 3.13 Hz, 2H), 2.93-3.06 (m, 2H), 1.31 (d, J=6.50 Hz, 6H).

### Example 62

### 3-[4-chloro-6-(3-piperidyl)-2-pyridyl]-N-methyl-pyrazolo[1,5-a]pyridin-5-amine

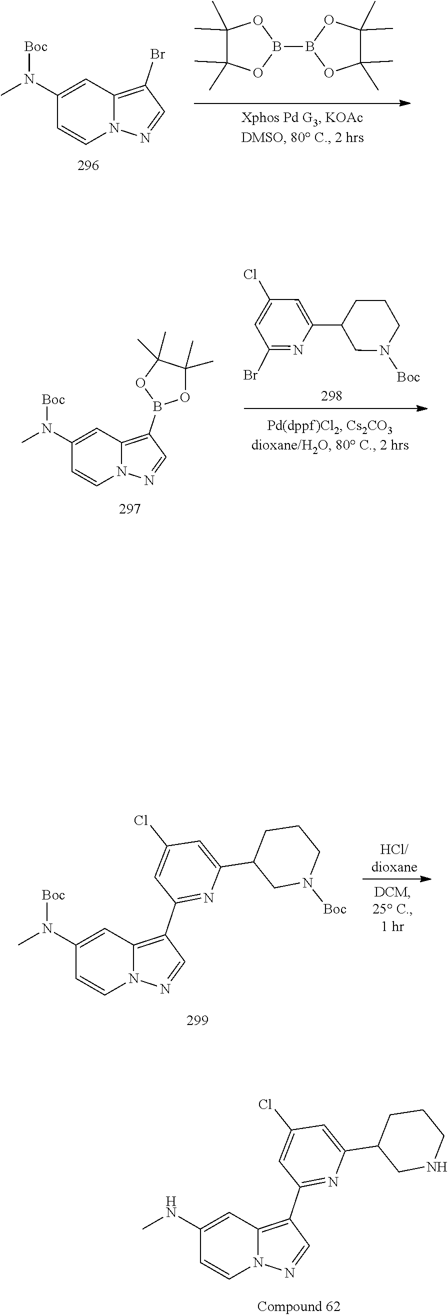

Step A: A mixture of tert-butyl (3-bromopyrazolo[1,5-a]pyridine-5-yl)(methyl)carbamate 296 (100 mg, 306.6 μmol), 4,4,5,5-tetramethyl-2-(4,4,5,5-tetramethyl-1,3,2-dioxaborolan-2-yl)-1,3,2-dioxaborolane (155 mg, 613.1 μmol), KOAc (60 mg, 613.1 μmol), and XPhos Pd G3 (25 mg, 30.7 μmol) in DMSO (3 mL) is degassed and purged with N₂ three times. The mixture is stirred at 80° C. for 2 hr under N₂. The reaction mixture is quenched with H₂O, and the mixture is extracted with EtOAc three times. The combined organic layers are washed with brine, dried over Na₂SO₄, filtered and concentrated under reduced pressure to give a residue which is purified by flash silica gel chromatography (eluent of 0-10% ethyl acetate/petroleum ether gradient) to yield the title compound tert-butyl N-methyl-N-[3-(4,4,5,5-tetramethyl-1,3,2-dioxaborolan-2-yl)pyrazolo[1,5-a]pyridin-5-yl]carbamate 297 (70 mg) as a white solid. LCMS (ESI MS) m/z=374.1

Step B: A mixture of tert-butyl N-methyl-N-[3-(4,4,5,5-tetramethyl-1,3,2-dioxaborolan-2-yl)pyrazolo[1,5-a]pyridin-5-yl]carbamate 297 (50 mg, 133.1 μmol), tert-butyl 3-(6-bromo-4-chloropyridin-2-yl)piperidine-1-carboxylate 298 (59 mg, 159.7 μmol), Cs₂CO₃ (86 mg, 266.18 μmol), and Pd(dppf)Cl₂·CH₂Cl₂ (10 mg, 13.31 μmol) in dioxane (1 mL) and H₂O (0.3 mL) is degassed and purged with N₂ three times. The mixture is stirred at 80° C. for 2 hr under N₂. The reaction is quenched with water and extracted with EtOAc three times. The combined organic layers are dried over anhydrous Na₂SO₄, filtered and concentrated under reduced pressure to yield tert-butyl 5-(5-fluoro-6-pyrazolo[1,5-a]pyridin-3-yl-2-pyridyl)-3,6-dihydro-2H-pyridine-1-carboxylate 299 (40 mg) as a white solid. LCMS (ESI MS) m/z=542.3 [M+H]+ 1.

Step C: To a solution of 5-(5-fluoro-6-pyrazolo[1,5-a]pyridin-3-yl-2-pyridyl)-3,6-dihydro-2H-pyridine-1-carboxylate 299 (40 mg, 73.8 μmol) in DCM (0.5 mL) is added HCl/dioxane (4M, 0.5 mL). The mixture is stirred at 25° C. for 1 hr. The reaction mixture is concentrated under vacuum to give a crude product which is purified by prep-HPLC (column: Xtimate C18 150*40 mm*10 um; mobile phase: [water (FA)-ACN]; gradient: 0%-34% B over 25 min) to yield the title compound 3-[4-chloro-6-(3-piperidyl)-2-pyridyl]-N-methyl-pyrazolo[1,5-a]pyridin-5-amine (Compound 62) (3 mg, 10%) as a brown oil. LCMS (ESI MS) m/z=342.0 [M+H]+1.

¹H NMR (400 MHz, methanol-d₄) δ=8.56 (br s, 1H), 8.30 (s, 1H), 8.19 (br d, J=7.50 Hz, 1H), 7.60 (s, 1H), 7.30 (br s, 2H), 7.08 (s, 1H), 6.49 (br dd, J=7.38, 2.25 Hz, 2H), 3.69 (br d, J=11.13 Hz, 2H), 3.40-3.50 (m, 2H), 3.37 (br s, 1H), 3.33 (br s, 2H), 3.24 (br d, J=9.88 Hz, 1H), 2.94-3.08 (m, 3H), 2.25 (br d, J=10.88 Hz, 1H), 2.05-2.12 (m, 1H), 1.98 (br d, J=10.76 Hz, 1H).

### Example 63

### 5-chloro-3-[3-chloro-6-[(3S,5R)-3,5-dimethylpiperazin-1-yl]-2-pyridyl]pyrazolo[1,5-a]pyridine

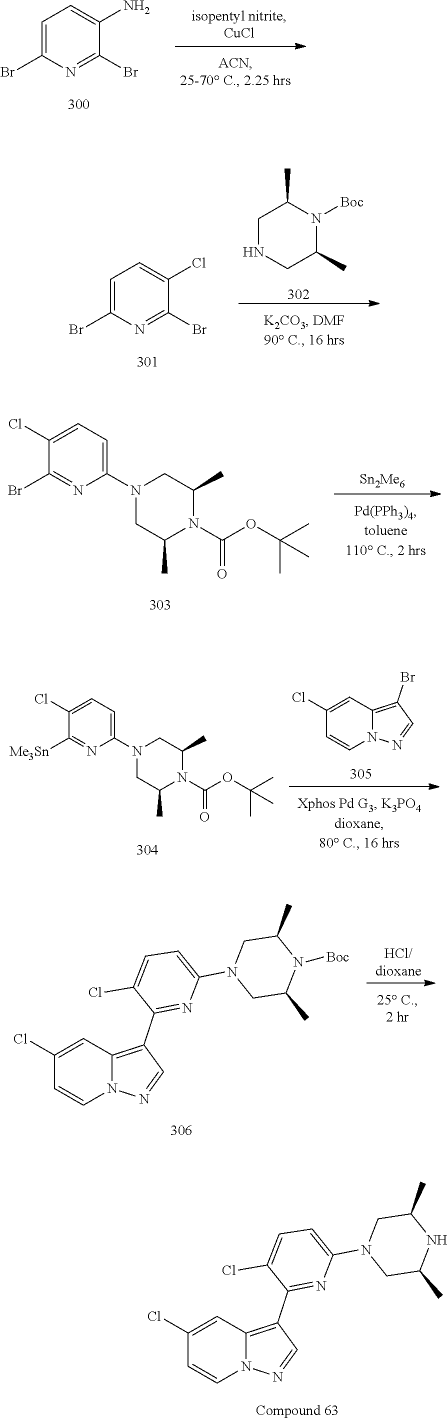

Step A: A mixture of 2,6-dibromopyridin-3-amine 300 (2.5 g, 9.92 mmol) and CuCl (2.46 g, 24.8 mmol) in ACN (30 mL) is stirred for 15 min at 25° C. Isopentyl nitrite (2.33 g, 19.85 mmol) is added. The mixture is stirred for 2 hr at 70° C. The reaction mixture is concentrated under vacuum, and the residue is purified by flash silica gel chromatography (eluent of 5% ethyl acetate/petroleum ether gradient) to yield the title compound 5-chloro-3-[3-chloro-6-[(3S,5R)-3,5-dimethylpiperazin-1-yl]-2-pyridyl]pyrazolo[1,5-a]pyridine 301 (1.7 g) as a white solid. LCMS (ESI MS) m/z=269.8 [M+H]+ 1.

Step B: A mixture of 5-chloro-3-[3-chloro-6-[(3S,5R)-3,5-dimethylpiperazin-1-yl]-2-pyridyl]pyrazolo[1,5-a]pyridine 301 (2 g, 7.37 mmol), 302 (1.11 g, 5.16 mmol) and K₂CO₃ (2.04 g, 14.74 mmol) in DMF (20 mL) is stirred at 90° C. for 16 hrs. The reaction mixture is diluted with H₂O and extracted with EtOAc three times. The combined organic layers are washed with brine, dried over Na₂SO₄, filtered and concentrated under reduced pressure to give a residue which is purified by flash silica gel chromatography (eluent of 3-5% ethyl acetate/petroleum ether gradient) to yield the title compound tert-butyl (2S,6R)-4-(6-bromo-5-chloro-2-pyridyl)-2,6-dimethyl-piperazine-1-carboxylate 303 (430 mg) as a white solid. LCMS (ESI MS) m/z=404.0 [M+H]+ 1.

¹H NMR (400 MHz, DMSO-d₆) δ=7.68 (d, J=8.8 Hz, 1H), 6.94 (d, J=8.8 Hz, 1H), 4.10 (br d, J=12.4 Hz, 4H), 3.05 (br dd, J=13.2, 4.4 Hz, 2H), 1.42 (s, 9H), 1.12 (d, J=6.4 Hz, 6H).

Step C: A mixture of tert-butyl (2S,6R)-4-(6-bromo-5-chloro-2-pyridyl)-2,6-dimethyl-piperazine-1-carboxylate 303 (250 mg, 617.7 μmol), trimethyl(trimethylstannyl)stannane (0.94 g, 2.9 mmol) and Pd(PPh₃)₄ (143 mg, 123.5 μmol) in toluene (3 mL) is purged with N₂ three times. The mixture is stirred at 110° C. for 2 hr under N₂. The mixture is quenched with a saturated KF solution and stirred at 25° C. for 1 hr. The mixture is extracted with ethyl acetate three times. The combined organic layers are washed with brine, dried over Na₂SO₄ and evaporated under vacuum to give a residue which is purified by flash silica gel chromatography (eluent of 0-5% ethyl acetate/petroleum ether gradient) to yield the title compound tert-butyl (2S,6R)-4-(5-chloro-6-trimethylstannyl-2-pyridyl)-2,6-dimethyl-piperazine-1-carboxylate 34 (160 mg) as a white solid. LCMS (ESI MS) m/z=490.1 [M+H]+ 1.

Step D: A mixture of tert-butyl (2S,6R)-4-(5-chloro-6-trimethylstannyl-2-pyridyl)-2,6-dimethyl-piperazine-1-carboxylate 34 (160 mg, 327.4 μmol), 3-bromo-5-chloropyrazolo[1,5-a]pyridine 305 (113.6 mg, 491.2 μmol), XPhos Pd G3 (55.4 mg, 65.5 μmol) and K₃PO₄ (139.0 mg, 654.9 μmol) in dioxane (3 mL) is purged with N₂ three times. The mixture is stirred at 80° C. for 16 hr under N₂. The reaction mixture is concentrated under vacuum to give a residue which is purified by flash silica gel chromatography (eluent of 13% ethyl acetate/petroleum ether gradient) to yield the title compound tert-butyl (2S,6R)-4-[5-chloro-6-(5-chloropyrazolo[1,5-a]pyridin-3-yl)-2-pyridyl]-2,6-dimethyl-piperazine-1-carboxylate 306 (55 mg) as a yellow solid. LCMS (ESI MS) m/z=476.1 [M+H]+ 1.

Step E: A solution of tert-butyl (2S,6R)-4-[5-chloro-6-(5-chloropyrazolo[1,5-a]pyridin-3-yl)-2-pyridyl]-2,6-dimethyl-piperazine-1-carboxylate 306 (50 mg, 104.9 μmol) in HCl/dioxane (4 M, 7.5 mL) is stirred at 25° C. for 2 hr. The reaction mixture is filtered to give a residue which is purified by prep-HPLC (column: Xtimate C18 150*40 mm*10 um; mobile phase: [water (FA)-ACN]; gradient: 0%-38% B over 25 min) to yield the title compound 5-chloro-3-[3-chloro-6-[(3S,5R)-3,5-dimethylpiperazin-1-yl]-2-pyridyl]pyrazolo[1,5-a]pyridine (Compound 63) (9.7 mg, 24%) as a white solid. LCMS (ESI MS) m/z=376.1 [M+H]+ 1.

¹H NMR (400 MHz, methanol-d₄) δ=8.73 (s, 1H), 8.59 (d, J=7.2 Hz, 1H), 8.51 (s, 1H), 8.34 (d, J=2.4 Hz, 1H), 7.67 (d, J=8.8 Hz, 1H), 7.01 (dd, J=7.2, 2.4 Hz, 1H), 6.82 (d, J=9.2 Hz, 1H), 4.50 (dd, J=13.6, 2.6 Hz, 2H), 3.36-3.46 (m, 2H), 2.87 (dd, J=13.6, 11.4 Hz, 2H), 1.40 (d, J=6.4 Hz, 6H).

### Example 64

### 5-chloro-3-[6-[(3S,5R)-3,5-dimethylpiperazin-1-yl]-3-fluoro-2-pyridyl]pyrazolo[1,5-a]pyridine

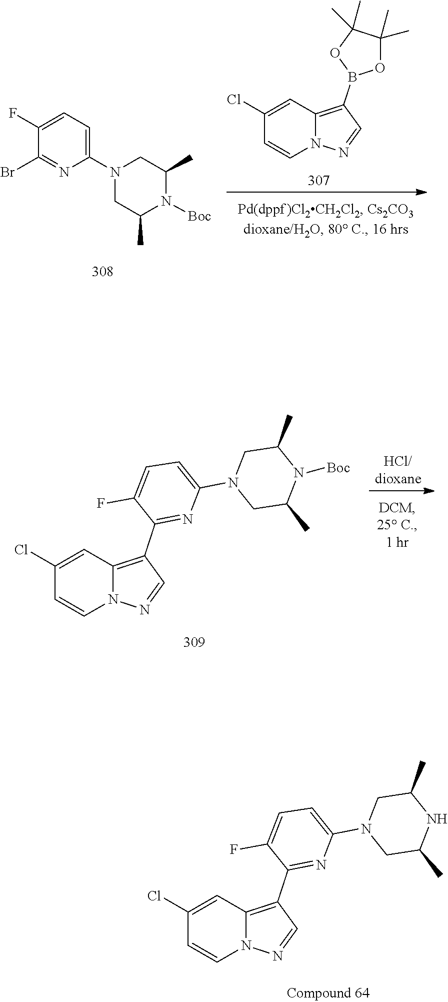 [SMILES: C[C@@H]1CN(c2ccc(F)c(Br)n2)C[C@H](C)N1C(=O)OC(C)(C)C ; CC1(C)OB(c2cnn3ccc(Cl)cc23)OC1(C)C ; C[C@@H]1CN(c2ccc(F)c(-c3cnn4ccc(Cl)cc34)n2)C[C@H](C)N1C(=O)OC(C)(C)C ; C[C@@H]1CN(c2ccc(F)c(-c3cnn4ccc(Cl)cc34)n2)C[C@H](C)N1]

Step A: A mixture of 5-chloro-3-(4,4,5,5-tetramethyl-1,3,2-dioxaborolan-2-yl)pyrazolo[1,5-a]pyridine 307 (36 mg, 128.8 μmol), tert-butyl (2S,6R)-4-(6-bromo-5-fluoropyridin-2-yl)-2,6-dimethylpiperazine-1-carboxylate 308 (50 mg, 128.8 μmol), Pd(dppf)Cl₂·CH₂Cl₂ (21 mg, 25.7 μmol), and Cs₂CO₃ (84 mg, 257.5 μmol) in H₂O (0.1 mL) and dioxane (1 mL) is purged with N₂ three times. The mixture is stirred at 80° C. for 16 hr under N₂. The reaction mixture is concentrated under reduced pressure to give a residue which is purified by flash silica gel chromatography (eluent of 20-30% ethyl acetate/petroleum ether gradient) to yield the title compound tert-butyl (2S,6R)-4-[6-(5-chloropyrazolo[1,5-a]pyridin-3-yl)-5-fluoro-2-pyridyl]-2,6-dimethyl-piperazine-1-carboxylate 309 (40 mg, 67%) as a white solid. LCMS (ESI MS) m/z=460.0 [M+H]+ 1.

Step B: To a solution of tert-butyl (2S,6R)-4-[6-(5-chloropyrazolo[1,5-a]pyridin-3-yl)-5-fluoro-2-pyridyl]-2,6-dimethyl-piperazine-1-carboxylate 309 (30 mg, 65.2 μmol) in DCM (1 mL) is added HCl/dioxane (4M, 2.0 mL). The mixture is stirred at 25° C. for 1 hr. The reaction mixture is partitioned between DCM and H₂O. The organic phase is discarded, and the water phase is lyophilized to yield the title compound 5-chloro-3-[6-[(3S,5R)-3,5-dimethylpiperazin-1-yl]-3-fluoro-2-pyridyl]pyrazolo[1,5-a]pyridine (Compound 64) (3.7 mg, 14%) as a white solid. LCMS (ESI MS) m/z=360.1 [M+H]+ 1.

¹H NMR (400 MHz, DMSO-d₆) δ=8.87 (d, J=7.2 Hz, 1H), 8.55-8.48 (m, 2H), 7.67 (dd, J=9.2, 10.8 Hz, 1H), 7.15 (dd, J=2.4, 7.2 Hz, 1H), 6.90 (dd, J=2.0, 9.2 Hz, 1H), 4.49-4.44 (m, 1H), 4.33 (br d, J=11.2 Hz, 2H), 3.00-2.85 (m, 2H), 2.04-1.93 (m, 1H), 1.33 (br d, J=6.0 Hz, 6H).

### Example 65

### 3-[4-chloro-6-(3-piperidyl)-2-pyridyl]-5-isopropoxy-pyrazolo[1,5-a]pyridine

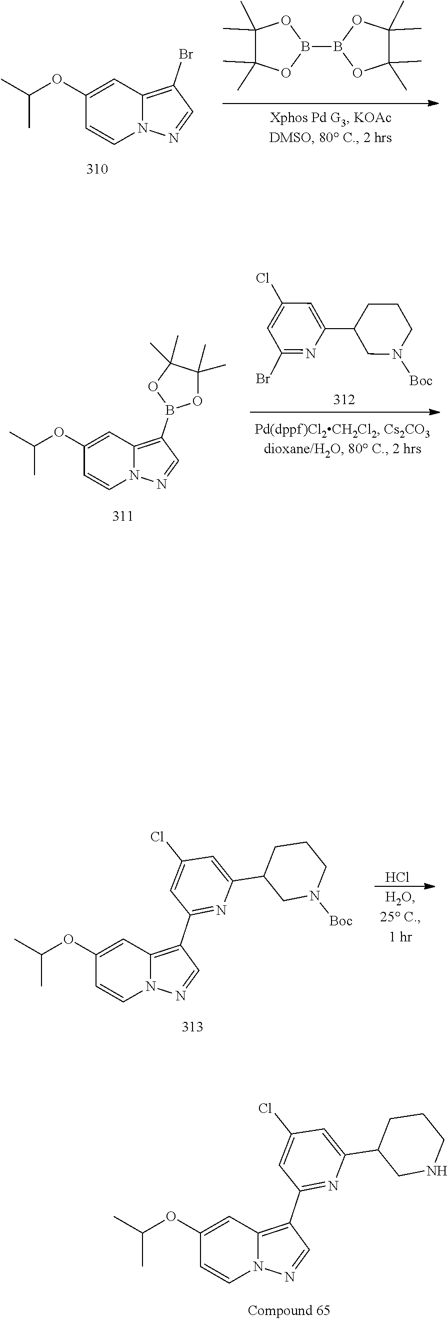

Step A: A mixture of 4,4,5,5-tetramethyl-2-(4,4,5,5-tetramethyl-1,3,2-dioxaborolan-2-yl)-1,3,2-dioxaborolane (995.4 mg, 3.92 mmol), 3-bromo-5-isopropoxypyrazolo[1,5-a]pyridine 310 (500 mg, 1.96 mmol), KOAc (384.7 mg, 3.92 mmol), and XPhos Pd G3 (165.9 mg, 196 μmol) in DMSO (8 mL) is purged with N₂ three times. The mixture is stirred at 80° C. for 2 hr under N₂ and then diluted with H₂O (10 mL). The resulting mixture is extracted with EtOAc three times. The combined organic phase is washed with brine three times, dried over anhydrous Na₂SO₄, filtered and concentrated to give a crude product which is purified by flash silica gel chromatography (eluent of 0-6% ethyl acetate/petroleum ether gradient) to give the title compound 5-isopropoxy-3-(4,4,5,5-tetramethyl-1,3,2-dioxaborolan-2-yl)pyrazolo[1,5-a]pyridine 311 as a white solid.

¹H NMR (400 MHz, methanol-d₄) δ=8.40 (d, J=7.6 Hz, 1H), 8.02 (s, 1H), 7.20 (d, J=2.4 Hz, 1H), 6.62 (dd, J=2.4, 7.6 Hz, 1H), 4.69 (td, J=6.0, 12.0 Hz, 1H), 1.39 (d, J=6.0 Hz, 6H), 1.36 (s, 12H).

Step B: A mixture of 5-isopropoxy-3-(4,4,5,5-tetramethyl-1,3,2-dioxaborolan-2-yl)pyrazolo[1,5-a]pyridine 311 (30 mg, 99.3 μmol), tert-butyl 3-(6-bromo-4-chloropyridin-2-yl)piperidine-1-carboxylate 312 (41 mg, 109.2 μmol), Pd(dppf)Cl₂·CH₂Cl₂ (16 mg, 19.9 μmol), and Cs₂CO₃ (38 mg, 119.1 μmol) in dioxane (1 mL) and H₂O (0.2 mL) is purged with N₂ three times. The mixture is stirred at 80° C. for 2 hr under N₂. The reaction mixture is concentrated under vacuum to give a crude product which is purified by flash silica gel chromatography (eluent of 0-9% ethyl acetate/petroleum ether gradient) to yield the title compound tert-butyl 3-[4-chloro-6-(5-isopropoxypyrazolo[1,5-a]pyridin-3-yl)-2-pyridyl]piperidine-1-carboxylate 313 as brown oil. LCMS (ESI MS) m/z=471.2 [M+H]+ 1.

Step C: To a solution of tert-butyl 3-[4-chloro-6-(5-isopropoxypyrazolo[1,5-a]pyridin-3-yl)-2-pyridyl]piperidine-1-carboxylate 313 (20 mg, 42.5 μmol) in H₂O (0.5 mL) is added HCl (12 M, 466 μL). The mixture is stirred at 25° C. for 1 hr. The reaction mixture is concentrated under vacuum to give a crude product which is purified by prep-HPLC (column: Xtimate C18 150*40 mm*10 um; mobile phase: [water (HCl)-ACN]; gradient: 10%-50% B over 25 min) to yield the title compound 3-[4-chloro-6-(3-piperidyl)-2-pyridyl]-5-isopropoxy-pyrazolo[1,5-a]pyridine (Compound 65) as a white solid. LCMS (ESI MS) m/z=371.2 [M+H]+ 1.

¹H NMR (400 MHz, methanol-d₄) δ=8.50-8.42 (m, 2H), 7.87 (d, J=2.8 Hz, 1H), 7.75 (d, J=1.6 Hz, 1H), 7.19 (d, J=1.6 Hz, 1H), 6.70 (dd, J=2.8, 7.6 Hz, 1H), 4.82-4.78 (m, 1H), 3.69 (br d, J=11.2 Hz, 1H), 3.50 (br d, J=12.4 Hz, 1H), 3.37-3.32 (m, 1H), 3.28-3.22 (m, 1H), 3.09-2.95 (m, 1H), 2.34-2.22 (m, 1H), 2.17-2.08 (m, 1H), 2.06-1.92 (m, 2H), 1.46 (dd, J=4.0, 6.0 Hz, 6H).

### Example 66

### 5-chloro-3-[6-(3,3,5,5-tetramethylpiperazin-1-yl)-2-pyridyl]pyrazolo[1,5-a]pyridine

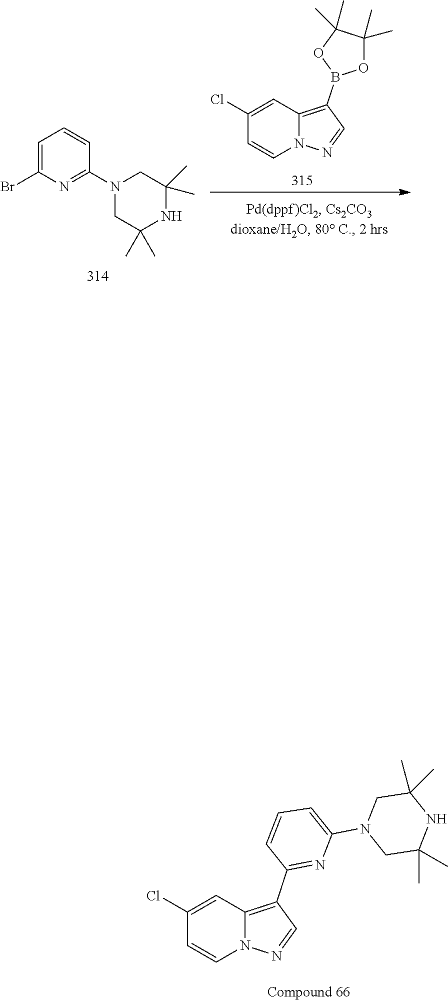 [SMILES: CC1(C)CN(c2cccc(Br)n2)CC(C)(C)N1 ; CC1(C)OB(c2cnn3ccc(Cl)cc23)OC1(C)C ; CC1(C)CN(c2cccc(-c3cnn4ccc(Cl)cc34)n2)CC(C)(C)N1]

A mixture of 1-(6-bromopyridin-2-yl)-3,3,5,5-tetramethylpiperazine 314 (70 mg, 234.73 μmol), 5-chloro-3-(4,4,5,5-tetramethyl-1,3,2-dioxaborolan-2-yl)pyrazolo[1,5-a]pyridine 315 (130 mg, 469.5 μmol), Pd(dppf)Cl₂ (19 mg, 23.4 μmol), and Cs₂CO₃ (152 mg, 469.4 μmol) in dioxane (3 mL) and H₂O (1 mL) is purged with N₂ three times. The mixture is stirred at 80° C. for 2 hr under N₂. The reaction mixture is diluted with H₂O, and the mixture is extracted with EtOAc three times. The combined organic layer is dried over Na₂SO₄, filtered and concentrated under reduced pressure to give a residue which is purified by prep-HPLC (column: Xtimate C18 150*40 mm*10 um; mobile phase: [water(NH₃H₂O+NH₄HCO₃)-ACN]; gradient: 38%-78% B over 25 min) to yield the title compound 5-chloro-3-[6-(3,3,5,5-tetramethylpiperazin-1-yl)-2-pyridyl]pyrazolo[1,5-a]pyridine (Compound 66) (25 mg, 26%) as a white solid. LCMS (ESI MS) m/z=370.2 [M+H]+ 1.

¹H NMR (400 MHz, methanol-d₄) δ=8.67 (s, 1H), 8.56 (br d, J=7.13 Hz, 1H), 8.47 (s, 1H), 7.56 (br t, J=7.88 Hz, 1H), 7.08 (br d, J=7.38 Hz, 1H), 6.97 (br d, J=6.88 Hz, 1H), 6.65 (br d, J=8.63 Hz, 1H), 3.53 (s, 4H), 1.31 (s, 12H).

### Example 67

### 2-(methylsulfonyl)-N-(3-(6-(piperidin-3-yl)pyridin-2-yl)pyrazolo[1,5-a]pyridin-5-yl)acetamide

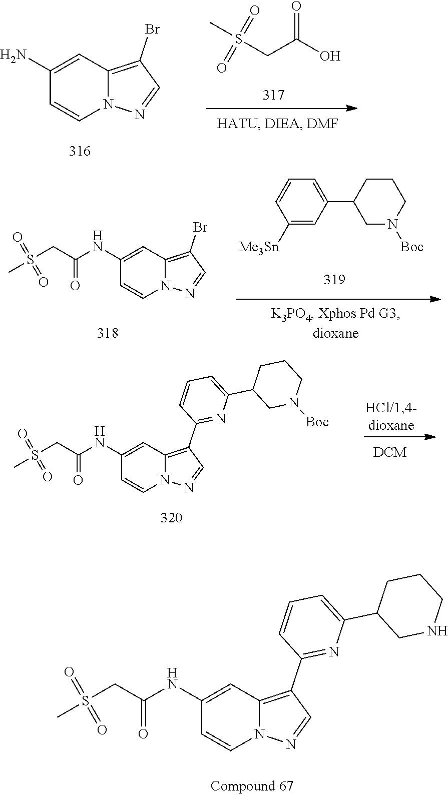

Step A: To a mixture of 3-bromopyrazolo[1,5-a]pyridin-5-amine 316 (200.0 mg, 0.94 mmol) and 2-(methylsulfonyl)acetic acid 317 (143.3 mg, 1.04 mmol) in DMF (5.0 mL) is added HATU (430.4 mg, 1.13 mmol) and DIPEA (487.6 mg, 3.77 mmol). The reaction mixture is stirred at room temperature for 1 hr. The reaction mixture is quenched with saturated NaCl solution and extracted with EtOAc. The combined organic phase is concentrated under reduced pressure, and the resulting residue is purified by column chromatography (silica gel, eluent of 0-30% ethyl acetate/petroleum ether gradient) to give the title compound N-(3-bromopyrazolo[1,5-a]pyridin-5-yl)-2-(methylsulfonyl)acetamide 318 (165 mg, 53% yield) as a white solid. LCMS (ESI) m/z=332 [M+H]+.

Step B: To a mixture of N-(3-bromopyrazolo[1,5-a]pyridin-5-yl)-2-(methylsulfonyl)acetamide 318 (100.0 mg, 0.30 mmol) and tert-butyl 3-(6-(trimethylstannyl)pyridin-2-yl)piperidine-1-carboxylate 319 (256.5 mg, 0.60 mmol) in dioxane (2 mL) is added K₃PO₄ (127.8 mg, 0.60 mmol) and Xphos Pd G3 (50.9 mg, 0.06 mmol). The reaction mixture is stirred at 80° C. for 3 hrs under nitrogen. The reaction mixture is filtered and concentrated in vacuo. The residue is purified by column chromatography (silica gel, eluent of 0-20% ethyl acetate/petroleum ether gradient) to give the title compound tert-butyl 3-(6-(5-(2-(methylsulfonyl)acetamido)pyrazolo[1,5-a]pyridin-3-yl)pyridin-2-yl)piperidine-1-carboxylate 320 (130.0 mg, 84% yield) as a yellow solid. LCMS (ESI) m/z=514.3 [M+H]+.

Step C: To a solution of tert-butyl 3-(6-(5-(2-(methylsulfonyl)acetamido)pyrazolo[1,5-a]pyridin-3-yl)pyridin-2-yl)piperidine-1-carboxylate 320 (140.0 mg, 0.27 mmol) in DCM (3.0 mL) is added HCl/dioxane (1.5 mL, 4M). The reaction mixture is stirred at room temperature for 1 hr under nitrogen. The reaction mixture is concentrated under reduced pressure. The residue is purified by prep-HPLC (YMC-ActusTriart C18, 20%-68% MeCN in water with 0.1% NH₄OH) to give the title compound 2-(methylsulfonyl)-N-(3-(6-(piperidin-3-yl)pyridin-2-yl)pyrazolo[1,5-a]pyridin-5-yl)acetamide (Compound 67) (39.1 mg, 35% yield) as a white solid. LCMS (ESI) m/z=414.2 [M+H]+.

¹H NMR (400 MHz, DMSO-d₆) δ=11.58 (s, 1H), 9.11 (s, 1H), 8.71 (d, J=7.5 Hz, 1H), 8.66 (s, 1H), 7.74-7.69 (m, 2H), 7.25-7.19 (m, 1H), 7.06 (dd, J=5.3, 3.2 Hz, 1H), 4.42 (d, J=2.5 Hz, 2H), 3.45 (d, J=11.0 Hz, 2H), 3.28-3.19 (m, 3H), 3.12-2.97 (m, 3H), 2.12-1.64 (m, 5H).

### Example 68

### 2-(methylsulfonamido)-N-(3-(6-(piperidin-3-yl)pyridin-2-yl)pyrazolo[1,5-a]pyridin-5-yl)acetamide

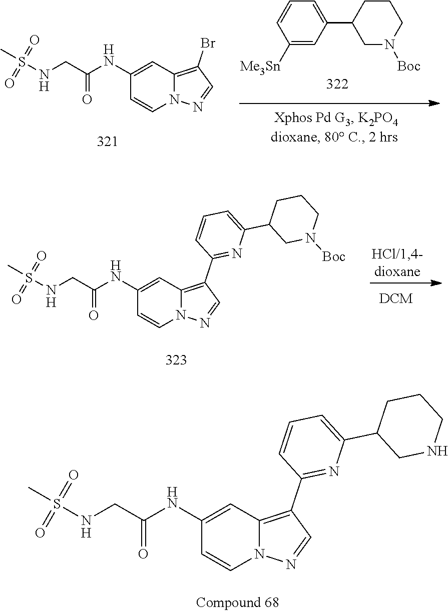

Step A: To a mixture of N-(3-bromopyrazolo[1,5-a]pyridin-5-yl)-2-(methylsulfonamido)acetamide 32 (60 mg, 0.17 mmol) and tert-butyl 3-(6-(trimethylstannyl)pyridin-2-yl)piperidine-1-carboxylate 322 (147.11 mg, 0.35 mmol) in dioxane (6.0 mL) is added K₃PO₄ (73.44 mg, 0.35 mmol) and Xphos Pd G3 (14.64 g, 0.017 mmol). The reaction mixture is stirred at room temperature at 80° C. for 2 hrs. The reaction mixture is poured into water and then extracted with ethyl acetate three times. The combined organic layers are washed with brine, dried over Na₂SO₄, filtered and concentrated under reduced pressure to give a residue. The residue is purified by column chromatography (silica gel, eluent of 0-30% MeOH/DCM gradient) to give the title compound 3-(6-(5-(2-(methylsulfonamido)acetamido)pyrazolo[1,5-a]pyridin-3-yl)pyridin-2-yl)piperidine-1-carboxylate 323 (43 mg, 47.1% yield) as a white solid. LCMS (ESI) m/z=529.2 [M+H]+.

Step B: A mixture of 3-(6-(5-(2-(methylsulfonamido)acetamido)pyrazolo[1,5-a]pyridin-3-yl)pyridin-2-yl)piperidine-1-carboxylate 323 (43.0 mg, 0.08 mmol) in HCl/1,4-dioxane (1 mL, 4M) is stirred at 25° C. for 3 hrs. The reaction mixture is concentrated under reduced pressure. The residue is purified by prep-HPLC (YMC-ActusTriart C18, 5%-55% MeCN in water with 0.1% FA) to give the title compound 2-(methylsulfonamido)-N-(3-(6-(piperidin-3-yl)pyridin-2-yl)pyrazolo[1,5-a]pyridin-5-yl)acetamide (Compound 68) (20 mg, 57% yield) as a yellow oil. LCMS (ESI) m/z=429.1 [M+H]+.

¹H NMR (400 MHz, CD₃OD) δ=9.51 (d, J=2.4 Hz, 1H), 8.54 (s, 1H), 8.47-8.37 (m, 2H), 7.72-7.65 (m, 1H), 7.63-7.56 (m, 1H), 7.06 (d, J=7.6 Hz, 1H), 6.78 (dd, J=7.6, 2.4 Hz, 1H), 4.02 (s, 2H), 3.90 (t, J=12 Hz, 1H), 3.64 (dd, J=12.8, 3.6 Hz, 1H), 3.54-3.40 (m, 2H), 3.27-3.14 (m, 1H), 3.07 (s, 3H), 2.17-2.00 (m, 3H), 1.98-1.88 (m, 1H).

### Example 69

### (R)-3-(6-(3-methylpiperazin-1-yl)pyridin-2-yl)pyrazolo[1,5-a]pyridine formate

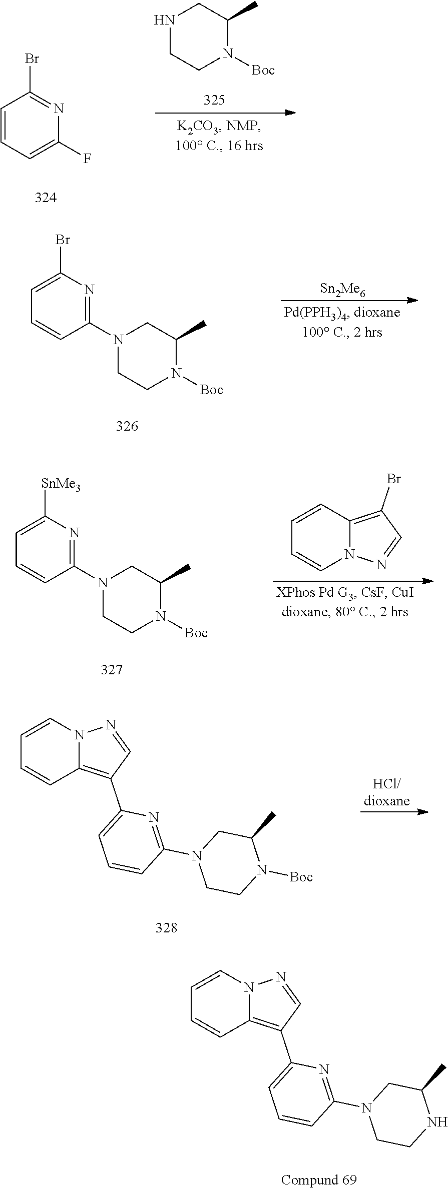

Step A: To a mixture of 2-bromo-6-fluoropyridine 324 (1 g, 5.68 mmol) in NMP (30 mL) is added tert-butyl (R)-2-methylpiperazine-1-carboxylate 325 (1.14 g, 5.68 mmol) and K₂CO₃ (3.93 g, 28.41 mmol). The mixture is stirred at 100° C. for 16 hours. The reaction mixture is poured into water and then extracted with ethyl acetate twice. The combined organic layers are washed with brine, dried over anhydrous Na₂SO₄. The mixture is filtered and concentrated under reduced pressure to give a residue. The residue is purified by column chromatography (silica gel, eluent of 0-25% ethyl acetate/petroleum ether gradient) to afford the title compound tert-butyl (R)-4-(6-bromopyridin-2-yl)-2-methylpiperazine-1-carboxylate 326 (1 g, 49%) as a yellow solid. LCMS (ESI) m/z=356.09 [M+H]+.

Step B: To a stirred mixture of tert-butyl (R)-4-(6-bromopyridin-2-yl)-2-methylpiperazine-1-carboxylate 326 (1 g, 2.81 mmol) in 1,4-dioxane (20 mL) is added Sn₂Me₆ (0.76 mL, 3.65 mmol) and Pd(PPh₃)₄ (0.32 g, 0.28 mmol). The resulting mixture is stirred at 110° C. for 2 hours. KF solution is added, and the mixture is stirred for 30 minutes. The mixture is filtered and concentrated under reduced pressure to give the title compound tert-butyl (R)-2-methyl-4-(6-(trimethylstannyl)pyridin-2-yl)piperazine-1-carboxylate 327 (1.53 g, crude) as a black oil which is used directly in the next step without further purification. LCMS (ESI) m/z=442.14 [M+H]+.

Step C: To a mixture of tert-butyl (R)-2-methyl-4-(6-(trimethylstannyl)pyridin-2-yl)piperazine-1-carboxylate 327 (1.53 g, 3.46 mmol) in 1,4-dioxane (60 mL) is added 3-bromopyrazolo[1,5-a]pyridine (432.2 mg, 2.19 mmol), K₃PO₄ (3.68 g, 17.32 mmol) and Xphos Pd G3 (0.29 g, 0.35 mmol). The resulting mixture is stirred at 80° C. for 2 hours under nitrogen. The reaction mixture is poured into water and then extracted with ethyl acetate three times. The combined organic layers are washed with brine, dried over anhydrous Na₂SO₄, and the mixture is filtered and concentrated under reduced pressure to give a residue. The residue is purified by column chromatography (silica gel, eluent of 0-25% ethyl acetate/petroleum ether gradient) to afford the title compound tert-butyl (R)-2-methyl-4-(6-(pyrazolo[1,5-a]pyridin-3-yl)pyridin-2-yl)piperazine-1-carboxylate 328 (261 mg, 19%) as a yellow solid. LCMS (ESI) m/z=394.22 [M+H]+.

Step D: A mixture of tert-butyl (R)-2-methyl-4-(6-(pyrazolo[1,5-a]pyridin-3-yl)pyridin-2-yl)piperazine-1-carboxylate 328 (261 mg, 0.66 mmol) in HCl/dioxane (3 mL, 4M) is stirred at room temperature for 2 hours. The reaction mixture is concentrated under reduced pressure to give a residue. The residue is purified by prep-HPLC (C18 250*21.2 mm, 10-95% MeCN in water with 0.1% FA) to give the title compound (R)-3-(6-(3-methylpiperazin-1-yl)pyridin-2-yl)pyrazolo[1,5-a]pyridine formate (Compound 69) (119.5 mg, 61% yield) as a white solid. LCMS (ESI) m/z=294 [M+H]+.

¹H NMR (400 MHz, CD₃OD) δ=8.60 (br s, 1H), 8.53-8.46 (m, 1H), 8.38 (s, 1H), 8.29 (d, J=8.8 Hz, 1H), 7.61-7.51 (m, 1H), 7.34-7.24 (m, 1H), 7.10 (d, J=7.6 Hz, 1H), 6.94-6.85 (m, 1H), 6.71-6.63 (m, 1H), 4.47-4.35 (m, 2H), 3.51 (d, J=11.2 Hz, 1H), 3.47-3.37 (m, 1H), 3.32-3.16 (m, 2H), 3.04 (dd, J=13.6, 10.4 Hz, 1H), 1.45-1.41 (m, 3H).

### Example 70

### (S)-3-(6-(3-methylpiperazin-1-yl)pyridin-2-yl)pyrazolo[1,5-a]pyridine formate

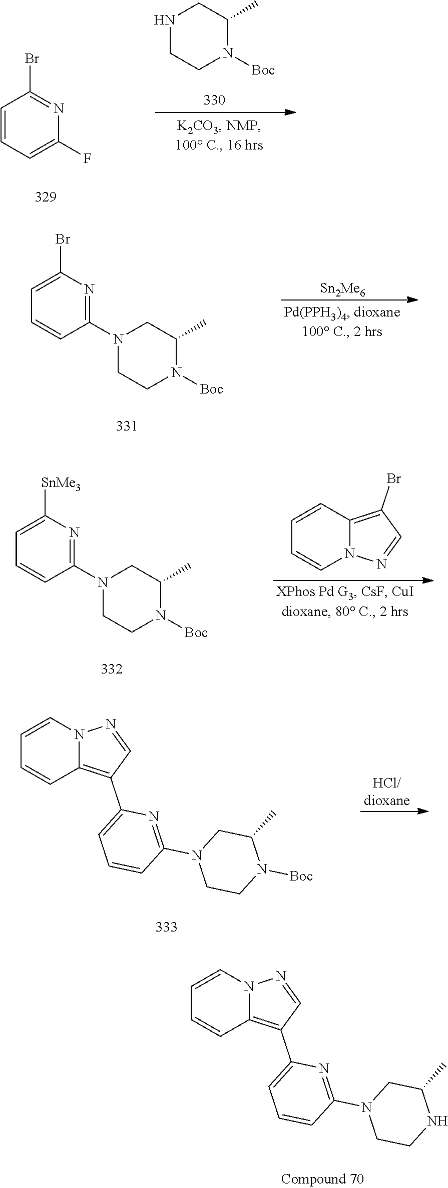

Step A: To a mixture of 2-bromo-6-fluoropyridine 2 (1 g, 5.68 mmol) in NMP (30 mL) is added tert-butyl (S)-2-methylpiperazine-1-carboxylate 3 (1.14 g, 5.68 mmol) and K₂CO₃ (3.93 g, 28.41 mmol). The mixture is stirred at 100° C. for 16 hours. The reaction mixture is poured into water and then extracted with ethyl acetate twice. The combined organic layers are washed with brine, dried over anhydrous Na₂SO₄. The mixture is filtered and concentrated under reduced pressure to give a residue. The residue is purified by column chromatography (silica gel, eluent of 0-25% ethyl acetate/petroleum ether gradient) to afford the title compound tert-butyl (S)-4-(6-bromopyridin-2-yl)-2-methylpiperazine-1-carboxylate 331 (1 g, 49%) as a yellow solid. LCMS (ESI) m/z=356.09 [M+H]+.

Step B: To a stirred mixture of tert-butyl (S)-4-(6-bromopyridin-2-yl)-2-methylpiperazine-1-carboxylate 331 (700 mg, 1.97 mmol) in 1,4-dioxane (15 mL) is added Sn₂Me₆ (0.5 mL, 2.40 mmol) and Pd(PPh₃)₄ (0.23 g, 0.20 mmol). The resulting mixture is stirred at 110° C. for 2 hours. KF solution is added, and the mixture is stirred for 30 minutes. The mixture is filtered and concentrated under reduced pressure to give the title compound tert-butyl (S)-2-methyl-4-(6-(trimethylstannyl)pyridin-2-yl)piperazine-1-carboxylate 332 (952.6 mg, crude) as a black oil which is used directly in the next step without further purification. LCMS (ESI) m/z=442.14 [M+H]+.

Step C: To a stirred mixture of tert-butyl (S)-2-methyl-4-(6-(trimethylstannyl)pyridin-2-yl)piperazine-1-carboxylate 332 (952.6 mg, 2.16 mmol) in 1,4-dioxane (50 mL) is added 3-bromopyrazolo[1,5-a]pyridine (468 mg, 2.38 mmol), K₃PO₄ (2.29 g, 10.79 mmol) and Xphos Pd G3 (182.8 mg, 0.22 mmol). The resulting mixture is stirred at 80° C. for 2 hours under nitrogen. The reaction mixture is poured into water and then extracted with ethyl acetate three times. The combined organic layers are washed with brine, dried over anhydrous Na₂SO₄, and the mixture is filtered and concentrated under reduced pressure to give a residue. The residue is purified by column chromatography (silica gel, eluent of 0-25% ethyl acetate/petroleum ether gradient) to afford the title compound tert-butyl (S)-2-methyl-4-(6-(pyrazolo[1,5-a]pyridin-3-yl)pyridin-2-yl)piperazine-1-carboxylate 3 (339 mg, 40%) as a yellow solid. LCMS (ESI) m/z=393.22 [M+H]+.

Step D: A mixture of tert-butyl (S)-2-methyl-4-(6-(pyrazolo[1,5-a]pyridin-3-yl)pyridin-2-yl)piperazine-1-carboxylate 3 (200 mg, 0.508 mmol) in HCl/dioxane (3 mL) is stirred at room temperature for 2 hours. The reaction mixture is concentrated under reduced pressure to give a residue which is purified by prep-HPLC (C18/S-5 um/12 nm, 10-95% MeCN in water with 0.1% FA) to give the title compound (S)-3-(6-(3-methylpiperazin-1-yl)pyridin-2-yl)pyrazolo[1,5-a]pyridine formate (Compound 70) (64.2 mg, 43% yield) as a white solid. LCMS (ESI) m/z=294 [M+H]+.

¹H NMR (400 MHz, CD₃OD) δ=8.58 (s, 1H), 8.52-8.45 (m, 1H), 8.39-8.33 (m, 1H), 8.30-8.22 (m, 1H), 7.59-7.49 (m, 1H), 7.32-7.22 (m, 1H), 7.12-7.04 (m, 1H), 6.93-6.84 (m, 1H), 6.70-6.62 (m, 1H), 4.46-4.33 (m, 2H), 3.51 (dd, J=12, 3.2 Hz, 1H), 3.47-3.39 (m, 1H), 3.36-3.16 (m, 2H), 3.10-2.99 (m, 1H), 1.46-1.43 (m, 3H).

### Example 71

### 5-chloro-3-(6-((3S,5R)-3,4,5-trimethylpiperazin-1-yl)pyridin-2-yl)pyrazolo[1,5-a]pyridine

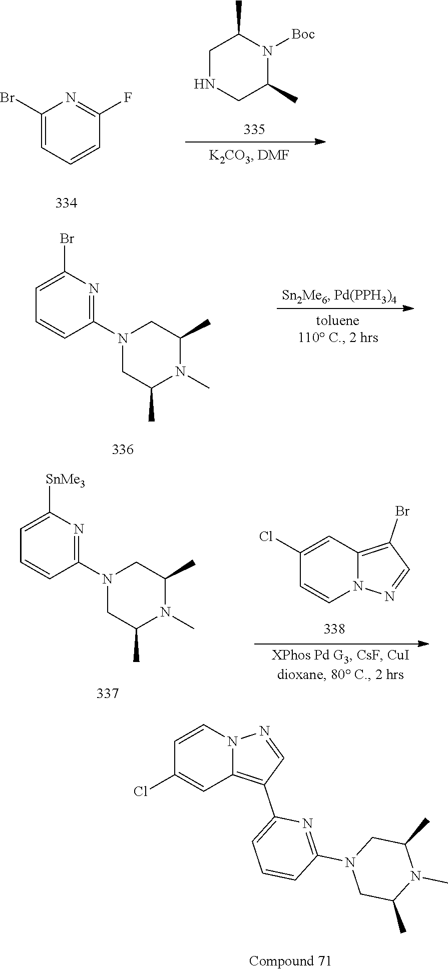

Step A: To a stirred mixture of 2-bromo-6-fluoropyridine 334 (1 g, 5.68 mmol) in DMF (25 mL) is added (2R,6S)-1,2,6-trimethylpiperazine 335 (0.73 g, 5.68 mmol) and K₂CO₃ (3.93 g, 28.4 mmol). The resulting mixture is stirred at 100° C. for 16 hours. The reaction mixture is poured into water and then extracted with ethyl acetate three times. The combined organic layers are washed with brine, dried over anhydrous Na₂SO₄, and the mixture is filtered and concentrated under reduced pressure to give a residue. The residue is purified by column chromatography (silica gel, eluent of 0-25% ethyl acetate/petroleum ether gradient) to afford the title compound (2S,6R)-4-(6-bromopyridin-2-yl)-1,2,6-trimethylpiperazine 336 (1.12 g, 69%) as a yellow solid. LCMS (ESI) m/z=284.07 [M+H]+.

Step B: To a stirred mixture of (2S,6R)-4-(6-bromopyridin-2-yl)-1,2,6-trimethylpiperazine 336 (1.12 g, 3.94 mmol) in 1,4-dioxane (50 mL) is added Sn₂Me₆ (1.68 g, 5.12 mmol) and Pd(PPh₃)₄ (0.45 g, 0.39 mmol). The resulting mixture is stirred at 110° C. for 2 hours under nitrogen. KF solution is added, and the mixture is stirred for 30 minutes. The mixture is filtered and concentrated under reduced pressure to give the title compound (2S,6R)-1,2,6-trimethyl-4-(6-(trimethylstannyl)pyridin-2-yl)piperazine 337 (1.5 g, crude) as a black oil which is used directly in the next step without further purification. LCMS (ESI) m/z=370.12 [M+H]+.

Step C: To stirred mixture of (2S,6R)-1,2,6-trimethyl-4-(6-(trimethylstannyl)pyridin-2-yl)piperazine 337 (200 mg, 0.54 mmol) in 1,4-dioxane (5 mL) is added 3-bromo-5-chloropyrazolo[1,5-a]pyridine 338 (125.4 mg, 0.54 mmol), K₃PO₄ (575 mg, 2.71 mmol) and Xphos pd G₃ (45.86 mg, 0.05 mmol). The resulting mixture is stirred at 80° C. for 2 hours. The reaction mixture is concentrated under reduced pressure to give a residue, and the residue is purified by prep-HPLC (C₁₈/S-5 um/12 nm, 30-95% MeCN in water with 0.1% TFA) to give the title compound 5-chloro-3-(6-((3S,5R)-3,4,5-trimethylpiperazin-1-yl)pyridin-2-yl)pyrazolo[1,5-a]pyridine (Compound 71) (5.9 mg, 3% yield) as a yellow solid. LCMS (ESI) m/z=356.16 [M+H]+.

¹H NMR (400 MHz, CD₃OD) δ=8.57 (d, J=7.2 Hz, 1H), 8.55-8.47 (m, 2H), 7.72-7.62 (m, 1H), 7.33-7.21 (m, 1H), 7.01-6.88 (m, 1H), 6.86-6.75 (m, 1H), 4.65-4.60 (m, 2H), 3.53-3.50 (m, 2H), 3.18-3.06 (m, 2H), 3.01-2.97 (m, 2H), 1.59-1.43 (m, 7H).

### Example 72

### N-(3-(6-(piperidin-3-yl)pyridin-2-yl)pyrazolo[1,5-a]pyridin-5-yl)picolinamide

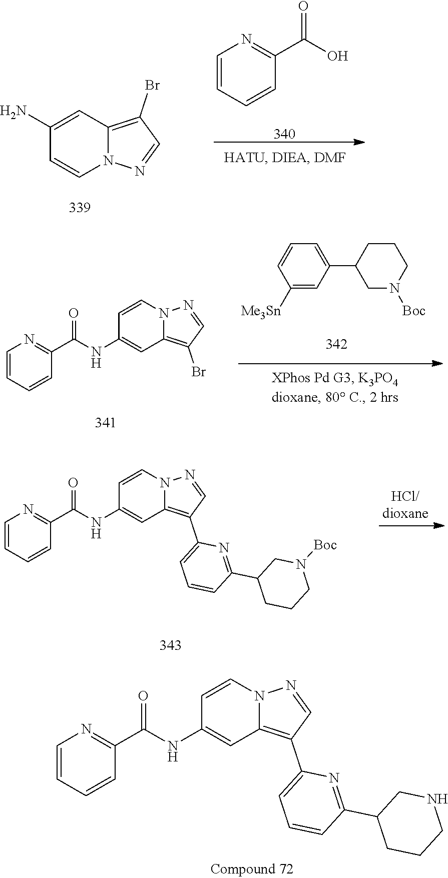

Step A: To a mixture of 3-bromopyrazolo[1,5-a]pyridin-5-amine 339 (500 mg, 2.36 mmol) and picolinic acid 340 (348.3 mg, 2.83 mmol) in DMF (10.0 mL) is added HATU (1075.9 mg, 2.83 mmol) and DIPEA (1219 mg, 9.43 mmol). The reaction mixture is stirred at room temperature for 2 hours. The reaction mixture is poured into water and extracted with ethyl acetate three times. The combined organic phase is concentrated under reduced pressure. The resulting residue is purified by column chromatography (silica gel, eluent of 0-30% ethyl acetate/petroleum ether gradient) to give the title compound N-(3-bromopyrazolo[1,5-a]pyridin-5-yl)picolinamide 341 (395 mg, 53% yield) as a white solid. LCMS (ESI) m/z=317 [M+H]+.

Step B: To a mixture of N-(3-bromopyrazolo[1,5-a]pyridin-5-yl)picolinamide 341 (80.0 mg, 0.25 mmol) and tert-butyl 3-(6-(trimethylstannyl)pyridin-2-yl)piperidine-1-carboxylate 342 (128.69 mg, 0.30 mmol) in dioxane (4 mL) is added K₃PO₄ (267.71 mg, 1.26 mmol) and Xphos Pd G₃ (21.35 mg, 0.03 mmol). The reaction mixture is stirred at 80° C. for 5 hours under nitrogen. The mixture is concentrated under reduced pressure. The residue is purified by column chromatography (silica gel, eluent of 0-20% MeOH/DCM gradient) to give the title compound tert-butyl 3-(6-(5-(picolinamido)pyrazolo[1,5-a]pyridin-3-yl)pyridin-2-yl)piperidine-1-carboxylate 3 (50 mg, 40% yield) as a yellow solid. LCMS (ESI) m/z=499.3 [M+H]+.

Step C: A mixture of tert-butyl 3-(6-(5-(picolinamido)pyrazolo[1,5-a]pyridin-3-yl)pyridin-2-yl)piperidine-1-carboxylate 3 (50 mg, 0.10 mmol) in HCl/dioxane (2 mL, 4M) is stirred at room temperature for 1 hour under nitrogen. The reaction mixture is concentrated under reduced pressure. The residue is purified by prep-HPLC (YMC-ActusTriart C18, 20%-68% MeCN in water with 0.1% FA) to give the title compound N-(3-(6-(piperidin-3-yl)pyridin-2-yl)pyrazolo[1,5-a]pyridin-5-yl)picolinamide (Compound 72) (2.6 mg, 6.5% yield) as a yellow solid. LCMS (ESI) m/z=399.4 [M+H]+.

¹H NMR (400 MHz, CD₃OD) δ=9.90 (d, J=2.0 Hz, 1H), 8.76 (d, J=4.4 Hz, 1H), 8.59-8.52 (m, 2H), 8.50 (s, 1H), 8.30 (d, J=7.6 Hz, 1H), 8.10-8.02 (m, 1H), 7.77-7.71 (m, 1H), 7.71-7.68 (m, 1H), 7.67-7.61 (m, 1H), 7.15-7.08 (m, 2H), 4.02 (t, J=12.0 Hz, 1H), 3.72-3.66 (m, 1H), 3.66-3.58 (m, 1H), 3.57-3.49 (m, 1H), 3.29-3.23 (m, 1H), 2.32-2.21 (m, 1H), 2.20-2.10 (m, 2H), 2.03-1.91 (m, 1H).

### Example 73

### N-(3-(6-(piperidin-3-yl)pyridin-2-yl)pyrazolo[1,5-a]pyridin-5-yl)-2-(pyridin-2-yloxy)acetamide

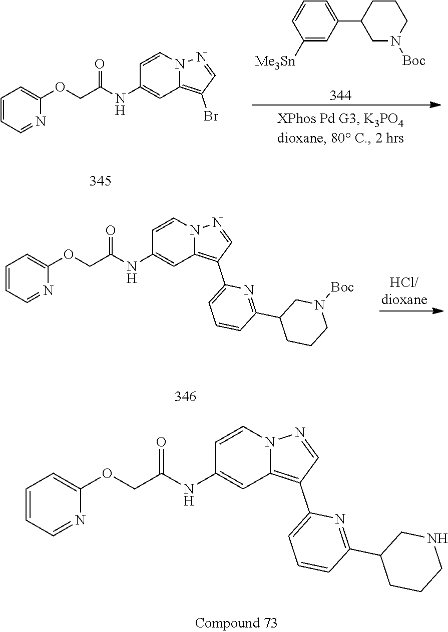

Step A: To a mixture of tert-butyl 3-(6-(trimethylstannyl)pyridin-2-yl)piperidine-1-carboxylate 344 (79.6 mg, 0.19 mmol) in dioxane (2 mL) is added N-(3-bromopyrazolo[1,5-a]pyridin-5-yl)-2-(pyridin-2-yloxy)acetamide 345 (50 mg, 0.14 mmol), XPhos Pd G₃ (24.3 mg, 0.029 mmol) and K₃PO₄ (91.7 mg, 0.43 mmol). The resulting mixture is stirred at 80° C. for 2 hours under N₂. The mixture is filtered and concentrated under reduced pressure to give a residue. The residue is purified by prep-TLC (DCM:MeOH=10:1) to afford the title compound tert-butyl 3-(6-(5-(2-(pyridin-2-yloxy)acetamido)pyrazolo[1,5-a]pyridin-3-yl)pyridin-2-yl)piperidine-1-carboxylate 346 (55 mg, 72% yield) as a yellow solid. LCMS (ESI MS) m/z=529 [M+H]+.

Step B: A mixture of tert-butyl 3-(6-(5-(2-(pyridin-2-yloxy)acetamido)pyrazolo[1,5-a]pyridin-3-yl)pyridin-2-yl)piperidine-1-carboxylate 346 (55 mg, 0.10 mmol) in DCM (2 mL) and HCl/dioxane (3 mL) is stirred at room temperature for 0.5 hour. The reaction mixture is concentrated under reduced pressure to give a residue which is purified by prep-HPLC (C18 250*21 mm MeCN in water with 0.1% FA) to afford the title compound N-(3-(6-(piperidin-3-yl)pyridin-2-yl)pyrazolo[1,5-a]pyridin-5-yl)-2-(pyridin-2-yloxy)acetamide (Compound 73) (9.4 mg, 21% yield) as a yellow solid. LCMS (ESI MS) m/z=429 [M+H]+.

¹H NMR (400 MHz, CD₃OD) δ=9.74 (d, J=2.4 Hz, 1H), 8.52 (d, J=7.2 Hz, 1H), 8.48 (d, J=5.6 Hz, 1H), 7.72-7.67 (m, 2H), 7.67-7.61 (m, 2H), 7.03 (dd, J=7.2, 1.2 Hz, 1H), 6.77 (dd, J=7.6, 2.4 Hz, 1H), 6.63 (d, J=8.8 Hz, 1H), 6.52-6.42 (m, 1H), 5.00 (d, J=16.0 Hz, 1H), 4.90 (t, J=10.0 Hz, 1H), 3.72 (t, J=12.0 Hz, 1H), 3.39 (d, J=10.4 Hz, 1H), 3.12 (ddd, J=11.6, 10.0, 3.6 Hz, 3H), 2.20 (d, J=13.6 Hz, 1H), 2.00 (d, J=9.6 Hz, 1H), 1.83 (d, J=14.4 Hz, 2H).

### Example 74

### 3-cyano-N-(3-(6-(piperidin-3-yl)pyridin-2-yl)pyrazolo[1,5-a]pyridin-5-yl)propanamide

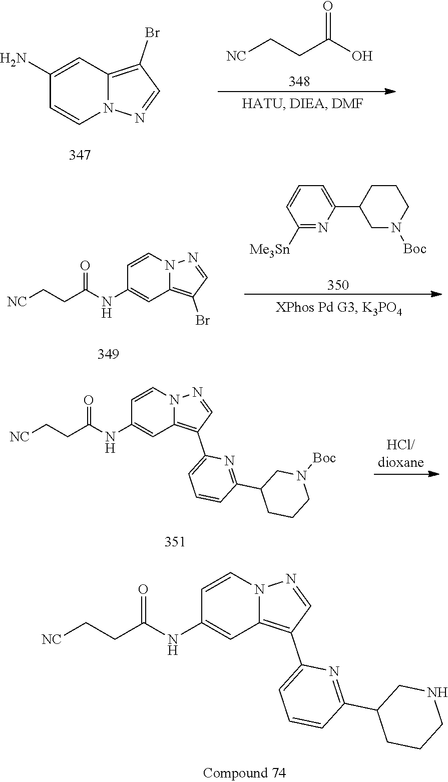

Step A: To a mixture of 3-bromopyrazolo[1,5-a]pyridin-5-amine 347 (300 mg, 1.42 mmol) and 3-cyanopropanoic acid 348 (140.2 mg, 1.42 mmol) in DMF (6.0 mL) is added HATU (807.1 mg, 2.12 mmol) and DIPEA (731.5 mg, 5.66 mmol). The reaction mixture is stirred at 50° C. for 16 hours. The reaction mixture is poured into water and extracted with ethyl acetate three times. The combined organic layers are washed with brine, dried over anhydrous Na₂SO₄, and the mixture is filtered and concentrated under reduced pressure to give a residue. The residue is purified by column chromatography (silica gel, eluent of 0-30% MeOH/DCM gradient) to give the title compound N-(3-bromopyrazolo[1,5-a]pyridin-5-yl)-3-cyanopropanamide 349 (142 mg, 34% yield) as a solid. LCMS (ESI) m/z=292.9 [M+H]+.

Step B: To a mixture of N-(3-bromopyrazolo[1,5-a]pyridin-5-yl)-3-cyanopropanamide 349 (140 mg, 0.48 mmol) and tert-butyl 3-(6-(trimethylstannyl)pyridin-2-yl)piperidine-1-carboxylate 350 (243.7 mg, 0.57 mmol) in dioxane (5 mL) is added K₃PO₄ (506.9 mg, 2.39 mmol) and Xphos Pd G₃ (40.4 mg, 0.05 mmol). The reaction mixture is stirred at 80° C. for 5 hours under nitrogen. The mixture is filtered and concentrated under reduced pressure. The residue is purified by column chromatography (silica gel, eluent of 0-20% MeOH/DCM gradient) to give the title compound tert-butyl 3-(6-(5-(3-cyanopropanamido)pyrazolo[1,5-a]pyridin-3-yl)pyridin-2-yl)piperidine-1-carboxylate 35 (37 mg, 16% yield) as a yellow solid. LCMS (ESI) m/z=475.2 [M+H]+.

Step C: To a solution of tert-butyl 3-(6-(5-(3-cyanopropanamido)pyrazolo[1,5-a]pyridin-3-yl)pyridin-2-yl)piperidine-1-carboxylate 351 (37 mg, 0.08 mmol) in DCM (1.0 mL) is added HCl/dioxane (1.0 mL, 4M). The reaction mixture is stirred at room temperature for 1 hour under nitrogen. The reaction mixture is concentrated under reduced pressure. The residue is purified by prep-HPLC (YMC-ActusTriart C18, 20%-68% MeCN in water with 0.1% TFA) to give the title compound 3-cyano-N-(3-(6-(piperidin-3-yl)pyridin-2-yl)pyrazolo[1,5-a]pyridin-5-yl)propanamide (Compound 74) (8.3 mg, 28% yield) as a yellow solid. LCMS (ESI) m/z=375.2 [M+H]+.

¹H NMR (400 MHz, CD₃OD) δ=9.66 (d, J=2.4 Hz, 1H), 8.51-8.44 (m, 2H), 7.72 (t, J=7.6 Hz, 1H), 7.66 (d, J=8.0 Hz, 1H), 7.08 (d, J=7.2 Hz, 1H), 6.77-6.70 (m, 1H), 3.91 (t, J=12.0 Hz, 1H), 3.65-3.53 (m, 2H), 3.51-3.41 (m, 1H), 3.28-3.15 (m, 1H), 2.90-2.84 (m, 2H), 2.84-2.78 (m, 2H), 2.30-2.15 (m, 1H), 2.15-2.12 (m, 1H), 2.12-2.04 (m, 1H), 2.00-1.77 (m, 1H).

### Example 75

### 4-cyano-N-(3-(6-(piperidin-3-yl)pyridin-2-yl)pyrazolo[1,5-a]pyridin-5-yl)butanamide

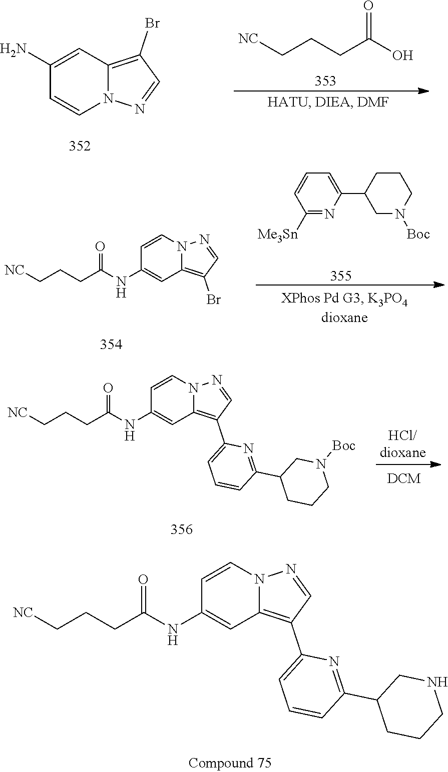

Step A: To a mixture of 3-bromopyrazolo[1,5-a]pyridin-5-amine 352 (400 mg, 1.40 mmol) and 4-cyanobutanoic acid 353 (158.7 mg, 1.40 mmol) in DMF (5.0 mL) is added HATU (800.6 mg, 2.11 mmol) and DIPEA (1.08 mg, 8.42 mmol). The reaction mixture is stirred at room temperature for 16 hours. The reaction mixture is poured into water and then extracted with ethyl acetate three times. The combined organic layers re washed with brine, dried over anhydrous Na₂SO₄, and the mixture is filtered and concentrated under reduced pressure to give a residue. The residue is purified by column chromatography (silica gel, eluent of 0-30% MeOH/DCM gradient) to give the title compound N-(3-bromopyrazolo[1,5-a]pyridin-5-yl)-4-cyanobutanamide 354 (210 mg, 49% yield) as a solid. LCMS (ESI) m/z=306.9 [M+H]+.

Step B: To a mixture of N-(3-bromopyrazolo[1,5-a]pyridin-5-yl)-4-cyanobutanamide 354 (160 mg, 0.52 mmol) and 355 (265.7 mg, 0.63 mmol) in dioxane (5 mL) is added K₃PO₄ (552.8 mg, 2.61 mmol) and Xphos Pd G₃ (44.1 mg, 0.05 mmol). The reaction mixture is stirred at 80° C. for 5 hours under nitrogen. The mixture is filtered and concentrated under reduced pressure. The residue is purified by column chromatography (silica gel, eluent of 0-20% MeOH/DCM gradient) to give the title compound tert-butyl 3-(6-(5-(4-cyanobutanamido)pyrazolo[1,5-a]pyridin-3-yl)pyridin-2-yl)piperidine-1-carboxylate 356 (70 mg, 27% yield) as a yellow solid. LCMS (ESI) m/z=489.2 [M+H]+.

Step C: To a solution of tert-butyl 3-(6-(5-(4-cyanobutanamido)pyrazolo[1,5-a]pyridin-3-yl)pyridin-2-yl)piperidine-1-carboxylate 356 (70 mg, 0.14 mmol) in DCM (1.0 mL) is added HCl/dioxane (2.0 mL, 4M). The reaction mixture is stirred at room temperature for 1 hour under nitrogen. The reaction mixture is concentrated under reduced pressure. The residue is purified by prep-HPLC (YMC-ActusTriart C18, 20%-68% MeCN in water with 0.1% FA) to give the title compound 4-cyano-N-(3-(6-(piperidin-3-yl)pyridin-2-yl)pyrazolo[1,5-a]pyridin-5-yl)butanamide (Compound 75) (20 mg, 35% yield) as a yellow solid. LCMS (ESI) m/z=389.3 [M+H]+.

¹H NMR (400 MHz, CD₃OD) δ=9.58 (d, J=2.0 Hz, 1H), 8.49 (s, 1H), 8.46-8.41 (m, 2H), 7.69 (t, J=7.6 Hz, 1H), 7.62 (d, J=7.2 Hz, 1H), 7.06 (d, J=7.2 Hz, 1H), 6.82-6.68 (m, 1H), 3.85 (t, J=12.0 Hz, 1H), 3.63-3.56 (m, 1H), 3.56-3.36 (m, 2H), 3.27-3.15 (m, 1H), 2.69-2.47 (m, 4H), 2.32-2.10 (m, 2H), 2.10-2.06 (m, 2H), 2.06-1.85 (m, 2H).

### Example 76

### N-(3-(6-(piperidin-3-yl)pyridin-2-yl)pyrazolo[1,5-a]pyridin-5-yl)-2-(pyridin-4-yloxy)acetamide

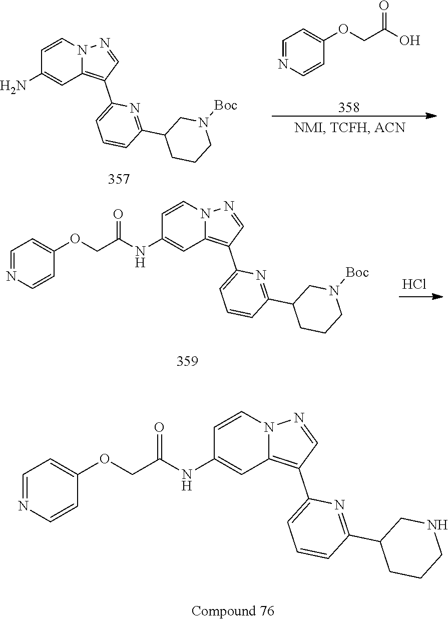 [SMILES: CC(C)(C)OC(=O)N1CCCC(c2cccc(-c3cnn4ccc(N)cc34)n2)C1 ; O=C(O)COc1ccncc1 ; CC(C)(C)OC(=O)N1CCCC(c2cccc(-c3cnn4ccc(NC(=O)COc5ccncc5)cc34)n2)C1 ; Cl ; O=C(COc1ccncc1)Nc1ccn2ncc(-c3cccc(C4CCCNC4)n3)c2c1]

Step A: To a mixture of tert-butyl 3-(6-(5-aminopyrazolo[1,5-a]pyridin-3-yl)pyridin-2-yl)piperidine-1-carboxylate 357 (50 mg, 0.13 mmol) and 2-(pyridin-4-yloxy)acetic acid (21.4 mg, 0.14 mmol) in ACN (3.0 mL) is added NMI (31.3 mg, 0.38 mmol) and TCFH (107 mg, 0.38 mmol). The reaction mixture is stirred at room temperature for 6 hours. The reaction mixture is poured into water and then extracted with ethyle acetate three times. The combined organic layers are washed with brine, dried over anhydrous Na₂SO₄, and the mixture is filtered and concentrated under reduced pressure to give a residue. The residue is purified by column chromatography (silica gel, eluent of 0-30% MeOH/DCM gradient) to give the title compound tert-butyl 3-(6-(5-(2-(pyridin-4-yloxy)acetamido)pyrazolo[1,5-a]pyridin-3-yl)pyridin-2-yl)piperidine-1-carboxylate 3 (20 mg, 30% yield) as a solid. LCMS (ESI) m/z=529 [M+H]+.

Step B: To a solution of tert-butyl 3-(6-(5-(2-(pyridin-4-yloxy)acetamido)pyrazolo[1,5-a]pyridin-3-yl)pyridin-2-yl)piperidine-1-carboxylate 3 (20 mg, 0.038 mmol) in DCM (1.0 mL) is added HCl/dioxane (1.0 mL, 4M). The reaction mixture is stirred at room temperature for 1 hour under nitrogen. The reaction mixture is concentrated under reduced pressure. The residue is purified by prep-HPLC (YMC-ActusTriart C18, 20%-68% MeCN in water with 0.1% TFA) to give the title compound N-(3-(6-(piperidin-3-yl)pyridin-2-yl)pyrazolo[1,5-a]pyridin-5-yl)-2-(pyridin-4-yloxy)acetamide (Compound 76) (2.4 mg, 15% yield) as a yellow solid. LCMS (ESI) m/z=429.3 [M+H]+.

¹H NMR (400 MHz, CD₃OD) δ=9.62 (d, J=2.0 Hz, 1H), 8.58-8.53 (m, 1H), 8.50 (s, 1H), 8.40 (d, J=7.6 Hz, 2H), 7.74-7.65 (m, 2H), 7.14 (d, J=7.2 Hz, 2H), 7.09-7.01 (m, 1H), 6.81 (dd, J=7.6, 2.4 Hz, 1H), 5.35 (s, 2H), 3.63 (t, J=12.0 Hz, 1H), 3.47-3.41 (m, 1H), 3.19-3.11 (m, 2H), 3.10-2.99 (m, 1H), 2.29-2.13 (m, 1H), 2.09-1.99 (m, 1H), 1.95-1.79 (m, 2H).

### Example 77

### 2-cyano-N-(3-(6-(piperidin-3-yl)pyridin-2-yl)pyrazolo[1,5-a]pyridin-5-yl)acetamide

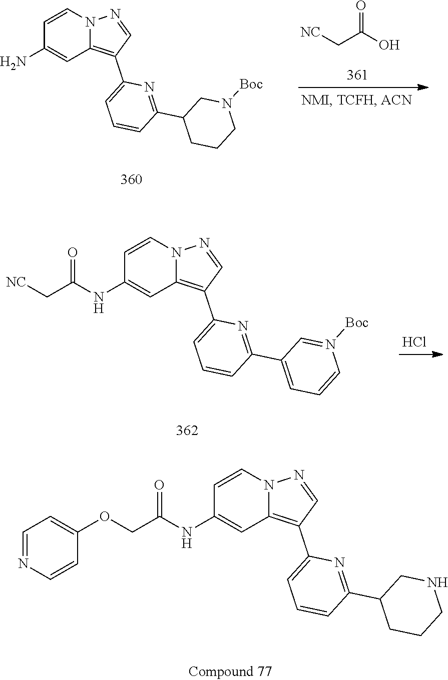

Step A: To a mixture of tert-butyl 3-(6-(5-aminopyrazolo[1,5-a]pyridin-3-yl)pyridin-2-yl)piperidine-1-carboxylate 360 (40 mg, 0.10 mmol) and 2-cyanoacetic acid (9.5 mg, 0.11 mmol) in ACN (3.0 mL) is added NMI (25 mg, 0.31 mmol) and TCFH (85.6 mg, 0.31 mmol). The reaction mixture is stirred at room temperature for 6 hours. The reaction mixture is poured into water and then extracted with ethyl acetate three times. The combined organic layers are washed with brine, dried over anhydrous Na₂SO₄, and the mixture is filtered and concentrated under reduced pressure to give a residue. The residue is purified by column chromatography (silica gel, eluent of 0-30% MeOH/DCM gradient) to give the title compound tert-butyl 3-(6-(5-(2-cyanoacetamido)pyrazolo[1,5-a]pyridin-3-yl)pyridin-2-yl)piperidine-1-carboxylate 62 (20 mg, 43% yield) as a solid. LCMS (ESI) m/z=461.2 [M+H]+.

Step B: To a solution of tert-butyl 3-(6-(5-(2-cyanoacetamido)pyrazolo[1,5-a]pyridin-3-yl)pyridin-2-yl)piperidine-1-carboxylate 362 (20 mg, 0.043 mmol) in DCM (1.0 mL) is added HCl/dioxane (1.0 mL, 4M). The reaction mixture is stirred at room temperature for 1 hour under nitrogen. The reaction mixture is concentrated under reduced pressure. The residue is purified by prep-HPLC (YMC-ActusTriart C18, 20%-68% MeCN in water with 0.1% FA) to give the title compound 2-cyano-N-(3-(6-(piperidin-3-yl)pyridin-2-yl)pyrazolo[1,5-a]pyridin-5-yl)acetamide (Compound 77) (1.5 mg, 9.6% yield) as a white solid. LCMS (ESI) m/z=361.2 [M+H]+.

¹H NMR (400 MHz, CD₃OD) δ=9.77 (d, J=2.4 Hz, 1H), 8.53-8.49 (m, 2H), 7.75-7.70 (m, 1H), 7.70-7.67 (m, 1H), 7.10-7.07 (m, 1H), 6.71 (dd, J=7.6, 2.4 Hz, 1H), 3.89 (t, J=12.0 Hz, 1H), 3.73-3.65 (m, 1H), 3.58-3.53 (m, 1H), 3.51-3.37 (m, 2H), 3.27-3.19 (m, 1H), 3.19-3.04 (m, 1H), 2.29-2.20 (m, 1H), 2.14-2.11 (m, 1H), 2.10-2.07 (m, 1H), 1.98-1.88 (m, 1H).

### Example 78

### N-(3-(6-(piperidin-3-yl)pyridin-2-yl)pyrazolo[1,5-a]pyridin-5-yl)-2-(pyridin-3-yl)acetamide

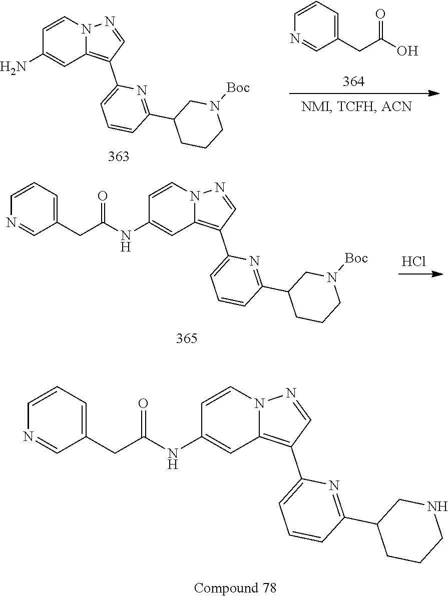 [SMILES: CC(C)(C)OC(=O)N1CCCC(c2cccc(-c3cnn4ccc(N)cc34)n2)C1 ; O=C(O)Cc1cccnc1 ; CC(C)(C)OC(=O)N1CCCC(c2cccc(-c3cnn4ccc(NC(=O)Cc5cccnc5)cc34)n2)C1 ; Cl ; O=C(Cc1cccnc1)Nc1ccn2ncc(-c3cccc(C4CCCNC4)n3)c2c1]

Step A: To a mixture of tert-butyl 3-(6-(5-aminopyrazolo[1,5-a]pyridin-3-yl)pyridin-2-yl)piperidine-1-carboxylate 363 (25 mg, 0.064 mmol) and 2-(pyridin-3-yl)acetic acid (8.7 mg, 0.064 mmol) in ACN (3.0 mL) is added NMI (15.7 mg, 0.19 mmol) and TCFH (53.5 mg, 0.19 mmol). The reaction mixture is stirred at room temperature for 6 hours. The reaction mixture is poured into water and then extracted with ethyl acetate three times. The combined organic layers are washed with brine, dried over anhydrous Na₂SO₄, and the mixture is filtered and concentrated under reduced pressure to give a residue. The residue is purified by column chromatography (silica gel, eluent of 0-30% MeOH/DCM gradient) to give the title compound tert-butyl 3-(6-(5-(2-(pyridin-3-yl)acetamido)pyrazolo[1,5-a]pyridin-3-yl)pyridin-2-yl)piperidine-1-carboxylate 365 (12 mg, 37% yield) as a solid. LCMS (ESI) m/z=513.2 [M+H]+.

Step B: To a solution of tert-butyl 3-(6-(5-(2-(pyridin-3-yl)acetamido)pyrazolo[1,5-a]pyridin-3-yl)pyridin-2-yl)piperidine-1-carboxylate 365 (12.0 mg, 0.023 mmol) in DCM (1.0 mL) is added HCl/dioxane (1.0 mL, 4M). The reaction mixture is stirred at room temperature for 1 hour under nitrogen. The reaction mixture is concentrated under reduced pressure. The residue is purified by prep-HPLC (YMC-ActusTriart C18, 20%-68% MeCN in water with 0.1% FA) to give the title compound N-(3-(6-(piperidin-3-yl)pyridin-2-yl)pyrazolo[1,5-a]pyridin-5-yl)-2-(pyridin-3-yl)acetamide (Compound 78) (6.0 mg, 62% yield) as a white solid. LCMS (ESI) m/z=413.3 [M+H]+.

¹H NMR (400 MHz, CD₃OD) δ=9.70 (d, J=2.0 Hz, 1H), 8.58 (s, 1H), 8.52-8.37 (m, 4H), 7.87 (d, J=7.6 Hz, 1H), 7.72-7.61 (m, 2H), 7.52-7.40 (m, 1H), 7.04 (d, J=7.2 Hz, 1H), 6.79 (dd, J=7.6, 2.4 Hz, 1H), 3.89 (s, 2H), 3.74 (t, J=12.0 Hz, 1H), 3.50-3.42 (m, 1H), 3.25-3.10 (m, 3H), 2.24-2.08 (m, 1H), 2.08-1.97 (m, 1H), 1.94-1.76 (m, 2H).

### Example 79

### N-(3-(6-(piperidin-3-yl)pyridin-2-yl)pyrazolo[1,5-a]pyridin-5-yl)-2-(pyridin-4-ylamino)acetamide

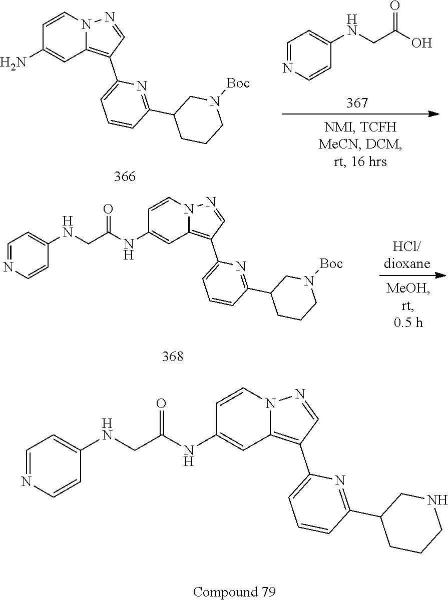 [SMILES: CC(C)(C)OC(=O)N1CCCC(c2cccc(-c3cnn4ccc(N)cc34)n2)C1 ; O=C(O)CNc1ccncc1 ; CC(C)(C)OC(=O)N1CCCC(c2cccc(-c3cnn4ccc(NC(=O)CNc5ccncc5)cc34)n2)C1 ; "HCl/dioxane" ; O=C(CNc1ccncc1)Nc1ccn2ncc(-c3cccc(C4CCCNC4)n3)c2c1]

Step A: To a solution of tert-butyl 3-(6-(5-aminopyrazolo[1,5-a]pyridin-3-yl)pyridin-2-yl)piperidine-1-carboxylate 366 (100 mg, 0.254 mmol), TCFH (356.3 mg, 1.27 mmol) and pyridin-4-ylglycine 367 (115.9 mg, 0.76 mmol) in MeCN (3 mL) is added a mixture of NMI (0.10 mL, 1.27 mmol) in DCM (3 mL). The mixture is stirred at room temperature for 16 hours. the mixture is poured into water and then extracted with ethyl acetate twice. The combined organic phase is dried over Na₂SO₄ and concentrated. The residue is purified by column chromatography (silica gel, eluent of 0-8% MeOH/DCM gradient) to give the title compound tert-butyl 3-(6-(5-(2-(pyridin-4-ylamino)acetamido)pyrazolo[1,5-a]pyridin-3-yl)pyridin-2-yl)piperidine-1-carboxylate 3 (50 mg, 37% yield) as a light yellow gum. LCMS: m/z 528 [M+H]+.

Step B: To a solution of tert-butyl 3-(6-(5-(2-(pyridin-4-ylamino)acetamido)pyrazolo[1,5-a]pyridin-3-yl)pyridin-2-yl)piperidine-1-carboxylate 3 (45 mg, 0.085 mmol) in MeOH (1 mL) is added HCl/dioxane (1 mL, 4 M). The mixture is stirred at room temperature for 0.5 hour. The reaction mixture is concentrated under vacuum. The residue is purified by prep-HPLC (C18, 250*21 mm MeCN in water with 0.1% FA) to give the title compound N-(3-(6-(piperidin-3-yl)pyridin-2-yl)pyrazolo[1,5-a]pyridin-5-yl)-2-(pyridin-4-ylamino)acetamide (Compound 79) (6.1 mg, 17% yield) as a yellow solid. LCMS: m/z 428 [M+H]+.

¹H NMR (400 MHz, CD₃OD) δ=9.61 (d, J=2.0 Hz, 1H), 8.65-8.39 (m, 4H), 8.11 (d, J=7.2 Hz, 2H), 7.82-7.62 (m, 2H), 7.05 (d, J=6.8 Hz, 1H), 6.91 (d, J=7.2 Hz, 2H), 6.86-6.76 (m, 1H), 5.20 (s, 2H), 3.67-3.58 (m, 1H), 3.48-3.39 (m, 1H), 3.17-3.10 (m, 2H), 2.25-2.12 (m, 1H), 2.09-1.99 (m, 1H), 1.95-1.77 (m, 2H), 1.40-1.28 (m, 1H).

### Example 80

### N-(3-(6-(piperidin-3-yl)pyridin-2-yl)pyrazolo[1,5-a]pyridin-5-yl)-2-(pyridin-3-ylamino)acetamide

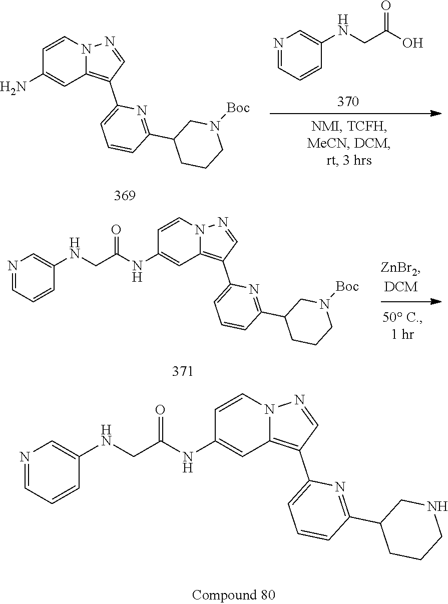

Step A: To a solution of tert-butyl 3-(6-(5-aminopyrazolo[1,5-a]pyridin-3-yl)pyridin-2-yl)piperidine-1-carboxylate 369 (100 mg, 0.254 mmol), TCFH (213.9 mg, 0.762 mmol) and pyridin-3-ylglycine 370 (38.7 mg, 0.254 mmol) in MeCN (3 mL) is added a mixture of NMI (0.06 mL, 0.76 mmol) in DCM (3 mL). The mixture is stirred at room temperature for 3 hours. The reaction mixture is concentrated under vacuum and purified by column chromatography (silica gel, eluent of 0-5% MeOH/DCM gradient) to give the title compound tert-butyl 3-(6-(5-(2-(pyridin-3-ylamino)acetamido)pyrazolo[1,5-a]pyridin-3-yl)pyridin-2-yl)piperidine-1-carboxylate 371 (70 mg, 52% yield) as a light yellow gum. LCMS: m/z=528 [M+H]+.

Step B: To a solution of tert-butyl 3-(6-(5-(2-(pyridin-3-ylamino)acetamido)pyrazolo[1,5-a]pyridin-3-yl)pyridin-2-yl)piperidine-1-carboxylate 371 (70 mg, 0.133 mmol) in DCM (3 mL) is added ZnBr₂ (298.8 mg, 1.33 mmol). The mixture is stirred at 50° C. for 1 hour. The reaction mixture is concentrated under vacuum. The residue is purified by prep-HPLC (C18, 250*20 mm, 5 um MeCN in water with 0.1% TFA) to give the title compound N-(3-(6-(piperidin-3-yl)pyridin-2-yl)pyrazolo[1,5-a]pyridin-5-yl)-2-(pyridin-3-ylamino)acetamide (Compound 80) (20.7 mg, 36% yield) as a yellow solid. LCMS: m/z 428 [M+H]+.

¹H NMR (400 MHz, CD₃OD) δ=9.58 (d, J=2.0 Hz, 1H), 8.52 (d, J=7.6 Hz, 1H), 8.49 (s, 1H), 8.17 (d, J=2.4 Hz, 1H), 8.05 (d, J=5.2 Hz, 1H), 7.86-7.76 (m, 2H), 7.73 (t, J=7.6 Hz, 1H), 7.67 (d, J=8.0 Hz, 1H), 7.08 (d, J=7.2 Hz, 1H), 6.84 (dd, J=7.6, 2.4 Hz, 1H), 4.26 (s, 2H), 3.84-3.73 (m, 1H), 3.60-3.50 (m, 1H), 3.24-3.15 (m, 2H), 2.20-2.02 (m, 2H), 1.91-1.78 (m, 2H), 1.37-1.26 (m, 1H).

### Example 81

### N-(3-(6-(piperidin-3-yl)pyridin-2-yl)pyrazolo[1,5-a]pyridin-5-yl)-2-(pyridin-2-yl)acetamide

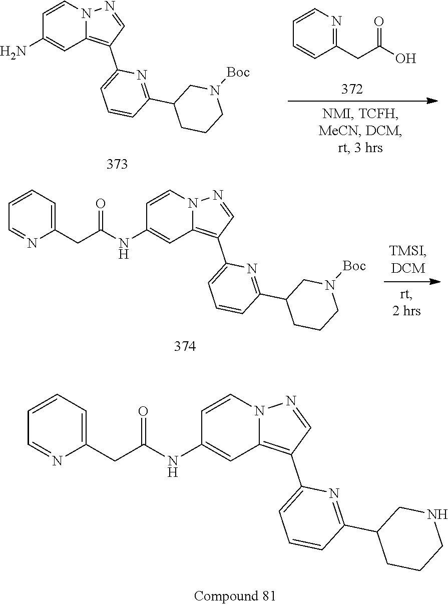

Step A: To a solution of 2-(pyridin-2-yl)acetic acid 372 (180 mg, 1.04 mmol), tert-butyl 3-(6-(5-aminopyrazolo[1,5-a]pyridin-3-yl)pyridin-2-yl)piperidine-1-carboxylate 373 (100 mg, 0.25 mmol) and TCFH (356.5 mg, 1.27 mmol) in MeCN (4 mL) is added a mixture of NMI (0.10 mL, 1.27 mmol) in DCM (3 mL). The mixture is stirred at room temperature for 1 hour. The reaction mixture is quenched by H₂O and extracted with DCM twice. The combined organic phase is dried over Na₂SO₄ and concentrated under vacuum. The residue is purified by column chromatography (silica gel, eluent of 0-5% MeOH/DCM gradient) to give the title compound tert-butyl 3-(6-(5-(2-(pyridin-2-yl)acetamido)pyrazolo[1,5-a]pyridin-3-yl)pyridin-2-yl)piperidine-1-carboxylate 374 (100 mg, 76.7%) as a yellow gum. LCMS: m/z 513 [M+H]+.

Step B: To a solution of tert-butyl 3-(6-(5-(2-(pyridin-2-yl)acetamido)pyrazolo[1,5-a]pyridin-3-yl)pyridin-2-yl)piperidine-1-carboxylate 374 (120 mg, 0.234 mmol) in DCM (5 mL) is added TMSI (0.100 mL, 0.70 mmol). The mixture is stirred at room temperature for 2 hours. The reaction mixture is concentrated under reduced pressure to give a residue. The residue is purified by prep-HPLC (C18, 250*20 mm, 5 um MeCN in water with 0.1% TFA) to give the title compound N-(3-(6-(piperidin-3-yl)pyridin-2-yl)pyrazolo[1,5-a]pyridin-5-yl)-2-(pyridin-2-yl)acetamide (Compound 81) (18.9 mg, 19.6% yield) as a yellow solid. LCMS: m/z 413.2 [M+H]+.

¹H NMR (400 MHz, CD₃OD) δ=9.59 (s, 1H), 8.81 (d, J=5.2 Hz, 1H), 8.50 (d, J=7.6 Hz, 1H), 8.46 (s, 1H), 8.39 (t, J=7.6 Hz, 1H), 7.93 (d, J=8.0 Hz, 1H), 7.87-7.79 (m, 1H), 7.70 (t, J=7.6 Hz, 1H), 7.64 (d, J=8.0 Hz, 1H), 7.05 (d, J=7.2 Hz, 1H), 6.83 (dd, J=7.2, 1.6 Hz, 1H), 3.80-3.66 (m, 1H), 3.55-3.43 (m, 1H), 3.31 (s, 2H), 3.21-3.11 (m, 2H), 3.10-2.98 (m, 1H), 2.17-1.98 (m, 2H), 1.92-1.78 (m, 2H).

### Example A

### LanthaScreen LRRK2 Biochemical Activity Assay

This Example illustrates that representative compounds of the invention inhibit LRRK2 kinase activity in vitro.

The ability of a compound of Formula (I) to inhibit LRRK2 kinase activity is measured using a LanthaScreen™ kinase activity assay. In general, in a LanthaScreen™ kinase activity assay, kinase, fluorescein-labeled substrate, and ATP are allowed to react. Then EDTA (to stop the reaction) and terbium-labeled antibody (to detect phosphorylated product) are added. In a LanthaScreen™ kinase reaction, the antibody associates with the phosphorylated fluorescein labeled substrate resulting in an increased TR-FRET value. The TR-FRET value is a dimensionless number that is calculated as the ratio of the acceptor (fluorescein) signal to the donor (terbium) signal. The amount of antibody that is bound to the tracer is directly proportional to the amount of phosphorylated substrate present, and in this manner, kinase activity can be detected and measured by an increase in the TR-FRET value.

Accordingly, in this Example, enzyme reactions are carried out in a kinase reaction buffer in the presence or absence of representative compounds of Formula (I) at various concentrations in 384-well plate at room temperature for 60 minutes. The kinase reaction buffer contains human recombinant LRRK2 protein (amino acids 970-2527, 5 nM), ATP (57 μM for LRRK2 wild-type, 134 μM for G2019S mutant protein), Fluorescein-LRRKtide substrate (0.4 mM), 50 mM Tris pH 7.5, 10 mM MgCl₂, 1 mM EGTA, 0.01% Brij™-35, and 2 mM DTT. The final reaction volume for each reaction is 10 μL. The reactions are initated by adding the substrate and stopped by the addition of 10 μL of a TR-FRET dilution buffer supplemented with a kinase quench buffer (10 mM EDTA final) and Tb-anti-pLRRKtide (2 nM final). The plate is further incubated at room temperature for another 60 minutes, and the fluorescent signals are read on an EnVision® multilabel plate reader (PerkinElmer, Waltham, MA) with excitation at 340 nm and emission at 495 and 520 nm. The assay signal is determined as a ratio of FRET-specific signal measured with emission filter at 520 nm to that of the signal measured with Tb-specific emission filter at 495 nm. IC₅₀ values for compound-mediated enzyme inhibition are calculated using appropriate programs in GraphPad Prism software by plotting the logarithm of the compound concentrations versus percent inhibition. All reagents are purchased from Thermo Fisher Scientific.

Table 2 shows the inhibition of LRRK2 wild-type kinase activity by representative compounds of Formula (I). IC₅₀ values of less than or equal to 10 nM are represented by “+++”; IC₅₀ values between 11 nM and 100 nM are represented by “++”; and IC₅₀ values between 101 nM and 400 nM are represented by “+”.

TABLE 2
Inhibition of LRRK2 wild-type kinase activity
by representative compounds of Formula (I)

| Example | IC₅₀ (nM) |
|---|---|
| 1 | + |
| 2 | + |
| 3 | + |
| 4 | ++ |
| 5 | ++ |
| 6 | + |
| 7 | +++ |
| 8 | ++ |
| 9 | ++ |
| 10 | ++ |
| 11 | ++ |
| 12 | + |
| 13 | ++ |
| 14 | + |
| 15 | + |
| 16 | + |
| 17 | ++ |
| 18 | ++ |
| 19 | + |
| 20 | +++ |
| 21 | ++ |
| 22 | ++ |
| 23 | + |
| 24 | + |
| 25 | + |
| 26 | + |
| 27 | + |
| 28 | ++ |
| 29 | ++ |
| 30 | + |
| 31 | + |
| 32 | ++ |
| 33 | + |
| 34 | ++ |
| 35 | ++ |
| 36 | ++ |
| 37 | + |
| 38 | ++ |
| 39 | ++ |
| 40 | ++ |
| 41 | ++ |
| 42 | ++ |
| 43 | ++ |
| 44 | ++ |
| 45 | + |
| 46 | ++ |
| 47 | + |
| 48 | + |
| 49 | ++ |
| 50 | ++ |
| 51 | ++ |
| 52 | ++ |
| 53 | ++ |
| 54 | ++ |
| 55 | +++ |
| 56 | +++ |
| 57 | +++ |
| 58 | +++ |
| 59 | +++ |
| 60 | + |
| 61 | +++ |
| 62 | ++ |
| 63 | +++ |
| 64 | ++ |
| 65 | +++ |
| 66 | +++ |
| 67 | ++ |
| 68 | ++ |
| 69 | ++ |
| 70 | ++ |
| 71 | ++ |
| 72 | ++ |
| 73 | ++ |
| 74 | ++ |
| 75 | ++ |
| 76 | ++ |
| 77 | ++ |
| 78 | ++ |
| 79 | ++ |
| 80 | ++ |
| 81 | ++ |

### Example B

### LRRK2 Cellular Activity Assay

This Example illustrates that representative compounds of the invention inhibit LRRK2 kinase activity in vitro.

The ability of a compound of Formula (I) to inhibit LRRK2 kinase activity is measured using a cell-based quantitative immunocytochemistry LRRK2 kinase inhibition assay. In general, a cell-based quantitative immunocytochemistry LRRK2 kinase inhibition assay based on LRRK2-Ser935 phosphorylation as the primary readout is developed using the LI-COR® Odyssey® near-infrared technology (also denoted as In-Cell Western™). The assay is performed in 384 well microplate format with LRRK2 wide-type and LRRK2 G2019S mutants expressed in HEK293T cells.

Accordingly, HEK293T cells are transduced with pcDNA5-FRT/TO-FLAG LRRK2 full-length wild-type and LRRK2 G2019S mutants using Lipofectamine™ 3000 and plated in a 384-well assay plate. Cells are incubated with representative compounds of Formular (I) at various concentrations in a 37° C. incubator with a humidified atmosphere of 5% CO₂ for 90 minutes. Cells are fixed by adding 50 μL/well of 8% PFA at room temperature for 1 hr. Phosphorylation of human LRRK2 Ser935 is detected by primary Anti-LRRK2 (phospho S935) antibody (1:2000, Abcam) and infrared-labeled goat-anti-rabbit secondary antibody (1:500, LI-COR®) in the fixed cell, and fluorescent signal from each well is quantified by LICOR® Odyssey® imager Scanner.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

### Itemized List of Embodiments

1. A compound of Formula (I):

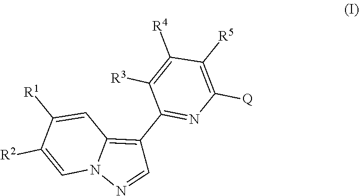

or a pharmaceutically acceptable salt, prodrug, solvate, hydrate, isomer, deuterated form, or tautomer thereof, wherein:
R¹ is hydrogen, C₁-C₆ alkyl, C₃-C₆ cycloalkyl, halogen, halo C₁-C₆ alkyl, —OR⁶, cyano, —NR⁷R⁸, or —NHC(O)R⁹, wherein
   R⁶ is hydrogen, C₁-C₆ alkyl, C₃-C₆ cycloalkyl, C₃-C₆ cycloalkyl C₁-C₆ alkyl, 3-6 membered heterocyclyl, or 3-6 membered heterocyclyl C₁-C₆ alkyl;
   each of R⁷ and R⁸ is independently hydrogen, C₁-C₆ alkyl, or together with the nitrogen to which they are attached form a 3-8 membered monocyclic heterocyclyl group; and
   R⁹ is
      C₆-C₁₀ aryl optionally substituted with C₁-C₆ alkyl, halogen, halo C₁-C₆ alkyl, —OR^{9A}, cyano, or —NR^{9B}R^{9C}, wherein
         R^{9A} is hydrogen or C₁-C₆ alkyl, and
         each of R^{9B} and R^{9C} is independently hydrogen or C₁-C₆ alkyl;
      5- or 6-membered heteraryl comprising one nitrogen atom wherein the heteroaryl is optionally substituted with C₁-C₆ alkyl, halogen, halo C₁-C₆ alkyl, —OR^{9A}, cyano, or —NR^{9B}R^{9C}, or
   C₁-C₆ alkyl optionally substituted with halogen, —SO₂CH₃, cyano, 5- or 6-membered heteroaryl, —OR^{9D}, or —NR^{9E}R^{9F}, wherein
      R^{9D} is hydrogen, C₁-C₆ alkyl, or 5- or 6-membered heteroaryl comprising one nitrogen atom and optionally substituted with C₁-C₆ alkyl, halogen, halo C₁-C₆ alkyl, —OR^{9A}, cyano, or —NR^{9B}R^{9C}, and
      each of R^{9E} and R^{9F} is independently hydrogen, C₁-C₆ alkyl, —SO₂CH₃, or 5- or 6-membered heteroaryl comprising one nitrogen atom;
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen; and
Q is a 3-8 membered heterocyclyl group, wherein
   the heterocyclyl group includes at least one nitrogen atom;
   the heterocyclyl group is optionally fused to a non-aromatic ring containing 3-6 ring members of which 1 or 2 are optionally nitrogen, oxygen, or sulfur atoms and the remainder are carbons;
   the heterocyclyl group is optionally substituted with 1-4 R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.
      2. A compound according to embodiment 1, wherein R¹ is hydrogen.
      3. A compound according to embodiment 1, wherein R¹ is methyl.
      4. A compound according to embodiment 1, wherein R¹ is cyclopropyl.
      5. A compound according to embodiment 1, wherein R¹ is fluoro, chloro or bromo.
      6. A compound according to embodiment 1, wherein R¹ is trifluoromethyl.
      7. A compound according to embodiment 1, wherein R¹ is —OR⁶ and R⁶ is hydrogen.
      8. A compound according to embodiment 1, wherein R¹ is —OR⁶ and R⁶ is isopropyl.
      9. A compound according to embodiment 1, wherein R¹ is —OR⁶ and R⁶ is cyclopropylmethyl.
      10. A compound according to embodiment 1, wherein R¹ is —OR⁶ and R⁶ is methyloxetane.
      11. A compound according to embodiment 1, wherein R¹ is cyano.
      12. A compound according to embodiment 1, wherein R¹ is —NR⁷R⁸, and wherein each of R⁷ and R⁸ is independently hydrogen.
      13. A compound according to embodiment 1, wherein R¹ is —NR⁷R⁸, and wherein each of R⁷ and R⁸ is independently C₁-C₃ alkyl.
      14. A compound according to embodiment 1, wherein R¹ is —NR⁷R⁸, and wherein R⁷ and R⁸ together with the nitrogen to which they are attached form a 3-7 membered monocyclic heterocyclyl group.
      15. A compound according to embodiment 1, wherein R¹ is morpholine.
      16. A compound according to embodiment 1, wherein R¹ is —NHC(O)R⁹ and R⁹ is phenyl.
      17. A compound according to embodiment 1, wherein R¹ is —NHC(O)R⁹ and R⁹ is 5- or 6-membered heteroaryl.
      18. A compound according to embodiment 17, wherein R⁹ is 2-pyridyl.
      19. A compound according to embodiment 1, wherein R¹ is —NHC(O)R⁹ and R⁹ is C₁-C₃ alkyl substituted with —NHSO₂CH₃.
      20. A compound according to embodiment 1, wherein R¹ is —NHC(O)R⁹ and R⁹ is C₁-C₃ alkyl substituted with —SO₂CH₃.
      21. A compound according to embodiment 1, wherein R¹ is —NHC(O)R⁹ and R⁹ is C₁-C₃ alkyl substituted with cyano.
      22. A compound according to embodiment 1, wherein R¹ is —NHC(O)R⁹ and R⁹ is C₁-C₃ alkyl substituted with 5- or 6-membered heteroaryl.
      23. A compound according to embodiment 22, wherein R⁹ is 2-pyridyl methyl or 3-pyridyl methyl.
      24. A compound according to embodiment 1, wherein R¹ is —NHC(O)R⁹ and R⁹ is C₁-C₃ alkyl substituted with —OR^{9D}, and wherein R^{9D} is hydrogen, C₁-C₆ alkyl, or 5- or 6-membered heteroaryl.
      25. A compound according to embodiment 24, wherein R⁹ is methyl substituted with —OR^{9D} and R^{9D} is 5- or 6-membered heteroaryl.
      26. A compound according to embodiment 25, wherein R^{9D} is pyridyl.
      27. A compound according to embodiment 1, wherein R¹ is —NHC(O)R⁹ and R⁹ is C₁-C₃ alkyl substituted with —NR^{9E}R^{9F}, wherein each of R^{9E} and R^{9F} is independently hydrogen, C₁-C₆ alkyl, or 5- or 6-membered heteroaryl.
      28. A compound according to embodiment 27, wherein R⁹ is methyl substituted with —NR^{9E}R^{9F}, and wherein R^{9E} is hydrogen and R^{9F} is pyridyl.
      29. A compound according to embodiment 27, wherein R⁹ is methyl substituted with —NR^{9E}R^{9F}, and wherein R^{9E} is pyridyl and R^{9F} is hydrogen.
      30. A compound according to any of embodiments 1-29, wherein R² is hydrogen.
      31. A compound according to any of embodiments 1-29, wherein R² is —NHC(O)R¹⁰, and wherein R¹⁰ is hydrogen or C₁-C₃ alkyl substituted with —NHSO₂CH₃.
      32. A compound according to any of embodiments 1-29, wherein R² is —C(O)NHR¹¹, and wherein R¹¹ is hydrogen or C₁-C₃ alkyl substituted with —NHSO₂CH₃.
      33. A compound according to any of embodiments 1-32, wherein R³ is hydrogen.
      34. A compound according to any of embodiments 1-32, wherein R³ is fluoro, chloro or bromo.
      35. A compound according to any of embodiments 1-34, wherein R⁴ is hydrogen.
      36. A compound according to any of embodiments 1-34, wherein R⁴ is fluoro, chloro or bromo.
      37. A compound according to any of embodiments 1-34, wherein R⁴ is C₁-C₃ alkoxy.
      38. A compound according to any of embodiments 1-37, wherein R⁵ is hydrogen.
      39. A compound according to any of embodiments 1-37, wherein R⁵ is fluoro, chloro or bromo.
      40. A compound according to any of embodiments 1-39, wherein Q is a 3-7 membered heterocyclyl group including at least one nitrogen atom.
      41. A compound according to any of embodiments 1-39, wherein Q is a 3-7 membered heterocyclyl group, wherein the heterocyclyl group includes at least one nitrogen atom and is fused to a non-aromatic ring containing 3-6 ring members of which 1 or 2 are optionally nitrogen atoms and the remainder are carbons.
      42. A compound according to any of embodiments 1-39, wherein Q is a 3-7 membered heterocyclyl group, wherein the heterocyclyl group includes at least one nitrogen atom and is substituted with one or two R¹² groups, wherein each R¹² is independently C₁-C₃ alkyl.
      43. A compound according to any of embodiments 1-39, wherein Q is a 3-7 membered heterocyclyl group, wherein the heterocyclyl group includes at least one nitrogen atom and is substituted with one R¹³ group, wherein R¹³ is hydrogen, C₁-C₃ alkyl, —SO₂R¹⁴, or —C(O)R¹⁵ and wherein R¹⁴ is hydrogen or C₁-C₃ alkyl, and R¹⁵ is hydrogen or C₁-C₃ alkyl substituted with —NHSO₂CH₃.
      44. A compound according to any of embodiments 1-39, wherein Q is a 3-7 membered heterocyclyl group, wherein the heterocyclyl group includes at least one nitrogen atom and is substituted with one or two R¹² groups and with one R¹³ group, wherein
each R¹² is independently C₁-C₃ alkyl; and
R¹³ is hydrogen, C₁-C₃ alkyl, —SO₂R¹⁴, or —C(O)R¹⁵, wherein R¹⁴ is hydrogen or C₁-C₃ alkyl,
and R¹⁵ is hydrogen or C₁-C₃ alkyl substituted with —NHSO₂CH₃.
   45. A compound of formula (II):

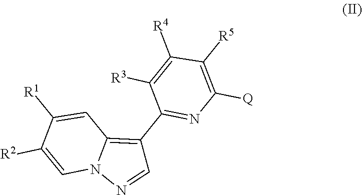

or a pharmaceutically acceptable salt, prodrug, solvate, hydrate, isomer, deuterated form, or tautomer thereof, wherein:
R¹ is fluoro, chloro, or bromo;
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen; and
Q is a 3-8 membered heterocyclyl group, wherein
   the heterocyclyl group includes at least one nitrogen atom;
   the heterocyclyl group is optionally fused to a non-aromatic ring containing 3-6 ring members of which 1 or 2 are optionally nitrogen, oxygen or sulfur atoms and the remainder are carbons;
   the heterocyclyl group is optionally substituted with one to four R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.
      46. A compound of formula (III):

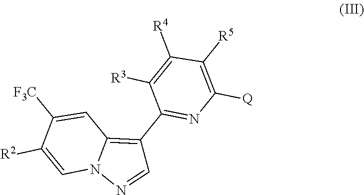

or a pharmaceutically acceptable salt, prodrug, solvate, hydrate, isomer, deuterated form, or tautomer thereof, wherein:
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen; and
Q is a 3-8 membered heterocyclyl group, wherein
   the heterocyclyl group includes at least one nitrogen atom;
   the heterocyclyl group is optionally fused to a non-aromatic ring containing 3-6 ring members of which 1 or 2 are optionally nitrogen, oxygen or sulfur atoms and the remainder are carbons;
   the heterocyclyl group is optionally substituted with one to four R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.
      47. A compound of formula (IV):

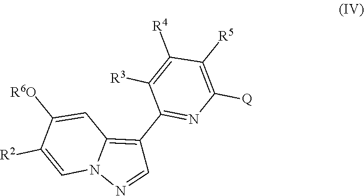

or a pharmaceutically acceptable salt, prodrug, solvate, hydrate, isomer, deuterated form, or tautomer thereof, wherein:
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen;
R⁶ is hydrogen, C₁-C₆ alkyl, C₃-C₆ cycloalkyl, C₃-C₆ cycloalkyl C₁-C₆ alkyl, 3-6 membered heterocyclyl, or 3-6 membered heterocyclyl C₁-C₆ alkyl; and
Q is a 3-8 membered heterocyclyl group, wherein
   the heterocyclyl group includes at least one nitrogen atom;
   the heterocyclyl group is optionally fused to a non-aromatic ring containing 3-6 ring members of which 1 or 2 are optionally nitrogen, oxygen or sulfur atoms and the remainder are carbons;
   the heterocyclyl group is optionally substituted with one to four R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.
      48. A compound of formula (V):

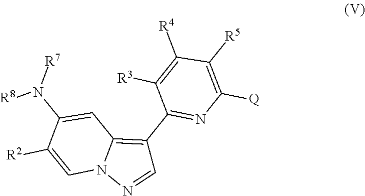

or a pharmaceutically acceptable salt, prodrug, solvate, hydrate, isomer, deuterated form, or tautomer thereof, wherein:
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen;
R⁷ and R⁸ are independently hydrogen or C₁-C₆ alkyl, or together with the nitrogen to which they are attached form a 3-8 membered monocyclic heterocyclyl group; and
Q is a 3-8 membered heterocyclyl group, wherein
   the heterocyclyl group includes at least one nitrogen atom;
   the heterocyclyl group is optionally fused to a non-aromatic ring containing 3-6 ring members of which 1 or 2 are optionally nitrogen, oxygen or sulfur atoms and the remainder are carbons;
   the heterocyclyl group is optionally substituted with one to four R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.
      49. A compound of formula (VI):

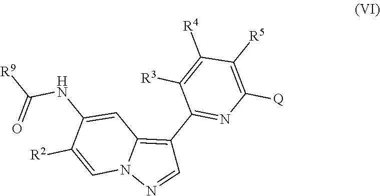

or a pharmaceutically acceptable salt, prodrug, solvate, hydrate, isomer, deuterated form, or tautomer thereof, wherein:
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen;
R⁹ is C₆-C₁₀ aryl, 5- or 6-membered heteroaryl, or C₁-C₆ alkyl, wherein
   the C₆-C₁₀ aryl is optionally substituted with C₁-C₆ alkyl, halogen, halo C₁-C₆ alkyl, —OR^{9A}, cyano, or —NR^{9B}R^{9C}, wherein
      R^{9A} is hydrogen or C₁-C₆ alkyl, and
      each of R^{9B} and R^{9C} is independently hydrogen or C₁-C₆ alkyl;
   the 5- or 6-membered heteraryl comprises one nitrogen atom and is optionally substituted with C₁-C₆ alkyl, halogen, halo C₁-C₆ alkyl, —OR^{9A}, cyano, or —NR^{9B}R^{9C}, and
   the C₁-C₆ alkyl is optionally substituted with halogen, —SO₂CH₃, cyano, 5- or 6-membered heteroaryl, —OR^{9D}, or —NR^{9E}R^{9F}, wherein
      R^{9D} is hydrogen, C₁-C₆ alkyl, or 5- or 6-membered heteroaryl comprising one nitrogen atom and optionally substituted with C₁-C₆ alkyl, halogen, halo C₁-C₆ alkyl, —OR^{9A}, cyano, or —NR^{9B}R^{9C}, and
      each of R^{9E} and R^{9F} is independently hydrogen, C₁-C₆ alkyl, —SO₂CH₃, or 5- or 6-membered heteroaryl comprising one nitrogen atom; and
Q is a 3-8 membered heterocyclyl group, wherein
   the heterocyclyl group includes at least one nitrogen atom;
   the heterocyclyl group is optionally fused to a non-aromatic ring containing 3-6 ring members of which 1 or 2 are optionally nitrogen, oxygen or sulfur atoms and the remainder are carbons;
   the heterocyclyl group is optionally substituted with one to four R¹² groups, wherein each R¹² is independently C₁-C₆ alkyl, halogen, halo C₁-C₆ alkyl, or C₁-C₆ alkoxy; and
   the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.
      50. A compound according to any of embodiments 1-49, wherein Q is a group of the formula:

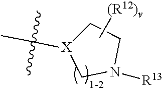

wherein
each v represents 0, 1, 2, 3 or 4;
each R¹² is independently hydrogen, C₁-C₆ alkyl, halogen, halo C₁-C₆ alkyl, or C₁-C₆ alkoxy;
R¹³ is hydrogen, C₁-C₆ alkyl, —SO₂R¹⁴, or —C(O)R¹⁵, wherein R¹⁴ is hydrogen or C₁-C₆ alkyl, and R¹⁵ is hydrogen or C₁-C₆ alkyl substituted with —NHSO₂CH₃; and
X is CR¹⁶ or nitrogen, wherein R¹⁶ is hydrogen or C₁-C₆ alkyl.
   51. A compound according to embodiment 1, which is:
3-(6-(piperidin-4-yl)pyridin-2-yl)pyrazolo[1,5-a]pyridine;
3-(6-(1-methylpiperidin-4-yl)pyridin-2-yl)pyrazolo[1,5-a]pyridine;
3-(6-(pyrrolidin-3-yl)pyridin-2-yl)pyrazolo[1,5-a]pyridine;
5-chloro-3-(6-(pyrrolidin-3-yl)pyridin-2-yl)pyrazolo[1,5-a]pyridine;
Rac-5-chloro-3-(6-(piperidin-3-yl)pyridin-2-yl)pyrazolo[1,5-a]pyridine;
(+)-5-chloro-3-(6-(piperidin-3-yl)pyridin-2-yl)pyrazolo[1,5-a]pyridine;
(−)-5-chloro-3-(6-(piperidin-3-yl)pyridin-2-yl)pyrazolo[1,5-a]pyridine;
5-bromo-3-(6-(piperidin-3-yl)pyridin-2-yl)pyrazolo[1,5-a]pyridine;
5-methyl-3-(6-(piperidin-3-yl)pyridin-2-yl)pyrazolo[1,5-a]pyridine;
3-(6-(piperidin-3-yl)pyridin-2-yl)-5-(trifluoromethyl)pyrazolo[1,5-a]pyridine;
5-methoxy-3-(6-(piperidin-3-yl)pyridin-2-yl)pyrazolo[1,5-a]pyridine;
3-(6-(piperidin-3-yl)pyridin-2-yl)pyrazolo[1,5-a]pyridin-5-ol;
5-isopropoxy-3-(6-(piperidin-3-yl)pyridin-2-yl)pyrazolo[1,5-a]pyridine;
5-(cyclopropylmethoxy)-3-(6-(piperidin-3-yl)pyridin-2-yl)pyrazolo[1,5-a]pyridine;
5-(oxetan-3-ylmethoxy)-3-(6-(piperidin-3-yl)pyridin-2-yl)pyrazolo[1,5-a]pyridine;
4-(3-(6-(piperidin-3-yl)pyridin-2-yl)pyrazolo[1,5-a]pyridin-5-yl)morpholine;
3-(6-(piperidin-3-yl)pyridin-2-yl)pyrazolo[1,5-a]pyridine-5-carbonitrile;
5-cyclopropyl-3-(6-(piperidin-3-yl)pyridin-2-yl)pyrazolo[1,5-a]pyridine;
3-(6-(piperidin-3-yl)pyridin-2-yl)pyrazolo[1,5-a]pyridin-5-amine;
N-methyl-3-(6-(piperidin-3-yl)pyridin-2-yl)pyrazolo[1,5-a]pyridin-5-amine;
N-ethyl-3-(6-(piperidin-3-yl)pyridin-2-yl)pyrazolo[1,5-a]pyridin-5-amine;
3-(methylsulfonamido)-N-(3-(6-(piperidin-3-yl)pyridin-2-yl)pyrazolo[1,5-a]pyridin-5-yl)propanamide;
3-(methylsulfonamido)-N-(3-(6-(piperidin-3-yl)pyridin-2-yl)pyrazolo[1,5-a]pyridin-6-yl)propanamide;
N-(2-(methylsulfonamido)ethyl)-3-(6-(piperidin-3-yl)pyridin-2-yl)pyrazolo[1,5-a]pyridine-6-carboxamide;
N-(2-oxo-2-(3-(6-(pyrazolo[1,5-a]pyridin-3-yl)pyridin-2-yl)piperidin-1-yl)ethyl)methanesulfonamide;
3-(6-(1-(methylsulfonyl)piperidin-3-yl)pyridin-2-yl)pyrazolo[1,5-a]pyridine;
3-(4-methoxy-6-(piperidin-3-yl)pyridin-2-yl)pyrazolo[1,5-a]pyridine;
3-(4-chloro-6-(piperidin-3-yl)pyridin-2-yl)pyrazolo[1,5-a]pyridine;
3-(5-chloro-6-(piperidin-3-yl)pyridin-2-yl)pyrazolo[1,5-a]pyridine;
3-(3-chloro-6-(piperidin-3-yl)pyridin-2-yl)pyrazolo[1,5-a]pyridine;
3-(4-fluoro-6-(piperidin-3-yl)pyridin-2-yl)pyrazolo[1,5-a]pyridine;
3-(3-fluoro-6-(piperidin-3-yl)pyridin-2-yl)pyrazolo[1,5-a]pyridine;
3-(5-fluoro-6-(piperidin-3-yl)pyridin-2-yl)pyrazolo[1,5-a]pyridine;
5-chloro-3-(4-chloro-6-(piperidin-3-yl)pyridin-2-yl)pyrazolo[1,5-a]pyridine;
5-chloro-3-(4-fluoro-6-(piperidin-3-yl)pyridin-2-yl)pyrazolo[1,5-a]pyridine;
3-(4-chloro-6-(piperidin-3-yl)pyridin-2-yl)-5-(trifluoromethyl)pyrazolo[1,5-a]pyridine;
3-(6-(piperazin-1-yl)pyridin-2-yl)pyrazolo[1,5-a]pyridine; N-methyl-3-(6-(piperazin-1-yl)pyridin-2-yl)pyrazolo[1,5-a]pyridin-5-amine;
3-(4-chloro-6-(piperazin-1-yl)pyridin-2-yl)-N-methylpyrazolo[1,5-a]pyridin-5-amine;
3-(6-(piperazin-1-yl)pyridin-2-yl)-5-(trifluoromethyl)pyrazolo[1,5-a]pyridine;
3-(4-chloro-6-(piperazin-1-yl)pyridin-2-yl)-5-(trifluoromethyl)pyrazolo[1,5-a]pyridine;
5-chloro-3-(6-(piperazin-1-yl)pyridin-2-yl)pyrazolo[1,5-a]pyridine;
5-chloro-3-(4-chloro-6-(piperazin-1-yl)pyridin-2-yl)pyrazolo[1,5-a]pyridine;
5-chloro-3-(4-fluoro-6-(piperazin-1-yl)pyridin-2-yl)pyrazolo[1,5-a]pyridine;
3-(6-(pyrazolo[1,5-a]pyridin-3-yl)pyridin-2-yl)-3,6-diazabicyclo[3.1.1]heptane;
3-(6-(5-chloropyrazolo[1,5-a]pyridin-3-yl)pyridin-2-yl)-3,6-diazabicyclo[3.1.1]heptane;
3-(4-chloro-6-(5-chloropyrazolo[1,5-a]pyridin-3-yl)pyridin-2-yl)-3,6-diazabicyclo[3.1.1]heptane;
2-(6-(pyrazolo[1,5-a]pyridin-3-yl)pyridin-2-yl)-2,5-diazabicyclo[2.2.2]octane;
3-(6-((3S,5R)-3,5-dimethylpiperazin-1-yl)pyridin-2-yl)pyrazolo[1,5-a]pyridine;
3-(6-((3R,5R)-3,5-dimethylpiperazin-1-yl)pyridin-2-yl)pyrazolo[1,5-a]pyridine;
3-(4-chloro-6-((3S,5R)-3,5-dimethylpiperazin-1-yl)pyridin-2-yl)pyrazolo[1,5-a]pyridine;
3-(6-((3S,5R)-3,5-dimethylpiperazin-1-yl)-4-fluoropyridin-2-yl)pyrazolo[1,5-a]pyridine;
3-(6-((3S,5R)-3,5-dimethylpiperazin-1-yl)pyridin-2-yl)-N-methylpyrazolo[1,5-a]pyridin-5-amine;
3-(4-chloro-6-((3S,5R)-3,5-dimethylpiperazin-1-yl)pyridin-2-yl)-N-methylpyrazolo[1,5-a]pyridin-5-amine;
3-(6-((3S,5R)-3,5-dimethylpiperazin-1-yl)pyridin-2-yl)-5-(trifluoromethyl)pyrazolo[1,5-a]pyridine;
3-(4-chloro-6-((3S,5R)-3,5-dimethylpiperazin-1-yl)pyridin-2-yl)-5-(trifluoromethyl)pyrazolo[1,5-a]pyridine;
5-chloro-3-(6-((3S,5R)-3,5-dimethylpiperazin-1-yl)pyridin-2-yl)pyrazolo[1,5-a]pyridine;
5-chloro-3-(6-((3S,5R)-3,5-dimethylpiperazin-1-yl)-4-fluoropyridin-2-yl)pyrazolo[1,5-a]pyridine;
5-chloro-3-(4-chloro-6-((3S,5R)-3,5-dimethylpiperazin-1-yl)pyridin-2-yl)pyrazolo[1,5-a]pyridine;
5-chloro-3-(5-chloro-6-((3S,5R)-3,5-dimethylpiperazin-1-yl)pyridin-2-yl)pyrazolo[1,5-a]pyridine;
5-chloro-3-(6-((3S,5R)-3,5-dimethylpiperazin-1-yl)-5-fluoropyridin-2-yl)pyrazolo[1,5-a]pyridine;
3-(4-chloro-6-(piperidin-3-yl)pyridin-2-yl)-N-methylpyrazolo[1,5-a]pyridin-5-amine;
5-chloro-3-(3-chloro-6-((3S,5R)-3,5-dimethylpiperazin-1-yl)pyridin-2-yl)pyrazolo[1,5-a]pyridine;
5-chloro-3-(6-((3S,5R)-3,5-dimethylpiperazin-1-yl)-3-fluoropyridin-2-yl)pyrazolo[1,5-a]pyridine;
3-(4-chloro-6-(piperidin-3-yl)pyridin-2-yl)-5-isopropoxypyrazolo[1,5-a]pyridine;
5-chloro-3-(6-(3,3,5,5-tetramethylpiperazin-1-yl)pyridin-2-yl)pyrazolo[1,5-a]pyridine;
2-(methylsulfonyl)-N-(3-(6-(piperidin-3-yl)pyridin-2-yl)pyrazolo[1,5-a]pyridin-5-yl)acetamide;
2-(methylsulfonamido)-N-(3-(6-(piperidin-3-yl)pyridin-2-yl)pyrazolo[1,5-a]pyridin-5-yl)acetamide;
(R)-3-(6-(3-methylpiperazin-1-yl)pyridin-2-yl)pyrazolo[1,5-a]pyridine;
(S)-3-(6-(3-methylpiperazin-1-yl)pyridin-2-yl)pyrazolo[1,5-a]pyridine; 5-chloro-3-(6-((3S,5R)-3,4,5-trimethylpiperazin-1-yl)pyridin-2-yl)pyrazolo[1,5-a]pyridine;
N-(3-(6-(piperidin-3-yl)pyridin-2-yl)pyrazolo[1,5-a]pyridin-5-yl)picolinamide;
N-(3-(6-(piperidin-3-yl)pyridin-2-yl)pyrazolo[1,5-a]pyridin-5-yl)-2-(pyridin-2-yloxy)acetamide;
3-cyano-N-(3-(6-(piperidin-3-yl)pyridin-2-yl)pyrazolo[1,5-a]pyridin-5-yl)propanamide;
4-cyano-N-(3-(6-(piperidin-3-yl)pyridin-2-yl)pyrazolo[1,5-a]pyridin-5-yl)butanamide;
N-(3-(6-(piperidin-3-yl)pyridin-2-yl)pyrazolo[1,5-a]pyridin-5-yl)-2-(pyridin-4-yloxy)acetamide;
2-cyano-N-(3-(6-(piperidin-3-yl)pyridin-2-yl)pyrazolo[1,5-a]pyridin-5-yl)acetamide;
N-(3-(6-(piperidin-3-yl)pyridin-2-yl)pyrazolo[1,5-a]pyridin-5-yl)-2-(pyridin-3-yl)acetamide;
N-(3-(6-(piperidin-3-yl)pyridin-2-yl)pyrazolo[1,5-a]pyridin-5-yl)-2-(pyridin-4-ylamino)acetamide;
N-(3-(6-(piperidin-3-yl)pyridin-2-yl)pyrazolo[1,5-a]pyridin-5-yl)-2-(pyridin-3-ylamino)acetamide;
N-(3-(6-(piperidin-3-yl)pyridin-2-yl)pyrazolo[1,5-a]pyridin-5-yl)-2-(pyridin-2-yl)acetamide;
   or a pharmaceutically acceptable salt thereof.
      52. A pharmaceutical composition comprising a compound or salt according to any of embodiments 1-51 together with a pharmaceutically acceptable carrier, excipient, or diluent.
      53. A method of treating a disease or disorder associated with LRRK2 kinase activity, the method comprising administering to a patient in need thereof a therapeutically effective amount of a compound of any one of embodiments 1-51 or a pharmaceutical composition of embodiment 52.
      54. The method of embodiment 53, wherein the disease or disorder is Parkinson's disease, Alzheimer's disease, multiple sclerosis, HIV-induced dementia, amyotrophic lateral sclerosis, ischemic stroke, traumatic brain injury, or spinal cord injury.
      55. A method of inhibiting LRRK2 kinase activity, the method comprising administering to a patient in need thereof a therapeutically effective amount of a compound of any one of embodiments 1-51 or a pharmaceutical composition of embodiment 52.
      56. A method of treating Parkinson's disease, comprising administering to a patient in need thereof a therapeutically effective amount of a compound of any one of embodiments 1-51 or a pharmaceutical composition of embodiment 52.

What is claimed is:

1. A compound of Formula (I): 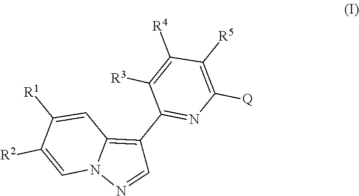
or a pharmaceutically acceptable salt, prodrug, solvate, hydrate, isomer, deuterated form, or tautomer thereof, wherein:
R¹ is hydrogen, C₁-C₆ alkyl, C₃-C₆ cycloalkyl, halogen, halo C₁-C₆ alkyl, —OR⁶, cyano, —NR⁷R⁸, or —NHC(O)R⁹, wherein
R⁶ is hydrogen, C₁-C₆ alkyl, C₃-C₆ cycloalkyl, C₃-C₆ cycloalkyl C₁-C₆ alkyl, 3-6 membered heterocyclyl, or 3-6 membered heterocyclyl C₁-C₆ alkyl;
each of R⁷ and R⁸ is independently hydrogen, C₁-C₆ alkyl, or together with the nitrogen to which they are attached form a 3-8 membered monocyclic heterocyclyl group; and
R⁹ is
C₆-C₁₀ aryl optionally substituted with C₁-C₆ alkyl, halogen, halo C₁-C₆ alkyl, —OR^{9A}, cyano, or —NR^{9B}R^{9C}, wherein
R^{9A} is hydrogen or C₁-C₆ alkyl, and
each of R^{9B} and R^{9C} is independently hydrogen or C₁-C₆ alkyl;
5- or 6-membered heteraryl comprising one nitrogen atom wherein the heteroaryl is optionally substituted with C₁-C₆ alkyl, halogen, halo C₁-C₆ alkyl, —OR^{9A}, cyano, or —NR^{9B}R^{9C}, or
C₁-C₆ alkyl optionally substituted with halogen, —SO₂CH₃, cyano, 5- or 6-membered heteroaryl, —OR^{9D}, or —NR^{9E}R^{9F}, wherein
R^{9D} is hydrogen, C₁-C₆ alkyl, or 5- or 6-membered heteroaryl comprising one nitrogen atom and optionally substituted with C₁-C₆ alkyl, halogen, halo C₁-C₆ alkyl, —OR^{9A}, cyano, or —NR^{9B}R^{9C}, and
each of R^{9E} and R^{9F} is independently hydrogen, C₁-C₆ alkyl, —SO₂CH₃, or 5- or 6-membered heteroaryl comprising one nitrogen atom;
R² is hydrogen, —NHC(O)R¹⁰, or —C(O)NHR¹¹, wherein each of R¹⁰ and R¹¹ is independently hydrogen or C₁-C₆ alkyl substituted with —NHSO₂CH₃;
R³ is hydrogen or halogen;
R⁴ is hydrogen, C₁-C₆ alkyl, halogen, halo C₁-C₆ alkyl, or C₁-C₆ alkoxy;
R⁵ is hydrogen or halogen; and
Q is a 3-8 membered heterocyclyl group, wherein
the heterocyclyl group includes at least one nitrogen atom;
the heterocyclyl group is optionally fused to a non-aromatic ring containing 3-6 ring members of which 1 or 2 are optionally nitrogen, oxygen, or sulfur atoms and the remainder are carbons;
the heterocyclyl group is optionally substituted with 1-4 R¹² groups, wherein each R¹² is independently C₁-C₆ alkyl, halogen, halo C₁-C₆ alkyl, or C₁-C₆ alkoxy; and
the heterocyclyl group is optionally substituted with one R¹³ group, wherein R¹³ is hydrogen, C₁-C₆ alkyl, —SO₂R¹⁴, or —C(O)R¹⁵, and wherein R¹⁴ is hydrogen or C₁-C₆ alkyl, and R¹⁵ is hydrogen or C₁-C₆ alkyl substituted with —NHSO₂CH₃.

2. A compound according to claim 1, wherein R¹ is hydrogen, methyl, cyclpropyl, fluoro, chloro, bromo, cyano, morpholine, or trifluoromethyl.

3. A compound according to claim 1, wherein R¹ is —OR⁶ and R⁶ is hydrogen, isopropyl, cyclopropylmethyl, or methyloxetane.

4. A compound according to claim 1, wherein R¹ is —NR⁷R⁸, and wherein each of R⁷ and R⁸ is independently hydrogen or C₁-C₃ alkyl, or R⁷ and R⁸ together with the nitrogen to which they are attached form a 3-7 membered monocyclic heterocyclyl group.

5. A compound according to claim 1, wherein R¹ is —NHC(O)R⁹ and R⁹ is phenyl, 5- or 6-membered heteroaryl, 2-pyridyl, C₁-C₃ alkyl substituted with —NHSO₂CH₃, C₁-C₃ alkyl substituted with —SO₂CH₃, C₁-C₃ alkyl substituted with cyano, C₁-C₃ alkyl substituted with 5- or 6-membered heteroaryl.

6. A compound according to claim 1, wherein R¹ is —NHC(O)R⁹ and R⁹ is C₁-C₃ alkyl substituted with —OR^{9D}, and wherein R^{9D} is hydrogen, C₁-C₆ alkyl, or 5- or 6-membered heteroaryl.

7. A compound according to claim 1, wherein R¹ is —NHC(O)R⁹ and R⁹ is C₁-C₃ alkyl substituted with —NR^{9E}R^{9F}, wherein each of R^{9E} and R^{9F} is independently hydrogen, C₁-C₆ alkyl, or 5- or 6-membered heteroaryl.

8. A compound according to claim 1, wherein R² is
(i) hydrogen;
(ii) —NHC(O)R¹⁰, and wherein R¹⁰ is hydrogen or C₁-C₃ alkyl substituted with —NHSO₂CH₃; or
(iii) —C(O)NHR¹¹, and wherein R¹ is hydrogen or C₁-C₃ alkyl substituted with —NHSO₂CH₃.

9. A compound according to claim 1, wherein R³ is hydrogen, fluoro, chloro or bromo.

10. A compound according to claim 1, wherein R⁴ is hydrogen, fluoro, chloro, bromo or C₁-C₃ alkoxy.

11. A compound according to claim 1, wherein R⁵ is hydrogen, fluoro, chloro or bromo.

12. A compound according to claim 1, wherein Q is a 3-7 membered heterocyclyl group including at least one nitrogen atom.

13. A compound according to claim 1, wherein Q is a 3-7 membered heterocyclyl group, wherein the heterocyclyl group includes at least one nitrogen atom and is fused to a non-aromatic ring containing 3-6 ring members of which 1 or 2 are optionally nitrogen atoms and the remainder are carbons.

14. A compound according to claim 1, wherein Q is a 3-7 membered heterocyclyl group, wherein the heterocyclyl group includes at least one nitrogen atom and is substituted with one or two R¹² groups, wherein each R¹² is independently C₁-C₃ alkyl.

15. A compound according to claim 1, wherein Q is a 3-7 membered heterocyclyl group, wherein the heterocyclyl group includes at least one nitrogen atom and is substituted with one R¹³ group, wherein R¹³ is hydrogen, C₁-C₃ alkyl, —SO₂R¹⁴, or —C(O)R¹⁵, and wherein R¹⁴ is hydrogen or C₁-C₃ alkyl, and R¹⁵ is hydrogen or C₁-C₃ alkyl substituted with —NHSO₂CH₃.

16. A compound according to claim 1, wherein Q is a 3-7 membered heterocyclyl group, wherein the heterocyclyl group includes at least one nitrogen atom and is substituted with one or two R¹² groups and with one R¹³ group, wherein
each R¹² is independently C₁-C₃ alkyl; and
R¹³ is hydrogen, C₁-C₃ alkyl, —SO₂R¹⁴, or —C(O)R¹⁵, wherein R¹⁴ is hydrogen or C₁-C₃ alkyl,
and R¹⁵ is hydrogen or C₁-C₃ alkyl substituted with —NHSO₂CH₃.

17. A compound according to claim 1, which is:
3-(6-(piperidin-4-yl)pyridin-2-yl)pyrazolo[1,5-a]pyridine;
3-(6-(1-methylpiperidin-4-yl)pyridin-2-yl)pyrazolo[1,5-a]pyridine;
3-(6-(pyrrolidin-3-yl)pyridin-2-yl)pyrazolo[1,5-a]pyridine;
5-chloro-3-(6-(pyrrolidin-3-yl)pyridin-2-yl)pyrazolo[1,5-a]pyridine;
Rac-5-chloro-3-(6-(piperidin-3-yl)pyridin-2-yl)pyrazolo[1,5-a]pyridine;
(+)-5-chloro-3-(6-(piperidin-3-yl)pyridin-2-yl)pyrazolo[1,5-a]pyridine;
(−)-5-chloro-3-(6-(piperidin-3-yl)pyridin-2-yl)pyrazolo[1,5-a]pyridine;
5-bromo-3-(6-(piperidin-3-yl)pyridin-2-yl)pyrazolo[1,5-a]pyridine;
5-methyl-3-(6-(piperidin-3-yl)pyridin-2-yl)pyrazolo[1,5-a]pyridine;
3-(6-(piperidin-3-yl)pyridin-2-yl)-5-(trifluoromethyl)pyrazolo[1,5-a]pyridine;
5-methoxy-3-(6-(piperidin-3-yl)pyridin-2-yl)pyrazolo[1,5-a]pyridine;
3-(6-(piperidin-3-yl)pyridin-2-yl)pyrazolo[1,5-a]pyridin-5-ol;
5-isopropoxy-3-(6-(piperidin-3-yl)pyridin-2-yl)pyrazolo[1,5-a]pyridine;
5-(cyclopropylmethoxy)-3-(6-(piperidin-3-yl)pyridin-2-yl)pyrazolo[1,5-a]pyridine;
5-(oxetan-3-ylmethoxy)-3-(6-(piperidin-3-yl)pyridin-2-yl)pyrazolo[1,5-a]pyridine;
4-(3-(6-(piperidin-3-yl)pyridin-2-yl)pyrazolo[1,5-a]pyridin-5-yl)morpholine;
3-(6-(piperidin-3-yl)pyridin-2-yl)pyrazolo[1,5-a]pyridine-5-carbonitrile;
5-cyclopropyl-3-(6-(piperidin-3-yl)pyridin-2-yl)pyrazolo[1,5-a]pyridine;
3-(6-(piperidin-3-yl)pyridin-2-yl)pyrazolo[1,5-a]pyridin-5-amine;
N-methyl-3-(6-(piperidin-3-yl)pyridin-2-yl)pyrazolo[1,5-a]pyridin-5-amine;
N-ethyl-3-(6-(piperidin-3-yl)pyridin-2-yl)pyrazolo[1,5-a]pyridin-5-amine;
3-(methylsulfonamido)-N-(3-(6-(piperidin-3-yl)pyridin-2-yl)pyrazolo[1,5-a]pyridin-5-yl)propanamide;
3-(methylsulfonamido)-N-(3-(6-(piperidin-3-yl)pyridin-2-yl)pyrazolo[1,5-a]pyridin-6-yl)propanamide;
N-(2-(methylsulfonamido)ethyl)-3-(6-(piperidin-3-yl)pyridin-2-yl)pyrazolo[1,5-a]pyridine-6-carboxamide;
N-(2-oxo-2-(3-(6-(pyrazolo[1,5-a]pyridin-3-yl)pyridin-2-yl)piperidin-1-yl)ethyl)methanesulfonamide;
3-(6-(1-(methylsulfonyl)piperidin-3-yl)pyridin-2-yl)pyrazolo[1,5-a]pyridine;
3-(4-methoxy-6-(piperidin-3-yl)pyridin-2-yl)pyrazolo[1,5-a]pyridine;
3-(4-chloro-6-(piperidin-3-yl)pyridin-2-yl)pyrazolo[1,5-a]pyridine;
3-(5-chloro-6-(piperidin-3-yl)pyridin-2-yl)pyrazolo[1,5-a]pyridine;
3-(3-chloro-6-(piperidin-3-yl)pyridin-2-yl)pyrazolo[1,5-a]pyridine;
3-(4-fluoro-6-(piperidin-3-yl)pyridin-2-yl)pyrazolo[1,5-a]pyridine;
3-(3-fluoro-6-(piperidin-3-yl)pyridin-2-yl)pyrazolo[1,5-a]pyridine;
3-(5-fluoro-6-(piperidin-3-yl)pyridin-2-yl)pyrazolo[1,5-a]pyridine;
5-chloro-3-(4-chloro-6-(piperidin-3-yl)pyridin-2-yl)pyrazolo[1,5-a]pyridine;
5-chloro-3-(4-fluoro-6-(piperidin-3-yl)pyridin-2-yl)pyrazolo[1,5-a]pyridine;
3-(4-chloro-6-(piperidin-3-yl)pyridin-2-yl)-5-(trifluoromethyl)pyrazolo[1,5-a]pyridine;
3-(6-(piperazin-1-yl)pyridin-2-yl)pyrazolo[1,5-a]pyridine;
N-methyl-3-(6-(piperazin-1-yl)pyridin-2-yl)pyrazolo[1,5-a]pyridin-5-amine;
3-(4-chloro-6-(piperazin-1-yl)pyridin-2-yl)-N-methylpyrazolo[1,5-a]pyridin-5-amine;
3-(6-(piperazin-1-yl)pyridin-2-yl)-5-(trifluoromethyl)pyrazolo[1,5-a]pyridine;
3-(4-chloro-6-(piperazin-1-yl)pyridin-2-yl)-5-(trifluoromethyl)pyrazolo[1,5-a]pyridine;
5-chloro-3-(6-(piperazin-1-yl)pyridin-2-yl)pyrazolo[1,5-a]pyridine;
5-chloro-3-(4-chloro-6-(piperazin-1-yl)pyridin-2-yl)pyrazolo[1,5-a]pyridine;
5-chloro-3-(4-fluoro-6-(piperazin-1-yl)pyridin-2-yl)pyrazolo[1,5-a]pyridine;
3-(6-(pyrazolo[1,5-a]pyridin-3-yl)pyridin-2-yl)-3,6-diazabicyclo[3.1.1]heptane;
3-(6-(5-chloropyrazolo[1,5-a]pyridin-3-yl)pyridin-2-yl)-3,6-diazabicyclo[3.1.1]heptane;
3-(4-chloro-6-(5-chloropyrazolo[1,5-a]pyridin-3-yl)pyridin-2-yl)-3,6-diazabicyclo[3.1.1]heptane;
2-(6-(pyrazolo[1,5-a]pyridin-3-yl)pyridin-2-yl)-2,5-diazabicyclo[2.2.2]octane;
3-(6-((3S,5R)-3,5-dimethylpiperazin-1-yl)pyridin-2-yl)pyrazolo[1,5-a]pyridine;
3-(6-((3R,5R)-3,5-dimethylpiperazin-1-yl)pyridin-2-yl)pyrazolo[1,5-a]pyridine;
3-(4-chloro-6-((3S,5R)-3,5-dimethylpiperazin-1-yl)pyridin-2-yl)pyrazolo[1,5-a]pyridine;
3-(6-((3S,5R)-3,5-dimethylpiperazin-1-yl)-4-fluoropyridin-2-yl)pyrazolo[1,5-a]pyridine;
3-(6-((3S,5R)-3,5-dimethylpiperazin-1-yl)pyridin-2-yl)-N-methylpyrazolo[1,5-a]pyridin-5-amine;
3-(4-chloro-6-((3S,5R)-3,5-dimethylpiperazin-1-yl)pyridin-2-yl)-N-methylpyrazolo[1,5-a]pyridin-5-amine;
3-(6-((3S,5R)-3,5-dimethylpiperazin-1-yl)pyridin-2-yl)-5-(trifluoromethyl)pyrazolo[1,5-a]pyridine;
3-(4-chloro-6-((3S,5R)-3,5-dimethylpiperazin-1-yl)pyridin-2-yl)-5-(trifluoromethyl)pyrazolo[1,5-a]pyridine;
5-chloro-3-(6-((3S,5R)-3,5-dimethylpiperazin-1-yl)pyridin-2-yl)pyrazolo[1,5-a]pyridine;
5-chloro-3-(6-((3S,5R)-3,5-dimethylpiperazin-1-yl)-4-fluoropyridin-2-yl)pyrazolo[1,5-a]pyridine;
5-chloro-3-(4-chloro-6-((3S,5R)-3,5-dimethylpiperazin-1-yl)pyridin-2-yl)pyrazolo[1,5-a]pyridine;
5-chloro-3-(5-chloro-6-((3S,5R)-3,5-dimethylpiperazin-1-yl)pyridin-2-yl)pyrazolo[1,5-a]pyridine;
5-chloro-3-(6-((3S,5R)-3,5-dimethylpiperazin-1-yl)-5-fluoropyridin-2-yl)pyrazolo[1,5-a]pyridine;
3-(4-chloro-6-(piperidin-3-yl)pyridin-2-yl)-N-methylpyrazolo[1,5-a]pyridin-5-amine;
5-chloro-3-(3-chloro-6-((3S,5R)-3,5-dimethylpiperazin-1-yl)pyridin-2-yl)pyrazolo[1,5-a]pyridine;
5-chloro-3-(6-((3S,5R)-3,5-dimethylpiperazin-1-yl)-3-fluoropyridin-2-yl)pyrazolo[1,5-a]pyridine;
3-(4-chloro-6-(piperidin-3-yl)pyridin-2-yl)-5-isopropoxypyrazolo[1,5-a]pyridine;
5-chloro-3-(6-(3,3,5,5-tetramethylpiperazin-1-yl)pyridin-2-yl)pyrazolo[1,5-a]pyridine;
2-(methylsulfonyl)-N-(3-(6-(piperidin-3-yl)pyridin-2-yl)pyrazolo[1,5-a]pyridin-5-yl)acetamide;
2-(methylsulfonamido)-N-(3-(6-(piperidin-3-yl)pyridin-2-yl)pyrazolo[1,5-a]pyridin-5-yl)acetamide;
(R)-3-(6-(3-methylpiperazin-1-yl)pyridin-2-yl)pyrazolo[1,5-a]pyridine;
(S)-3-(6-(3-methylpiperazin-1-yl)pyridin-2-yl)pyrazolo[1,5-a]pyridine;
5-chloro-3-(6-((3S,5R)-3,4,5-trimethylpiperazin-1-yl)pyridin-2-yl)pyrazolo[1,5-a]pyridine;
N-(3-(6-(piperidin-3-yl)pyridin-2-yl)pyrazolo[1,5-a]pyridin-5-yl)picolinamide;
N-(3-(6-(piperidin-3-yl)pyridin-2-yl)pyrazolo[1,5-a]pyridin-5-yl)-2-(pyridin-2-yloxy)acetamide;
3-cyano-N-(3-(6-(piperidin-3-yl)pyridin-2-yl)pyrazolo[1,5-a]pyridin-5-yl)propanamide;
4-cyano-N-(3-(6-(piperidin-3-yl)pyridin-2-yl)pyrazolo[1,5-a]pyridin-5-yl)butanamide;
N-(3-(6-(piperidin-3-yl)pyridin-2-yl)pyrazolo[1,5-a]pyridin-5-yl)-2-(pyridin-4-yloxy)acetamide;
2-cyano-N-(3-(6-(piperidin-3-yl)pyridin-2-yl)pyrazolo[1,5-a]pyridin-5-yl)acetamide;
N-(3-(6-(piperidin-3-yl)pyridin-2-yl)pyrazolo[1,5-a]pyridin-5-yl)-2-(pyridin-3-yl)acetamide;
N-(3-(6-(piperidin-3-yl)pyridin-2-yl)pyrazolo[1,5-a]pyridin-5-yl)-2-(pyridin-4-ylamino)acetamide;
N-(3-(6-(piperidin-3-yl)pyridin-2-yl)pyrazolo[1,5-a]pyridin-5-yl)-2-(pyridin-3-ylamino)acetamide;
N-(3-(6-(piperidin-3-yl)pyridin-2-yl)pyrazolo[1,5-a]pyridin-5-yl)-2-(pyridin-2-yl)acetamide;
or a pharmaceutically acceptable salt thereof.

18. A pharmaceutical composition comprising a compound or salt according to claim 1 together with a pharmaceutically acceptable carrier, excipient, or diluent.

19. A method of treating a disease or disorder associated with LRRK2 kinase activity, the method comprising administering to a patient in need thereof a therapeutically effective amount of a compound of claim 1 or a pharmaceutical composition comprising a compound or salt according to claim 1 together with a pharmaceutically acceptable carrier, excipient, or diluent.

20. The method of claim 19, wherein the disease or disorder is Parkinson's disease, Alzheimer's disease, multiple sclerosis, HIV-induced dementia, amyotrophic lateral sclerosis, ischemic stroke, traumatic brain injury, or spinal cord injury.

21. A method of inhibiting LRRK2 kinase activity, the method comprising administering to a patient in need thereof a therapeutically effective amount of a compound claim 1 or a pharmaceutical composition comprising a compound or salt according to claim 1 together with a pharmaceutically acceptable carrier, excipient, or diluent.

22. A method of treating Parkinson's disease, comprising administering to a patient in need thereof a therapeutically effective amount of a compound of claim 1 or a pharmaceutical composition comprising a compound or salt according to claim 1 together with a pharmaceutically acceptable carrier, excipient, or diluent.
